(19) United States
(12) Patent Application Publication
(10) Pub. No.: US 2020/0157092 A1
(43) Pub. Date: May 21, 2020

(54) [1,2,4]-TRIAZOLO [1,5-A]-PYRIMIDINYL DERIVATIVES SUBSTITUTED WITH PIPERIDINE, MORPHOLINE OR PIPERAZINE AS OGA INHIBITORS

(71) Applicant: Janssen Pharmaceutlca NV, Beerse (BE)

(72) Inventors: José Manuel Bartolomé-Nebreda, Toledo (ES); Andrés Avelino Trabanco-Suárez, Olias del Rey (ES); Manuel Jesús Alcázar-Vaca, Toledo (ES)

(21) Appl. No.: 16/611,536

(22) PCT Filed: Feb. 27, 2018
(86) PCT No.: PCT/EP2018/054763

§ 371 Date: Nov. 7, 2019

(30) Foreign Application Priority Data
Feb. 27, 2017 (EP) 17158044.2

(51) Int. Cl. C07D 471/04

(52) U.S. Cl. CPC C07D 471/04

(57) ABSTRACT

The present invention relates to O-GlcNAc hydrolase (OGA) inhibitors. The invention is also directed to pharmaceutical compositions comprising such compounds, to processes for preparing such compounds and compositions, and to the use of such compounds and compositions for the prevention and treatment of disorders in which inhibition of OGA is beneficial, such as tauopathies, in particular Alzheimer's disease or progressive supranuclear palsy; and neurodegenerative diseases accompanied by a tau pathology, in particular amyotrophic lateral sclerosis or frontotemporal lobe dementia caused by C9ORF72 mutations.

### FIELD OF THE INVENTION

The present invention relates to O-GlcNAc hydrolase (OGA) inhibitors, having the structure shown in Formula (I)

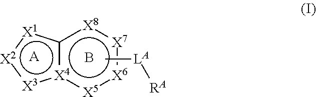

wherein the radicals are as defined in the specification. The invention is also directed to pharmaceutical compositions comprising such compounds, to processes for preparing such compounds and compositions, and to the use of such compounds and compositions for the prevention and treatment of disorders in which inhibition of OGA is beneficial, such as tauopathies, in particular Alzheimer's disease or progressive supranuclear palsy; and neurodegenerative diseases accompanied by a tau pathology, in particular amyotrophic lateral sclerosis or frontotemporal lobe dementia caused by C9ORF72 mutations.

### BACKGROUND OF THE INVENTION

O-GlcNAcylation is a reversible modification of proteins where N-acetyl-D-glucosamine residues are transferred to the hydroxyl groups of serine- and threonine residues yield O-GlcNAcylated proteins. More than 1000 of such target proteins have been identified both in the cytosol and nucleus of eukaryotes. The modification is thought to regulate a huge spectrum of cellular processes including transcription, cytoskeletal processes, cell cycle, proteasomal degradation, and receptor signalling.

O-GlcNAc transferase (OGT) and O-GlcNAc hydrolase (OGA) are the only two proteins described that add (OGT) or remove (OGA) O-GlcNAc from target proteins. OGA was initially purified in 1994 from spleen preparation and 1998 identified as antigen expressed by meningiomas and termed MGEA5, consists of 916 amino (102915 Dalton) as a monomer in the cytosolic compartment of cells. It is to be distinguished from ER- and Golgi-related glycosylation processes that are important for trafficking and secretion of proteins and different to OGA have an acidic pH optimum, whereas OGA display highest activity at neutral pH.

The OGA catalytic domain with its double aspartate catalytic center resides in the N-terminal part of the enzyme which is flanked by two flexible domains. The C-terminal part consists of a putative HAT (histone acetyl transferase domain) preceded by a stalk domain. It has yet still to be proven that the HAT-domain is catalytically active.

O-GlcNAcylated proteins as well as OGT and OGA themselves are particularly abundant in the brain and neurons suggesting this modification plays an important role in the central nervous system. Indeed, studies confirmed that O-GlcNAcylation represents a key regulatory mechanism contributing to neuronal communication, memory formation and neurodegenerative disease. Moreover, it has been shown that OGT is essential for embryogenesis in several animal models and ogt null mice are embryonic lethal. OGA is also indispensible for mammalian development. Two independent studies have shown that OGA homozygous null mice do not survive beyond 24-48 hours after birth. Oga deletion has led to defects in glycogen mobilization in pups and it caused genomic instability linked cell cycle arrest in MEFs derived from homozygous knockout embryos. The heterozygous animals survived to adulthood however they exhibited alterations in both transcription and metabolism.

It is known that perturbations in O-GlcNAc cycling impact chronic metabolic diseases such as diabetes, as well as cancer. Oga heterozygosity suppressed intestinal tumorigenesis in an Apc−/+ mouse cancer model and the Oga gene (MGEA5) is a documented human diabetes susceptibility locus.

In addition, O-GlcNAc-modifications have been identified on several proteins that are involved in the development and progression of neurodegenerative diseases and a correlation between variations of O-GlcNAc levels on the formation of neurofibrillary tangle (NFT) protein by Tau in Alzheimer's disease has been suggested. In addition, O-GlcNAcylation of alpha-synuclein in Parkinson's disease has been described.

In the central nervous system six splice variants of tau have been described. Tau is encoded on chromosome 17 and consists in its longest splice variant expressed in the central nervous system of 441 amino acids. These isoforms differ by two N-terminal inserts (exon 2 and 3) and exon 10 which lie within the microtubule binding domain. Exon 10 is of considerable interest in tauopathies as it harbours multiple mutations that render tau prone to aggregation as described below. Tau protein binds to and stabilizes the neuronal microtubule cytoskeleton which is important for regulation of the intracellular transport of organelles along the axonal compartments. Thus, tau plays an important role in the formation of axons and maintenance of their integrity. In addition, a role in the physiology of dendritic spines has been suggested as well.

Tau aggregation is either one of the underlying causes for a variety of so called tauopathies like PSP (progressive supranuclear palsy), Down's syndrome (DS), FTLD (frontotemporal lobe dementia), FTDP-17 (frontotemporal dementia with Parkinsonism-17), Pick's disease (PD), CBD (corticobasal degeneration), agryophilic grain disease (AGD), and AD (Alzheimer's disease). In addition, tau pathology accompanies additional neurodegenerative diseases like amyotrophic lateral sclerosis (ALS) or FTLD cause by C9ORF72 mutations. In these diseases, tau is post-translationally modified by excessive phosphorylation which is thought to detach tau from microtubules and makes it prone to aggregation. O-GlcNAcylation of tau regulates the extent of phosphorylation as serine or threonine residues carrying O-GlcNAc-residues are not amenable to phosphorylation. This effectively renders tau less prone to detaching from microtubules and reduces aggregation into neurotoxic tangles which ultimately lead to neurotoxicity and neuronal cell death. This mechanism may also reduce the cell-to-cell spreading of tau-aggregates released by neurons via along interconnected circuits in the brain which has recently been discussed to accelerate pathology in tau-related dementias. Indeed hyperphosphorylated tau isolated from brains of AD-patients showed significantly reduced O-GlcNAcylation levels.

An OGA inhibitor administered to JNPL3 tau transgenic mice successfully reduced NFT formation and neuronal loss without apparent adverse effects. This observation has been confirmed in another rodent model of tauopathy where the expression of mutant tau found in FTD can be induced (tg4510). Dosing of a small molecule inhibitor of OGA was efficacious in reducing the formation of tau-aggregation and attenuated the cortical atrophy and ventricle enlargement.

Moreover, the O-GlcNAcylation of the amyloid precursor protein (APP) favours processing via the non-amyloidogenic route to produce soluble APP fragment and avoid cleavage that results in the AD associated amyloid-beta (Aβ) formation.

Maintaining O-GlcNAcylation of tau by inhibition of OGA represents a potential approach to decrease tau-phosphorylation and tau-aggregation in neurodegenerative diseases mentioned above thereby attenuating or stopping the progression of neurodegenerative tauopathy-diseases.

US 2010/022517 (Richards Lori et al.) discloses an ophthalmic formulation comprising at least one inhibitor of Rho-associated protein kinase, and discloses N-(1-((2,3-dihydrobenzo[b][1,4]dioxin-6-yl)methyl)piperidin-3-yl)-1H-indazol-5-amine; WO 2015/164508 (Dart Neuroscience LLC) discloses substituted [1,2,4]triazolo[1,5-a]pyrimidin-yl compounds as PDE2 inhibitors, in particular, compounds such as 2-[(3-{5-methyl-[1,2,4]triazolo[1,5-a]pyrimidin-7-yl}piperidin-1-yl)methyl]quinoline, 1-(2H-1,3-benzodioxol-5-ylmethyl)-3-{5-methyl-[1,2,4]triazolo[1,5-a]pyrimidin-7-yl}piperidine, and 1-methyl-2-[(3-{5-methyl-[1,2,4]triazolo[1,5-a]pyrimidin-7-yl}piperidin-1-yl)methyl]-1H-1,3-benzodiazole; WO 2014/159234 (Merck Patent GmbH) discloses mainly 4-phenyl or benzyl-piperidine and piperazine compounds substituted at the 1-position with an acetamide-thiazolylmethyl or acetamidoxazolylmethyl substituent and the compound N-[5-(3-phenyl-1-piperidyl)methyl]thiazol-2-yl]acetamide; WO 2012/062759 (Janssen Pharmaceuticals Inc) discloses 1,2,4-triazolo[4,3-a]pyridine compounds as mGluR2 positive allosteric modulators.

There is still a need for OGA inhibitors with an advantageous balance of properties, for example with improved potency, better selectivity, brain penetration and/or better side effect profile. It has now been found that compounds according to the present invention exhibit OGA inhibitory activity and a good balance of properties.

### SUMMARY OF THE INVENTION

The present invention is directed to compounds of Formula (I)

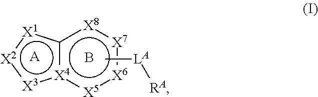

and the tautomers and the stereoisomeric forms thereof, wherein
A-B represent a 9-membered bicyclic heteroaryl system having from 1 to 4 nitrogen atoms, wherein
X¹ and X³ are each independently selected from the group consisting of CR^{XA}, N, and NR^{YA};

### X² is CH;

### X⁴ is C or N; and

X⁵, X⁶, X⁷, and X⁸ are each independently selected from the group consisting of C, CR^{XB} and N;
with the proviso that at least one of X¹ and X³ is N or NR^{YA};
wherein each R^{XA}, and R^{XB}, when present, is independently selected from the group consisting of hydrogen; halo; —CN; C₁₋₄alkyl optionally substituted with 1, 2 or 3 independently selected halo substituents; and C₁₋₄alkyloxy optionally substituted with 1, 2 or 3 independently selected halo substituents;
each R^{YA}, when present, is independently selected from the group consisting of hydrogen and C₁₋₄alkyl optionally substituted with 1, 2 or 3 independently selected halo substituents;
L^{A} is bound to any available carbon atom at the 6-membered B ring of the A-B bicycle, and is selected from the group consisting of a bond, CHR¹, O, and NR¹; wherein
R¹ is selected from the group consisting of hydrogen and C₁₋₄alkyl optionally substituted with 1, 2 or 3 independently selected halo substituents;
R^{A} is a radical (a-1) when L^{A} is a bond, CHR¹, O, or NR¹; or is a radical selected from the group consisting of (a-2) and (a-3) when L^{A} is a bond or CHR¹

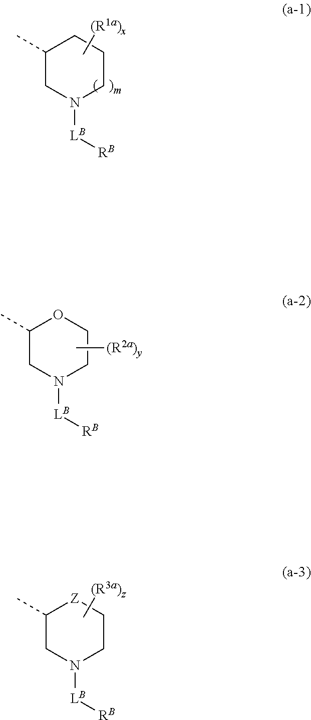

wherein
m represents 0, 1 or 2;
x, y and z, each independently represent 0, 1 or 2;
each R^{1a} and R^{2a} when present, is bound to any available carbon atom and is independently selected from the group consisting of halo and C₁₋₄alkyl optionally substituted with 1, 2, or 3 independently selected halo substituents; or two R^{1a} or two R^{2a} substituents are bound to the same carbon atom and form together a cyclopropylidene radical;
Z is N when substituted with R^{3a}, or NH;
each R^{3a} is bound to any available carbon atom or nitrogen atom when present, and is independently selected from C₁₋₃alkyl optionally substituted with 1, 2 or 3 independently selected halo substituents; or two R^{3a} substituents are bound to the same carbon atom and form together a cyclopropylidene radical;
L^{B} is selected from the group consisting of >CHR² and >SO₂;
wherein R² is selected from the group consisting of hydrogen, and C₁₋₄alkyl optionally substituted with 1, 2 or 3 independently selected halo substituents; and
R^{B} represents a heterocyclic ring or ring system selected from the group consisting of (b-1), (b-2), (b-3), (b-4), (b-5), (b-6), (b-7), (b-8), (b-9), (b-10), (b-11) and (b-12)

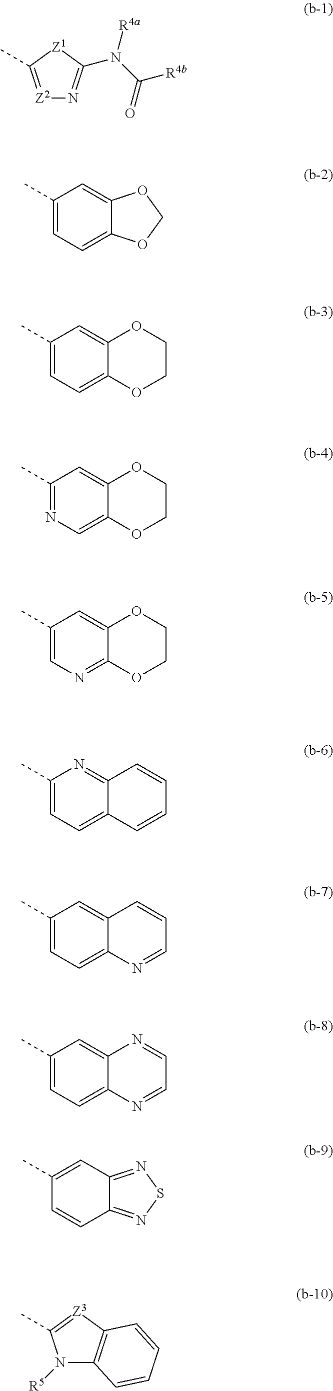

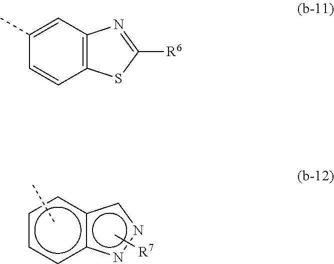

wherein
Z¹ is O, NR^{1z} or S; wherein R^{1z} is hydrogen or C₁₋₄alkyl;
Z² and Z³ each independently represent CH or N;
R^{4b} is C₁₋₄alkyl;
R^{4a}, R⁵, R⁶ and R⁷ each independently represent hydrogen or C₁₋₄alkyl; or
-L^{B}-R^{B} is a radical of formula (b-13)

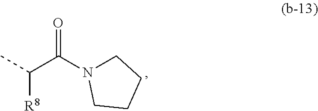

wherein R⁸ is hydrogen or C₁₋₄alkyl;
and the pharmaceutically acceptable salts and the solvates thereof.

Illustrative of the invention is a pharmaceutical composition comprising a pharmaceutically acceptable carrier and any of the compounds described above. An illustration of the invention is a pharmaceutical composition made by mixing any of the compounds described above and a pharmaceutically acceptable carrier. Illustrating the invention is a process for making a pharmaceutical composition comprising mixing any of the compounds described above and a pharmaceutically acceptable carrier.

Exemplifying the invention are methods of preventing or treating a disorder mediated by the inhibition of O-GlcNAc hydrolase (OGA), comprising administering to a subject in need thereof a prophylactically or a therapeutically effective amount of any of the compounds or pharmaceutical compositions described above.

Further exemplifying the invention are methods of inhibiting OGA, comprising administering to a subject in need thereof a prophylactically or a therapeutically effective amount of any of the compounds or pharmaceutical compositions described above.

An example of the invention is a method of preventing or treating a disorder selected from a tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, comprising administering to a subject in need thereof, a prophylactically or a therapeutically effective amount of any of the compounds or pharmaceutical compositions described above.

Another example of the invention is any of the compounds described above for use in preventing or treating a tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, in a subject in need thereof.

### DETAILED DESCRIPTION OF THE INVENTION

The present invention is directed to compounds of Formula (I) as defined hereinbefore, and pharmaceutically acceptable addition salts and solvates thereof. The compounds of Formula (I) are inhibitors of O-GlcNAc hydrolase (OGA) and may be useful in the prevention or treatment of tauopathies, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or may be useful in the prevention or treatment of neurodegenerative diseases accompanied by a tau pathology, in particular a neurodegenerative disease selected from amyotrophic lateral sclerosis or frontotemporal lobe dementia caused by C9ORF72 mutations.

In an embodiment, the invention is directed to compounds of Formula (I) as referred to herein, and the tautomers and the stereoisomeric forms thereof, wherein

A-B represent a 9-membered bicyclic heteroaryl system having from 1 to 4 nitrogen atoms, wherein
X¹ and X³ are each independently selected from the group consisting of CR^{XA}, N, and NR^{YA};

### X² is CH;

### X⁴ is C or N; and

X⁵, X⁶, X⁷, and X⁸ are each independently selected from the group consisting of C, CR^{XB} and N;
with the proviso that at least one of X¹ and X³ is N or NR^{YA};
wherein each R^{XA}, and R^{XB}, when present, is independently selected from the group consisting of hydrogen; halo; —CN; C₁₋₄alkyl optionally substituted with 1, 2 or 3 independently selected halo substituents; and C₁₋₄alkyloxy optionally substituted with 1, 2 or 3 independently selected halo substituents;
each R^{YA}, when present, is independently selected from the group consisting of hydrogen and C₁₋₄alkyl optionally substituted with 1, 2 or 3 independently selected halo substituents;
L^{A} is bound to any available carbon atom at the 6-membered B ring of the A-B bicycle, and is selected from the group consisting of a bond, CHR¹, O, and NR¹; wherein
R¹ is selected from the group consisting of hydrogen and C₁₋₄alkyl optionally substituted with 1, 2 or 3 independently selected halo substituents;
R^{A} is a radical (a-1) when L^{A} is a bond, CHR¹, O, or NR¹; or is a radical selected from the group consisting of (a-2) and (a-3) when L^{A} is a bond or CHR¹

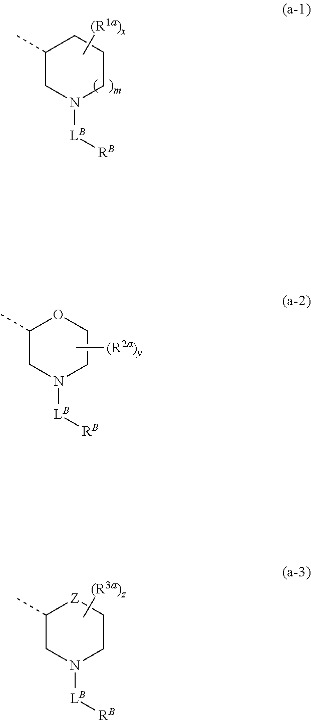

wherein
m represents 0 or 1;
x, y and z, each independently represent 0, 1 or 2;
each R^{1a} and R^{2a} when present, is bound to any available carbon atom and is independently selected from the group consisting of halo and C₁₋₄alkyl optionally substituted with 1, 2, or 3 independently selected halo substituents; or two R^{1a} or two R^{2a} substituents are bound to the same carbon atom and form together a cyclopropylidene radical;
Z is N when substituted with R^{3a}, or NH;
each R^{3a} is bound to any available carbon atom or nitrogen atom when present, and is independently selected from C₁₋₃alkyl optionally substituted with 1, 2 or 3 independently selected halo substituents; or two R^{3a} substituents are bound to the same carbon atom and form together a cyclopropylidene radical;
L^{B} is selected from the group consisting of >CHR² and >SO₂;
wherein R² is selected from the group consisting of hydrogen, and C₁₋₄alkyl optionally substituted with 1, 2 or 3 independently selected halo substituents; and
R^{B} represents a heterocyclic ring or ring system selected from the group consisting of (b-1), (b-2), (b-3), (b-4), (b-5), (b-6), (b-7), (b-8), (b-9), (b-10), (b-11) and (b-12)

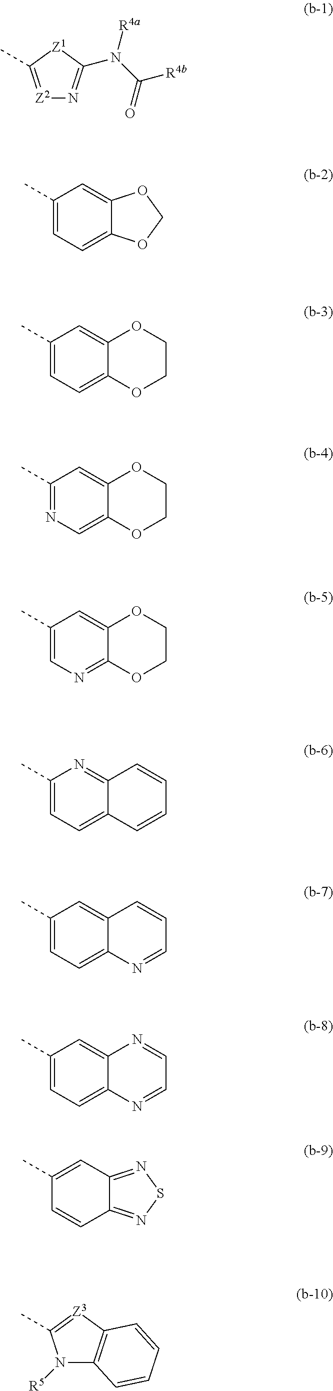

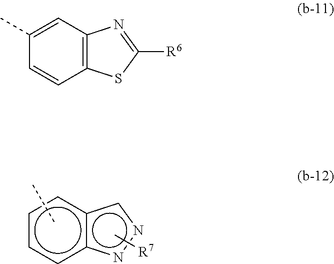

wherein
Z¹ is O, NR^{1z} or S; wherein R^{1z} is hydrogen or C₁₋₄alkyl;
Z² and Z³ each independently represent CH or N;
R^{4b} is C₁₋₄alkyl;
R^{4a}, R⁵, R⁶ and R⁷ each independently represent hydrogen or C₁₋₄alkyl; or
-L^{B}-R^{B} is a radical of formula (b-13)

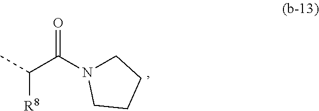

wherein R⁸ is hydrogen or C₁₋₄alkyl;
and the pharmaceutically acceptable salts and the solvates thereof.

In an embodiment, the invention is directed to compounds of Formula (I) as referred to herein, and the tautomers and the stereoisomeric forms thereof, wherein

X¹ is selected from the group consisting of CH, N, and NR^{YA};

### X² is CH;

### X³ is CH or N;

### X⁴ is C or N;

X⁵ is C, CR^{XB} or N;
X⁶ is C, CH, C(CH₃) or C(halo);
X⁷ is C, CR^{XB} or N; and

### X⁸ is C, CH or N;

with the proviso that at least one of X¹ and X³ is N or NR^{YA};
and the pharmaceutically acceptable salts and the solvates thereof.

In an embodiment, the invention is directed to compounds of Formula (I) as referred to herein, and the tautomers and the stereoisomeric forms thereof, wherein

X¹ is selected from the group consisting of CH, N, and NR^{YA};

### X² is CH;

### X³ is CH or N;

### X⁴ is C or N;

X⁵ is C, CR^{XB} or N;

### X⁶ is C, CH, or C(halo);

X⁷ is C, CR^{XB} or N; and

### X⁸ is C, CH or N;

with the proviso that at least one of X¹ and X³ is N or NR^{YA};
and the pharmaceutically acceptable salts and the solvates thereof.

In a further embodiment, the invention is directed to compounds of Formula (I) as referred to herein, and the tautomers and the stereoisomeric forms thereof, wherein R^{B} is a radical selected from the group consisting of (b-1), (b-2), (b-3) and (b-8); or

-L^{B}-R^{B} is a radical of formula (b-13) as defined herein;
and the pharmaceutically acceptable salts and the solvates thereof.

In a further embodiment, the invention is directed to compounds of Formula (I) as referred to herein, and the tautomers and the stereoisomeric forms thereof, wherein R^{B} is a radical selected from the group consisting of (b-1), (b-2), (b-3) and (b-8)

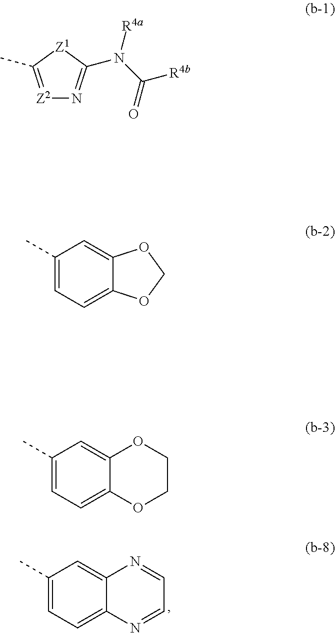

wherein

### Z¹ is S;

### Z² is CH;

R^{4a} is H or CH₃;
R^{4b} is C₁₋₄alkyl; or
-L^{B}-R^{B} is a radical of formula (b-13), wherein R⁸ is hydrogen or C₁₋₄alkyl;
and the pharmaceutically acceptable salts and the solvates thereof.

In a further embodiment, the invention is directed to compounds of Formula (I) as referred to herein, and the tautomers and the stereoisomeric forms thereof, wherein

X¹ is selected from the group consisting of CH, N, and NR^{YA}; wherein R^{YA}, when present, is hydrogen or C₁₋₄alkyl;

### X² is CH;

### X³ is CH or N;

### X⁴ is C or N;

X⁵ is C, CR^{XB} or N; wherein R^{XB}, when present, is hydrogen or C₁₋₄alkyl optionally substituted with 1, 2 or 3 independently selected halo substituents;
X⁶ is C, CH, C(CH₃), or C(halo);
X⁷ is C, CR^{XB} or N; wherein R^{XB}, when present, is hydrogen or C₁₋₄alkyl optionally substituted with 1, 2 or 3 independently selected halo substituents; and

### X⁸ is C, CH or N;

with the proviso that at least one of X¹ and X³ is N or NR^{YA};
L^{A} is bound to any available carbon atom at the 6-membered B ring of the A-B bicycle, and is selected from the group consisting of a bond, CHR¹, and NR¹; wherein
R¹ is hydrogen or C₁₋₄alkyl;
R^{A} is a radical (a-1) when L^{A} is a bond, CHR¹, NR¹; or is a radical selected from the group consisting of (a-2) and (a-3) when L^{A} is a bond or CHR¹

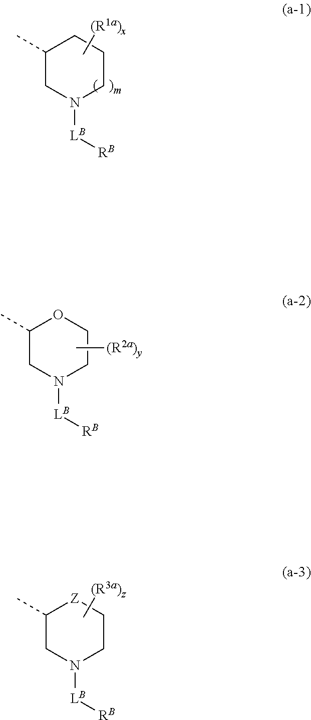

wherein
m represents 0 or 1;
x is 0, 1 or 2;
y and z, each independently represent 0;
each R^{1a} when present, is bound to any available carbon atom and is C₁₋₄alkyl optionally substituted with 1, 2, or 3 independently selected halo substituents; or two R^{1a} substituents are bound to the same carbon atom and form together a cyclopropylidene radical;

### Z is NH;

L^{B} is selected from the group consisting of >CHR² and >SO₂; wherein R² is hydrogen or C₁₋₄alkyl; and
R^{B} is a radical selected from the group consisting of (b-1), (b-2), (b-3) and (b-8)

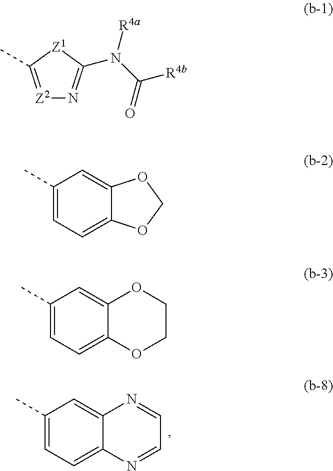

wherein

### Z¹ is S;

### Z² is CH;

R^{4a} is H or CH₃;
R^{4b} is C₁₋₄alkyl; or
-L^{B}-R^{B} is a radical of formula (b-13), wherein R⁸ is hydrogen or C₁₋₄alkyl;
and the pharmaceutically acceptable salts and the solvates thereof.

In a particular embodiment, the invention is directed to compounds of Formula (I) as referred to herein, and the tautomers and the stereoisomeric forms thereof, wherein

X¹ is selected from the group consisting of CH, N, and NR^{YA}; wherein R^{YA}, when present, is hydrogen or C₁₋₄alkyl;

### X² is CH;

### X³ is CH or N;

### X⁴ is C or N;

X⁵ is C, CR^{XB} or N; wherein R^{XB}, when present, is hydrogen or C₁₋₄alkyl optionally substituted with 1, 2 or 3 independently selected halo substituents;

### X⁶ is C, CH, or C(halo);

X⁷ is C, CR^{XB} or N; wherein R^{XB}, when present, is hydrogen or C₁₋₄alkyl optionally substituted with 1, 2 or 3 independently selected halo substituents; and

### X⁸ is C, CH or N;

with the proviso that at least one of X¹ and X³ is N or NR^{YA};
L^{A} is bound to any available carbon atom at the 6-membered B ring of the A-B bicycle, and is selected from the group consisting of a bond, CHR¹, and NR¹; wherein
R¹ is hydrogen or C₁₋₄alkyl;
R^{A} is a radical (a-1) when L^{A} is a bond, CHR¹, NR¹; or is a radical selected from the group consisting of (a-2) and (a-3) when L^{A} is a bond or CHR¹

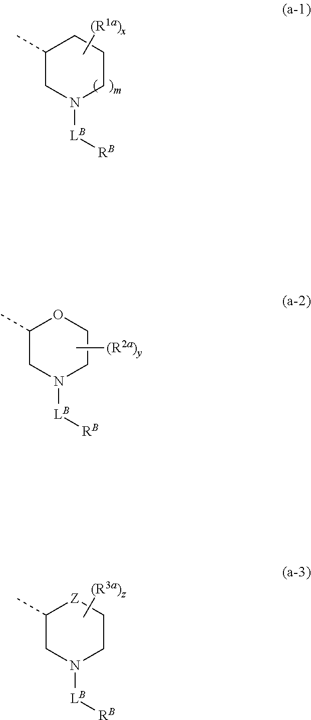

wherein
m represents 0 or 1;
x is 0, 1 or 2;
y and z, each independently represent 0;
each R^{1a} when present, is bound to any available carbon atom and is C₁₋₄alkyl optionally substituted with 1, 2, or 3 independently selected halo substituents; or two R^{1a} substituents are bound to the same carbon atom and form together a cyclopropylidene radical;

### Z is NH;

L^{B} is selected from the group consisting of >CHR² and >SO₂; wherein R² is hydrogen or C₁₋₄alkyl; and
R^{B} is a radical selected from the group consisting of (b-1), (b-2), (b-3) and (b-8)

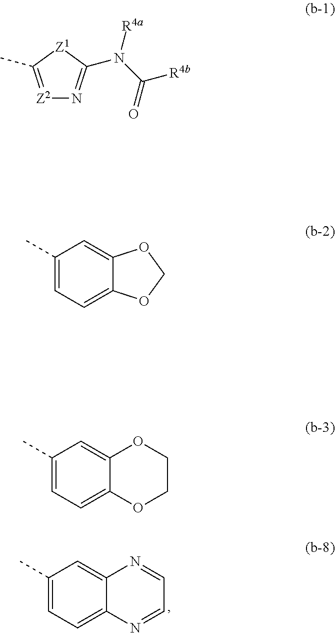

wherein

### Z¹ is S;

### Z² is CH;

R^{4a} is H or CH₃;
R^{4b} is C₁₋₄alkyl; or
-L^{B}-R^{B} is a radical of formula (b-13), wherein R⁸ is hydrogen or C₁₋₄alkyl;
and the pharmaceutically acceptable salts and the solvates thereof.

In a further embodiment, the invention is directed to compounds of Formula (I) as referred to herein, and the tautomers and the stereoisomeric forms thereof, wherein L^{A} is a bond or CH₂;

and the pharmaceutically acceptable salts and the solvates thereof.

In a further embodiment, the invention is directed to compounds of Formula (I) as referred to herein, and the tautomers and the stereoisomeric forms thereof, wherein L^{A} is a bond, CH₂ or NH; and R^{A} is (a-1);

and the pharmaceutically acceptable salts and the solvates thereof.

In a further embodiment, the invention is directed to compounds of Formula (I) as referred to herein, and the tautomers and the stereoisomeric forms thereof, wherein R^{A} is (a-1); m is 0 or 1; and x is 0, 1 or 2, and R^{1a} is methyl or two R^{1a} substituents are bound to the same carbon atom and form together a cyclopropylidene radical;

and the pharmaceutically acceptable salts and the solvates thereof.

In a further embodiment, the invention is directed to compounds of Formula (I) as referred to herein, and the tautomers and the stereoisomeric forms thereof, wherein R^{A} is (a-1); m is 0 or 1; and x is 0;

and the pharmaceutically acceptable salts and the solvates thereof.

In a further embodiment, the invention is directed to compounds of Formula (I) as referred to herein, and the tautomers and the stereoisomeric forms thereof, wherein L^{B} is CH₂;

and the pharmaceutically acceptable salts and the solvates thereof.

In a further embodiment, the invention is directed to compounds of Formula (I) as referred to herein, and the tautomers and the stereoisomeric forms thereof, wherein R^{B} is (b-1);

and the pharmaceutically acceptable salts and the solvates thereof.

In a further embodiment, the compounds of Formula (I) are in particular compounds of Formula (I-A), (I-B), (I-C), or (I-D), more in particular, compounds of Formulae (I-A), (I-B) or (I-C)

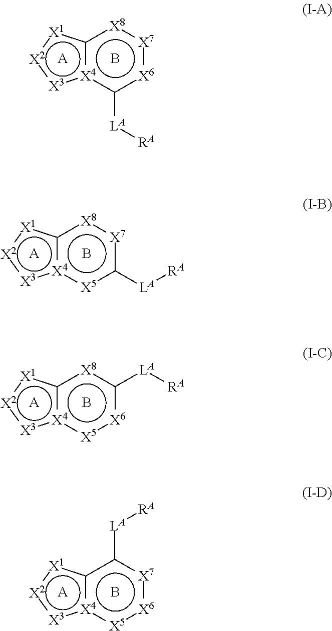

wherein all variables are as described in Formula (I) herein.

In a further embodiment, the compounds of Formula (I) are in particular compounds of Formula (I′)

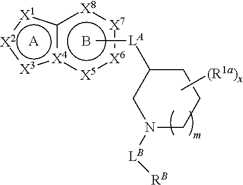

wherein all variables are as described in Formula (I) herein.

In another embodiment, the compounds of the invention are in particular compounds of Formula (IA), wherein

X¹ is N, NH or N(CH₃);

### X² is CH;

### X³ is CH or N;

### X⁴ is C or N;

### X⁶ is CH or CF;

X⁷ is C, CH, C(CH₃) or C(CHF₂);

### X⁸ is CH or N;

and all other variables are as described in Formula (I) herein.

In another embodiment, the compounds of the invention are in particular compounds of Formula (IA), wherein

X¹ is N, NH or N(CH₃);

### X² is CH;

### X³ is CH or N;

### X⁴ is C or N;

### X⁶ is CH;

X⁷ is C, CH, C(CH₃) or C(CHF₂);

### X⁸ is CH or N;

and all other variables are as described in Formula (I) herein.

In another embodiment, the compounds of the invention are in particular compounds of Formula (IA), wherein

### X¹ is N;

### X² is CH;

### X³ is CH or N;

### X⁴ is N;

### X⁶ is CH;

X⁷ is CH, C(CH₃) or N;

### X⁸ is CH or N;

and all other variables are as described in Formula (I) herein.

In another embodiment, the compounds of the invention are in particular compounds of Formula (IA), wherein

X¹, X², and X⁶ are each CH;
X³ is NH or N(CH₃);

### X⁴ is C;

X⁷ is C(CH₃);

### X⁸ N;

and all other variables are as described in Formula (I) herein.

In another embodiment, the compounds of the invention are in particular compounds of Formula (IA), wherein

X¹, X², X⁶ and X⁸ are CH;
X³ and X⁴ are N;
X⁷ is C, CH, C(CH₃) or C(CHF₂);
and all other variables are as described in Formula (I) herein.

In a further embodiment, the compounds of the invention are in particular compounds of Formula (IA′)

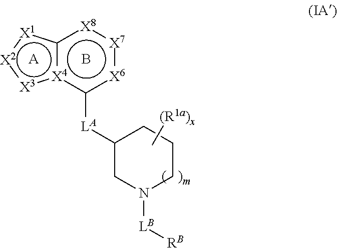

wherein all variables are as described in Formula (IA) herein.

In another embodiment, the compounds of the invention are in particular compounds of Formula (IB), wherein

X¹ is N, NH or N(CH₃);

### X² is CH;

### X³ is CH or N;

### X⁴ is C or N;

X⁵ is C, CH, C(CHF₂), or N;
X⁷ is C, CH, C(CH₃) or C(CHF₂);

### X⁸ is CH or N;

and all other variables are as described in Formula (I) herein.

In another embodiment, the compounds of the invention are in particular compounds of Formula (IB), wherein

X¹ is CH or C(CH₃);

### X² is CH;

### X³ is CH;

### X⁴ is C;

### X⁵ is CH;

X⁷ is C(CH₃);

### X⁸ is N;

and all other variables are as described in Formula (I) herein.

In another embodiment, the compounds of the invention are in particular compounds of Formula (IB), wherein

X¹, X² and X⁸ are CH;
X³ and X⁴ are N;
X⁵ is C, CH, and C(CHF₂);
X⁷ is C, CH, C(CH₃) or C(CHF₂);
and all other variables are as described in Formula (I) herein.

In another embodiment, the compounds of the invention are in particular compounds of Formula (IB), wherein

X¹, X², X⁵ and X⁷ are CH;
X³ and X⁴ are N;
and all other variables are as described in Formula (I) herein.

In a further embodiment, the compounds of the invention are in particular compounds of Formula (IB′)

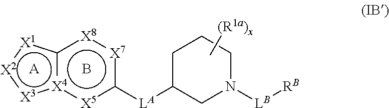

wherein all variables are as described in Formula (IB) herein.

In another embodiment, the compounds of the invention are in particular compounds of Formula (IC), wherein

X¹ is N, NH or N(CH₃);

### X² is CH;

### X³ is CH or N;

### X⁴ is C or N;

X⁵ is C, CH, C(CHF₂) or N;

### X⁶ is C or CH;

### X⁸ is CH or N;

and all other variables are as described in Formula (I) herein.

In another embodiment, the compounds of the invention are in particular compounds of Formula (IC), wherein

X¹ is NH or N(CH₃);
X², X³, X⁶ and X⁸ are each CH;

### X⁴ is C;

### X⁵ is N;

and all other variables are as described in Formula (I) herein.

In another embodiment, the compounds of the invention are in particular compounds of Formula (IC), wherein

X¹, X² and X⁸ are CH;
X³ and X⁴ are N;
X⁵ is C, CH, or C(CHF₂);

### X⁶ is C or CH;

and all other variables are as described in Formula (I) herein.

In another embodiment, the compounds of the invention are in particular compounds of Formula (ID), wherein

### X¹ is NH;

### X² is CH;

### X³ is CH;

### X⁴ is C;

### X⁵ is CH or N;

X⁶ is CH or C(CH₃);

### X⁷ is CH or N;

and all other variables are as described in Formula (I) herein.

In a further embodiment, the compounds of the invention are in particular compounds of Formula (IC′)

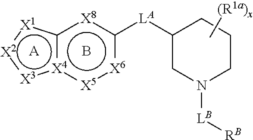

wherein all variables are as described in Formula (IC) herein.

In a further embodiment, the compounds of Formula (I) are in particular compounds of Formula (ID′)

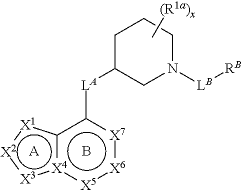

wherein all variables are as described in Formula (ID) herein.

In yet a further embodiment, the bicycle A-B is

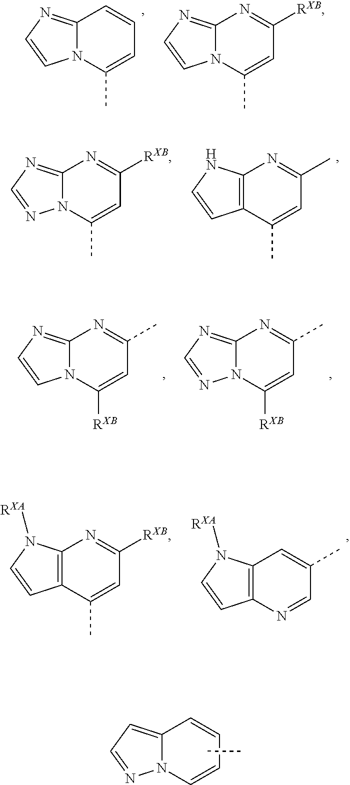

wherein R^{XA} is H or CH₃, and R^{XB} is H, CH₃ or CHF₂.

In yet a further a further embodiment, the bicycle A-B is

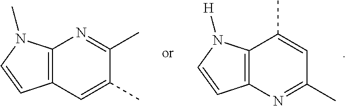

In another embodiment, the compound of Formula (I) is

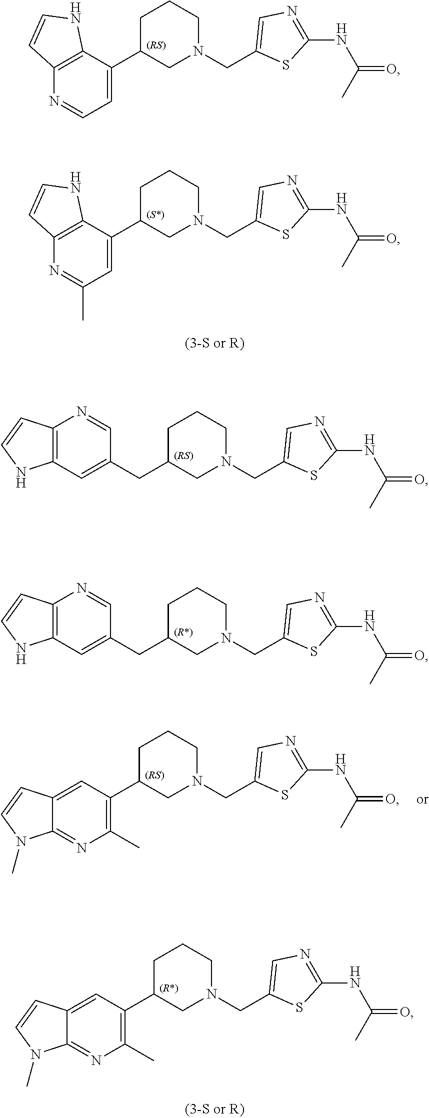 [SMILES: CC(=O)Nc1ncc(CN2CCCC(c3ccnc4cc[nH]c34)C2)s1 ; CC(=O)Nc1ncc(CN2CCCC(c3cc(C)nc4cc[nH]c34)C2)s1 ; CC(=O)Nc1ncc(CN2CCCC(Cc3cnc4cc[nH]c4c3)C2)s1 ; CC(=O)Nc1ncc(CN2CCCC(Cc3cnc4cc[nH]c4c3)C2)s1 ; CC(=O)Nc1ncc(CN2CCCC(c3cc4ccn(C)c4nc3C)C2)s1 ; CC(=O)Nc1ncc(CN2CCCC(c3cc4ccn(C)c4nc3C)C2)s1]

or a pharmaceutically acceptable salt or a solvate thereof.

### Definitions

“Halo” shall denote fluoro, chloro and bromo; “C₁₋₄alkyl” shall denote a straight or branched saturated alkyl group having 1, 2, 3 or 4 carbon atoms, respectively e.g. methyl, ethyl, 1-propyl, 2-propyl, butyl, 1-methyl-propyl, 2-methyl-1-propyl, 1,1-dimethylethyl, and the like; “C₁₋₄alkyloxy” shall denote an ether radical wherein C₁₋₄alkyl is as defined before.

The term “subject” as used herein, refers to an animal, preferably a mammal, most preferably a human, who is or has been the object of treatment, observation or experiment. As used herein, the term “subject” therefore encompasses patients, as well as asymptomatic or presymptomatic individuals at risk of developing a disease or condition as defined herein.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. The term “prophylactically effective amount” as used herein, means that amount of active compound or pharmaceutical agent that substantially reduces the potential for onset of the disease or disorder being prevented.

As used herein, the term “composition” is intended to encompass a product comprising the specified ingredients in the specified amounts, as well as any product which results, directly or indirectly, from combinations of the specified ingredients in the specified amounts.

Hereinbefore and hereinafter, the term “compound of Formula (I)” is meant to include the addition salts, the solvates and the stereoisomers thereof.

The terms “stereoisomers” or “stereochemically isomeric forms” hereinbefore or hereinafter are used interchangeably.

The invention includes all stereoisomers of the compound of Formula (I) either as a pure stereoisomer or as a mixture of two or more stereoisomers.

Enantiomers are stereoisomers that are non-superimposable mirror images of each other. A 1:1 mixture of a pair of enantiomers is a racemate or racemic mixture. Diastereomers (or diastereoisomers) are stereoisomers that are not enantiomers, i.e. they are not related as mirror images. If a compound contains a double bond, the substituents may be in the E or the Z configuration. If a compound contains a disubstituted cycloalkyl group, the substituents may be in the cis or trans configuration. Therefore, the invention includes enantiomers, diastereomers, racemates, E isomers, Z isomers, cis isomers, trans isomers and mixtures thereof.

The absolute configuration is specified according to the Cahn-Ingold-Prelog system. The configuration at an asymmetric atom is specified by either R or S. Resolved compounds whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other isomers. Thus, when a compound of Formula (I) is for instance specified as (R), this means that the compound is substantially free of the (S) isomer; when a compound of Formula (I) is for instance specified as E, this means that the compound is substantially free of the Z isomer; when a compound of Formula (I) is for instance specified as cis, this means that the compound is substantially free of the trans isomer.

For use in medicine, the addition salts of the compounds of this invention refer to non-toxic “pharmaceutically acceptable addition salts”. Other salts may, however, be useful in the preparation of compounds according to this invention or of their pharmaceutically acceptable addition salts. Suitable pharmaceutically acceptable addition salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable addition salts thereof may include alkali metal salts, e.g., sodium or potassium salts; alkaline earth metal salts, e.g., calcium or magnesium salts; and salts formed with suitable organic ligands, e.g., quaternary ammonium salts.

Representative acids which may be used in the preparation of pharmaceutically acceptable addition salts include, but are not limited to, the following: acetic acid, 2,2-dichloroactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, capric acid, caproic acid, caprylic acid, cinnamic acid, citric acid, cyclamic acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, beta-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, trifluoromethylsulfonic acid, and undecylenic acid. Representative bases which may be used in the preparation of pharmaceutically acceptable addition salts include, but are not limited to, the following: ammonia, L-arginine, benethamine, benzathine, calcium hydroxide, choline, dimethylethanol-amine, diethanolamine, diethylamine, 2-(diethylamino)-ethanol, ethanolamine, ethylene-diamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

The names of compounds were generated according to the nomenclature rules agreed upon by the Chemical Abstracts Service (CAS) or according to the nomenclature rules agreed upon by the International Union of Pure and Applied Chemistry (IUPAC).

### Preparation of the Final Compounds

The compounds according to the invention can generally be prepared by a succession of steps, each of which is known to the skilled person. In particular, the compounds can be prepared according to the following synthesis methods.

The compounds of Formula (I) may be synthesized in the form of racemic mixtures of enantiomers which can be separated from one another following art-known resolution procedures. The racemic compounds of Formula (I) may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

### Preparation of the Compounds

### Experimental Procedure 1

The final compounds according to Formula (I), wherein R^{B} is (b-1), herein referred to as compounds of Formula (I-a), can be prepared by reacting an intermediate compound of Formula (II-a) with a compound of Formula (XIX) according to reaction scheme (1). The reaction is performed in a suitable reaction-inert solvent, such as, for example, dichloromethane, in the presence of a suitable base, such as, for example, triethylamine, under thermal conditions 0° C. or room temperature, for example for 1 hour. In reaction scheme (1) all variables are defined as in Formula (I) and wherein

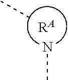

represents the optionally substituted heterocyclyl moiety at R^{A} (i.e., pyrrolidinyl, piperidinyl or azepanyl from (a-1), morpholinyl from (a-2) or piperazinyl from (a-3), as defined herein).

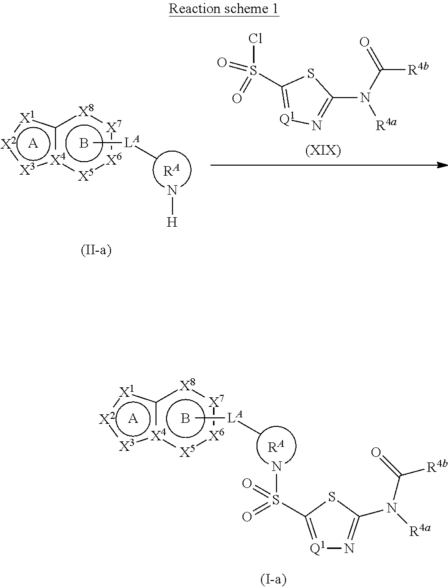

### Experimental Procedure 2

Additionally, final compounds of Formula (I), wherein L^{B} is CHR², herein referred to as compounds of Formula (I-b), can be prepared by reacting an intermediate compound of Formula (II-a) with a compound of Formula (XX) according to reaction scheme (2). The reaction is performed in a suitable reaction-inert solvent, such as, for example, dichloromethane, a metal hydride, such as, for example sodium triacetoxyborohydride, sodium cyanoborohydride or sodium borohydride and may require the presence of a suitable base, such as, for example, triethylamine, and/or a Lewis acid, such as, for example titanium tetraisopropoxide or titanium tetrachloride, under thermal conditions, such as, 0° C. or room temperature, or 140° C., for example for 1 hour or 24 hours. In reaction scheme (2) all variables are defined as in Formula (I) and wherein

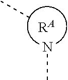

represents the optionally substituted heterocyclyl moiety at R^{A} (i.e., pyrrolidinyl, piperidinyl or azepanyl from (a-1), morpholinyl from (a-2) or piperazinyl from (a-3), as defined herein).

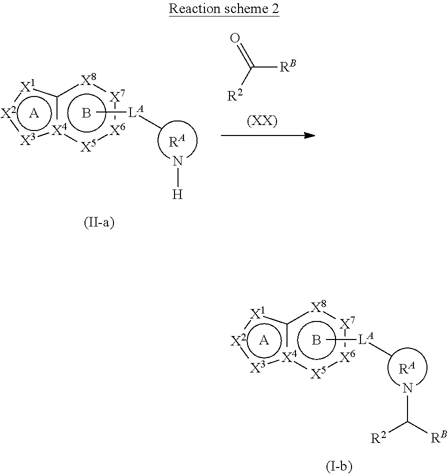

### Experimental Procedure 3

Alternatively, final compounds of Formula (I-b) can be prepared by reacting an intermediate compound of Formula (II-a) with a compound of Formula (XXI) according to reaction scheme (3). The reaction is performed in a suitable reaction-inert solvent, such as, for example, acetonitrile, a suitable base, such as, for example, trimethylamine or diisopropylethylamine, under thermal conditions, such as, 0° C. or room temperature, or 75° C., for example for 1 hour or 24 hours. In reaction scheme (3) all variables are defined as in Formula (I), halo is chloro, bromo or iodo and wherein

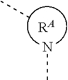

represents the optionally substituted heterocyclyl moiety at R^{A} (i.e., pyrrolidinyl, piperidinyl or azepanyl from (a-1), morpholinyl from (a-2) or piperazinyl from (a-3), as defined herein).

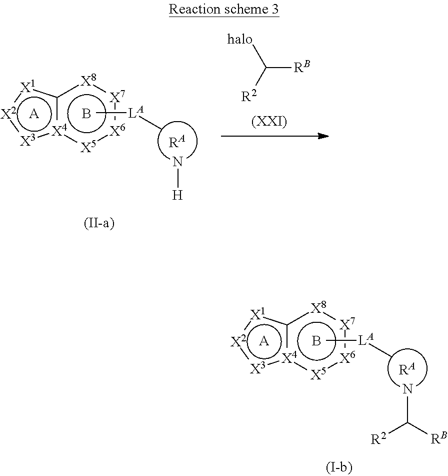

### Experimental Procedure 4

Additionally, final compounds of Formula (I), wherein L^{A} is NH, herein referred to as (I-c), can be prepared by reacting an intermediate compound of Formula (III-a) with a compound of Formula (IV) according to reaction scheme (4). The reaction is performed in a suitable reaction-inert solvent, such as, for example, acetonitrile, a suitable base, such as, for example, trimethylamine or diisopropylethylamine, under thermal conditions, such as, for example, 100° C., for example for 1 hour or 24 hours. In reaction scheme (4) all variables are defined as in Formula (I), L^{A} is NH and halo is chloro, bromo or iodo.

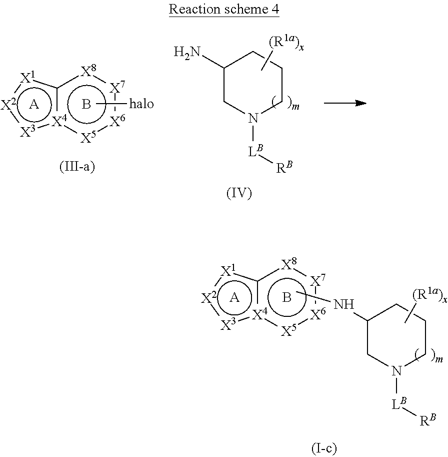

### Experimental Procedure 5

Additionally, final compounds of Formula (I-d) can be prepared cleaving a protecting group in an intermediate compound of Formula (V) according to reaction scheme (5). The reaction is performed in a suitable reaction-inert solvent, such as, for example, dichloromethane, a suitable acid, such as, for example, trifluoroacetic acid, under thermal conditions, such as, for example, room temperature, for example for 1 hour or 24 hours. In reaction scheme (5) all variables are defined as in Formula (I) wherein X³ is NH. PG¹ is a suitable protecting group of the nitrogen function such as, for example, 2-(trimethylsilyl)ethoxy methyl (SEM).

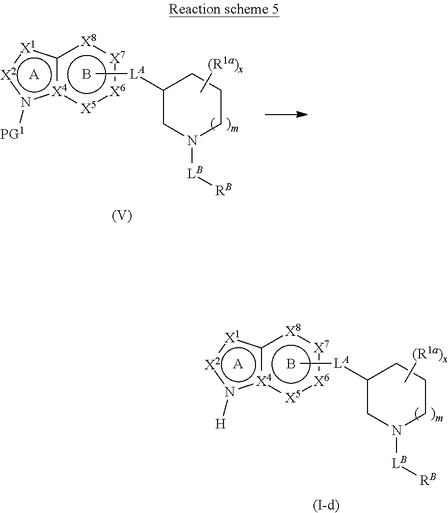

### Experimental Procedure 6

Intermediate compounds of Formula (II-a) can be prepared cleaving a protecting group in an intermediate compound of Formula (VI) according to reaction scheme (6). In reaction scheme (6) all variables are defined as in Formula (I),

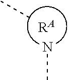

represents the optionally substituted heterocyclyl moiety at R^{A} (i.e., pyrrolidinyl, piperidinyl or azepanyl from (a-1), morpholinyl from (a-2) or piperazinyl from (a-3), as defined herein) and PG² is a suitable protecting group of the nitrogen function such as, for example, tert-butoxycarbonyl (Boc), ethoxycarbonyl, benzyl, benzyloxycarbonyl (Cbz). Suitable methods for removing such protecting groups are widely known by the person skilled in the art and comprise but are not limited to:
Boc deprotection. Treatment with a protic acid, such as, for example, trifluoroacetic acid, in a reaction inert solvent, such as, for example, dichloromethane.
Ethoxycarbonyl deprotection. Treatment with a strong base, such as, for example, sodium hydroxide, in a reaction inert solvent such as for example wet tetrahydrofuran.
Benzyl deprotection. Catalytic hydrogenation in the presence of a suitable catalyst, such as, for example palladium on carbon, in a reaction inert solvent, such as for example, ethanol.
Benzyloxycarbonyl deprotection. Catalytic hydrogenation in the presence of a suitable catalyst, such as, for example palladium on carbon, in a reaction inert solvent, such as for example, ethanol.

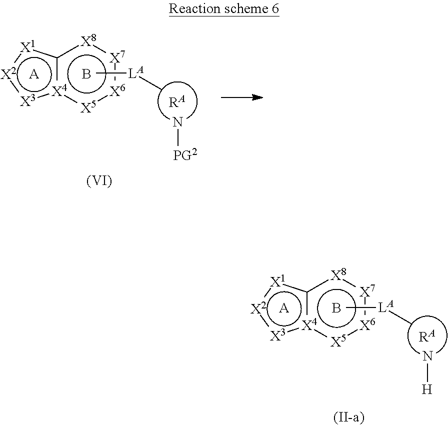

### Experimental Procedure 7

Intermediate compounds of Formula (VI-a) can be prepared by reacting an intermediate compound of Formula (VII) with a compound of Formula (XXII) according to reaction scheme (7). The reaction is performed in a suitable reaction-inert solvent, such as, for example, acetic acid, under thermal conditions, such as, for example, 120° C., for example for 30 minutes or 1 hour. In reaction scheme (7) all variables are defined as in Formula (I) wherein X¹, X⁴ and X⁸ are N, X⁵ and X⁶ is CH, X⁷ is CR^{XB}, L^{A} is a bond and

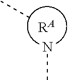

represents the optionally substituted heterocyclyl moiety at R^{A} (i.e., pyrrolidinyl, piperidinyl or azepanyl from (a-1), morpholinyl from (a-2) or piperazinyl from (a-3), as defined herein). PG² is defined as in Formula (VI).

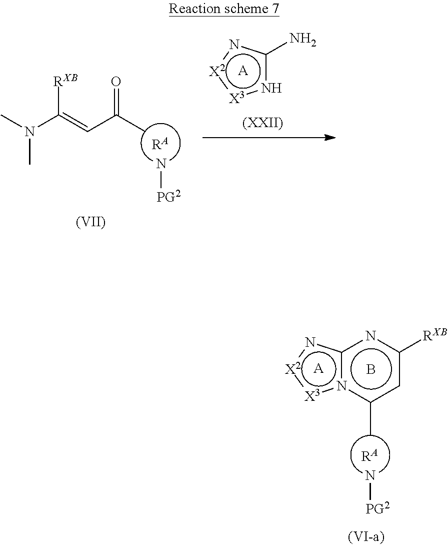

### Experimental Procedure 8

Additionally, intermediate compounds of Formula (VI-a) and (VI-b) can be prepared by reacting an intermediate compound of Formula (VIII) with a compound of Formula (XXII) according to reaction scheme (8). The reaction is performed in a suitable reaction-inert solvent, such as, for example, acetic acid or DMF, under thermal conditions, such as, for example, 60° C., for example for 3 days. In reaction scheme (8) all variables are defined as in Formula (I) wherein X¹, X⁴ and X⁸ are N, X⁵ is C in compound (VI-a) and CR^{XB} in compound (VI-b), X⁶ is CH, X⁷ is CR^{XB} in compound (VI-a) and C in compound (VI-b), L^{A} is a bond and

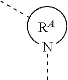

represents the optionally substituted heterocyclic moiety at R^{A} (i.e., pyrrolidinyl, piperidinyl or azepanyl from (a-1), morpholinyl from (a-2) or piperazinyl from (a-3), as defined herein). PG² is defined as in Formula (VI).

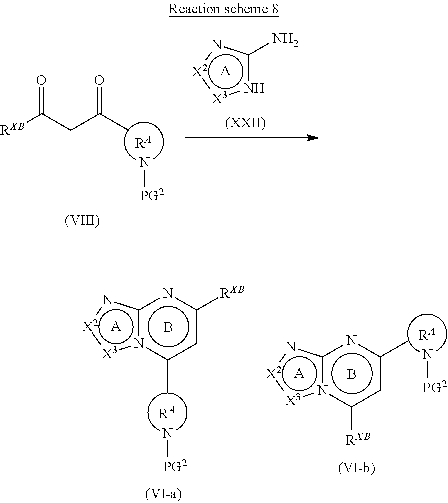

### Experimental Procedure 9

Intermediate compounds of Formula (VII) can be prepared by reacting an intermediate compound of Formula (IX) with a compound of Formula (XXIII) according to reaction scheme (9). The reaction is performed under thermal conditions, such as, for example, 100° C., for example for 3 hours. In reaction scheme (9) all variables are defined as in Formula (I) wherein

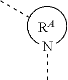

represents the optionally substituted heterocyclyl moiety at R^{A} (i.e., pyrrolidinyl, piperidinyl or azepanyl from (a-1), morpholinyl from (a-2) or piperazinyl from (a-3), as defined herein). PG² is defined as in Formula (VI).

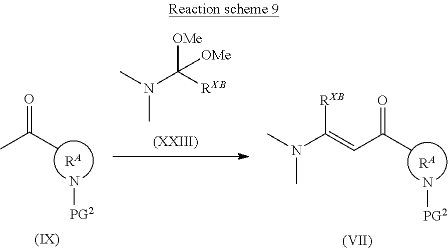

### Experimental Procedure 10

Intermediate compounds of Formula (VIII) can be prepared by reacting an intermediate compound of Formula (IX) with a compound of Formula (XXIV) according to reaction scheme (10). The reaction is performed in a suitable reaction-inert solvent, such as, for example, toluene, a suitable base, such as, for example, potassium tert-butoxide under thermal conditions, such as, for example, −5° C. or 0° C. or room temperature, for example for 2 or 3 hours. In reaction scheme (10) all variables are defined as in Formula (I) wherein

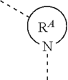

represents the optionally substituted heterocyclyl moiety at R^{A} (i.e., pyrrolidinyl, piperidinyl or azepanyl from (a-1), morpholinyl from (a-2) or piperazinyl from (a-3), as defined herein). PG² is defined as in Formula (VI).

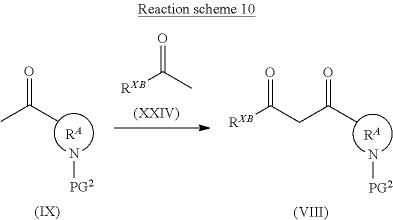

### Experimental Procedure 11

Intermediate compounds of Formula (IX) can be prepared by reacting an intermediate compound of Formula (X) with methylmagnesium bromide according to reaction scheme (11). The reaction is performed in a suitable reaction-inert solvent, such as, for example, tetrahydrofuran, under thermal conditions, such as, for example, 0° C. or room temperature, for example for 1 hour. In reaction scheme (11) all variables are defined as in Formula (I) wherein

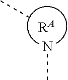

represents the optionally substituted heterocyclyl moiety at R^{A} (i.e., pyrrolidinyl, piperidinyl or azepanyl from (a-1), morpholinyl from (a-2) or piperazinyl from (a-3), as defined herein). PG² is defined as in Formula (VI).

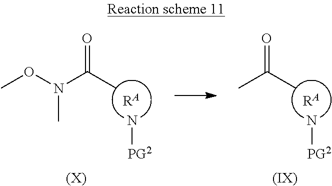

### Experimental Procedure 12

Intermediate compounds of Formula (X) can be prepared by reacting an intermediate compound of Formula (XI) with N,O-dimethyl hydroxylamine hydrochloride according to reaction scheme (12). The reaction is performed in a suitable reaction-inert solvent, such as, for example, tetrahydrofuran or acetonitrile, a suitable reagent for the preparation of Weinreb amides, such as, for example, carbonyldiimidazole, a suitable base, such as, for example, triethylamine, under thermal conditions, such as, for example, room temperature, for example for 3 hours. In reaction scheme (12) all variables are defined as in Formula (I) wherein

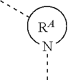

represents the optionally substituted heterocyclyl moiety at R^{A} (i.e., pyrrolidinyl, piperidinyl or azepanyl from (a-1), morpholinyl from (a-2) or piperazinyl from (a-3), as defined herein). PG² is defined as in Formula (VI).

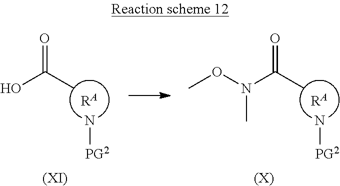

### Experimental Procedure 13

Additionally, intermediate compounds of Formula (VI-a) can be prepared by a decarboxylative reaction of an intermediate compound of Formula (XII) according to reaction scheme (13). The reaction is performed in a mixture of suitable reaction-inert solvents, such as, for example, MeOH or EtOH and H₂O, a suitable base, such as, for example, lithium hydroxide, under thermal conditions, such as, for example, 40° C., for example for 24 hours. In reaction scheme (13) all variables are defined as in Formula I wherein X¹, X⁴ and X⁸ are N, X⁵ and X⁶ are C(H), X⁷ is CR^{X}, L^{A} is a bond, and

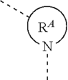

represents the optionally substituted heterocyclyl moiety at R^{A} (i.e., pyrrolidinyl, piperidinyl or azepanyl from (a-1), morpholinyl from (a-2) or piperazinyl from (a-3), as defined herein). PG² is defined as in Formula (VI).

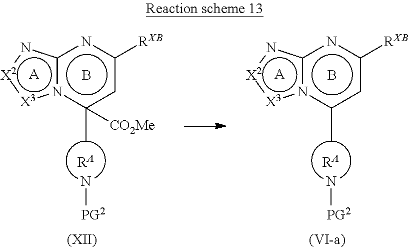

### Experimental Procedure 14

Intermediate compounds of Formula (XII) can be prepared by reacting an intermediate compound of Formula (III-b) with a compound of Formula (XXV) according to reaction scheme (14). The reaction is performed in a suitable reaction-inert solvent, such as, for example, tetrahydrofuran, a suitable base, such as, for example, lithium diisopropylamide, under thermal conditions, such as, for example, −78° C. or −60° C., for example for 1 or 2 hours. In reaction scheme (14) all variables are defined as in Formula (I) wherein X¹, X⁴ and X⁸ are N, X⁵ and X⁶ are C(H), X⁷ is CR^{XB}, L^{A} is a bond,

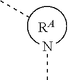

represents the optionally substituted heterocyclyl moiety at R^{A} (i.e., pyrrolidinyl, piperidinyl or azepanyl from (a-1), morpholinyl from (a-2) or piperazinyl from (a-3), as defined herein) and halo is chloro. PG² is defined as in Formula (VI).

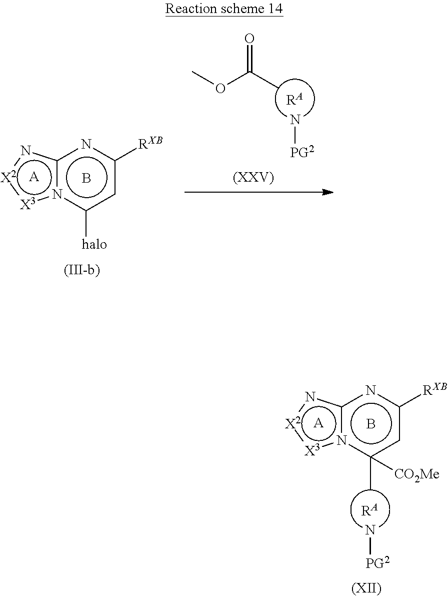

### Experimental Procedure 15

Intermediate compounds of Formula (VI-c) can be prepared by “Negishi coupling” reaction of a halo compound of Formula (III-a) with an organozinc compound of Formula (XIII) according to reaction scheme (15). The reaction is performed in a suitable reaction-inert solvent, such as, for example, tetrahydrofuran, and a suitable catalyst, such as, for example, Pd(OAc)₂, a suitable ligand for the transition metal, such as, for example, 2-dicyclohexylphosphino-2′,6′-diisopropoxybiphenyl [CAS: 787618-22-8], under thermal conditions, such as, for example, room temperature, for example for 1 hour. In reaction scheme (15) all variables are defined as in Formula (I), and halo is preferably iodo. PG² is defined as in Formula (VI).

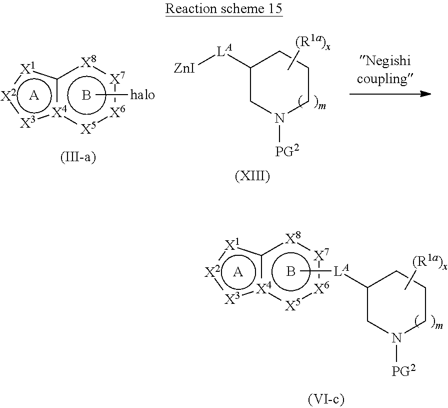

### Experimental Procedure 16

Intermediate compounds of Formula (XIII) can be prepared by reaction of a halo compound of Formula (XIV) with zinc according to reaction scheme (16). The reaction is performed in a suitable reaction-inert solvent, such as, for example, tetrahydrofuran, and a suitable salt, such as, for example, lithium chloride, under thermal conditions, such as, for example, 40° C., for example in a continuous-flow reactor. In reaction scheme (16) all variables are defined as in Formula (I), and halo is preferably iodo. PG² is defined as in Formula (VI).

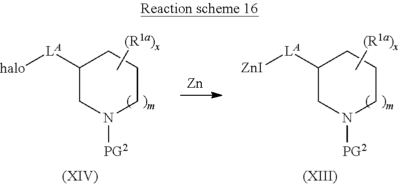

### Experimental Procedure 17

Intermediate compounds of Formula (VI-d) can be prepared under reductive conditions of a compound of Formula (XV) according to reaction scheme (17). The reaction is performed in a suitable reaction-inert solvent, such as, for example, ethanol, and a suitable catalyst, such as, for example, palladium on activated carbon, under thermal conditions, such as, for example, at room temperature and 1 atmosphere of hydrogen, for example for 18 hours. In reaction scheme (17) all variables are defined as in Formula (I) and L^{A} is a bond. PG² is defined as in Formula (VI).

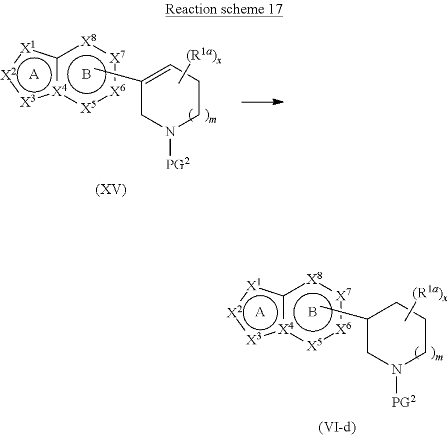

### Experimental Procedure 18

Intermediate compounds of Formula (XV) can be prepared by “Suzuki coupling” reaction of a halo compound of Formula (III-a) with a boronic ester compound of Formula (XVI) according to reaction scheme (18). The reaction is performed in a suitable reaction-inert solvent, such as, for example, dioxane, and a suitable Pd-complex catalyst, such as, for example, Pd(PPh₃)₄, a suitable base, such as, for example, aqueous sodium carbonate under thermal conditions, such as, for example, at 130° C. under microwave irradiation, for example for 30 minutes. In reaction scheme (18) all variables are defined as in Formula (I), L^{A} is a bond and halo is chloro, bromo or iodo. PG² is defined as in Formula (VI).

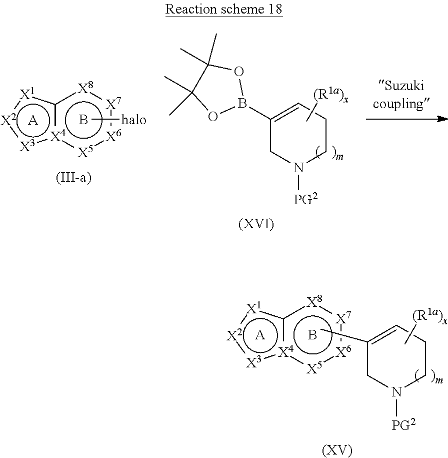

### Experimental Procedure 19

Intermediate compounds of Formula (IV) can be prepared by cleaving a protecting group in an intermediate compound of Formula (XVII) according to reaction scheme (19). In reaction scheme (19) all variables are defined as in Formula (I). PG² is a suitable protecting group of the nitrogen function such as, for example, tert-butoxycarbonyl (Boc), ethoxycarbonyl, benzyl, benzyloxycarbonyl (Cbz). Suitable methods for removing such protecting groups are described in experimental procedure 6.

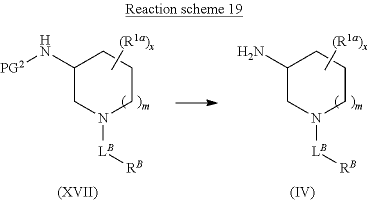

### Experimental Procedure 20

Intermediate compounds of Formula (XVII) can be prepared by reacting a compound of Formula (XVIII) with a compound of Formula (XX) according to reaction scheme (20). The reaction is performed in a suitable reaction-inert solvent, such as, for example, dichloromethane, a metal hydride, such as, for example sodium triacetoxyborohydride, sodium cyanoborohydride or sodium borohydride and may require the presence of a suitable base, such as, for example, trimethylamine or diisopropylethylamine, under thermal conditions, such as, 0° C. or room temperature, or 140° C., for example for 1 hour or 24 hours. In reaction scheme (20) all variables are defined as in Formula (I). PG² is defined as in Formula (VI)

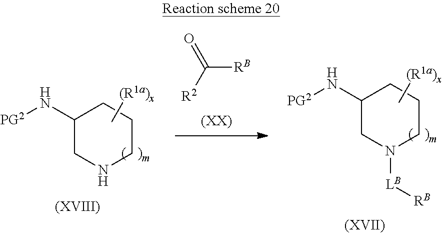

### Experimental Procedure 21

Intermediate compounds of Formula (V) can be prepared by reacting a compound of Formula (II-b) with a compound of Formula (XX) according to reaction scheme (21). The reaction is performed in a suitable reaction-inert solvent, such as, for example, dichloromethane or MeOH, a metal hydride, such as, for example sodium triacetoxyborohydride, sodium cyanoborohydride or sodium borohydride and may require the presence of a suitable base, such as, for example, trimethylamine or diisopropylethylamine, under thermal conditions, such as, 0° C. or room temperature, or 140° C., for example for 1 hour or 24 hours. In reaction scheme (21) all variables are defined as in Formula (I) wherein X³ is N. PG¹ is defined as in Formula (V).

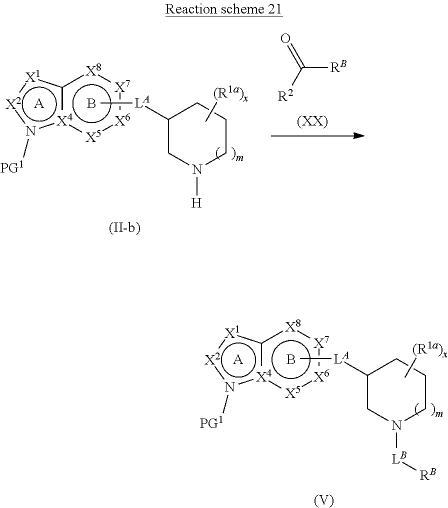

### Experimental Procedure 22

Intermediate compounds of Formula (II-b) can be prepared by cleaving a protecting group PG² in an intermediate compound of Formula (VI-e) according to reaction scheme (22). In reaction scheme (22) all variables are defined as in Formula (I) wherein X³ is N. PG¹ is defined as in Formula (V). PG² is a suitable protecting group of the nitrogen function such as, for example, tert-butoxycarbonyl (Boc), ethoxycarbonyl, benzyl, benzyloxycarbonyl (Cbz). Suitable methods for removing such protecting groups are described in experimental procedure 6.

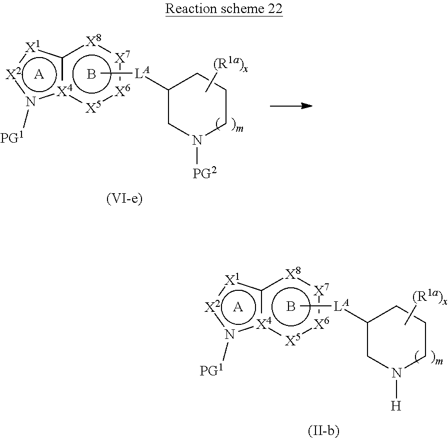

### Experimental Procedure 23

Intermediate compounds of Formula (VI-e) can be prepared by “Negishi coupling” reaction of a halo compound of Formula (III-c) with and organozinc compound of Formula (XIII) according to reaction scheme (23). The reaction is performed in a suitable reaction-inert solvent, such as, for example, tetrahydrofuran, and a suitable catalyst, such as, for example, Pd(OAc)₂, a suitable ligand for the transition metal, such as, for example, 2-dicyclohexylphosphino-2′,6′-diisopropoxybiphenyl [CAS: 787618-22-8], under thermal conditions, such as, for example, room temperature, for example for 1 hour. In reaction scheme (23) all variables are defined as in Formula (I) wherein X³ is N and halo is preferably iodo. PG¹ is defined as in Formula (V). PG² is defined as in Formula (VI).

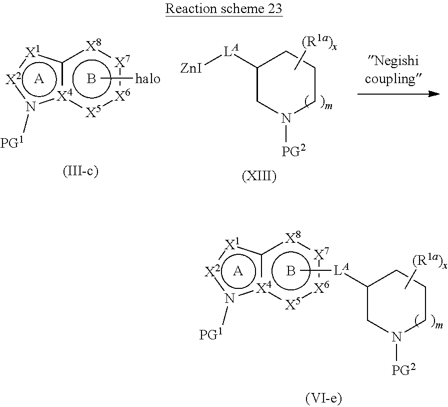

Intermediates of Formula, (III-a), (III-b), (III-c), (XI), (XIV), (XVI), (XVIII), (XIX), (XX), (XXI), (XXII), (XXIII) (XXIV) and (XXV) are commercially available or can be prepared by procedures known to those skilled in the art.

### Pharmacology

The compounds of the present invention and the pharmaceutically acceptable compositions thereof inhibit O-GlcNAc hydrolase (OGA) and therefore may be useful in the treatment or prevention of diseases involving tau pathology, also known as tauopathies, and diseases with tau inclusions. Such diseases include, but are not limited to Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Sträussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

As used herein, the term “treatment” is intended to refer to all processes, wherein there may be a slowing, interrupting, arresting or stopping of the progression of a disease or an alleviation of symptoms, but does not necessarily indicate a total elimination of all symptoms. As used herein, the term “prevention” is intended to refer to all processes, wherein there may be a slowing, interrupting, arresting or stopping of the onset of a disease.

The invention also relates to a compound according to the general Formula (I), a stereoisomeric form thereof or a pharmaceutically acceptable acid or base addition salt thereof, for use in the treatment or prevention of diseases or conditions selected from the group consisting of Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Sträussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

The invention also relates to a compound according to the general Formula (I), a stereoisomeric form thereof or a pharmaceutically acceptable acid or base addition salt thereof, for use in the treatment, prevention, amelioration, control or reduction of the risk of diseases or conditions selected from the group consisting of Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Sträussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

In particular, the diseases or conditions may in particular be selected from a tauopathy, more in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or the diseases or conditions may in particular be neurodegenerative diseases accompanied by a tau pathology, more in particular a neurodegenerative disease selected from amyotrophic lateral sclerosis or frontotemporal lobe dementia caused by C9ORF72 mutations.

Preclinical states in Alzheimer's and tauopathy diseases:

In recent years the United States (US) National Institute for Aging and the International Working Group have proposed guidelines to better define the preclinical (asymptomatic) stages of AD (Dubois B, et al. Lancet Neurol. 2014; 13:614-629; Sperling, R A, et al. Alzheimers Dement. 2011; 7:280-292). Hypothetical models postulate that Aβ accumulation and tau-aggregation begins many years before the onset of overt clinical impairment. The key risk factors for elevated amyloid accumulation, tau-aggregation and development of AD are age (ie, 65 years or older), APOE genotype, and family history. Approximately one third of clinically normal older individuals over 75 years of age demonstrate evidence of Aβ or tau accumulation on PET amyloid and tau imaging studies, the latter being less advanced currently. In addition, reduced Abeta-levels in CSF measurements are observed, whereas levels of non-modified as well as phosphorylated tau are elevated in CSF. Similar findings are seen in large autopsy studies and it has been shown that tau aggregates are detected in the brain as early as 20 years of age and younger. Amyloid-positive (Aβ+) clinically normal individuals consistently demonstrate evidence of an “AD-like endophenotype” on other biomarkers, including disrupted functional network activity in both functional magnetic resonance imaging (MRI) and resting state connectivity, fluorodeoxyglucose ¹⁸F (FDG) hypometabolism, cortical thinning, and accelerated rates of atrophy. Accumulating longitudinal data also strongly suggests that Aβ+ clinically normal individuals are at increased risk for cognitive decline and progression to mild cognitive impairment (MCI) and AD dementia. The Alzheimer's scientific community is of the consensus that these Aβ+ clinically normal individuals represent an early stage in the continuum of AD pathology. Thus, it has been argued that intervention with a therapeutic agent that decreases Aβ production or the aggregation of tau is likely to be more effective if started at a disease stage before widespread neurodegeneration has occurred. A number of pharmaceutical companies are currently testing BACE inhibition in prodromal AD.

Thanks to evolving biomarker research, it is now possible to identify Alzheimer's disease at a preclinical stage before the occurrence of the first symptoms. All the different issues relating to preclinical Alzheimer's disease such as, definitions and lexicon, the limits, the natural history, the markers of progression and the ethical consequences of detecting the disease at the asymptomatic stage, are reviewed in Alzheimer's & Dementia 12 (2016) 292-323.

Two categories of individuals may be recognized in preclinical Alzheimer's disease or tauopathies. Cognitively normal individuals with amyloid beta or tau aggregation evident on PET scans, or changes in CSF Abeta, tau and phospho-tau are defined as being in an “asymptomatic at risk state for Alzheimer's disease (AR-AD)” or in a “asymptomatic state of tauopathy”. Individuals with a fully penetrant dominant autosomal mutation for familial Alzheimer's disease are said to have “presymptomatic Alzheimer's disease”. Dominant autosomal mutations within the tau-protein have been described for multiple forms of tauopathies as well.

Thus, in an embodiment, the invention also relates to a compound according to the general Formula (I), a stereoisomeric form thereof or a pharmaceutically acceptable acid or base addition salt thereof, for use in control or reduction of the risk of preclinical Alzheimer's disease, prodromal Alzheimer's disease, or tau-related neurodegeneration as observed in different forms of tauopathies.

As already mentioned hereinabove, the term “treatment” does not necessarily indicate a total elimination of all symptoms, but may also refer to symptomatic treatment in any of the disorders mentioned above. In view of the utility of the compound of Formula (I), there is provided a method of treating subjects such as warm-blooded animals, including humans, suffering from or a method of preventing subjects such as warm-blooded animals, including humans, suffering from any one of the diseases mentioned hereinbefore.

Said methods comprise the administration, i.e. the systemic or topical administration, preferably oral administration, of a prophylactically or a therapeutically effective amount of a compound of Formula (I), a stereoisomeric form thereof, a pharmaceutically acceptable addition salt or solvate thereof, to a subject such as a warm-blooded animal, including a human.

Therefore, the invention also relates to a method for the prevention and/or treatment of any of the diseases mentioned hereinbefore comprising administering a prophylactically or a therapeutically effective amount of a compound according to the invention to a subject in need thereof.

The invention also relates to a method for modulating O-GlcNAc hydrolase (OGA) activity, comprising administering to a subject in need thereof, a prophylactically or a therapeutically effective amount of a compound according to the invention and as defined in the claims or a pharmaceutical composition according to the invention and as defined in the claims.

A method of treatment may also include administering the active ingredient on a regimen of between one and four intakes per day. In these methods of treatment the compounds according to the invention are preferably formulated prior to administration. As described herein below, suitable pharmaceutical formulations are prepared by known procedures using well known and readily available ingredients.

The compounds of the present invention, that can be suitable to treat or prevent any of the disorders mentioned above or the symptoms thereof, may be administered alone or in combination with one or more additional therapeutic agents. Combination therapy includes administration of a single pharmaceutical dosage formulation which contains a compound of Formula (I) and one or more additional therapeutic agents, as well as administration of the compound of Formula (I) and each additional therapeutic agent in its own separate pharmaceutical dosage formulation. For example, a compound of Formula (I) and a therapeutic agent may be administered to the patient together in a single oral dosage composition such as a tablet or capsule, or each agent may be administered in separate oral dosage formulations.

A skilled person will be familiar with alternative nomenclatures, nosologies, and classification systems for the diseases or conditions referred to herein. For example, the fifth edition of the Diagnostic & Statistical Manual of Mental Disorders (DSM-5™) of the American Psychiatric Association utilizes terms such as neurocognitive disorders (NCDs) (both major and mild), in particular, neurocognitive disorders due to Alzheimer's disease. Such terms may be used as an alternative nomenclature for some of the diseases or conditions referred to herein by the skilled person.

### Pharmaceutical Compositions

The present invention also provides compositions for preventing or treating diseases in which inhibition of O-GlcNAc hydrolase (OGA) is beneficial, such as Alzheimer's disease, progressive supranuclear palsy, Down's syndrome, frontotemporal lobe dementia, frontotemporal dementia with Parkinsonism-17, Pick's disease, corticobasal degeneration, agryophilic grain disease, amyotrophic lateral sclerosis or frontotemporal lobe dementia caused by C9ORF72 mutations, said compositions comprising a therapeutically effective amount of a compound according to formula (I) and a pharmaceutically acceptable carrier or diluent.

While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of this invention may be prepared by any methods well known in the art of pharmacy. A therapeutically effective amount of the particular compound, in base form or addition salt form, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for systemic administration such as oral, percutaneous or parenteral administration; or topical administration such as via inhalation, a nose spray, eye drops or via a cream, gel, shampoo or the like. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

The exact dosage and frequency of administration depends on the particular compound of Formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

The present compounds can be used for systemic administration such as oral, percutaneous or parenteral administration; or topical administration such as via inhalation, a nose spray, eye drops or via a cream, gel, shampoo or the like. The compounds are preferably orally administered. The exact dosage and frequency of administration depends on the particular compound according to Formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

The amount of a compound of Formula (I) that can be combined with a carrier material to produce a single dosage form will vary depending upon the disease treated, the mammalian species, and the particular mode of administration. However, as a general guide, suitable unit doses for the compounds of the present invention can, for example, preferably contain between 0.1 mg to about 1000 mg of the active compound. A preferred unit dose is between 1 mg to about 500 mg. A more preferred unit dose is between 1 mg to about 300 mg. Even more preferred unit dose is between 1 mg to about 100 mg. Such unit doses can be administered more than once a day, for example, 2, 3, 4, 5 or 6 times a day, but preferably 1 or 2 times per day, so that the total dosage for a 70 kg adult is in the range of 0.001 to about 15 mg per kg weight of subject per administration. A preferred dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The invention also provides a kit comprising a compound according to the invention, prescribing information also known as “leaflet”, a blister package or bottle, and a container. Furthermore, the invention provides a kit comprising a pharmaceutical composition according to the invention, prescribing information also known as “leaflet”, a blister package or bottle, and a container. The prescribing information preferably includes advice or instructions to a patient regarding the administration of the compound or the pharmaceutical composition according to the invention. In particular, the prescribing information includes advice or instruction to a patient regarding the administration of said compound or pharmaceutical composition according to the invention, on how the compound or the pharmaceutical composition according to the invention is to be used, for the prevention and/or treatment of a tauopathy in a subject in need thereof. Thus, in an embodiment, the invention provides a kit of parts comprising a compound of Formula (I) or a stereoisomeric for thereof, or a pharmaceutically acceptable salt or a solvate thereof, or a pharmaceutical composition comprising said compound, and instructions for preventing or treating a tauopathy. The kit referred to herein can be, in particular, a pharmaceutical package suitable for commercial sale.

For the compositions, methods and kits provided above, one of skill in the art will understand that preferred compounds for use in each are those compounds that are noted as preferred above. Still further preferred compounds for the compositions, methods and kits are those compounds provided in the non-limiting Examples below.

### EXPERIMENTAL PART

Hereinafter, the term “m.p.” means melting point, “min” means minutes, “AcOH” means acetic acid, “ACN” means acetonitrile, “aq.” means aqueous, “DMAP” means 4-dimethylaminopyridine, “DIPE” means diisopropyl ether, “DMF” means dimethylformamide, “r.t.” or “RT” means room temperature, “rac” or “RS” means racemic, “sat.” means saturated, “SFC” means supercritical fluid chromatography, “SFC-MS” means supercritical fluid chromatography/mass spectrometry, “LC-MS” means liquid chromatography/mass spectrometry, “HPLC” means high-performance liquid chromatography, “PrOH” means isopropyl alcohol, “RP” means reversed phase, “Rₜ” means retention time (in minutes), “[M+H]⁺” means the protonated mass of the free base of the compound, “wt” means weight, “TEMPO” means 2,2,6,6-tetramethylpiperidine 1-oxyl, “THF” means tetrahydrofuran, “Et₂O” means diethyl ether, “EtOAc” means ethyl acetate, “DCM” means dichloromethane, “MeOH” means methanol, “sat” means saturated, “soltn” or “sol.” means solution, “HATU” means 1-[Bis(dimethylamino)methylene]-1H-1,2,3-triazolo[4,5-b]pyridinium 3-oxid hexafluorophosphate, “MTBE” means methyl-tert-butylether, “EtOH” means ethanol, “VCD” means vibrational circular dichroism, and “THF” means tetrahydrofuran.

Whenever the notation “RS” is indicated herein, it denotes that the compound is a racemic mixture at the indicated centre, unless otherwise indicated. The stereochemical configuration for centres in some compounds has been designated “R” or “S” when the mixture(s) was separated; for some compounds, the stereochemical configuration at indicated centres has been designated as “*R” or “*S” when the absolute stereochemistry is undetermined although the compound itself has been isolated as a single stereoisomer and is enantiomerically/diastereomerically pure. The enantiomeric excess of compounds reported herein was determined e.g., by analysis of the racemic mixture by supercritical fluid chromatography (SFC) followed by SFC comparison of the separated enantiomer(s).

In intermediates/compounds wherein bonds are indicated either with a bold wedge or a wedge of parallel lines while the stereocentres are designated RS, the representation indicates that the sample is a mixture of stereoisomers, one stereoisomer having the indicated substituents or groups projected above or below the plane of the drawing as represented, one stereoisomer having the substituents or groups in the opposite projection below or above the plane of the drawing, e.g.

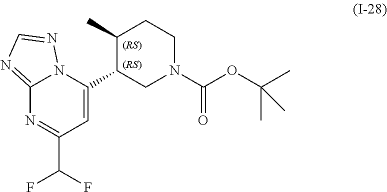 [SMILES: C[C@H]1CCN(C(=O)OC(C)(C)C)C[C@@H]1c1cc(C(F)F)nc2ncnn12]

represents a 50:50 mixture of

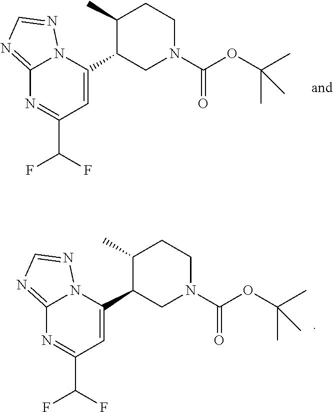 [SMILES: C[C@H]1CCN(C(=O)OC(C)(C)C)C[C@@H]1c1cc(C(F)F)nc2ncnn12 ; C[C@@H]1CCN(C(=O)OC(C)(C)C)C[C@H]1c1cc(C(F)F)nc2ncnn12]

The absolute configuration of chiral centres (indicated as R and/or S) was determined by VCD in intermediates/compounds or could be rationalized. The synthesis of final compounds started from intermediates of known absolute configuration in agreement with literature precedent or obtained from appropriate synthetic procedures. The assignment of the absolute configuration of additional stereocentres could then be assigned by standard NMR methods.

Microwave assisted reactions were performed in a single-mode reactor: Initiator™ Sixty EXP microwave reactor (Biotage AB), or in a multimode reactor: MicroSYNTH Labstation (Milestone, Inc.).

Thin layer chromatography (TLC) was carried out on silica gel 60 F254 plates (Merck) using reagent grade solvents. Open column chromatography was performed on silica gel, particle size 60 Å, mesh=230-400 (Merck) using standard techniques.

Automated flash column chromatography was performed using ready-to-connect cartridges, on irregular silica gel, particle size 15-40 μm (normal phase disposable flash columns) on different flash systems: either a SPOT or LAFLASH systems from Armen Instrument, or PuriFlash® 430evo systems from Interchim, or 971-FP systems from Agilent, or Isolera 1SV systems from Biotage.

The absolute stereochemical configuration for some of the compounds was determined using vibrational circular dichroism (VCD). They were measured on a Bruker Equinox 55 equipped with a PMA 37, in a KBr liquid cell using CD₂Cl₂ as solvent (PEM: 1350 cm−1, LIA: 1 mV, resolution: 4 cm⁻¹). A description on the use of VCD for the determination of absolute configuration can be found in Dyatkin A. B. et. al, Chirality, 14:215-219 (2002). Ab initio calculations: A thorough conformational search is performed at molecular mechanics level using Macromodel to do a mixed torsional/low-mode sampling with the OPLS-2005 force field. The located minima were optimized using Jaguar on the B3LYP/6-31G** level with a Poisson-Boltzmann continuum solvation model to mimic a dichloromethane solvent. All conformations within 10 kJ/mol interval were used to simulate VCD and IR spectrum. Dipole and rotational strengths were calculated at the same B3LYP/6-31G** level, using Jaguar. The calculated VCD spectra, generated after scaling the frequencies with a factor of 0.97, converting to a Lorentzian bandshape, and summing up the contribution of each conformer assuming a Boltzmann ensemble, were visually compared with the experimental spectra for assigning the correct stereo chemistry.

### A. Preparation of the Intermediates

### Preparation of Intermediate 1

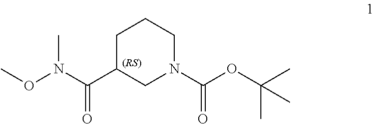 [SMILES: CON(C)C(=O)C1CCCN(C(=O)OC(C)(C)C)C1]

1,1′-Carbonyldiimidazole (CAS: 530-62-1, 48 g, 0.30 mol) was added to a solution of 1-(tert-butoxycarbonyl)-3-piperidinecarboxylic acid (CAS: 84358-12-3, 50 g, 0.22 mol) in THF (250 mL) and the mixture was stirred at rt for 1 h. In another flask, triethylamine (31.78 g, 0.31 mol) was added to a suspension of N-methoxymethanamine hydrochloride (1:1) (29.27 g, 0.30 mol) in CH₃CN (200 mL) and the mixture was stirred at rt for 1 h. Both mixtures were combined and stirred at rt for 16 h. Then the solvents were evaporated in vacuo. The residue was dissolved in DCM and washed with water, acetic acid (20% solution) and finally with Na₂CO₃ (aq. sat. soltn.). The organic layer was separated, dried (MgSO₄), filtered, evaporated in vacuo and co-evaporated with toluene in vacuo affording intermediate 1 (59 g, 99%)

### Preparation of Intermediate 2

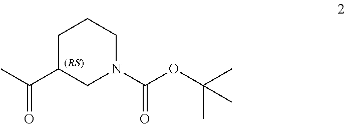 [SMILES: CC(=O)C1CCCN(C(=O)OC(C)(C)C)C1]

Methylmagnesium bromide (1.4 M in toluene/THF 75/25, 268 mL) was added dropwise to a solution of intermediate 1 (59 g, 0.22 mol) in THF (250 mL) at 0° C. and under nitrogen atmosphere and with the temperature not exceeding 15° C. After the addition, the reaction mixture was stirred at rt for 1 h. Then the mixture was poured on ice with 100 mL AcOH. The product was extracted with Et₂O and the organic layer was washed with a 5% NaHCO₃ solution. The organic layer was dried (MgSO₄), filtered and evaporated in vacuo affording intermediate 2 (50 g, quantitative).

### Preparation of Intermediate 3

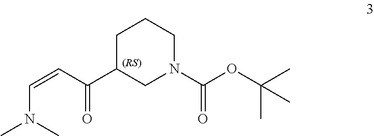 [SMILES: CN(C)/C=C\C(=O)C1CCCN(C(=O)OC(C)(C)C)C1]

Intermediate 2 (50 g, 0.22 mol) was added to N,N-dimethylformamide dimethyl acetal (110 mL) and the mixture was refluxed for 4 days. The reaction mixture was evaporated, additional N,N-dimethylformamide dimethyl acetal was added and the mixture was refluxed for an additional 4 hours. The solvent was evaporated and the product was purified by flash chromatography (silica, 1% MeOH in DCM, 2%). The desired fractions were collected and concentrated in vacuo affording intermediate 3 (60 g, 97%).

### Preparation of Intermediates 4, 5 and 6

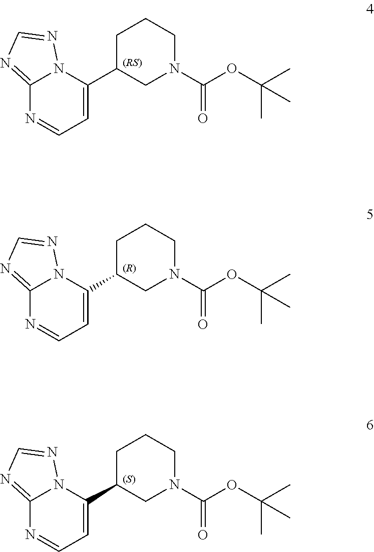

3-Amino-1,2,4-triazole (CAS: 61-82-5, 12.28 g, 0.15 mol) was added to a solution of intermediate 3 (33 g, 0.12 mol) in AcOH (75 mL). The reaction mixture was stirred at 130° C. for 1 h. Then the mixture was cooled on an ice bath and left stirring at rt overnight. The reaction mixture was concentrated in vacuo, co-evaporated with toluene and diluted with Et₂O (0.7 L) and poured onto ice (0.5 L). The layers were separated and the aqueous layer was extracted again with Et₂O (3×0.3 L). The combined organic layers were treated with brine, dried (MgSO₄), filtered and evaporated in vacuo affording intermediate 4 as a brown oil (25 g, 70%).

Intermediate 4 (25 g) was then separated into enantiomers via chiral SFC (Stationary phase: Chiralpak Diacel OJ 20×250 mm, Mobile phase: CO₂, MeOH) yielding intermediate 5 (10 g) and intermediate 6 (10 g).

### Preparation of Intermediate 7

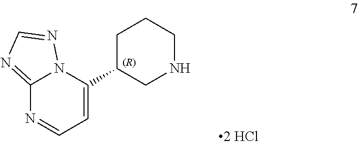

Intermediate 5 (3.8 g, 12.53 mmol) was dissolved in MeOH (100 mL) and treated with HCl (100 mL; 6 N in 2-propanol) at rt and the mixture was further stirred at rt for 16 h.

The volatiles were evaporated in vacuo and the solid was triturated with DIPE/ⁱPrOH (5/1, 100 mL) to give after drying (vacuum oven, 60° C., 16 h) the bishydrochloric acid salt of intermediate 7 (3.17 g, 92%).

### Preparation of Intermediate 8

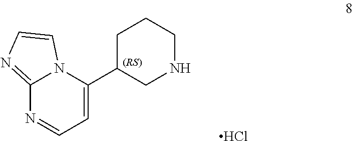

2-Aminoimidazole sulfate (CAS: 1450-93-7, 6.083 g, 23.02 mmol) was added to a solution of intermediate 3 (13 g, 46.04 mmol) in AcOH (100 mL) at reflux. The reaction mixture was stirred at reflux for 6 h. Then the solvent was evaporated in vacuo, Na₂CO₃ (aq. sat. soltn.) was added and the product was extracted with DCM. The organic layer was separated, dried (MgSO₄), filtered and evaporated in vacuo. The residue was treated with HCl (6 N in 2-propanol). The product was crystallized and the crystals were filtered off and dried yielding the dihydrochloric acid salt of intermediate 8 (2.08 g, 16%).

### Preparation of Intermediate 9

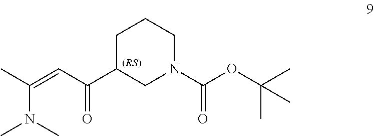 [SMILES: C/C(=C/C(=O)C1CCCN(C(=O)OC(C)(C)C)C1)N(C)C]

A mixture of intermediate 2 (2 g, 8.80 mmol) and N,N-dimethylacetamide dimethyl acetal (3.52 g, 26.40 mmol) was stirred at reflux for 3 h. The reaction mixture was evaporated in vacuo and used as such in the next reaction step (the product decomposes on silica).

### Preparation of Intermediate 10

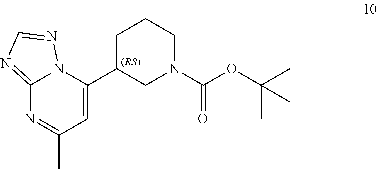 [SMILES: Cc1cc(C2CCCN(C(=O)OC(C)(C)C)C2)n2ncnc2n1]

3-Amino-1,2,4-triazole (CAS: 61-82-5, 0.71 g, 8.43 mmol) and intermediate 9 (2.5 g, 8.43 mmol) in AcOH (50 mL) were stirred at 120° C. for 30 minutes. Then the solvent was evaporated in vacuo and Na₂CO₃ (aq. sat. soltn.) was added and the product was extracted with DCM. The organic layer was separated, dried (MgSO₄), filtered and evaporated in vacuo. The product was purified by flash chromatography (silica, 1% MeOH in DCM, 2%). The desired fractions were collected and concentrated in vacuo affording intermediate 10 (2.14 g, 80%).

### Preparation of Intermediate 11

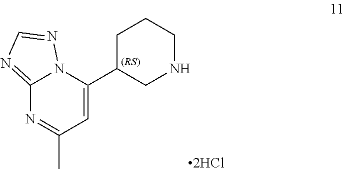 [SMILES: Cc1cc(C2CCCNC2)n2ncnc2n1]

HCl (50 mL; 4 N in 2-propanol) was added to a mixture of intermediate 10 (2.14 g, 6.74 mmol) in MeOH (50 mL) and the mixture was further stirred at rt for 1 h. The product crystallized from the reaction mixture and was filtered off and dried yielding the bishydrochloric acid salt of intermediate 11 (1.07 g, 55%).

### Preparation of Intermediate 12

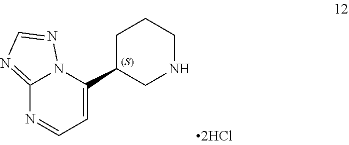

Intermediate 6 (3.9 g, 12.86 mmol) was dissolved in MeOH (100 mL) and treated with HCl (100 mL; 6 N in 2-propanol) at rt and the mixture was further stirred at rt for 16 h. The volatiles were evaporated in vacuo and the solid was triturated with DIPE/ⁱPrOH (250 mL/50 mL) to give after drying (vacuum oven, 60° C., 16 h) the bishydrochloric acid salt of intermediate 12 (3.18 g, 90%).

### Preparation of Intermediate 13

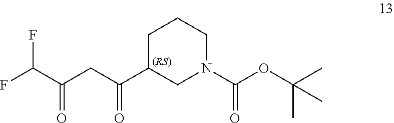 [SMILES: CC(C)(C)OC(=O)N1CCCC(C(=O)CC(=O)C(F)F)C1]

A mixture of intermediate 2 (20 g, 0.088 mol) in benzene (200 mL) was stirred at 0° C. under nitrogen atmosphere. Potassium tert-butoxide (12 g) followed by ethyl difluoroacetate (13.14 g, 0.106 mol) were added at 0-5° C. The reaction mixture was stirred at rt for 5 h. Then 10% H₂SO₄ (10 mL) was added dropwise until pH=7 keeping the temperature at 15-20° C. by cooling. The mixture was extracted with EtOAc (2×50 mL). The organic layer was separated and washed with brine (2×20 mL), dried (MgSO₄), filtered and evaporated in vacuo yielding intermediate 13 (26 g, 97%).

### Preparation of Intermediates 14 and 15

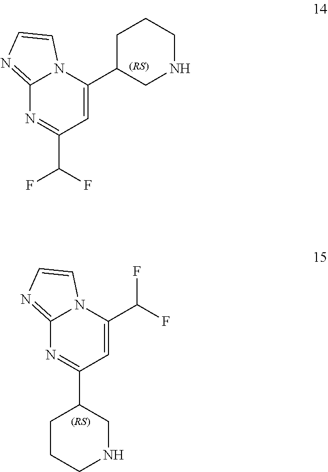 [SMILES: FC(F)c1cc(C2CCCNC2)n2ccnc2n1 ; FC(F)c1cc(C2CCCNC2)nc2nccn12]

2-Aminoimidazole sulfate (CAS: 1450-93-7, 22.5 g, 0.085 mol) was added to a solution of intermediate 13 (26 g, 0.085 mol) in AcOH (600 mL) at reflux. The reaction mixture was stirred at reflux for 6 h. Then the solvent was evaporated in vacuo. The residue was purified by RP HPLC (Vydac Denali C18—10 μm, 250 g, 5 cm). Mobile phase (0.05% TFA solution in water+5% CH₃CN, MeOH). The desired fractions were collected and concentrated in vacuo giving two fractions, which were filtered over silica with DCM+5% MeOH—NH₃ yielding intermediate 14 (6.5 g, 30%) and intermediate 15 (7.9 g, 37%) as free bases.

Intermediate 15 (5 g) as free base was dissolved in ⁱPrOH, acidified with HCl (6 N in 2-propanol) and stirred at rt for 20 h. The solid precipitated, it was filtered off, and dried (vacuum oven, 70° C.) yielding the bishydrochloric acid salt of intermediate 15 (4.9 g).

### Preparation of Intermediate 16

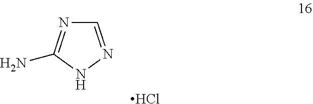

HCl (500 mL; 6 N in 2-propanol) and HCl (500 mL, 1M in water) were added to a mixture of 3-amino-1,2,4-triazole (CAS: 61-82-5, 50 g, 0.59 mol) in MeOH (500 mL). The reaction mixture was stirred at rt for 30 minutes. Then the solvents were evaporated in vacuo. The product was dried (vacuum oven, 60° C., 2 h) yielding the hydrochloric acid salt of intermediate 16 (69.6 g, 97%).

### Preparation of Intermediates 17 and 18 and 19

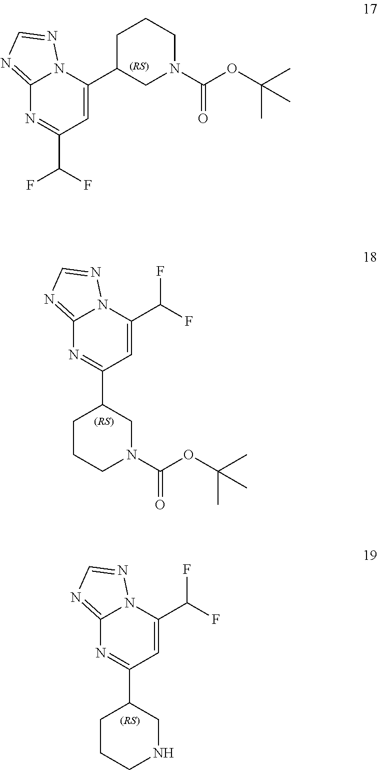 [SMILES: CC(C)(C)OC(=O)N1CCCC(c2cc(C(F)F)nc3ncnn23)C1 ; CC(C)(C)OC(=O)N1CCCC(c2cc(C(F)F)n3ncnc3n2)C1 ; FC(F)c1cc(C2CCCNC2)nc2ncnn12]

Procedure a: Intermediate 16 (55.44 g, 0.46 mol) was added to a solution of intermediate 13 (70.21 g, 0.23 mol) in DMF (800 mL). The reaction mixture was stirred at 60° C. for 20 h. The reaction mixture was cooled on an ice bath. Then DMF (300 mL) was added followed by triethylamine (160. 31 mL, 1.15 mol). Then a solution of di-tert-butyl dicarbonate (51.17 g, 0.23 mol) in DMF (300 mL) was added dropwise and the reaction mixture was stirred at rt for 1 h. Then the solvent was evaporated in vacuo. The residue was stirred in DCM (730 mL) and 20% NH₄OH (300 mL) for 30 minutes. The organic layer was separated, washed with water, dried (MgSO₄), filtered and evaporated in vacuo. The residue was purified by flash chromatography (silica, EtOAc in heptane 30/70). The desired fractions were collected and concentrated in vacuo. The residue was taken into DIPE and stirred at rt overnight. The solid that precipitated was filtered affording intermediate 17 (45.11 g, 55.5%). The filtrate was evaporated in vacuo yielding intermediate 18 (25 g, as mixture of compounds 17 and 18).

Procedure b: Intermediate 16 (15.42 g, 183.41 mmol) was added to a mixture of intermediate 13 (56 g, 183.41 mmol) in AcOH (300 mL). The mixture was refluxed for 2 h. Then the solvents were evaporated in vacuo. The residue was dissolved in DCM and washed with 10% Na₂CO₃ aq. sol. until neutral pH. The organic layer was separated, dried (MgSO₄), filtered and evaporated in vacuo. The residue was purified by flash chromatography (silica gel, eluent: DCM, 2% MeOH—NH₃). The desired fractions were collected and concentrated in vacuo affording intermediate 18 (45 g, 69% yield). The aqueous layer was evaporated in vacuo. The residue was stirred in DCM and filtered. The filtrate was dried over MgSO₄, filtered and evaporated in vacuo. The residue was purified by flash chromatography (silica gel, eluent: DCM, 10% MeOH—NH₃). The desired fractions were collected and concentrated in vacuo affording intermediate 19 (11 g, 24% yield).

Alternatively, intermediate 19 can be prepared through a procedure analogous to that described for the synthesis of intermediate 20 starting from intermediate 18.

### Preparation of Intermediate 20

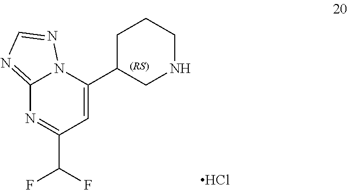 [SMILES: FC(F)c1cc(C2CCCNC2)n2ncnc2n1 ; Cl]

HCl/i-PrOH (200 mL) was added to a mixture of intermediate 17 (15 g, 42.45 mmol) in MeOH (400 mL). The reaction mixture was stirred at rt for 20 h. Then the mixture was evaporated in vacuo. The residue was stirred with DIPE. The solid that precipitated was filtered off to give after drying (vacuum oven, 70° C.) the bishydrochloric acid salt of intermediate 20 (14 g, quantitative).

### Preparation of Intermediate 21

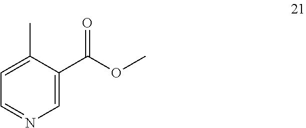

Procedure a: 4-Methyl-3-pyridinecarboxylic acid hydrochloride (1:1) (40 g, 230.4 mmol) was added to a refluxing mixture of sulphuric acid (20 mL) and MeOH (400 mL). The mixture was refluxed overnight, then it was evaporated and the resulting slurry was added to a cold solution of NaHCO₃ (64 g) in water (360 mL). The product was extracted with DCM and the OL was dried over MgSO₄, filtered and evaporated, yielding intermediate 21 (28.70 g, 83%)

Procedure b: A metal reactor was charged with 3-bromo-4-methyl-pyridine (200 g, 0.116 mol) and a mixture of DMF/MeOH (1 L/1 L). To this Et₃N (400 g, 0.395 mol), palladium (II) acetate (8 g, 0.036 mol) and 1,1′-bis(diphenylphosphino)ferrocene (16 g, 0.029 mol) were added. The reactor was closed and pressurized with CO gas (3 MPa) and the reaction mixture was stirred and heated overnight at 140° C. The RM was cooled, filtered and concentrated in vacuo. The residue was purified by flash column chromatography over silica gel (gradient eluent: EtOAc/Petroleum ether from 1/1 to 1/0). The product fractions were collected and the solvent was evaporated to afford the desired intermediate 21 (90 g, 51%).

### Preparation of Intermediate 22

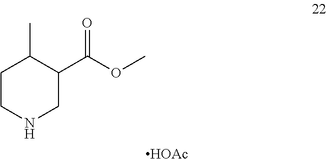

Procedure a: A hydrogenation flask was charged with AcOH (500 mL) and then PtO₂ (15.02 g, 66.2 mmol) was added. Intermediate 21 (50 g, 330.8 mmol) was added and the mixture was hydrogenated at 50° C. for 7 days. The RM was filtered over Dicalite® and the filtrate was evaporated to yield intermediate 22 (52 g), which was used in the next step without further purification.

Procedure b: Platinum oxide (5 g, 0.022 mol) was added to a solution of intermediate 21 (90 g, 0.595 mol) and AcOH (1 L). The RM was stirred and hydrogenated for 5 days at 50° C. under a pressure of 3.5 kPa. The cooled RM was concentrated in vacuo to give intermediate 22 as the acetic acid salt (140 g, 97%, 90% purity determined by ¹H-NMR).

### Preparation of Intermediate 23

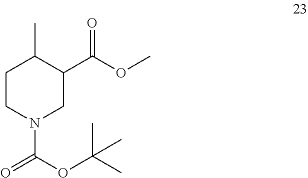 [SMILES: COC(=O)C1CN(C(=O)OC(C)(C)C)CCC1C]

Procedure a: To a solution of intermediate 22 (52 g, 330.8 mmol) in DCM (869 mL), DIPEA (85.5 g, 661.5 mmol) and DMAP (4.04 g, 33.08 mmol) were added. Then di-tert-butyl dicarbonate (72.19 g, 330.8 mmol) was added to this solution in small portions and the reaction was stirred at RT for 1 h. The RM was washed with water and brine and the organic layer was dried over MgSO₄, filtered and evaporated. The product was purified by flash chromatograph (silica gel, eluent: DCM, 1% MeOH in DCM, 2%, 4%). The desired fractions were evaporated, yielding intermediate 23 (64.1 g, 75%).

Procedure b: To a stirred and cooled (0° C.) solution of intermediate 22 (140 g, 0.595 mol) in DCM (1.5 L) was added sequentially di-tert-butyl dicarbonate (130 g, 0.596 mol), Et₃N (225 g, 1.74 mol) and DMAP (10 g, 0.082 mol) and stirring was continued at RT for 2 h. The reaction mixture was poured onto H₂O (500 mL) and extracted with DCM (2×100 mL). The organic layers were separated, dried (Na₂SO₄), and the solvent was evaporated to give crude intermediate 23 (150 g, 90%, 90% purity determined by ¹H-NMR) which was used as such in the next step.

### Preparation of Intermediate 24

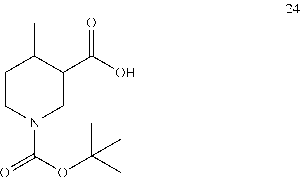 [SMILES: CC1CCN(C(=O)OC(C)(C)C)CC1C(=O)O]

Procedure a: Intermediate 23 (64.1 g, 249.1 mmol) was stirred in MeOH (500 mL) at RT. NaOH (2 M, 747.3 mL) was added and the mixture was stirred for 2 h at RT. The RM was acidified with HCl 1N and the product was extracted with Et₂O. The OL was washed with brine and dried over MgSO₄, filtered and evaporated, yielding intermediate 24 (59.70 g) as a white solid.

Procedure b: To a stirred solution of intermediate 23 (150 g, 90% pure, 0.524 mol) in MeOH (0.9 L) was added a solution of a 2M NaOH solution (1.8 mol). After 14 h at RT, the RM was extracted with MTBE (2×0.8 L). The aqueous layer was acidified with 10% citric acid and then extracted with EtOAc (4×1 L). The combined organic layers were dried over Na₂SO₄, filtered and concentrated in vacuo to give crude intermediate 24 (142 g, 90% purity determined by ¹H-NMR, 100%) which was used as such in the next step.

### Preparation of Intermediate 25

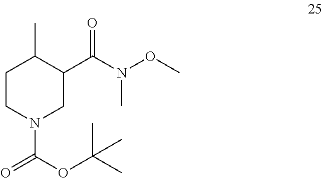 [SMILES: CON(C)C(=O)C1CN(C(=O)OC(C)(C)C)CCC1C]

Procedure a: To a solution of intermediate 24 (59.7 g, 0.25 mol) in THF (800 mL), was added di-1H-imidazol-1-yl-methanone (54 g, 0.33 mol) and the mixture was stirred at RT for 1 h. In another flask, to a suspension of N-methoxy-methanamine hydrochloride (1:1) (32.93 g, 0.34 mol) in ACN (500 mL), was added trimethylamine (35.75 g, 0.35 mol). Both mixtures were combined and stirred at 50° C. while monitoring. The intermediate product crystallized out of the RM and did not react with N-methoxy-methanamine to form the desired product. DCM was added until the intermediate dissolved. The reaction was left stirring for 1 week at 80° C. The solvents were evaporated. The residue was dissolved in DCM and washed with water, 20% AcOH solution and finally with a saturated NaHCO₃ solution. The OL was dried over MgSO₄, filtered and evaporated. The product was purified by flash chromatography (silica gel, eluent: 2% MeOH in DCM, 4%). The pure fractions were evaporated, yielding intermediate 25 (70 g, quantitative).

Procedure b: To a stirred and ice-cooled solution of intermediate 24 (140 g, 0.518 mol) in DCM (2 L) was added N,O-dimethylhydroxylamine (113 g, 1.16 mol) and Et₃N (113 g, 1.79 mol). Then HATU (235 g, 0.618 mol) was added and stirring was continued for 14 h. The solvent was evaporated and a NaHCO₃ solution (0.5 L) was added and then extracted with DCM (3×1 L). The combined organic layers were separated, dried over Na₂SO₄, filtered and concentrated in vacuo. The residue was purified by silica gel flash chromatography eluting with 1-10% EtOAc in petroleum ether to afford intermediate 25 (152 g, 100%).

### Preparation of Intermediate

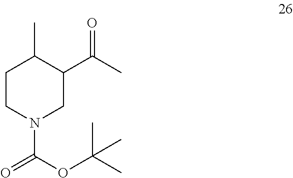 [SMILES: CC(=O)C1CN(C(=O)OC(C)(C)C)CCC1C]

Procedure a: Intermediate 25 (70 g, 244.4 mmol) in THF (250 mL) was charged in a flask under N₂ and cooled to −15° C. Methylmagnesium bromide (1.4 M in toluene/THF 75/25, 206 mL) was added dropwise, with the temperature not exceeding 0° C. After addition, the RM was stirred at RT for 1 h. Then the RM was poured on ice with 20 mL AcOH. The product was extracted with Et₂O and the OL was washed with a 5% NaHCO₃ solution. The OL was dried over MgSO₄, filtered and evaporated to give intermediate 26 (53.35 g, 90%)

Procedure b: To a stirred and cooled solution (0° C.) of intermediate 25 (150 g, 0.524 mol) in THF (2 L) was added dropwise a 3M methylmagnesium bromide solution in THF (0.75 L, 2.25 mol) and stirring was continued at RT for 2 h. The reaction mixture was poured onto aqueous NH₄Cl solution and extracted with DCM. The combined organic layers were dried over Na₂SO₄, filtered and concentrated in vacuo. The residue was purified by silica gel chromatography eluting with 1-5% EtOAc in petroleum ether to afford intermediate 26 (120 g, 95%).

### Preparation of Intermediate 27

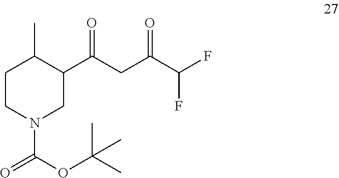 [SMILES: CC1CCN(C(=O)OC(C)(C)C)CC1C(=O)CC(=O)C(F)F]

Intermediate 26 (53.35 g, 0.22 mol) was stirred in toluene (1500 mL) at 0° C. under N₂. Potassium tert-butoxide (34.14 g) was added at 0-5° C. and 2,2-difluoro-acetic acid ethyl ester (33.01 g, 0.27 mol) was added dropwise at 0-5° C. The RM was stirred at RT for 2 h, then washed with 10% H₂SO₄ in water and the OL was dried on MgSO₄, filtered and evaporated, yielding intermediate 27 (70.50 g, quantitative).

### Preparation of Intermediate 28

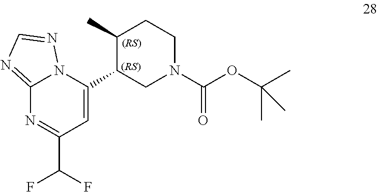 [SMILES: C[C@H]1CCN(C(=O)OC(C)(C)C)C[C@@H]1c1cc(C(F)F)nc2ncnn12]

Intermediate 27 (70.5 g, 220.8 mmol), 1H-1,2,4-triazol-5-amine hydrochloride (1:1) (53.22 g, 441.52 mmol) and DMF (1500 mL) were stirred at 80° C. for 24 h. Et₃N (20 g) and di-tert-butyl dicarbonate (20 g) were added. The mixture was stirred for 30 min, evaporated and then dissolved in EtOAc, washed with water and brine. The OL was dried over MgSO₄, filtered and evaporated. Four isomers were observed. The first fraction crystallized from Et₂O. The crystals were filtered off and dried, yielding intermediate 28 (24.60 g, 30%). The mother liquor yielded a second fraction of the compound. The crystals were filtered off and dried, yielding intermediate 28 (2.53 g, 3%).

### Preparation of Intermediates 29, 30 and 31

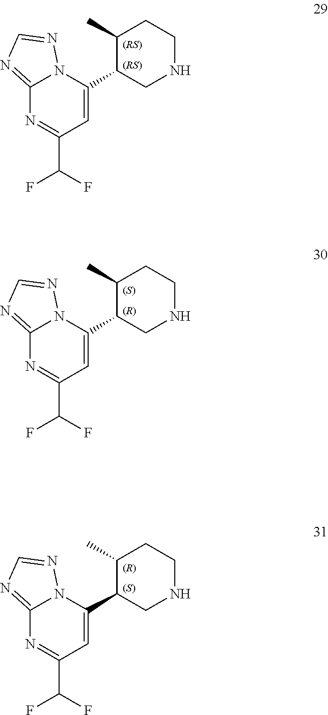

To a solution of intermediate 28 (24.6 g, 67 mmol) in MeOH (350 mL), was added HCl-iPrOH (350 mL) and the RM was stirred for 2 h at RT. The RM was evaporated and the product was crystallized from EtOH. The crystals were filtered off and dried, yielding 20.33 g of a crude, to which water, Na₂CO₃ and DCM were added. The OL was dried over MgSO₄, filtered and evaporated, yielding 12.80 g of intermediate 29. This free base was separated into enantiomers by purification by Prep SFC (Stationary phase: Chiralpak Diacel AD 30×250 mm; mobile phase: CO₂, ((MeOH-iPrOH 50/50) with 0.4% iPrNH₂), yielding intermediate 30 (5 g, 19%, Rₜ=7.57 min) and intermediate 31 (5.13 g, 19%, Rₜ=9.36 min).

Intermediates 30 and 31 were isolated as free bases or alternatively, they were dissolved in MeOH, followed by addition of HCl/i-PrOH and the mixture evaporated. The hydrochloride salts (in each instance, .HCl) were crystallized from ACN, filtered off and dried.

### Preparation of Intermediate

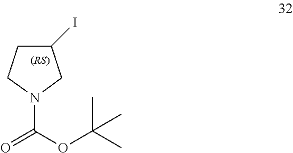

Iodine (7.45 g, 29.37 mmol) was added portionwise to a solution of (R)-(−)-N-Boc-3-pyrrolidinol (CAS: 103057-44-9; 5 g, 26.70 mmol), imidazole (2.73 g, 40.06 mmol) and triphenylphosphine (7.70 g, 29.37 mmol) in THF (50 mL) at 0° C. (ice bath). The mixture was stirred at rt for 3 h. The excess of iodine was quenched with Na₂S₂O₃ (10%, aq. soltn.). The mixture was extracted with EtOAc. The organic layer was separated, dried (Na₂SO₄), filtered and the solvent evaporated in vacuo. The residue was purified by flash chromatography (silica, EtOAc in DCM from 0/100 to 20/80).

The desired fractions were collected and concentrated in vacuo affording intermediate 32 as a colourless oil (7.21 g, 91%).

### Preparation of Intermediate 33

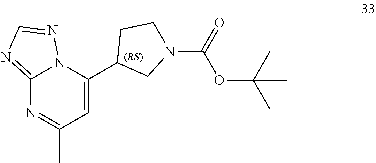 [SMILES: Cc1cc(C2CCN(C(=O)OC(C)(C)C)C2)n2ncnc2n1]

A solution of intermediate 32 (1.12 g, 3.77 mmol) and lithium chloride (7.53 mL, 3.77 mmol) in THF (1 ml) was pumped through a column containing activated Zn (15.24 g, 233.06 mmol) at 40° C. with flow of 0.5 mL/min. The outcome was collected over a stirred solution of 7-chloro-5-methyl-[1,2,4]triazolo[1,5-a]pyrimidine (CAS: 24415-66-5; 254 mg, 1.51 mmol) and bis(tri-tert-butylphosphine)palladium(0) (CAS: 53199-31-8; 38.5 mg, 0.075 mmol) in THF (1.5 mL) at rt under nitrogen atmosphere. The mixture was stirred at rt overnight. The crude was quenched with NH₃ (10%, aq. soltn.) and extracted with EtOAc. The organic layer was separated, dried (Na₂SO₄), filtered and the solvent evaporated in vacuo. The residue was purified by flash chromatography (silica, EtOAc in DCM from 0/100 to 100/0). The desired fractions were collected and concentrated in vacuo affording intermediate 33 as a brown oil (203 mg, 43%). For the above reaction Zn was activated as follows: A solution of TMSCl (2M) and 1-bromo-2-choroethane (0.3M) in THF (10 mL) was passed through the column containing Zn at 40° C. with a flow of 1 mL/min.

### Preparation of Intermediate 34

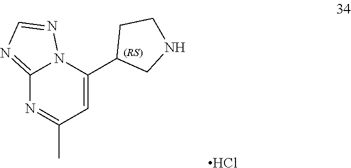

HCl (0.41 mL; 4 M in dioxane) was added to a stirred solution of intermediate 33 (70 mg, 0.23 mmol) in 1,4-dioxane (0.5 mL) at rt and the mixture was further stirred at rt for 150 minutes. The volatiles were evaporated in vacuo to give the hydrochloric acid salt of intermediate 34 (47 mg, quantitative).

### Preparation of Intermediate 35

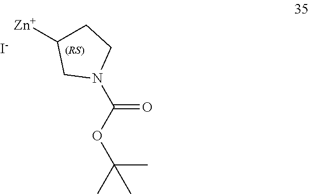

A solution of intermediate 32 (600 mg, 2.02 mmol) and lithium chloride (0.5 M in THF; 4 mL, 2 mmol) in THF (1.2 mL) was pumped through a column containing activated Zn (15 g, 229.39 mmol) at 40° C. with flow of 0.5 mL/min. The organozinc reagent was recovered pumping lithium chloride (0.5 M in THF; 4 mL, 2 mmol) in THF (1.2 mL) at 40° C. with flow of 0.5 mL/min. The outcome solution was collected under N₂ atmosphere to yield intermediate 35 (0.2 M after titration with 12) that was used without any further manipulation.

For the above reaction Zn was activated as follows: A solution of TMSCl (2M) and 1-bromo-2-choroethane (0.3M) in THF (10 mL) was passed through the column containing Zn at 40° C. with a flow of 1 mL/min.

### Preparation of Intermediate 36

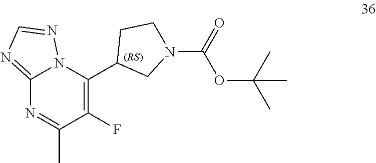 [SMILES: Cc1nc2ncnn2c(C2CCN(C(=O)OC(C)(C)C)C2)c1F]

Intermediate 35 (3.3 mL; 0.16 M in THF) was added to a solution of 7-chloro-6-fluoro-5-methyl-[1,2,4]triazolo[1,5-a]pyrimidine (prepared through the procedure described in PCT Int. Appl., 2010018868; 39 mg, 0.21 mmol) and bis(tri-tert-butylphosphine)palladium(0) (CAS: 53199-31-8; 5.4 mg, 0.01 mmol) in THF (0.2 mL) at rt under nitrogen atmosphere. The mixture was stirred at rt overnight. The crude was quenched with water and extracted with EtOAc. The organic layer was separated, dried (Na₂SO₄), filtered and the solvent evaporated in vacuo. The residue was purified by flash chromatography (silica, EtOAc in heptane from 0/100 to 100/0). The desired fractions were collected and concentrated in vacuo affording intermediate 36 as a yellow oil (24 mg, 35%).

### Preparation of Intermediate 37

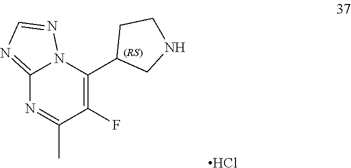 [SMILES: Cc1nc2ncnn2c(C2CCNC2)c1F ; Cl]

HCl (0.198 mL; 4 M in dioxane) was added to a stirred solution of intermediate 36 (35.6 mg, 0.11 mmol) in 1,4-dioxane (0.25 mL) at rt and the mixture was further stirred at rt for 3 h. The volatiles were evaporated in vacuo to give the hydrochloric acid salt of intermediate 37 (28.5 mg, 99%).

### Preparation of Intermediate 38

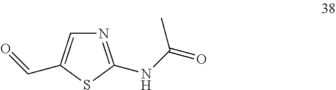

Acetyl chloride (6 mL, 84.38 mmol) was added to a solution of 2-amino-5-formylthiazole (10 g, 78 mmol) and diisopropylamine (45 mL, 261.1 mmol) in DCM (100 mL) at 0° C. The resulting mixture was allowed to warm to rt and further stirred at rt for 17 h. NH₄Cl (aq. sat. soltn.) was added and the mixture was extracted with EtOAc. The organic layer was separated, dried over MgSO₄, filtered and concentrated in vacuo. The residue thus obtained was purified by flash column chromatography (silica; dry load, EtOAc in DCM 0/100 to 50/50) and the desired fractions were concentrated in vacuo to yield intermediate 38 as yellow solid (8.6 g, 65% yield).

### Preparation of Intermediate 39

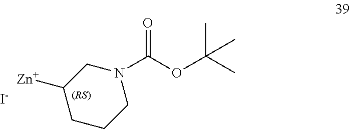

A solution of tert-butyl 3-iodopiperidine-1-carboxylate (313 mg, 1 mmol) in THF (2 mL) was pumped through a column containing activated Zn at 40° C. with flow of 0.5 mL/min. The outcome solution was collected under N₂ atmosphere to yield intermediate 39 that was used without any further manipulation.

For the above reaction Zn was activated as follows: A solution of TMSCl (2M) and 1-bromo-2-choroethane (0.3M) in THF (10 mL) was passed through the column containing Zn at 40° C. with a flow of 1 mL/min.

### Preparation of Intermediate 40

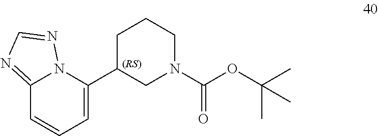 [SMILES: CC(C)(C)OC(=O)N1CCCC(c2cccc3ncnn23)C1]

Intermediate 39 (249.65 mg, 1 mmol) was added to a mixture of 5-bromoimidazo[1,2-a]pyridine (CAS: 69214-09-1; 100 mg, 0.51 mmol), 2-dicyclohexylphosphino-2′,6′-diisopropoxy-1,1′-biphenyl (CAS: 787618-22-8; 12 mg, 0.03 mmol) and Pd(OAc)₂ (11 mg, 0.05 mmol) under nitrogen atmosphere. The mixture was stirred at rt for 17 h. Then NaHCO₃ (aq. sat. soltn.) was added and the mixture was extracted with EtOAc. The organic layer was separated, dried (MgSO₄), filtered and the solvent evaporated in vacuo. The residue was purified by flash chromatography (silica, EtOAc in heptane from 0/100 to 100/0). The desired fractions were collected and concentrated in vacuo affording intermediate 40 as a yellow oil (24 mg, 16%).

### Preparation of Intermediate 41

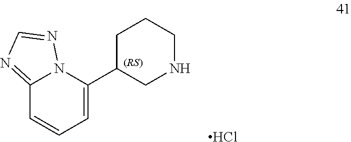 [SMILES: c1cc(C2CCCNC2)n2ncnc2c1 ; Cl]

HCl (0.3 mL; 4 M in dioxane) was added to a stirred solution of intermediate 40 (40 mg, 0.13 mmol) in MeOH (1 mL) at rt and the mixture was further stirred at rt for 24 h. The volatiles were evaporated in vacuo to give the hydrochloric acid salt of intermediate 41 (43 mg, 72%).

### Preparation of Intermediate 42

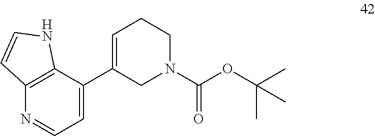 [SMILES: CC(C)(C)OC(=O)N1CCC=C(c2ccnc3cc[nH]c23)C1]

A mixture of 7-chloro-1H-pyrrolo[3,2-b]pyridine (prepared through the procedure described in J. Med. Chem., 2014, 57 (13), 5728-5737; 50 mg, 0.33 mmol), tert-butyl 5-(4,4,5,5-tetramethyl-1,3,2-dioxaborolan-2-yl)-3,6-dihydro-2H-pyridine-1-carboxylate (CAS: 1251537-34-4; 111.5 mg, 0.36 mmol) and Pd(PPh₃)₄ (CAS: 14221-01-3; 38 mg, 0.03 mmol) in 1,4-dioxane (1.29 mL) and Na₂CO₃ (0.75 mL; aq. sat. soltn.) in a sealed tube and under nitrogen atmosphere was stirred at 150° C. for 30 minutes under microwave irradiation. The reaction mixture was diluted with EtOAc and washed with water. The organic layer was separated, dried (MgSO₄), filtered and the solvent evaporated in vacuo. The residue was purified by flash chromatography (silica, EtOAc in DCM from 0/100 to 100/0). The desired fractions were collected and concentrated in vacuo affording intermediate 42 as a pale yellow solid (82 mg, 84% yield).

### Preparation of Intermediate 43

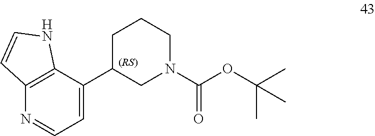 [SMILES: CC(C)(C)OC(=O)N1CCCC(c2ccnc3cc[nH]c23)C1]

To a solution of intermediate 42 (125 mg, 0.42 mmol) in EtOH (10 mL), 10% palladium on active carbon (125 mg, 0.12 mmol) was added under nitrogen atmosphere. Then the mixture was evacuated and backfilled with hydrogen three times. The mixture was stirred under hydrogen atmosphere for 18 h. The reaction mixture was filtered through a pad of Celite® and rinsed with EtOH. The filtrate was concentrated in vacuo to yield intermediate 43 (100 mg, 79% yield) that was used without any further manipulation.

### Preparation of Intermediate 44

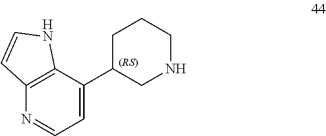 [SMILES: c1cc(C2CCCNC2)c2[nH]ccc2n1]

HCl (0.83 mL; 4 M in dioxane) was added to a stirred solution of intermediate 43 (100 mg, 0.33 mmol) in 1,4-dioxane (0.84 mL) at rt and the mixture was further stirred at rt for 1 h. The volatiles were evaporated in vacuo. The residue was purified by ion exchange chromatography (Isolute® SCX2 eluting first with MeOH and then with 7N NH₃ in MeOH). The desired fractions were collected and concentrated in vacuo affording product 44 as a white solid (65 mg, 97% yield).

### Preparation of Intermediate 45

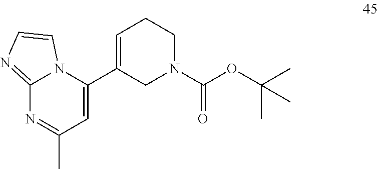 [SMILES: Cc1cc(C2=CCCN(C(=O)OC(C)(C)C)C2)n2ccnc2n1]

A mixture of 5-chloro-7-methyl-imidazo[1,2-a]pyrimidine (prepared through the procedure described in J. Med. Chem., 2012, 55 (17), 7425-7436; 543 mg, 3.24 mmol), tert-butyl 5-(4,4,5,5-tetramethyl-1,3,2-dioxaborolan-2-yl)-3,6-dihydro-2H-pyridine-1-carboxylate (CAS: 1251537-34-4; 1001.8 mg, 3.24 mmol) and Pd(PPh₃)₄ (CAS: 14221-01-3; 374 mg, 0.32 mmol) in 1,4-dioxane (9.7 mL) and NaHCO₃ (8.1 mL; 1.2 M) in a sealed tube and under nitrogen atmosphere was stirred at 150° C. for 15 minutes under microwave irradiation. The reaction mixture was diluted with EtOAc and washed with water. The organic layer was separated, dried (Na₂SO₄), filtered and the solvent evaporated in vacuo. The residue was purified by flash chromatography (silica, MeOH in DCM from 0/100 to 10/90). The desired fractions were collected and concentrated in vacuo affording intermediate 45 as a colorless oil (256 mg, 44% yield).

### Preparation of Intermediate 46

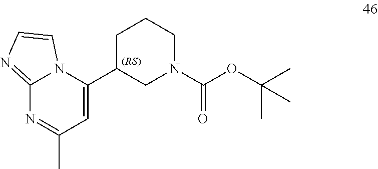 [SMILES: Cc1cc(C2CCCN(C(=O)OC(C)(C)C)C2)n2ccnc2n1]

Hydrogen was bubbled into a suspension of intermediate 45 (256 mg, 0.81 mmol) and 10% palladium on active carbon (173 mg, 0.16 mmol) in MeOH (6.6 mL). Then the mixture was stirred at rt for 2 h. The reaction mixture was filtered through a pad of Celite® and the filtrate was concentrated in vacuo to yield intermediate 46 as a colorless oil (263 mg, quantitative).

### Preparation of Intermediate 47

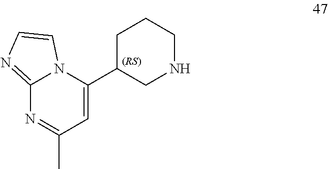 [SMILES: Cc1cc(C2CCCNC2)n2ccnc2n1]

HCl (2.77 mL; 4 M in ⁱPrOH) was added to a stirred solution of intermediate 46 (263 mg, 0.83 mmol) in MeOH (5 mL) at rt and the mixture was further stirred at rt for 16 h. The volatiles were evaporated in vacuo. The residue was purified by ion exchange chromatography (Isolute® SCX2 eluting first with MeOH and then with 7N NH₃ in MeOH). The desired fractions were collected and concentrated in vacuo affording product 47 as a pale yellow oil (132 mg, 73% yield).

### Preparation of Intermediate 48

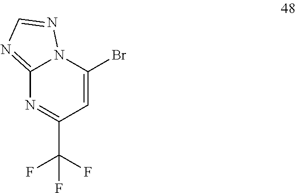 [SMILES: FC(F)(F)c1cc(Br)n2ncnc2n1]

POBr₃ (1.54 g, 5.39 mmol) was added to a suspension of 5-(trifluoromethyl)-[1,2,4]triazolo[1,5-a]pyrimidin-7-ol (prepared through the procedure described in J. Med. Chem., 2011, 54 (11), 3935-3949; 440 mg, 2.16 mmol) in CH₃CN (8 mL). The mixture was stirred at 150° C. for 5 minutes under microwave irradiation. The excess of POBr₃ was quenched with ice-water and the mixture was extracted with DCM. The organic layer was separated, dried (Na₂SO₄), filtered and the solvent evaporated in vacuo. The residue was purified by flash chromatography (silica, EtOAc in DCM from 0/100 to 25/75). The desired fractions were collected and concentrated in vacuo affording intermediate 48 as an off white solid (357 mg, 62% yield).

### Preparation of Intermediate 49

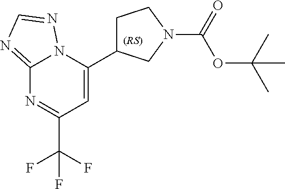 [SMILES: CC(C)(C)OC(=O)N1CCC(c2cc(C(F)(F)F)nc3ncnn23)C1]

Intermediate 35 (1.87 mmol, 0.3 M in THF) was added to a mixture of intermediate 48 (200 mg, 0.75 mmol), Pd(OAc)₂ (19.14 mg, 0.037 mmol) and 2-dicyclohexylphosphino-2′,6′-di-i-propoxy-1,1′-biphenyl (also known as RuPhos) (CAS: 787618-22-8; 34.95 mg, 0.075 mmol) in THF (0.5 mL). The resulting mixture was stirred at rt overnight. The crude was quenched with water and extracted with EtOAc. The organic layer was separated, dried over Na₂SO₄, filtered and concentrated in vacuo. The residue thus obtained was purified by flash column chromatography (silica; EtOAc in DCM 0/100 to 40/60) and the desired fractions were concentrated in vacuo. The residue obtained was purified again by flash column chromatography (silica; EtOAc in heptane 0/100 to 100/0). The desired fractions were concentrated in vacuo to yield intermediate 49 as a colorless oil (110 mg, 39% yield).

### Preparation of Intermediate 50

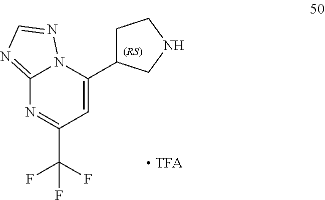 [SMILES: FC(F)(F)c1cc(C2CCNC2)n2ncnc2n1]

TFA (0.7 mL) was added to a stirred solution of intermediate 49 (123 mg, 0.345 mmol) in DCM (1 mL). The mixture was stirred for 2 h. The volatiles were evaporated in vacuo yielding the trifluoroacetate salt of intermediate 50 (89 mg, 99% yield) that was used without any further manipulation.

### Preparation of Intermediate 51

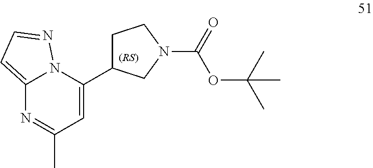 [SMILES: Cc1cc(C2CCN(C(=O)OC(C)(C)C)C2)n2nccc2n1]

Intermediate 35 (1.35 mmol, 0.3 M in THF) was added to a mixture of 7-chloro-5-methylpyrazolo[1,5-a]pyrimidine (CAS: 16082-27-2; 90.5 mg, 0.54 mmol), Pd(OAc)₂ (13.8 mg, 0.027 mmol) and 2-dicyclohexylphosphino-2′,6′-di-i-propoxy-1,1′-biphenyl (also known as RuPhos) (CAS: 787618-22-8; 25.2 mg, 0.054 mmol) in THF (0.36 mL). The resulting mixture was stirred at rt overnight. The crude was quenched with water and extracted with EtOAc. The organic layer was separated, dried over Na₂SO₄, filtered and concentrated in vacuo. The residue thus obtained was purified by flash column chromatography (silica; EtOAc in heptane 0/100 to 60/40). The desired fractions were concentrated in vacuo. The residue was purified by RP HPLC (Stationary phase: C18 XBridge 30×100 mm 5 um), Mobile phase: Gradient from 74% 0.1% NH₄CO₃H/NH₄OH pH 9 solution in water, 26% CH₃CN to 58% 0.1% NH₄CO₃H/NH₄OH pH 9 solution in water, 42% CH₃CN). The desired fractions were collected and concentrated in vacuo affording intermediate 51 as a colorless oil (38 mg, 21% yield).

### Preparation of Intermediate 52

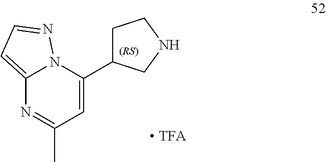

TFA (0.19 mL) was added to a stirred solution of intermediate 51 (38 mg, 0.13 mmol) in DCM (0.35 mL). The mixture was stirred for 2 h. The volatiles were evaporated in vacuo yielding the trifluoroacetate salt of intermediate 52 (25 mg, 99% yield) that was used without any further manipulation.

### Preparation of Intermediates 53 and 54

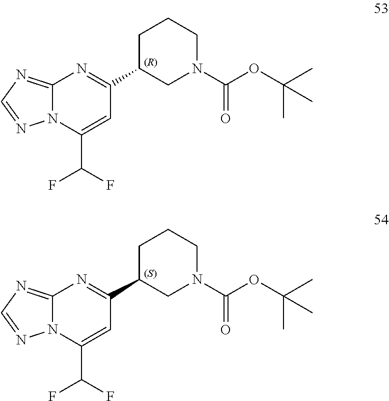

Intermediate 18 (45 g, 127.34 mmol) was separated into enantiomers by purification by Prep SFC (Stationary phase: Chiralpak Diacel AD 30×250 mm; mobile phase: CO₂, (MeOH with 0.2% iPrNH₂), yielding intermediate 53 (21 g, 47%) and intermediate 54 (20 g, 44%)

### Preparation of Intermediate 55

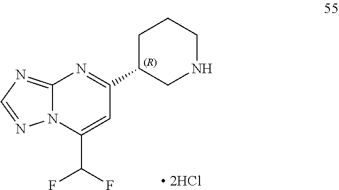

HCl-ⁱPrOH (200 mL) was added to a mixture of intermediate 53 (21 g, 59.43 mmol) in MeOH (500 mL). The mixture was stirred at reflux for 30 minutes. The solvents were evaporated in vacuo to half of the volume. The precipitated thus obtained was filtered off, dried in vacuum at 70° C. affording the bishydrochloric salt of intermediate 55 (17.5 g, 90% yield).

### Preparation of Intermediate 56

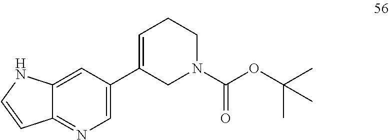 [SMILES: CC(C)(C)OC(=O)N1CCC=C(c2cnc3cc[nH]c3c2)C1]

A mixture of 6-bromo-1H-pyrrolo[3,2-b]pyridine (CAS: 944937-53-5; 75 mg, 0.38 mmol), tert-butyl 5-(4,4,5,5-tetramethyl-1,3,2-dioxaborolan-2-yl)-3,6-dihydro-2H-pyridine-1-carboxylate (CAS: 1251537-34-4; 129.5 mg, 0.42 mmol) and Pd(PPh₃)₄ (CAS: 14221-01-3; 44 mg, 0.04 mmol) in 1,4-dioxane (1.5 mL) and Na₂CO₃ (0.75 mL; aq. sat. soltn.) in a sealed tube and under nitrogen atmosphere was stirred at 150° C. for 30 minutes under microwave irradiation. The reaction mixture was diluted with EtOAc and washed with water. The organic layer was separated, dried (Na₂SO₄), filtered and the solvent evaporated in vacuo. The residue was purified by flash chromatography (silica, EtOAc in DCM from 0/100 to 100/0). The desired fractions were collected and concentrated in vacuo affording intermediate 56 as a pale yellow solid (100 mg, 88% yield).

### Preparation of Intermediate 57

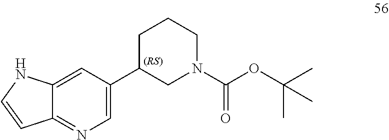 [SMILES: CC(C)(C)OC(=O)N1CCCC(c2cnc3cc[nH]c3c2)C1]

A solution of intermediate 56 (100 mg, 0.33 mmol) in EtOH (10 mL), 10% palladium on active carbon (100 mg, 0.09 mmol) was added under nitrogen atmosphere. Then the mixture was evacuated and backfilled with hydrogen three times. The mixture was stirred under hydrogen atmosphere for 18 h. The reaction mixture was filtered through a pad of Celite® and rinsed with EtOH. The filtrate was concentrated in vacuo to yield intermediate 57 (50 mg, 50% yield) that was used without any further manipulation.

### Preparation of Intermediate 58

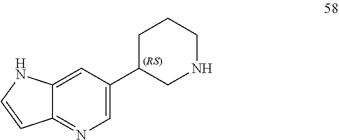 [SMILES: c1cc2ncc(C3CCCNC3)cc2[nH]1]

HCl (0.41 mL; 4 M in dioxane) was added to a stirred solution of intermediate 57 (50 mg, 0.17 mmol) in 1,4-dioxane (0.42 mL) at rt and the mixture was further stirred at rt for 1 h. The volatiles were evaporated in vacuo. The residue was purified by ion exchange chromatography (Isolute® SCX2 eluting first with MeOH and then with 7N NH₃ in MeOH). The desired fractions were collected and concentrated in vacuo affording product 58 as a white solid (33 mg, 99% yield).

### Preparation of Intermediate 59

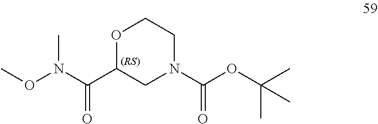 [SMILES: CON(C)C(=O)C1CN(C(=O)OC(C)(C)C)CCO1]

A solution of 1,1′-carbonyldiimidazole (CAS: 530-62-1, 32.26 g, 0.20 mol) in THF (350 mL) was added to a solution of 4-tert-butoxycarbonylmorpholine-2-carboxylic acid (CAS: 189321-66-2; 40 g, 0.17 mol) in THF (350 mL) at 0° C. The mixture was stirred at rt for 1 h. Then the mixture was cooled to 0° C. and a suspension of triethylamine (36.06 mL, 0.26 mol) and N-methoxymethanamine hydrochloride (1:1) (19.40 g, 0.20 mol) in CH₃CN (452 mL) was added dropwise at 0° C. and the mixture was stirred at rt for 16 h. Then the solvents were evaporated in vacuo. The residue was dissolved in DCM and washed with water (3×500 mL), acetic acid (20% solution) and finally with NaHCO₃ (aq. sat. soltn.). The organic layer was separated, dried (MgSO₄), filtered, evaporated in vacuo affording intermediate 59 (46.4 g, 98% yield)

### Preparation of Intermediate 60

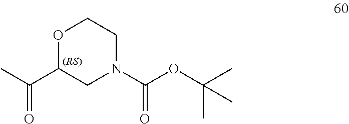 [SMILES: CC(=O)C1CN(C(=O)OC(C)(C)C)CCO1]

Methylmagnesium bromide (1.4 M in toluene/THF 75/25, 181 mL) was added dropwise to a solution of intermediate 59 (46.4 g, 0.17 mol) in THF (500 mL) at 0° C. and under nitrogen atmosphere and with the temperature not exceeding 10° C. After the addition, the reaction mixture was stirred at rt for 1 h. Then the mixture was quenched with a sat. NH₄Cl solution. The product was extracted with EtOAc and the organic layer was washed with a sat. NH₄Cl solution. The organic layer dried (MgSO₄), filtered and evaporated in vacuo affording intermediate 60 as an orange oil (41 g, quantitative)

### Preparation of Intermediate 61

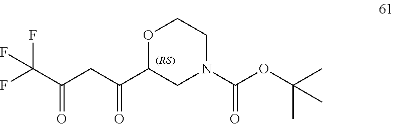 [SMILES: CC(C)(C)OC(=O)N1CCOC(C(=O)CC(=O)C(F)(F)F)C1]

Potassium tert-butoxide (CAS: 865-47-4; 4.89 g, 43.62 mmol) and ethyl trifluoroacetate (CAS; 383-63-1; 5.19 mL, 43.62 mmol) were added to a mixture of intermediate 60 (4 g, 17.45 mmol) in toluene (64 mL) at 0° C. and under nitrogen atmosphere. Then the mixture was stirred at rt for 16 h. Then a sat. NH₄Cl solution was added. The product was extracted with EtOAc. The organic layer was separated, dried (MgSO₄), filtered and evaporated in vacuo affording intermediate 61 as a yellow oil (5.2 g, 92% yield) that was used without any further manipulation.

### Preparation of Intermediate 62

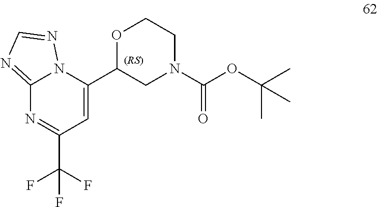 [SMILES: CC(C)(C)OC(=O)N1CCOC(c2cc(C(F)(F)F)nc3ncnn23)C1]

Intermediate 16 (774.5 mg, 6.42 mmol) was added to a mixture of intermediate 61 (1.9 g, 5.84 mmol) in DMF (50 mL). The mixture was stirred at 60° C. for 72 h. Then a sat. NaCl solution was added. The product was extracted with EtOAc. The organic layer was separated, dried (Na₂SO₄), filtered and evaporated in vacuo affording intermediate 62 as a yellow oil (2.2 g, quantitative).

### Preparation of Intermediate 63

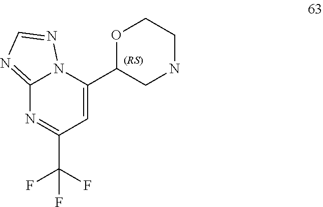 [SMILES: FC(F)(F)c1cc(C2CNCCO2)n2ncnc2n1]

Intermediate 62 (2.2 g, 5.89 mmol) was dissolved in ⁱPrOH (45 mL) and treated with HCl (2.95 mL; 6 M in 2-propanol) at rt and the mixture was further stirred at 100° C. for 1 h. The volatiles were evaporated in vacuo. The crude product was purified by flash chromatography (silica, 7M solution of ammonia in MeOH in DCM from 0/100 to 4/96). The desired fractions were collected and concentrated in vacuo affording intermediate 63 (0.58 g, 36%).

### Preparation of Intermediate 64

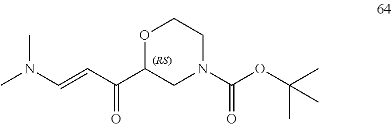 [SMILES: CN(C)/C=C/C(=O)C1CN(C(=O)OC(C)(C)C)CCO1]

A mixture of intermediate 60 (25 g, 109.04 mmol) in N,N-dimethylformamide dimethyl acetal (58 mL) was stirred at reflux overnight. The solvents were evaporated in vacuo affording intermediate 64 (30 g, 97% yield).

### Preparation of Intermediates 65, 66 and 67

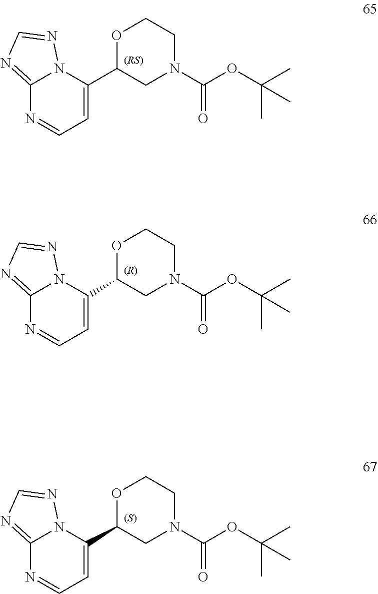

A solution of intermediate 64 (30 g, 105.50 mmol) and intermediate 16 (11.09 g, 131.88 mmol) in AcOH (52 mL) was stirred at reflux for 1 h. Then water was added and the mixture was extracted with Et₂O. The organic layer was washed with Na₂CO₃ (aq. sat. soltn.) and then with brine. The organic layer was separated, dried (MgSO₄), filtered and evaporated in vacuo. The residue was purified by flash chromatography (silica gel, eluent: DCM, 1% MeOH in DCM, 2%, 4%). The desired fractions were collected and concentrated in vacuo affording intermediate 65 (19 g, 59% yield). Intermediate 65 (2.83 g) was separated into enantiomers by purification by Prep SFC (Stationary phase: Chiralpak Diacel OJ-H 20×250 mm; mobile phase: CO₂, (MeOH)) yielding the corresponding enantiomers 66 and 67. Partial racemization was observed and both fractions were purified again by Prep SFC (Stationary phase: Chiralpak Diacel OJ-H 20×250 mm; mobile phase: (CO₂, MeOH) affording intermediate 66 (1 g) and intermediate 67 (1 g).

### Preparation of Intermediates 68

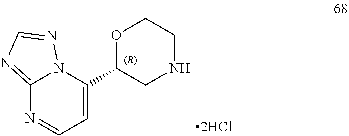

HCl (30 mL; 1 M in Et₂O) was added to a stirred solution of intermediate 66 (1 g, 3.27 mmol) in MeOH (200 mL) at rt and the mixture was further stirred at rt overnight. The volatiles were evaporated in vacuo affording the bishydrochloric acid salt of intermediate 68 (0.8 g, 88% yield).

### Preparation of Intermediates 69

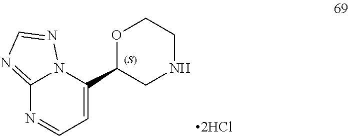

HCl (30 mL; 1 M in Et₂O) was added to a stirred solution of intermediate 67 (1 g, 3.27 mmol) in MeOH (200 mL) at rt and the mixture was further stirred at rt overnight. The volatiles were evaporated in vacuo. The product thus obtained was partly racemised and the free base was made because of solubility problems with the HCl salt before the purification by Prep SFC (Stationary phase: Chiralpak Diacel OJ 20×250 mm; mobile phase: (CO₂, MeOH) affording intermediate 69 which was treated with HCl in Et₂O and evaporated at 35° C. affording the bishydrochloric acid salt of intermediate 69 (0.5 g)

### Preparation of Intermediate 70

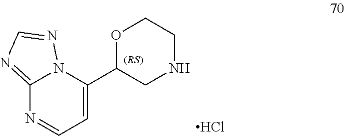 [SMILES: c1cc(C2CNCCO2)n2ncnc2n1 ; Cl]

HCl (6 M in iPrOH; 41 mL) was added to a stirred solution of intermediate 65 (6.7 g, 21.94 mmol) in DCM (32 mL). The mixture was stirred at rt for 16 h. The solvents were evaporated in vacuo affording the bishydrochloric acid salt of intermediate 70 (5.8 g, 95% yield).

### Preparation of Intermediate 71

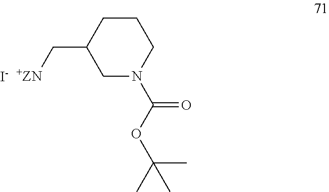

A solution of 3-iodomethylpiperidine-1-carboxylic acid tert-butyl ester (CAS: 253177-03-6; 3.58 g, 11 mmol) and LiCl (26 mL, 13 mmol, 0.5 M solution in THF) was pumped through a column containing activated Zn (12 g, 183.5 mmol) at 40° C. with flow of 0.5 mL/min. The outcome solution was collected under N₂ atmosphere to yield intermediate 71 (0.33 M after titration with 12) as a clear solution that was used without any further manipulation.

For the above reaction Zn was activated as follows: A solution of TMSCl (2 M) and 1-bromo-2-choroethane (0.3 M) in THF (10 mL) was passed through the column containing Zn at 40° C. with a flow of 1 mL/min.

### Preparation of Intermediate 72

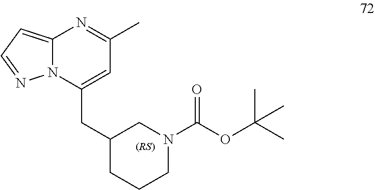 [SMILES: Cc1cc(CC2CCCN(C(=O)OC(C)(C)C)C2)n2nccc2n1]

Intermediate 71 (5.36 mL; 0.33 M in THF) was added to a solution of 7-chloro-5-methyl-pyrazolo[1,5-a]pyrimidine (CAS 16082-27-2; 118.7 mg, 0.71 mmol) and bis(tri-tert-butylphosphine)palladium(0) (CAS: 53199-31-8; 18.1 mg, 0.035 mmol) in THF (0.5 mL) at rt under nitrogen atmosphere. The mixture was stirred at rt overnight. The crude was quenched with water and extracted with EtOAc. The organic layer was separated, dried (Na₂SO₄), filtered and the solvent evaporated in vacuo. The residue was purified by flash chromatography (silica, EtOAc in heptane from 0/100 to 70/30). The desired fractions were collected and concentrated in vacuo affording intermediate 72 as an orange oil (49 mg, 20%).

### Preparation of Intermediate 73

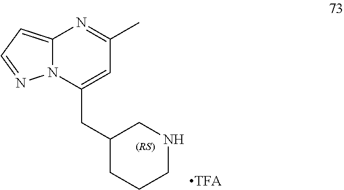 [SMILES: Cc1cc(CC2CCCNC2)n2nccc2n1 ; "·TFA"]

TFA (0.13 mL) was added to a stirred solution of intermediate 72 (49 mg, 0.11 mmol) in DCM (0.2 mL). The mixture was stirred for 3 h. The volatiles were evaporated in vacuo yielding the trifluoroacetate salt of intermediate 73 (25 mg, 96% yield) that was used without any further manipulation.

### Preparation of Intermediate 74

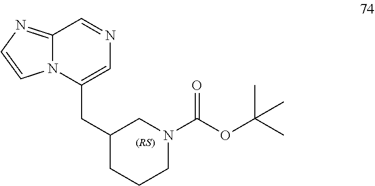 [SMILES: CC(C)(C)OC(=O)N1CCCC(Cc2cncc3nccn23)C1]

This compound was prepared through a procedure analogous to that described for the synthesis of intermediate 72 using 5-bromoimidazo[1,2-a]pyrazine (prepared through the procedure described in Org. Lett., 2015, 17 (12), 2886-2889) as starting material.

### Preparation of Intermediate 75

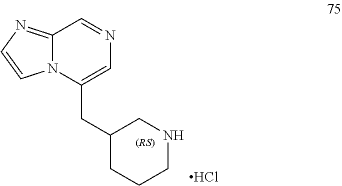 [SMILES: c1cn2c(CC3CCCNC3)cncc2n1 ; Cl]

HCl (0.19 mL; 4 M in dioxane) was added to a stirred solution of intermediate 74 (25 mg, 0.08 mmol) in MeOH (0.625 mL) at rt and the mixture was further stirred at rt for 24 h. The volatiles were evaporated in vacuo to give the hydrochloric acid salt of intermediate 75 as a brown solid (20 mg, quantitative).

### Preparation of Intermediate

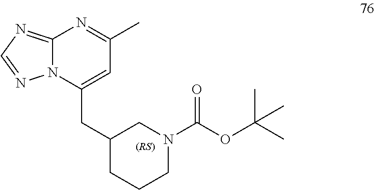 [SMILES: Cc1cc(CC2CCCN(C(=O)OC(C)(C)C)C2)n2ncnc2n1]

This compound was prepared through a procedure analogous to that described for the synthesis of intermediate 72 using 7-chloro-5-methyl-[1,2,4]triazolo[1,5-a]pyrimidine as starting material.

### Preparation of Intermediate 77

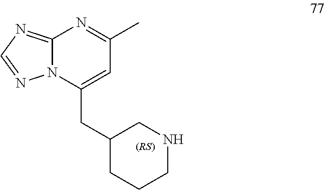 [SMILES: Cc1cc(CC2CCCNC2)n2ncnc2n1]

This compound was prepared through a procedure analogous to that described for the synthesis of intermediate 73 using intermediate 76 as starting material.

### Preparation of Intermediate 78

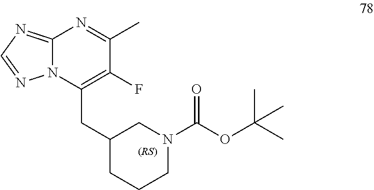 [SMILES: Cc1nc2ncnn2c(CC2CCCN(C(=O)OC(C)(C)C)C2)c1F]

This compound was prepared through a procedure analogous to that described for the synthesis of intermediate 72 using 7-chloro-6-fluoro-5-methyl-[1,2,4]triazolo[1,5-a]pyrimidine (prepared through the procedure described in PCT Int. App., 2010018868) as starting material

### Preparation of Intermediate 79

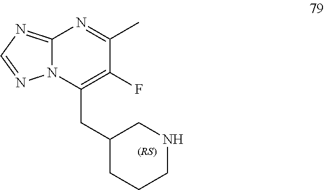 [SMILES: Cc1nc2ncnn2c(CC2CCCNC2)c1F]

This compound was prepared through a procedure analogous to that described for the synthesis of intermediate 73 using intermediate 78 as starting material.

### Preparation of Intermediate 80

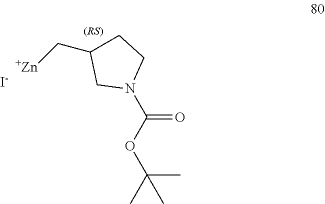

A solution of 3-iodomethylpyrrolidine-1-carboxylic acid tert-butyl ester (CAS: 479622-36-1; 4 g, 12.85 mmol) and LiCl (25 mL, 12.5 mmol, 0.5 M solution in THF) in THF (4 mL) was pumped through a column containing activated Zn (12 g, 183.5 mmol) at 40° C. with flow of 0.5 mL/min. The reagent was recovered pumping through the column THF (5 mL) at 40° C. with flow of 0.5 mL/min. The outcome solution was collected under N₂ atmosphere to yield intermediate 80 (0.3 M after titration with 12) that was used without any further manipulation.

For the above reaction Zn was activated as follows: A solution of TMSCl (2 M) and 1-bromo-2-choroethane (0.3 M) in THF (10 mL) was passed through the column containing Zn at 40° C. with a flow of 1 mL/min.

Intermediate (3S)-I-80 was prepared following the same reaction procedure as for 1-80 but starting from 3S-iodomethylpyrrolidine-1-carboxylic acid tert-butyl ester (CAS: 224168-68-7).

### Preparation of Intermediate 81

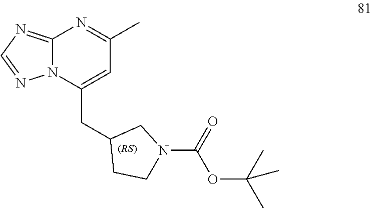 [SMILES: Cc1cc(CC2CCN(C(=O)OC(C)(C)C)C2)n2ncnc2n1]

This compound was prepared through a procedure analogous to that described for the synthesis of intermediate 72 using 7-chloro-5-methyl-[1,2,4]triazolo[1,5-a]pyrimidine and intermediate 80 as starting materials.

### Preparation of Intermediate 82

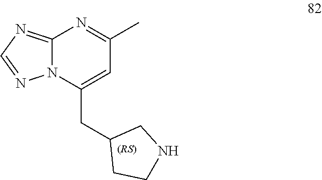 [SMILES: Cc1cc(CC2CCNC2)n2ncnc2n1]

This compound was prepared through a procedure analogous to that described for the synthesis of intermediate 73 using intermediate 81 as starting material.

### Preparation of Intermediate 83

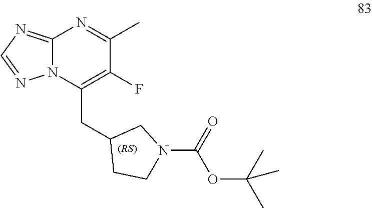 [SMILES: Cc1nc2ncnn2c(CC2CCN(C(=O)OC(C)(C)C)C2)c1F]

This compound was prepared through a procedure analogous to that described for the synthesis of intermediate 72 using intermediate 80 and 7-chloro-6-fluoro-5-methyl-[1,2,4]triazolo[1,5-a]pyrimidine (prepared through the procedure described in PCT Int. App., 2010018868) as starting materials.

### Preparation of Intermediate 84

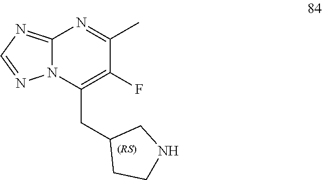 [SMILES: Cc1nc2ncnn2c(CC2CCNC2)c1F]

This compound was prepared through a procedure analogous to that described for the synthesis of intermediate 73 using intermediate 83 as starting material.

### Preparation of Intermediate 85

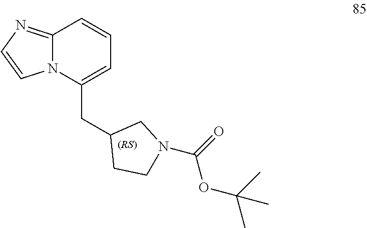 [SMILES: CC(C)(C)OC(=O)N1CCC(Cc2cccc3nccn23)C1]

This compound was prepared through a procedure analogous to that described for the synthesis of intermediate 72 using intermediate 80 and 5-bromoimidazo[1,2-a]pyridine (CAS: 69214-09-1) as starting materials.

### Preparation of Intermediate 86

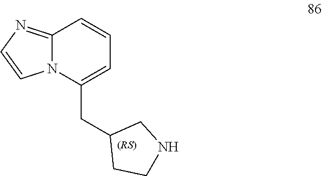 [SMILES: c1cc(CC2CCNC2)n2ccnc2c1]

This compound was prepared through a procedure analogous to that described for the synthesis of intermediate 75 using intermediate 85 as starting material.

### Preparation of Intermediate 87

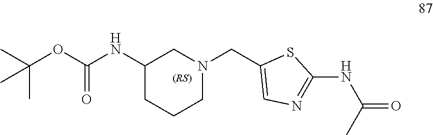 [SMILES: CC(=O)Nc1ncc(CN2CCCC(NC(=O)OC(C)(C)C)C2)s1]

Diisopropylethylamine (0.52 mL, 3 mmol) was added to a mixture of tert-butyl N-(3-piperidyl)carbamate (200 mg, 1 mmol) in DCM (30 mL) and the mixture was stirred at rt for 10 minutes. Then intermediate 38 (203.94 mg, 1.2 mmol) was added and the resulting suspension was stirred for 2 h. Then sodium triacetoxyborohydride (423.29 mg, 2 mmol) was added and the mixture was stirred at rt for 64 h. Then water was added and the organic layer was separated, dried (MgSO₄), filtered and concentrated in vacuo. The crude product was purified by flash chromatography (silica, MeOH in DCM from 0/100 to 15/85). The desired fractions were collected and concentrated in vacuo affording intermediate 87 as a pale yellow oil (327 mg, 92% yield).

### Preparation of Intermediate 88

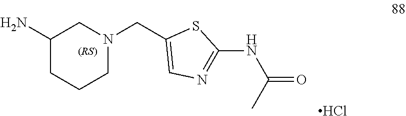 [SMILES: CC(=O)Nc1ncc(CN2CCCC(N)C2)s1 ; Cl]

HCl (4.61 mL; 4 M in dioxane) was added to a stirred solution of intermediate 87 (327 mg, 0.92 mmol) in 1,4-dioxane (25 mL) at rt and the mixture was further stirred at rt for 18 h. The volatiles were evaporated in vacuo and the residue thus obtained was treated with EtOAc to give a pale brown gum which was separated from the organic phase and dried in the oven to yield the bishydrochloric acid salt of intermediate 88 (350 mg, quantitative).

### Preparation of Intermediate 89

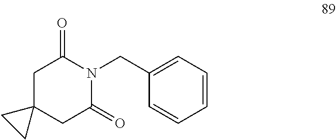 [SMILES: O=C1CC2(CC2)CC(=O)N1Cc1ccccc1]

K₂CO₃ (8.83 g, 63.88 mmol) followed by BnBr (6 g, 35.13 mmol) were added to a solution of 6-azaspiro[2.5]octane-5,7-dione (prepared through the procedure described in PCT Int. App., 2010026989; 4.44 g, 31.91 mmol) in acetone (113 mL). The mixture was stirred at 50° C. for 4 h. Then the mixture was cooled to rt and stirred at rt overnight. The white precipitate was filtered and the filtrate was concentrated in vacuo. The resulting residue was crystallized from heptane. The crystals were filtered off and dried affording intermediate 89 (6.06 g, 83% yield).

### Preparation of Intermediate 90

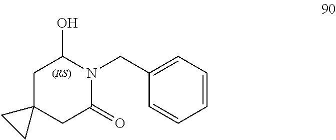 [SMILES: O=C1CC2(CC2)CC(O)N1Cc1ccccc1]

Diisobutylaluminum hydride (1 M in DCM; 99.44 mL, 99.44 mmol) was added to a stirred solution of intermediate 89 (11.4 g, 49.72 mmol) in DCM (252 mL) at −78° C. The mixture was stirred at −78° C. for 2 h. Then the mixture was quenched at −78° C. with Rochelle's salt (3M, 2 mL) and then it was allowed to warm to rt. The mixture was diluted with water. The white salts obtained were filtered through celite and washed with DCM. The filtrate was evaporated in vacuo. The residue was taken into EtOAc and water. The aqueous layer was separated and further extracted with EtOAc (×2). The combined organic layers were dried (MgSO₄), filtered and concentrated in vacuo. The crude product was purified by flash chromatography (silica, MeOH in DCM from 0/100 to 3/97). The desired fractions were collected and concentrated in vacuo affording intermediate 90 (9 g, 78% yield).

### Preparation of Intermediate 91

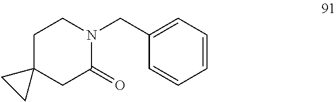 [SMILES: O=C1CC2(CCN1Cc1ccccc1)CC2]

TFA (19.95 mL) followed by triethylsilane (19.89 mL, 124.52 mmol) were added to a mixture of intermediate 90 (9 g, 38.91 mmol) in DCM (91 mL) at 0° C. Then the mixture was allowed to warm to rt and further stirred for 1 h. The reaction mixture was washed with NaHCO₃ (aq. sat. soltn.). The organic layer was separated, dried (MgSO₄), filtered and concentrated in vacuo. The crude product was purified by flash chromatography (silica, MeOH in DCM from 0/100 to 2/98). The desired fractions were collected and concentrated in vacuo affording intermediate 91 (6.69 g, 80% yield)

### Preparation of Intermediate 92

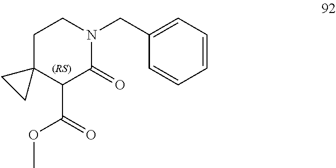 [SMILES: COC(=O)C1C(=O)N(Cc2ccccc2)CCC12CC2]

BuLi (2.5 M in hexanes; 18.57 mL, 46.42 mmol) was added to a mixture of diisopropylamine (4.9 g, 48.44 mmol) in THF (300 mL) at −78° C. The mixture was stirred at −30° C. for 10 minutes and then cooled to −78° C. Then intermediate 91 (8.69 g, 40.36 mmol) in some THF was added and the mixture was further stirred for 30 minutes. Then, methyl chloroformate (5.39 g, 56.51 mmol) was added and the mixture was allowed to warm to rt. Then, NH₄Cl (aq. sat. soltn.) was added and the product was extracted with Et₂O. The organic layer was separated and washed with brine. The organic layer was separated, dried (MgSO₄), filtered and concentrated in vacuo affording intermediate 92 (11 g, 99.7% yield) that was used without any further manipulation

### Preparation of Intermediate 93

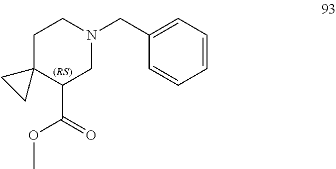 [SMILES: COC(=O)C1CN(Cc2ccccc2)CCC12CC2]

Trimethyloxonium tetrafluoroborate (CAS: 420-37-1; 17.86 g, 120.73 mmol) was added to a solution of intermediate 92 (11 g, 40.24 mmol) and 2,6-di-tert-butylpyridine (CAS: 585-48-8; 25.41 g, 132.81 mmol) in dry DCM (938 mL) under nitrogen atmosphere. The mixture was stirred at rt overnight. Then the solution was cooled to 0° C. and dry MeOH (626 mL) followed by sodium borohydride (15.22 g, 402.44 mmol) were added. The mixture was stirred at 0° C. for 30 minutes. Then the mixture was quenched by the addition of NaHCO₃ (aq. sat. soltn.) and the product was extracted with DCM. The organic layer was separated, dried (MgSO₄), filtered and concentrated in vacuo. The crude product was purified by flash chromatography (silica, MeOH in DCM from 0/100 to 3/97). The desired fractions were collected and concentrated in vacuo affording intermediate 93 (6.62 g, 63% yield).

### Preparation of Intermediate 94

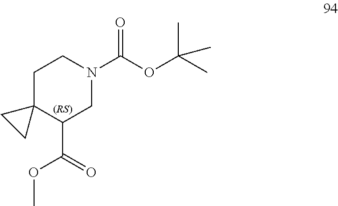 [SMILES: COC(=O)C1CN(C(=O)OC(C)(C)C)CCC12CC2]

A hydrogenation flask was charged with 10% palladium on carbon (4.07 g, 3.83 mmol) under nitrogen atmosphere. Then MeOH (892 mL) was added. Intermediate 93 (6.62 g, 25.53 mmol) followed by di-tert-butyl dicarbonate (6.89 g, 30.63 mmol) and Et₃N (4.43 mL, 31.91 mmol) were sequentially added. The reaction mixture was placed under hydrogen and stirred at rt. The reaction mixture was filtered off and evaporated in vacuo. The crude product was purified by flash chromatography (silica, MeOH in DCM from 0/100 to 2/98). The desired fractions were collected and concentrated in vacuo affording intermediate 94 (6.06 g, 88% yield).

### Preparation of Intermediate 95

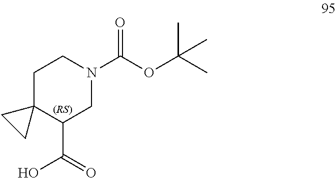 [SMILES: CC(C)(C)OC(=O)N1CCC2(CC2)C(C(=O)O)C1]

1 M NaOH (129 mL) was added to a mixture of intermediate 94 (5.8 g, 21.53 mmol) in MeOH (51 mL) and the mixture was stirred at rt overnight. Then the solvents were evaporated in vacuo. The residue was acidified with 1N HCl (129 mL) and the product was extracted with CHCl₃. The organic layer was separated, dried (MgSO₄), filtered and concentrated in vacuo. The product was crystallized from DIPE. The crystals were filtered off and dried affording intermediate 95 (5.29 g, 96% yield).

### Preparation of Intermediate 96

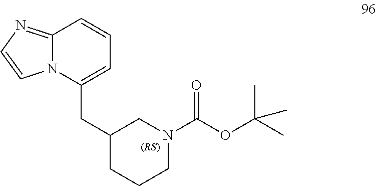 [SMILES: CC(C)(C)OC(=O)N1CCCC(Cc2cccc3nccn23)C1]

This compound was prepared through a procedure analogous to that described for the synthesis of intermediate 72 using 5-bromoimidazo[1,2-a]pyridine (CAS: 69214-09-1) as starting material.

### Preparation of Intermediate 97

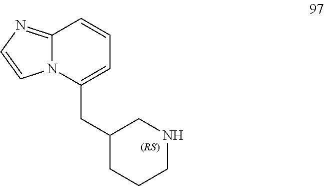 [SMILES: c1cc(CC2CCCNC2)n2ccnc2c1]

This compound was prepared through a procedure analogous to that described for the synthesis of intermediate 75 using intermediate 96 as starting material.

### Preparation of Intermediate 98

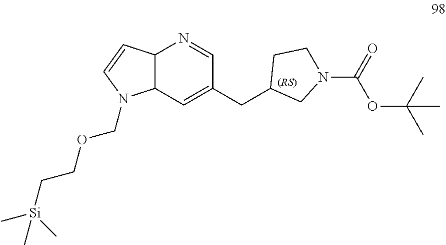 [SMILES: CC(C)(C)OC(=O)N1CCC(CC2=CC3C(C=CN3COCC[Si](C)(C)C)N=C2)C1]

This compound was prepared through a procedure analogous to that described for the synthesis of intermediate 51 using intermediate 80 and 2-[(6-bromo-3a,7a-dihydropyrrolo[3,2-b]pyridin-1-yl)methoxy]ethyl-trimethyl-silane (prepared through the procedure described in PCT Int. App., 2012037298) as starting materials.

### Preparation of Intermediate 99

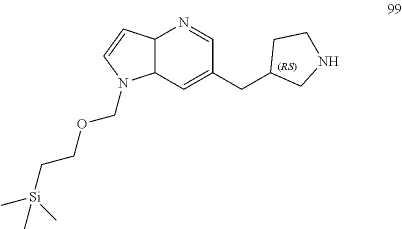 [SMILES: C[Si](C)(C)CCOCN1C=CC2N=CC(CC3CCNC3)=CC21]

This compound was prepared through a procedure analogous to that described for the synthesis of intermediate 47 using intermediate 98 as starting material.

### Preparation of Intermediate 100

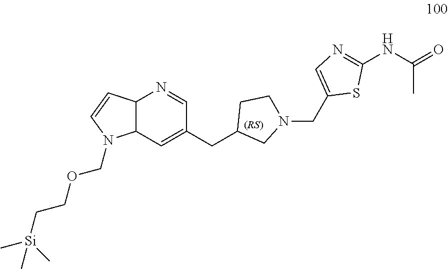 [SMILES: CC(=O)Nc1ncc(CN2CCC(CC3=CC4C(C=CN4COCC[Si](C)(C)C)N=C3)C2)s1]

Sodium triacetoxyborohydride (116.99 mg, 0.55 mmol) was added to a solution of intermediate 99 (122 mg, 0.37 mmol), intermediate 38 (75.15 mg, 0.44 mmol) in MeOH (0.62 mL) and DCM (0.62 mL). The mixture was stirred at rt for 16 h. Then the mixture was concentrated in vacuo. The crude product was purified by flash chromatography (silica, 7M solution of ammonia in MeOH in DCM from 0/100 to 3/97). The desired fractions were collected and concentrated in vacuo affording intermediate 100 as a yellow oil (80 mg, 45% yield).

### Preparation of Intermediate 101

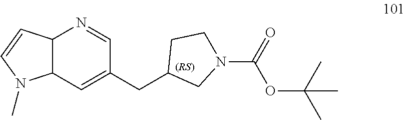 [SMILES: CN1C=CC2N=CC(CC3CCN(C(=O)OC(C)(C)C)C3)=CC21]

This compound was prepared through a procedure analogous to that described for the synthesis of intermediate 51 using intermediate 80 and 6-bromo-1-methyl-3a,7a-dihydropyrrolo[3,2-b]pyridine (prepared through the procedure described in PCT Int. App., 2015128333) as starting materials.

### Preparation of Intermediate 102

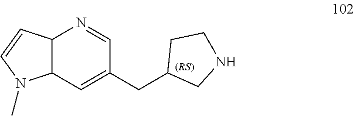 [SMILES: CN1C=CC2N=CC(CC3CCNC3)=CC21]

TFA (0.9 mL) was added to a stirred solution of intermediate 101 (149 mg, 0.47 mmol) in DCM (0.9 mL). The mixture was stirred for 16 h. The solvents were evaporated in vacuo. Then EtOAc was added and the mixture was washed with NaHCO₃ (aq. sat. soltn.). The product was isolated in the aqueous layer which was purified by ion exchange chromatography (Isolute® SCX2 eluting first with MeOH and then with 7N NH₃ in MeOH). The desired fractions were collected and concentrated in vacuo affording intermediate 102 (36 mg, 35% yield).

### Preparation of Intermediate 103

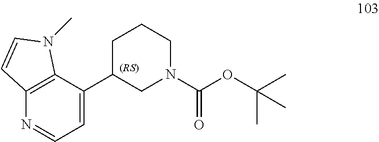 [SMILES: Cn1ccc2nccc(C3CCCN(C(=O)OC(C)(C)C)C3)c21]

NaH (21.9 mg, 0.55 mmol, 60% dispersion in mineral oil) was added to a stirred solution of intermediate 43 (150 mg, 0.5 mmol) in DMF (0.5 mL) at 0° C. The mixture was stirred at 0° C. for 30 minutes. Then the mixture was cooled to 0° C. and methyl iodide (0.034 mL, 0.55 mmol) was added. The mixture was stirred at 0° C. for 30 minutes and at rt for 1 h. The mixture was diluted with water and extracted with EtOAc. The organic layer was separated, washed with NaCl (aq. sat. soltn.), dried (Na₂SO₄), filtered and concentrated in vacuo. The crude product was purified by flash chromatography (silica, MeOH in DCM from 0/100 to 5/95). The desired fractions were collected and concentrated in vacuo affording intermediate 103 (54 mg, 34% yield).

### Preparation of Intermediate 104

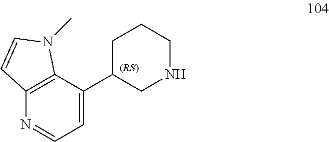 [SMILES: Cn1ccc2nccc(C3CCCNC3)c21]

This compound was prepared through a procedure analogous to that described for the synthesis of intermediate 34 using intermediate 103 as starting material.

### Preparation of Intermediate 105

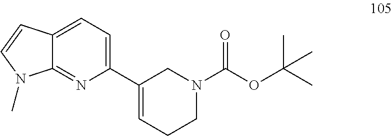 [SMILES: Cn1ccc2ccc(C3=CCCN(C(=O)OC(C)(C)C)C3)nc21]

This compound was prepared through a procedure analogous to that described for the synthesis of intermediate 56 using 6-chloro-1-methyl-pyrrolo[2,3-b]pyridine (prepared through the procedure described in J. Med. Chem., 58 (23), 9382-9394, 2015) as starting material.

### Preparation of Intermediate 106

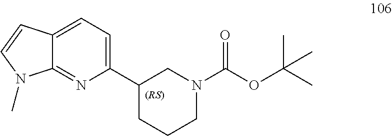 [SMILES: Cn1ccc2ccc(C3CCCN(C(=O)OC(C)(C)C)C3)nc21]

A solution of intermediate 105 (280 mg, 0.89 mmol) in EtOH (20 mL) was hydrogenated in a H-cube® reactor (1.3 mL/min., 70 mm Pd/C 10% cartridge, full H₂ mode, 50° C., 1 cycle). The solvent was evaporated in vacuo and the crude product was purified by flash chromatography (silica, EtOAc in heptane from 0/100 to 50/50). The desired fractions were collected and concentrated in vacuo affording intermediate 106 as a colorless oil (113 mg, 40% yield).

### Preparation of Intermediate 107

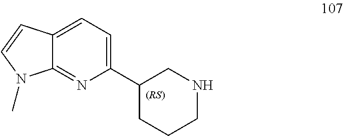 [SMILES: Cn1ccc2ccc(C3CCCNC3)nc21]

TFA (0.25 mL) was added to a stirred solution of intermediate 106 (104 mg, 0.33 mmol) in DCM (1 mL). The mixture was stirred for 4 h. The solvents were evaporated in vacuo. The crude product was purified by ion exchange chromatography (Isolute® SCX2 eluting first with MeOH and then with 7N NH₃ in MeOH). The desired fractions were collected and concentrated in vacuo affording intermediate 107 as a pale yellow oil (68.7 mg, 97% yield).

### Preparation of Intermediate 108

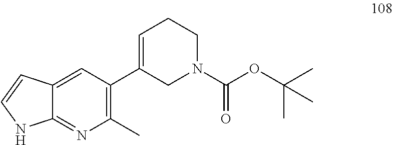 [SMILES: Cc1nc2[nH]ccc2cc1C1=CCCN(C(=O)OC(C)(C)C)C1]

This compound was prepared through a procedure analogous to that described for the synthesis of intermediate 56 using 5-bromo-6-methyl-1H-pyrrolo[2,3-b]pyridine (prepared through the procedure described PCT Int. App., 2007135398) as starting material.

### Preparation of Intermediate 109

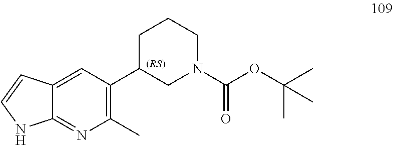 [SMILES: Cc1nc2[nH]ccc2cc1C1CCCN(C(=O)OC(C)(C)C)C1]

This compound was prepared through a procedure analogous to that described for the synthesis of intermediate 57 using intermediate 108 as starting material.

### Preparation of Intermediate 110

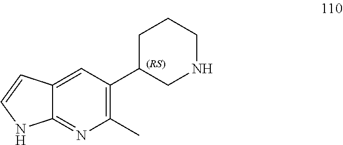 [SMILES: Cc1nc2[nH]ccc2cc1C1CCCNC1]

This compound was prepared through a procedure analogous to that described for the synthesis of intermediate 50 using intermediate 109 as starting material.

### Preparation of Intermediate 111

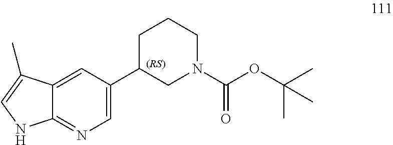 [SMILES: Cc1c[nH]c2ncc(C3CCCN(C(=O)OC(C)(C)C)C3)cc12]

This compound was prepared through a procedure analogous to that described for the synthesis of intermediate 103 using intermediate 57 as starting material.

### Preparation of Intermediate 112

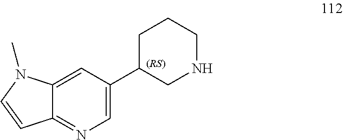 [SMILES: Cn1ccc2ncc(C3CCCNC3)cc21]

This compound was prepared through a procedure analogous to that described for the synthesis of intermediate 34 using intermediate 111 as starting material.

### Preparation of Intermediate 113

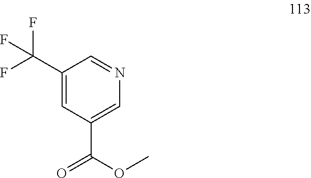

An autoclave was charged with 3-bromo-5-(trifluoromethyl)pyridine (CAS: 436799-33-6; 24 g, 106.2 mmol), palladium (II) acetate (1.25 g, 5.57 mmol), 1,3-bis(diphenylphosphino)propane (CAS: 6737-42-4; 1.19 g, 2.79 mmol), Et₃N (15.38 mL, 111 mmol) and MeOH (96 mL). Then it was flushed three times with CO and then pressurized with 3 MPa (30 bar) CO (3.13 g, 111.8 mmol). The reaction mixture was heated at 120° C. for 16 h. After cooling, the mixture was transferred to a flask and concentrated in vacuo. The residue was diluted with DCM and washed with water. The organic layer was separated, dried (MgSO₄), filtered and concentrated in vacuo. The crude product was purified by flash chromatography (silica, MeOH in DCM from 0/100 to 2/98). The desired fractions were collected and concentrated in vacuo affording intermediate 113 (18.81 g, 86% yield).

### Preparation of Intermediate 114

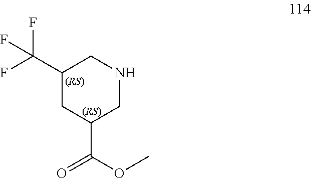

A hydrogenation flask was charged with AcOH (500 mL) and then 10% palladium on active carbon (19.52 g, 18.34 mmol) was added. Intermediate 113 (18.81 g, 91.69 mmol) was added and the mixture was hydrogenated at 50° C. overnight. The mixture was filtered over Dicalite® and the filtrate was evaporated to yield intermediate 114 (19.3 g, 99.7% yield), which was used in the next step without further purification.

### Preparation of Intermediate 115

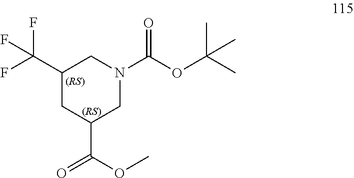 [SMILES: COC(=O)C1CC(C(F)(F)F)CN(C(=O)OC(C)(C)C)C1]

To a solution of intermediate 114 (19.3 g, 91.39 mmol) in DCM (240 mL), DIPEA (23.62 g, 182.78 mmol) and DMAP (1.12 g, 9.14 mmol) were added. Then di-tert-butyl dicarbonate (19.95 g, 91.39 mmol) was added to this solution in small portions and the reaction was stirred at RT for 1 h. The RM was washed with water and brine and the organic layer was dried over MgSO₄, filtered and evaporated. The product was purified by flash chromatograph (silica gel, eluent: DCM, 1% MeOH in DCM). The desired fractions were evaporated, yielding intermediate 115 (20.1 g, 71%).

### Preparation of Intermediate 116

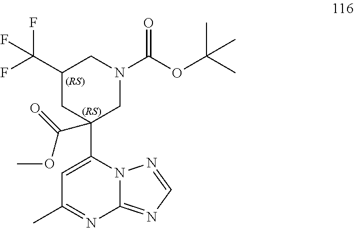 [SMILES: COC(=O)C1(c2cc(C)nc3ncnn23)CC(C(F)(F)F)CN(C(=O)OC(C)(C)C)C1]

LDA (2 M in cyclohexane/ethylbenzene/THF; 32.04 mL, 64.09 mmol) was added dropwise to a stirred solution of intermediate 115 (19 g, 61.03 mmol) in THF (244 mL) at −78° C. to −60° C. under nitrogen atmosphere. The mixture was stirred at −60° C. for 40 minutes. Then 7-chloro-5-methyl-[1,2,4]triazolo[1,5-a]pyrimidine (CAS: 24415-66-5; 10.29 g, 61.03 mmol) dissolved in the minimum amount of THF was added dropwise and the mixture was stirred at −60° C. to −70° C. for 1 h. The mixture was quenched with NH₄Cl (aq. sat. soltn.) and concentrated in vacuo. The residue was partitioned between EtOAc and water. The layers were separated and the aqueous layer was extracted with EtOAc (×2). The combined organic layers were dried (MgSO₄), filtered and concentrated in vacuo yielding intermediate 116 (27 g, 99.7% yield) used as such in the subsequent reaction step.

### Preparation of Intermediate 117

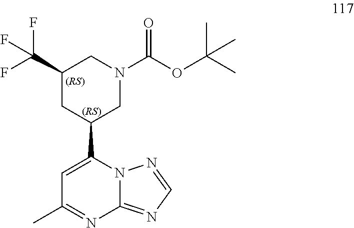 [SMILES: Cc1cc([C@H]2C[C@@H](C(F)(F)F)CN(C(=O)OC(C)(C)C)C2)n2ncnc2n1]

LiOH (21 g, 877.04 mmol) was added to a solution of intermediate 116 (27 g, 60.89 mmol) in MeOH (271 mL) and H₂O (271 mL). The mixture was stirred at 40° C. overnight. The mixture was concentrated in vacuo and the resulting aqueous residue was extracted with EtOAc (×3). The combined organic layers were dried (MgSO₄), filtered and concentrated in vacuo. The product was purified by flash chromatograph (silica gel, eluent: DCM, 2% MeOH in DCM). The desired fractions were evaporated and crystallized from Et₂O. The crystals were filtered off and dried affording intermediate 117 (14.91 g, 63% yield).

### Preparation of Intermediate 118

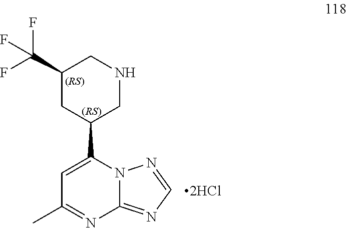 [SMILES: Cc1cc([C@@H]2CNC[C@H](C(F)(F)F)C2)n2ncnc2n1]

Intermediate 117 (14.91 g, 38.69 mmol) was dissolved in MeOH (408 mL) and treated with HCl (408 mL; 6 N in 2-propanol) at rt and the mixture was further stirred at rt overnight. The volatiles were evaporated in vacuo and the solid was crystallized from Et₂O. The crystals were filtered off and dried to give the bishydrochloric acid salt of intermediate 118 (15.01 g, quantitative).

### Preparation of Intermediates 119-130

The following compounds were prepared following a deprotection procedure like the one described for the preparation of intermediate 7 starting from the corresponding Boc-protected amine intermediates using hydrochloric acid or trifluoroacetic acid under standard reaction conditions known to the person skilled in the art.

| | BOC-PROTECTED INTERMEDIATE | |
|---|---|---|
| INTERMEDIATE AMINE | AMINE | ACID/SOLVENT |
| 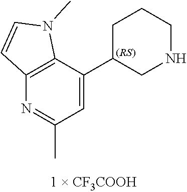 [SMILES: Cc1cc(C2CCCNC2)c2c(ccn2C)n1] | 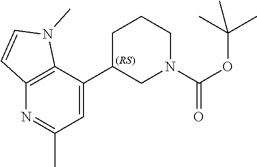 [SMILES: Cc1cc(C2CCCN(C(=O)OC(C)(C)C)C2)c2c(ccn2C)n1] | TFA/DCM |
| I-119 | I-131 | |
| 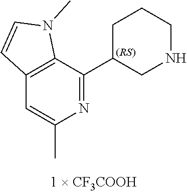 [SMILES: Cc1cc2ccn(C)c2c(C2CCCNC2)n1] | 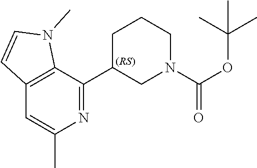 [SMILES: Cc1cc2ccn(C)c2c(C2CCCN(C(=O)OC(C)(C)C)C2)n1] | TFA/DCM |
| I-120 | I-132 | |
| 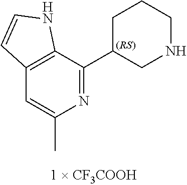 [SMILES: Cc1cc2cc[nH]c2c(C2CCCNC2)n1] | 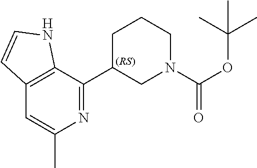 [SMILES: Cc1cc2cc[nH]c2c(C2CCCN(C(=O)OC(C)(C)C)C2)n1] | TFA/DCM |
| I-121 | I-133 | |
| 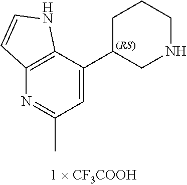 [SMILES: Cc1cc(C2CCCNC2)c2[nH]ccc2n1] | 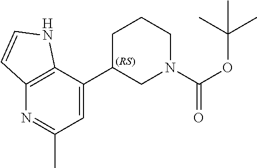 [SMILES: Cc1cc(C2CCCN(C(=O)OC(C)(C)C)C2)c2[nH]ccc2n1] | TFA/DCM |
| I-122 | I-134 | |
| 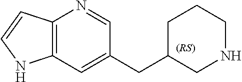 [SMILES: c1cc2ncc(CC3CCCNC3)cc2[nH]1] | 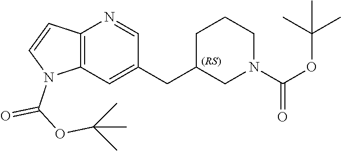 [SMILES: CC(C)(C)OC(=O)N1CCCC(Cc2cnc3ccn(C(=O)OC(C)(C)C)c3c2)C1] | HCl/1,4-dioxane |
| I-123 | I-135 | |
| 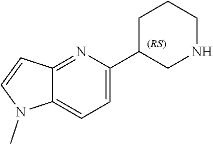 [SMILES: Cn1ccc2nc(C3CCCNC3)ccc21] | 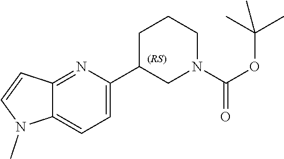 [SMILES: Cn1ccc2nc(C3CCCN(C(=O)OC(C)(C)C)C3)ccc21] | TFA/DCM |
| I-124 | I-136 | |
| 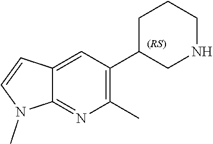 [SMILES: Cc1nc2c(ccn2C)cc1C1CCCNC1] | 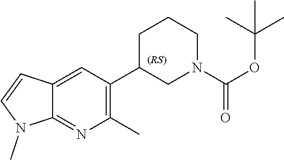 [SMILES: Cc1nc2c(ccn2C)cc1C1CCCN(C(=O)OC(C)(C)C)C1] | TFA/DCM |
| I-125 | I-137 | |
| 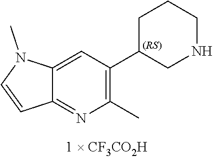 [SMILES: Cc1nc2ccn(C)c2cc1C1CCCNC1] | 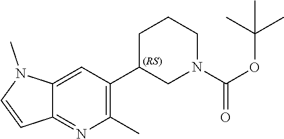 [SMILES: Cc1nc2ccn(C)c2cc1C1CCCN(C(=O)OC(C)(C)C)C1] | TFA/DCM |
| I-126 | I-139 | |
| 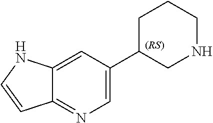 [SMILES: c1cc2ncc(C3CCCNC3)cc2[nH]1] | 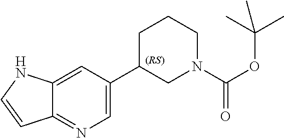 [SMILES: CC(C)(C)OC(=O)N1CCCC(c2cnc3cc[nH]c3c2)C1] | TFA/DCM |
| I-127 | I-140 | |
| 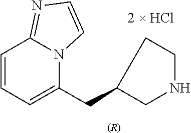 | 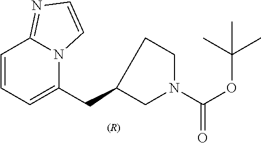 | HCl/1,4-dioxane |
| I-128 | I-141 | |
| 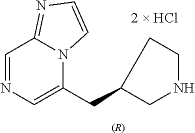 | 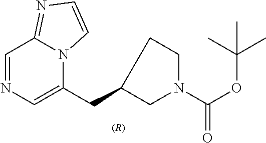 | HCl/1,4-dioxane |
| I-129 | I-142 | |
| 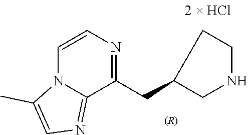 | 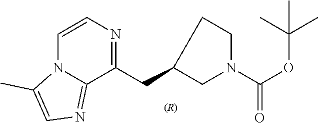 | HCl/1,4-dioxane |
| I-130 | I-138 | |
| 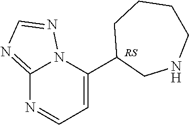 [SMILES: c1cc(C2CCCCNC2)n2ncnc2n1] | 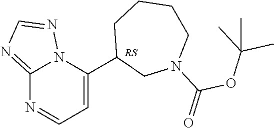 [SMILES: CC(C)(C)OC(=O)N1CCCCC(c2ccnc3ncnn23)C1] | HCl in iPrOH/MeOH |
| | 2HCl | |
| I-152 | I-151 | |

### Preparation of Intermediates 131, 132, 136, 137 and 139

The following compounds were prepared following a deprotection procedure like the one described for the preparation of intermediate 103 starting from the corresponding NH-azaindole intermediates using methyliodide under standard reaction conditions known to the person skilled in the art.

| BOC-PROTECTED INTERMEDIATE | INTERMEDIATE NH- |
|---|---|
| AMINE | AZAINDOLE |
| 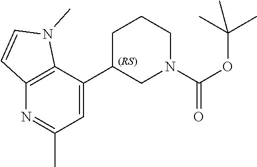 [SMILES: Cc1cc(C2CCCN(C(=O)OC(C)(C)C)C2)c2c(ccn2C)n1] | I-134 |
| I-131 | |
| 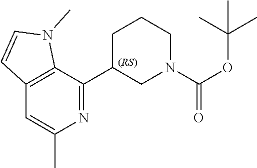 [SMILES: Cc1cc2ccn(C)c2c(C2CCCN(C(=O)OC(C)(C)C)C2)n1] | I-133 |
| I-132 | |
| 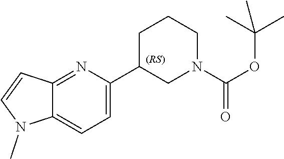 [SMILES: Cn1ccc2nc(C3CCCN(C(=O)OC(C)(C)C)C3)ccc21] | I-140 |
| I-136 | |
| 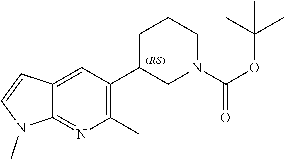 [SMILES: Cc1nc2c(ccn2C)cc1C1CCCN(C(=O)OC(C)(C)C)C1] | I-109 |
| I-137 | |
| 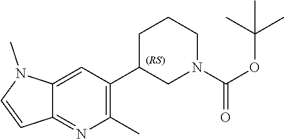 [SMILES: Cc1nc2ccn(C)c2cc1C1CCCN(C(=O)OC(C)(C)C)C1] | 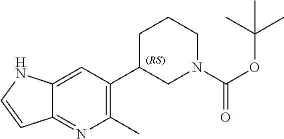 [SMILES: Cc1nc2cc[nH]c2cc1C1CCCN(C(=O)OC(C)(C)C)C1] |
| I-139 | I-143 |

### Preparation of Intermediates 133, 134, 140 and 143

The following compounds were prepared following a deprotection procedure like the one described for the preparation of intermediate 106 starting from the corresponding alkene under standard hydrogenation reaction conditions known to the person skilled in the art.

| BOC-PROTECTED | INTERMEDIATE NH- |
|---|---|
| INTERMEDIATE AMINE | AZAINDOLE |
| 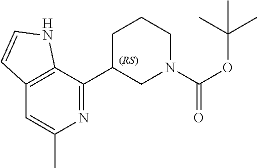 [SMILES: Cc1cc2cc[nH]c2c(C2CCCN(C(=O)OC(C)(C)C)C2)n1] | 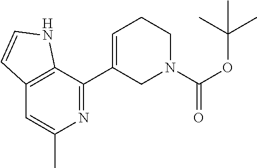 [SMILES: Cc1cc2cc[nH]c2c(C2=CCCN(C(=O)OC(C)(C)C)C2)n1] |
| I-133 | I-144 |
| 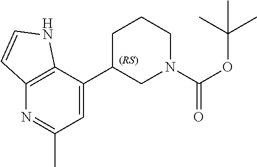 [SMILES: Cc1cc(C2CCCN(C(=O)OC(C)(C)C)C2)c2[nH]ccc2n1] | 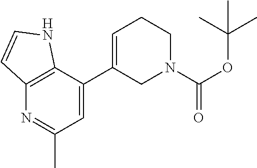 [SMILES: Cc1cc(C2=CCCN(C(=O)OC(C)(C)C)C2)c2[nH]ccc2n1] |
| I-134 | I-145 |
| 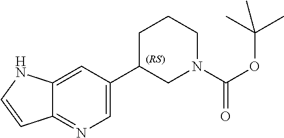 [SMILES: CC(C)(C)OC(=O)N1CCCC(c2cnc3cc[nH]c3c2)C1] | 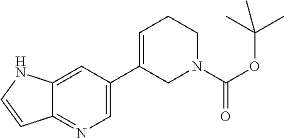 [SMILES: CC(C)(C)OC(=O)N1CCC=C(c2cnc3cc[nH]c3c2)C1] |
| I-140 | I-146 |
| 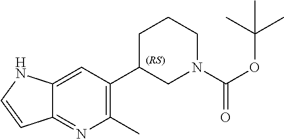 [SMILES: Cc1nc2cc[nH]c2cc1C1CCCN(C(=O)OC(C)(C)C)C1] | 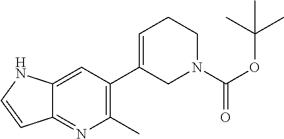 [SMILES: Cc1nc2cc[nH]c2cc1C1=CCCN(C(=O)OC(C)(C)C)C1] |
| I-143 | I-147 |

### Preparation of Intermediates 144-147

The following compounds were prepared following a deprotection procedure like the one described for the preparation of intermediate 42 starting from the corresponding halo-azaindole (halo: Cl or Br) intermediate and CAS 1251537-34-4 under standard palladium catalysed cross coupling reaction conditions known to the person skilled in the art.

| INTERMEDIATE NH- | |
|---|---|
| AZAINDOLE | HALO-AZAINDOLE |
| 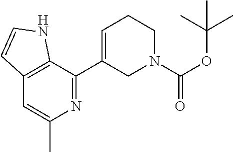 [SMILES: Cc1cc2cc[nH]c2c(C2=CCCN(C(=O)OC(C)(C)C)C2)n1] | CAS: 1379333-96-6 |
| I-144 | |
| 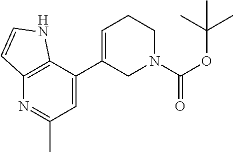 [SMILES: Cc1cc(C2=CCCN(C(=O)OC(C)(C)C)C2)c2[nH]ccc2n1] | CAS: 1486405-53-1 |
| I-145 | |
| 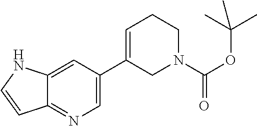 [SMILES: CC(C)(C)OC(=O)N1CCC=C(c2cnc3cc[nH]c3c2)C1] | CAS: 65156-94-7 |
| I-146 | |
| 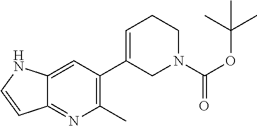 [SMILES: Cc1nc2cc[nH]c2cc1C1=CCCN(C(=O)OC(C)(C)C)C1] | 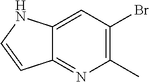 |
| I-147 | I-148 |

### Preparation of Intermediate 148

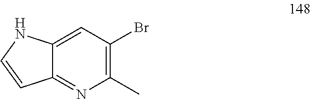

Intermediate 149 (430 mg, 1.67 mmol) was dissolved in acetic acid (7.16 mL) and the mixture was heated at 110° C. for 4 h. The volatiles were evaporated in vacuo and the residue thus obtained was purified by flash chromatography (SiO₂, MeOH in DCM, 0/100 to 2/98). The desired fractions were evaporated in vacio to yield intermediate 148 (330 mg, 93%) as an orange solid.

### Preparation of Intermediate 149

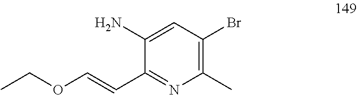

To a mixture of intermediate 150 (600 mg, 1.92 mmol) and trans-2-ethoxyvinylboronic acid pinacol ester (493.7 mg, 2.49 mmol) in DMF (5 mL) at rt and under N₂ atmosphere, lithium hydroxide (137.7 mg, 5.75 mmol) and [1,1′-bis(diphenylphosphino)ferrocene]dichloropalladium(II) complex with dichloromethane (CAS: 95464-05-4; 31.66 mg, 0.038 mmol) were added. The mixture was heated at 70° C. for 18 h. After cooling to rt water and EtOAc were added. The organic layer was separated and the volatiles were evaporated in vacuo. The residue thus obtained was purified by flash chromatography (SiO₂, EtOAc in heptane, 0/100 to 10/90). The desired fractions were evaporated in vacio to yield intermediate 149 (430 mg, 87%) as an oil.

### Preparation of Intermediate 150

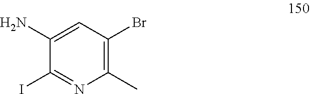

I₂ (570 mg, 2.25 mmol) and Ag₂SO₄ (733 mg, 2.35 mmol) were added to a mixture of 5-bromo-6-methylpyridin-3-amine (400 mg, 2.13 mmol) in EtOH (10 mL) at rt. The mixture was stirred at rt for 18 h. Then the solids were filtered off and the filtrate was were evaporated in vacuo. The residue thus obtained was taken up in DCM and NaOH (1N aqueous solution). The organic layer was separated, dried over Na₂SO₄, filtered and the volatiles were evaporated in vacuo to yield intermediate 150 (600 mg, 90%) as a solid.

### Preparation of Intermediates 138, 141 and 142

The following compounds were prepared following a deprotection procedure like the one described for the preparation of intermediate 72 starting from the corresponding halo-heterocycle (halo: Cl or Br) and organo zinc intermediate (3S)-80 under standard palladium catalysed cross coupling reaction conditions known to the person skilled in the art.

| | HALO- |
|---|---|
| | HETERO- |
| INTERMEDIATE | CYCLE |
| 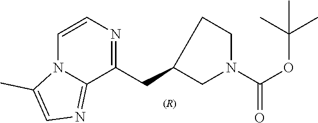 | CAS: 76537- 38-7 |
| I-138 | |
| 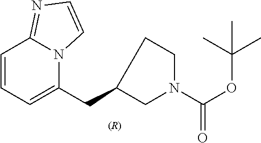 | CAS: 69214- 09-1 |
| I-141 | |
| 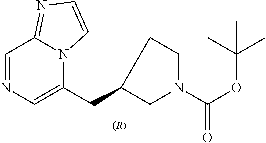 | CAS: 63744- 41-2 |
| I-142 | |

### Preparation of Intermediate 135

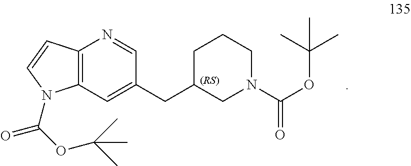 [SMILES: CC(C)(C)OC(=O)N1CCCC(Cc2cnc3ccn(C(=O)OC(C)(C)C)c3c2)C1]

A solution of 3-iodomethypiperidine-1-carboxylic acid tert-butyl ester (CAS: 253177-03-6; 0.4 g, 1.23 mmol) in LiCl (1.6 mL, 0.5M in THF) was pumped through a column containing activated Zinc at 0.5 mL/min and at 40° C. The outcome solution was collected over N₂ atmosphere to yield the organozinc intermediate 71 (in 4 mL of THF/LiCl). Then a solution of tert-butyl 6-bromo-1H-pyrrolo[3,2-b]pyridine-1-carboxylate (CAS: 1820711-82-7; 243.6 mg, 0.82 mmol) in LiCl (4 mL, 0.5M in THF) and the solution containing the organozinc intermediate 71, were pumped through a column containing siliacat DPP-Pd (1 g, loading 0.2-0.3 mmol/g) at 70° C. and 0.2 mL/min (each). The column was washed with 20 mL of THF. The outcome was diluted with EtOAc and washed with a mixture of sat. NH₄Cl and NH₄OH (4:1). The organic layer was separated, dried (MgSO₄), filtered and the filtrate concentrated in vacuo. The residue was purified by flash column chromatography (silica; EtOAc in heptane: 0/100 to 80/20). The desired fractions were collected and concentrated in vacuo to yield intermediate 135 (205 mg, 60%) as pale yellow oil.

### Preparation of Intermediate 153

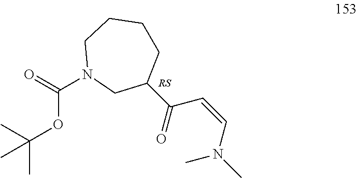 [SMILES: CN(C)/C=C\C(=O)C1CCCCN(C(=O)OC(C)(C)C)C1]

1,1-Dimethoxy-N,N-dimethyl-methanamine ([4637-24-5], 10 mL) was added to 3-acetylhexahydro-1H-azepine-1-carboxylic acid 1,1-dimethylethyl ester ([1782629-29-1], 4.21 g) and the mixture was refluxed overnight. The solvent was evaporated, yielding intermediate 153 (5 g), which was used without further purification.

### Preparation of Intermediate 151

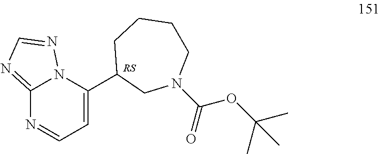 [SMILES: CC(C)(C)OC(=O)N1CCCCC(c2ccnc3ncnn23)C1]

A solution of intermediate 153 (5 g, 18.87 mmol) and 1H-1,2,4-triazol-5-amine (1.77 g, 21.09 mmol) in AcOH (10 mL) was stirred at reflux during 1 h. Water was added and the product was extracted with ether. The OL was washed with brine and dried (MgSO₄), filtered and evaporated. The product was purified by column chromatography (silicagel; eluent: DCM, 1%, 2% MeOH in DCM). The desired fractions were evaporated, yielding intermediate 151 (1.69 g, 32%).

### B. Preparation of the Final Compounds

### Preparation of Product 1

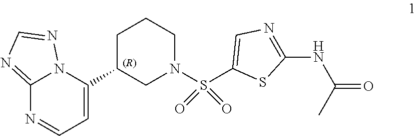

2-Acetylamino-thiazole-5-sulfonyl chloride (CAS: 654072-71-6, 50 mg, 0.21 mmol) was added portionwise to a stirred solution of intermediate 7 (61 mg, 0.22 mmol, bishydrochloric acid salt) and diisopropylethylamine (0.11 mL, 0.64 mmol) in DCM (7.81 mL) at 0° C. and the mixture was further stirred at 0° C. for 1 h. Then H₂O was added and the organic layer was separated, dried (MgSO₄), filtered and the solvents evaporated in vacuo. The product was purified by RP HPLC (Stationary phase: C18 XBridge 30×100 mm 5 μm, Mobile phase: Gradient from 81% 10 mM NH₄CO₃H pH 9 solution in water, 19% CH₃CN to 64% 10 mM NH₄CO₃H pH 9 solution in water, 36% CH₃CN). The desired fractions were collected and concentrated in vacuo affording product 1 as a white solid (81 mg, 96% yield).

### Preparation of Product 2

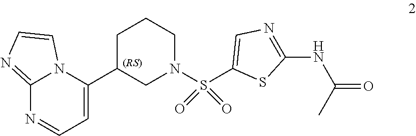

2-Acetylamino-thiazole-5-sulfonyl chloride (CAS: 654072-71-6, 44 mg, 0.18 mmol) was added portion wise to a stirred solution of intermediate 8 (50 mg, 0.18 mmol, bishydrochloric acid salt) and diisopropylethylamine (0.10 mL, 0.58 mmol) in DCM (0.51 mL) at 0° C. and the mixture was further stirred at 0° C. for 1 h. Then NaHCO₃ (aq. sat. soltn.) was added and the organic layer was separated, dried (MgSO₄), filtered and the solvents evaporated in vacuo. The crude product was washed with Et₂O and dried in the vacuum oven affording product 2 as a white solid (52 mg, 70% yield).

### Preparation of Product 3

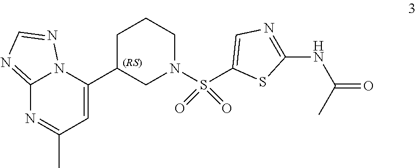

2-Acetylamino-thiazole-5-sulfonyl chloride (CAS: 654072-71-6, 41.5 mg, 0.17 mmol) was added portion wise to a stirred solution of intermediate 11 (50 mg, 0.17 mmol, bishydrochloric acid salt) and diisopropylethylamine (0.095 mL, 0.55 mmol) in DCM (0.48 mL) at 0° C. and the mixture was further stirred at 0° C. for 1 h. Then NaHCO₃ (aq. sat. soltn.) was added and organic layer was separated, dried (MgSO₄), filtered and the solvents evaporated in vacuo. The crude product was washed with Et₂O and dried in the vacuum oven affording product 3 as a white solid (42 mg, 58% yield).

### Preparation of Product 4

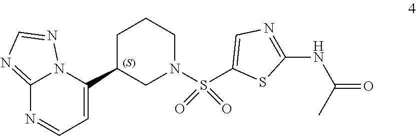

2-Acetylamino-thiazole-5-sulfonyl chloride (CAS: 654072-71-6, 50 mg, 0.21 mmol) was added portion wise to a stirred solution of intermediate 12 (65 mg, 0.23 mmol, bishydrochloric acid salt) and diisopropylethylamine (0.11 mL, 0.64 mmol) in DCM (0.5 mL) at 0° C. and the mixture was further stirred at 0° C. for 1 h. Then H₂O was added and organic layer was separated, dried (MgSO₄), filtered and the solvents evaporated in vacuo. The product was purified by RP HPLC (Stationary phase: C18 XBridge 30×100 mm 5 μm, Mobile phase: Gradient from 81% 10 mM NH₄CO₃H pH 9 solution in water, 19% CH₃CN to 64% 10 mM NH₄CO₃H pH 9 solution in water, 36% CH₃CN). The desired fractions were collected and concentrated in vacuo affording product 4 as a white solid (73 mg, 86% yield).

### Preparation of Product 5

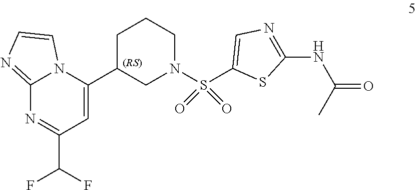

2-Acetylamino-thiazole-5-sulfonyl chloride (CAS: 654072-71-6, 52.5 mg, 0.22 mmol) was added portion wise to a stirred solution of intermediate 14 (50 mg, 0.20 mmol) and diisopropylethylamine (0.10 mL, 0.59 mmol) in DCM (0.5 mL) at 0° C. and the mixture was further stirred at 0° C. for 1 h. Then H₂O was added and the organic layer was separated, dried (MgSO₄), filtered and the solvents evaporated in vacuo. The crude product was triturated with Et₂O, filtered and dried in the vacuum oven affording product 5 as a white solid (57 mg, 63% yield).

### Preparation of Product 6

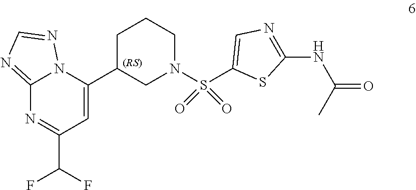

2-Acetylamino-thiazole-5-sulfonyl chloride (CAS: 654072-71-6, 52 mg, 0.22 mmol) was added portion wise to a stirred solution of intermediate 20 (50 mg, 0.20 mmol, bishydrochloric acid salt) and diisopropylethylamine (0.10 mL, 0.59 mmol) in DCM (0.5 mL) at 0° C. and the mixture was further stirred at 0° C. for 2 h. Then H₂O was added and the organic layer was separated, dried (MgSO₄), filtered and the solvents evaporated in vacuo. The product was purified by RP HPLC (Stationary phase: C18 XBridge 30×100 mm 5 um), Mobile phase: Gradient from 81% 10 mM NH₄CO₃H pH 9 solution in water, 19% CH₃CN to 64% 10 mM NH₄CO₃H pH 9 solution in water, 36% CH₃CN). The desired fractions were collected and concentrated in vacuo affording product 6 as a white solid (12 mg, 13% yield).

### Preparation of Product 7

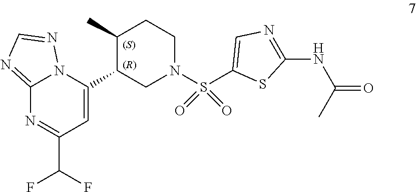

2-Acetylamino-thiazole-5-sulfonyl chloride (CAS: 654072-71-6, 50 mg, 0.21 mmol) was added portion wise to a stirred solution of intermediate 30 (71 mg, 0.23 mmol, bishydrochloric acid salt) and diisopropylethylamine (0.11 mL, 0.64 mmol) in DCM (0.5 mL) at 0° C. and the mixture was further stirred at 0° C. for 1 h. Then H₂O was added and the organic layer was separated, dried (MgSO₄), filtered and the solvents evaporated in vacuo. The product was purified by RP HPLC (Stationary phase: C18 XBridge 30×100 mm 5 μm, Mobile phase: Gradient from 47% 10 mM NH₄CO₃H pH 9 solution in water, 53% MeOH to 24% 10 mM NH₄CO₃H pH 9 solution in water, 76% MeOH). The desired fractions were collected and concentrated in vacuo affording product 7 as a yellow solid (12 mg, 12% yield).

### Preparation of Product 8

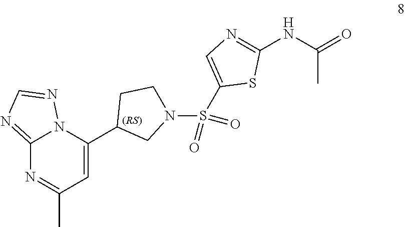

Diisopropylethylamine (0.20 mL, 1.14 mmol) was added to a solution of intermediate 34 (58 mg, 0.28 mmol) and 2-acetylamino-thiazole-5-sulfonyl chloride (CAS: 654072-71-6, 69 mg, 0.285 mmol) in DCM (0.5 mL) and the mixture was further stirred at room temperature for 2 h. Then the reaction was quenched with sat. NH₄Cl and extracted with DCM. The organic layer was separated, dried (Na₂SO₄), filtered and the solvents evaporated in vacuo. The product was purified by flash chromatography (silica, MeOH in DCM from 0/100 to 10/90). The desired fractions were collected and concentrated in vacuo. The residue was purified by RP HPLC (Stationary phase: C18 XBridge 30×100 mm 5 μm, Mobile phase: Gradient from 81% 0.1% NH₄CO₃H/NH₄OH pH 9 solution in water, 19% MeOH to 64% 0.1% NH₄CO₃H/NH₄OH pH 9 solution in water, 36% MeOH). The desired fractions were collected and concentrated in vacuo affording product 8 (13.5 mg, 10% yield).

### Preparation of Product 9

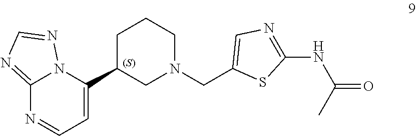

Triethylamine (1.01 mL, 7.26 mmol) was added to a stirred suspension of intermediate 12 (0.50 g, 1.82 mmol, bishydrochloric acid salt) in DCM (10 mL) in a sealed tube. The mixture was stirred at rt for 2 minutes. Then intermediate 38 (0.38 g, 2.24 mmol) followed by sodium triacetoxyborohydride (0.53 g, 2.53 mmol) were added. The mixture was stirred at rt for 22 h. Then additional sodium triacetoxyborohydride (0.23 g, 1.09 mmol) was added and the mixture was stirred at rt for 96 h. Then the mixture was diluted with NaHCO₃ (aq. sat. soltn.) and extracted with DCM. The organic layer was separated, dried (MgSO₄), filtered and concentrated in vacuo. The crude product was purified by flash chromatography (silica, 7M solution of ammonia in MeOH in DCM from 0/100 to 5/95). The desired fractions were collected and concentrated in vacuo. The desired fractions were collected and concentrated in vacuo affording product 9 as a yellow solid (227.6 mg, 35% yield).

### Preparation of Product 10

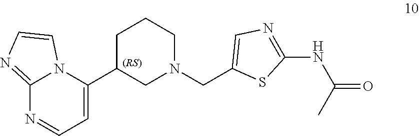

Sodium triacetoxyborohydride (47 mg, 0.22 mmol) was added to a solution of intermediate 8 (49 mg, 0.18 mmol, bishydrochloric acid salt), intermediate 38 (40 mg, 0.235 mmol) and triethylamine (0.08 mL, 0.58 mmol) in DCM (1 mL) under nitrogen atmosphere. The mixture was stirred at rt for 2 days. Then aqueous NaHCO₃ (aq. sat. soltn.) was added and the organic layer was separated, dried (MgSO₄), filtered and concentrated in vacuo. The crude product was purified by RP HPLC (Stationary phase: C18 XBridge 30×100 mm 5 μm, Mobile phase: Gradient from 47% 10 mM NH₄CO₃H pH 9 solution in water, 53% CH₃CN to 30% 10 mM NH₄CO₃H pH 9 solution in water, 70% CH₃CN). The desired fractions were collected and concentrated in vacuo affording product 10 as a yellow oil (26 mg, 41% yield).

### Preparation of Product 11

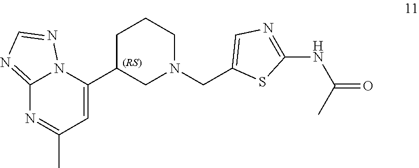

Sodium triacetoxyborohydride (40 mg, 0.19 mmol) was added to a solution of intermediate 11 (52 mg, 0.18 mmol, bishydrochloric acid salt), intermediate 38 (37 mg, 0.22 mmol) and triethylamine (0.07 mL, 0.52 mmol) in DCM (1 mL) under nitrogen atmosphere. The mixture was stirred at rt for 2 days. Then aqueous NaHCO₃ (aq. sat. soltn.) was added and the organic layer was separated, dried (MgSO₄), filtered and concentrated in vacuo. The crude product was purified by flash chromatography (silica, EtOAc in heptane from 0/100 to 80/20). The desired fractions were collected and concentrated in vacuo. The desired fractions were collected and concentrated in vacuo affording product 11 as a white solid (31 mg, 47% yield).

### Preparation of Product 12

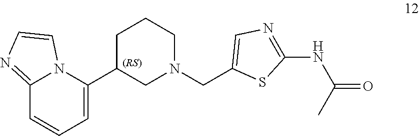

Sodium triacetoxyborohydride (46 mg, 0.22 mmol) was added to a solution of intermediate 41 (43 mg, 0.18 mmol, hydrochloric acid salt), intermediate 38 (37 mg, 0.22 mmol) and triethylamine (0.075 mL, 0.54 mmol) in DCM (1.1 mL) under nitrogen atmosphere. The mixture was stirred at rt for 4 days. Then aqueous NaHCO₃ (aq. sat. soltn.) was added and the organic layer was separated, dried (MgSO₄), filtered and concentrated in vacuo. The crude product was purified by RP HPLC (Stationary phase: C18 XBridge 30×150 mm 5 μm, Mobile phase: Gradient from 81% 10 mM NH₄CO₃H pH 9 solution in water, 19% CH₃CN to 64% 10 mM NH₄CO₃H pH 9 solution in water, 36% CH₃CN). The desired fractions were collected and concentrated in vacuo affording product 12 as a pale orange solid (6 mg, 9% yield).

### Preparation of Product 13

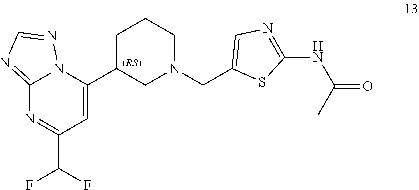

Intermediate 20 (50 mg, 0.15 mmol, bishydrochloric acid salt) was added to a stirred solution of N-[5-(chloromethyl)thiazol-2-yl]acetamide (prepared through the procedure described in WO2014/159234 A1; 35 mg, 0.15 mmol, hydrochloric acid salt) and triethylamine (0.11 mL, 0.77 mmol) in CH₃CN (0.5 mL). The mixture was stirred at rt for 2 h. Then, the mixture was filtered and the filtrate was diluted with EtOAc and washed with H₂O. The organic layer was separated, dried (Na₂SO₄), filtered and the solvents evaporated in vacuo affording product 13 as an orange solid (40 mg, 64% yield).

### Preparation of Product 14

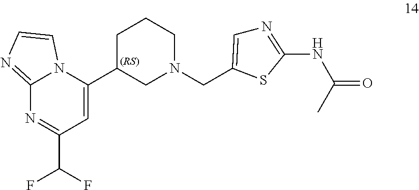 [SMILES: CC(=O)Nc1ncc(CN2CCCC(c3cc(C(F)F)nc4nccn34)C2)s1]

Intermediate 14 (50 mg, 0.15 mmol) was added to a stirred solution of N-[5-(chloromethyl)thiazol-2-yl]acetamide (prepared through the procedure described in WO2014/159234 A1; 35 mg, 0.15 mmol, hydrochloric acid salt) and triethylamine (0.06 mL, 0.46 mmol) in CH₃CN (0.5 mL). The mixture was stirred at rt for 2 h. Then, the mixture was filtered and the filtrate was diluted with EtOAc and washed with H₂O. The organic layer was separated, dried (Na₂SO₄), filtered and the solvents evaporated in vacuo. The crude product was purified by flash chromatography (silica, 7M solution of ammonia in MeOH in EtOAc from 0/100 to 5/95). The desired fractions were collected and concentrated in vacuo. The desired fractions were collected and concentrated in vacuo affording product 14 as a white solid (15 mg, 24% yield).

### Preparation of Product 15

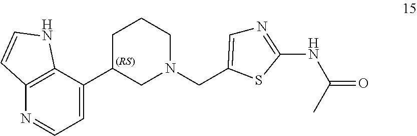 [SMILES: CC(=O)Nc1ncc(CN2CCCC(c3ccnc4cc[nH]c34)C2)s1]

Intermediate 38 (60.5 mg, 0.355 mmol) and sodium triacetoxyborohydride (205 mg, 0.97 mmol) were added to a solution of intermediate 44 (65 mg, 0.32 mmol) in DCM (5 mL). The mixture was stirred at rt for 18 h. Then aqueous NaHCO₃ (aq. sat. soltn.) was added and the organic layer was separated, dried (MgSO₄), filtered and concentrated in vacuo. The crude product was purified by RP HPLC (Stationary phase: C18 XBridge 30×100 mm 5 μm, Mobile phase: Gradient from 80% 0.1% NH₄CO₃H/NH₄OH pH 9 solution in water, 20% CH₃CN to 0% 0.1% NH₄CO₃H/NH₄OH pH 9 solution in water, 100% CH₃CN). The desired fractions were collected and concentrated in vacuo. The product was further purified by flash chromatography (silica, MeOH in DCM from 0/100 to 10/90). The desired fractions were collected and concentrated in vacuo. The product was dried under vacuum at 50° C. for 72 h affording product 15 as a light yellow solid (35 mg, 30.5% yield).

### Preparation of Product 16

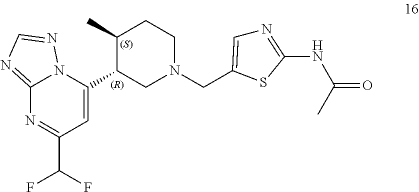

Sodium triacetoxyborohydride (46 mg, 0.22 mmol) was added to a solution of intermediate 30 (32 mg, 0.12 mmol), intermediate 38 (30 mg, 0.18 mmol) and triethylamine (0.06 mL, 0.43 mmol) in DCM (1 mL) under nitrogen atmosphere. The mixture was stirred at rt for 17 h. Then aqueous NaHCO₃ (aq. sat. soltn.) was added and the organic layer was separated, dried (MgSO₄), filtered and concentrated in vacuo. The crude product was purified by RP HPLC (Stationary phase: C18 XBridge 30×100 mm 5 μm, Mobile phase: Gradient from 60% 10 mM NH₄CO₃H pH 9 solution in water, 40% MeOH to 37% 10 mM NH₄CO₃H pH 9 solution in water, 63% MeOH). The desired fractions were collected and concentrated in vacuo affording product 16 as a yellow oil (8 mg, 16% yield).

### Preparation of Product 17

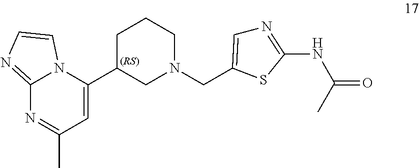 [SMILES: CC(=O)Nc1ncc(CN2CCCC(c3cc(C)nc4nccn34)C2)s1]

A suspension of intermediate 47 (45 mg, 0.21 mmol), intermediate 38 (46 mg, 0.27 mmol) in DCM (0.8 mL) was stirred at rt for 20 minutes. Then, sodium cyanoborohydride (39 mg, 0.62 mmol) and MeOH (0.02 mL) were added and the mixture was stirred at rt for 16 h. Then, MeOH was added and the mixture was stirred for 5 minutes. The mixture was evaporated in vacuo and the residue was purified by flash chromatography (silica, MeOH in DCM from 0/100 to 90/10). The desired fractions were collected and concentrated in vacuo. The residue was purified again by ion exchange chromatography (isolute SCX2 eluting first with MeOH and then with 7N NH₃ in MeOH). The desired fractions were collected and concentrated in vacuo affording product 17 as a white solid (21 mg, 27% yield).

### Preparation of Product 18

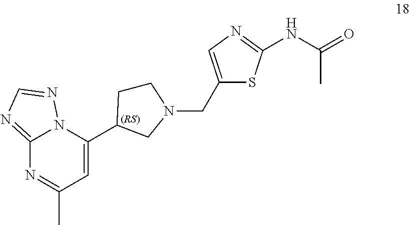 [SMILES: CC(=O)Nc1ncc(CN2CCC(c3cc(C)nc4ncnn34)C2)s1]

Intermediate 38 (119 mg, 0.70 mmol) was added to a solution of intermediate 34 (71 mg, 0.35 mmol) in DCM (2 mL) and the mixture was stirred at rt for 1 h. Then, the mixture was cooled to 0° C. and sodium triacetoxyborohydride (296 mg, 1.40 mmol) was added. The mixture was allowed to warm to rt and stirred overnight. Then, the reaction was quenched with aqueous NaHCO₃ (aq. sat. soltn.) until pH=7-8 and extracted with DCM (3×100 mL). The organic layer was separated, dried (Na₂SO₄), filtered and concentrated in vacuo. The crude product was purified by flash chromatography (silica, EtOAc in heptane from 0/100 to 100/0 and then MeOH in EtOAc from 0/100 to 10/90).

The desired fractions were collected and concentrated in vacuo and the residue was purified again by RP HPLC (Stationary phase: C18 XBridge 30×100 mm 5 μm, Mobile phase: Gradient from 88% 0.1% NH₄CO₃H/NH₄OH pH 9 solution in water, 12% CH₃CN to 72% 0.1% NH₄CO₃H/NH₄OH pH 9 solution in water, 29% CH₃CN). The desired fractions were collected and concentrated in vacuo affording product 18 as a white solid (5 mg, 4% yield).

### Preparation of Product 19

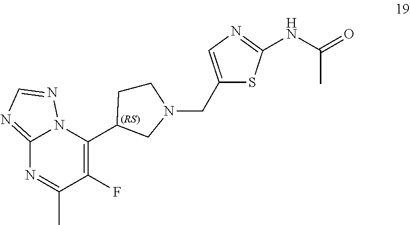 [SMILES: CC(=O)Nc1ncc(CN2CCC(c3c(F)c(C)nc4ncnn34)C2)s1]

Intermediate 38 (22 mg, 0.13 mmol) was added to a solution of intermediate 37 (14.45 mg, 0.065 mmol) in DCM (0.5 mL) and the mixture was stirred at rt for 1 h. Then, the mixture was cooled to 0° C. and sodium triacetoxyborohydride (28 mg, 0.13 mmol) was added. The mixture was allowed to warm to rt and stirred for 3 h. Then, additional sodium triacetoxyborohydride (28 mg, 0.13 mmol) was added and the reaction was further stirred for 2 h. The reaction was quenched with aqueous NaHCO₃ (aq. sat. soltn.) until pH=7-8 and extracted with DCM (3×100 mL). The organic layer was separated, dried (Na₂SO₄), filtered and concentrated in vacuo. The crude product was purified by flash chromatography (silica, EtOAc in heptane from 0/100 to 100/0 and then MeOH in EtOAc from 0/100 to 10/90). The desired fractions were collected and concentrated in vacuo affording product 19 as a yellow solid (9.6 mg, 39% yield).

### Preparation of Product 20

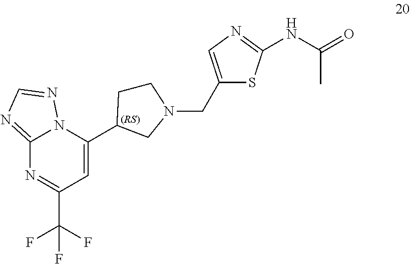 [SMILES: CC(=O)Nc1ncc(CN2CCC(c3cc(C(F)(F)F)nc4ncnn34)C2)s1]

Intermediate 38 (116 mg, 0.68 mmol) was added to a solution of intermediate 50 (88 mg, 0.34 mmol) in DCM (2 mL) and the mixture was stirred at rt for 1 h. Then, the mixture was cooled to 0° C. and sodium triacetoxyborohydride (290 mg, 1.37 mmol) was added. The mixture was allowed to warm to rt and stirred overnight. The reaction was quenched with NH₃/H₂O and extracted with EtOAc. The organic layer was separated, dried (Na₂SO₄), filtered and concentrated in vacuo. The crude product was purified by flash chromatography (silica, EtOAc in heptane from 0/100 to 100/0 and then MeOH in EtOAc from 0/100 to 10/90). The desired fractions were collected and concentrated in vacuo and the residue was purified again by RP HPLC (Stationary phase: C18 XBridge 30×100 mm 5 μm, Mobile phase: Gradient from 81% 0.1% NH₄CO₃H/NH₄OH pH 9 solution in water, 19% CH₃CN to 64% 0.1% NH₄CO₃H/NH₄OH pH 9 solution in water, 36% CH₃CN). The desired fractions were collected and concentrated in vacuo affording product 20 as a white solid (49.7 mg, 35% yield).

### Preparation of Product 21

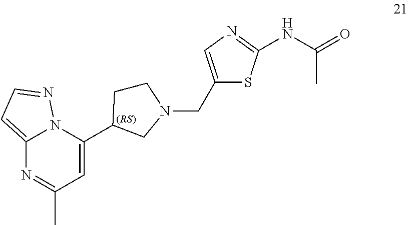 [SMILES: CC(=O)Nc1ncc(CN2CCC(c3cc(C)nc4ccnn34)C2)s1]

Intermediate 38 (43 mg, 0.25 mmol) was added to a solution of intermediate 52 (25 mg, 0.13 mmol) in DCM (0.7 mL) and the mixture was stirred at rt for 1 h. Then sodium triacetoxyborohydride (106 mg, 0.50 mmol) was added and the mixture was further stirred for 4 h. Then, additional sodium triacetoxyborohydride (53 mg, 0.25 mmol) was added and the reaction was further stirred overnight. The reaction was quenched with NH₃/H₂O and extracted with EtOAc. The organic layer was separated, dried (Na₂SO₄), filtered and concentrated in vacuo. The crude product was purified by flash chromatography (silica, EtOAc in heptane from 0/100 to 100/0 and then MeOH in EtOAc from 0/100 to 10/90). The desired fractions were collected and concentrated in vacuo and the residue was purified again by RP HPLC (Stationary phase: C18 XBridge 30×100 mm 5 μm, Mobile phase: Gradient from 81% 0.1% NH₄CO₃H/NH₄OH pH 9 solution in water, 19% CH₃CN to 64% 0.1% NH₄CO₃H/NH₄OH pH 9 solution in water, 36% CH₃CN). The desired fractions were collected and concentrated in vacuo affording product 21 as a white solid (2 mg, 4.5% yield).

### Preparation of Product 22

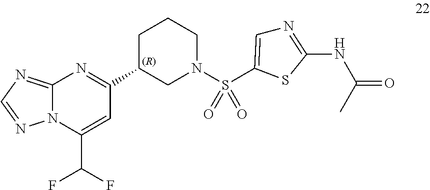

2-Acetylamino-thiazole-5-sulfonyl chloride (CAS: 654072-71-6, 52 mg, 0.22 mmol) was added portion wise to a stirred solution of intermediate 55 (50 mg, 0.20 mmol, bishydrochloric acid salt) and diisopropylethylamine (0.14 mL, 0.79 mmol) in DCM (0.5 mL) at 0° C. and the mixture was further stirred at 0° C. for 1 h. Then H₂O was added and the organic layer was separated, dried (Na₂SO₄), filtered and the solvents evaporated in vacuo. The resultant solid was triturated with Et₂O and filtered affording product 22 as a white solid (27 mg, 30% yield).

### Preparation of Product 23

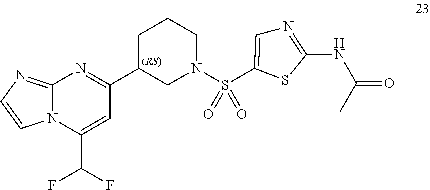

2-Acetylamino-thiazole-5-sulfonyl chloride (CAS: 654072-71-6, 52 mg, 0.22 mmol) was added portion wise to a stirred solution of intermediate 15 (50 mg, 0.20 mmol) and diisopropylethylamine (0.10 mL, 0.59 mmol) in DCM (0.5 mL) at 0° C. and the mixture was further stirred at 0° C. for 1 h. Then H₂O was added and the organic layer was separated, dried (Na₂SO₄), filtered and the solvents evaporated in vacuo. The resultant solid was triturated with CH₃CN and filtered affording product 23 as a white solid (22 mg, 24% yield).

### Preparation of Product 24

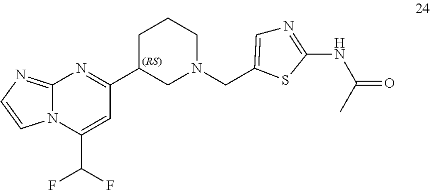

Intermediate 15 (50 mg, 0.20 mmol) was added to a stirred solution of N-[5-(chloromethyl)thiazol-2-yl]acetamide (prepared through the procedure described in WO2014/159234 A1; 45 mg, 0.20 mmol, hydrochloric acid salt) and triethylamine (0.08 mL, 0.59 mmol) in CH₃CN (0.6 mL). The mixture was stirred at rt for 2 h and then at 40° C. for 2 h. Then, the mixture was diluted with EtOAc and washed with H₂O. The organic layer was separated, dried (Na₂SO₄), filtered and the solvents evaporated in vacuo. The resultant solid was triturated with MeOH affording product 24 as a cream solid (38 mg, 47% yield).

### Preparation of Product 25

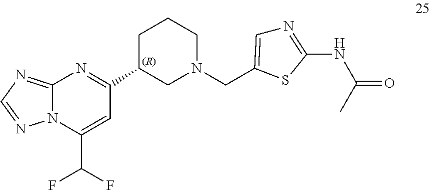

Intermediate 55 (50 mg, 0.20 mmol, bishydrochloric acid salt) was added to a stirred solution of N-[5-(chloromethyl)thiazol-2-yl]acetamide (prepared through the procedure described in WO2014/159234 A1; 45 mg, 0.20 mmol, hydrochloric acid salt) and triethylamine (0.11 mL, 0.79 mmol) in CH₃CN (0.6 mL). The mixture was stirred at rt for 2 h. Then, the mixture was diluted with EtOAc and washed with H₂O. The organic layer was separated, dried (Na₂SO₄), filtered and the solvents evaporated in vacuo. The crude product was purified by flash chromatography (silica, 7M solution of ammonia in MeOH in EtOAc from 0/100 to 5/95). The desired fractions were collected and concentrated in vacuo affording product 25 as a white solid (35 mg, 44% yield).

### Preparation of Product 26

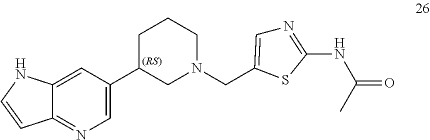

Intermediate 38 (31 mg, 0.18 mmol) and sodium triacetoxyborohydride (104 mg, 0.49 mmol) were added to a solution of intermediate 58 (33 mg, 0.16 mmol) in DCM (2.2 mL). The mixture was stirred at rt for 18 h. Then aqueous NaHCO₃ (aq. sat. soltn.) was added and the organic layer was separated, dried (MgSO₄), filtered and concentrated in vacuo. The crude product was purified by RP HPLC (Stationary phase: C18 XBridge 30×100 mm 5 μm, Mobile phase: Gradient from 80% 0.1% NH₄CO₃H/NH₄OH pH 9 solution in water, 20% CH₃CN to 0% 0.1% NH₄CO₃H/NH₄OH pH 9 solution in water, 100% CH₃CN). The desired fractions were collected and concentrated in vacuo. The product was further purified by flash chromatography (silica, MeOH in DCM from 0/100 to 10/90). The desired fractions were collected and concentrated in vacuo. The product was dried under vacuum at 50° C. for 72 h affording product 26 as a white solid (53 mg, 91% yield).

### Preparation of Product 27

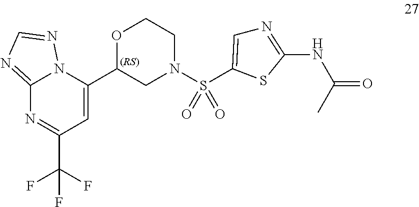 [SMILES: CC(=O)Nc1ncc(S(=O)(=O)N2CCOC(c3cc(C(F)(F)F)nc4ncnn34)C2)s1]

2-Acetylamino-thiazole-5-sulfonyl chloride (CAS: 654072-71-6, 50 mg, 0.21 mmol) was added to a stirred solution of intermediate 63 (74 mg, 0.21 mmol, bishydrochloric acid salt) and diisopropylethylamine (0.12 mL, 0.70 mmol) in DCM (0.5 mL) at 0° C. and the mixture was further stirred at 0° C. for 1 h. Then H₂O was added and the organic layer was separated, dried (MgSO₄), filtered and the solvents evaporated in vacuo. The product was purified by flash chromatography (silica, EtOAc in heptane from 0/100 to 80/20). The desired fractions were collected and concentrated in vacuo affording product 27 as a white solid (55 mg, 55% yield).

### Preparation of Products 28, 29 and 30

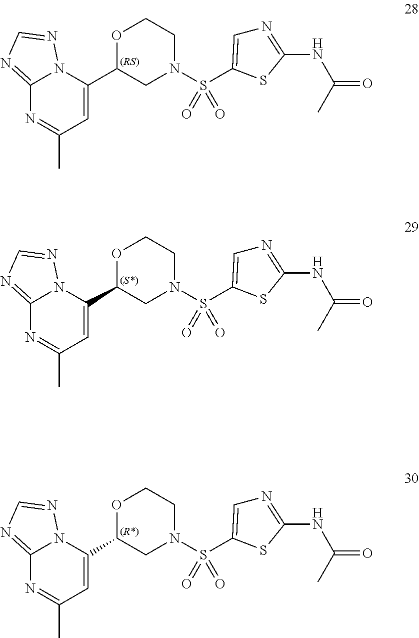 [SMILES: CC(=O)Nc1ncc(S(=O)(=O)N2CCOC(c3cc(C)nc4ncnn34)C2)s1 ; CC(=O)Nc1ncc(S(=O)(=O)N2CCO[C@H](c3cc(C)nc4ncnn34)C2)s1 ; CC(=O)Nc1ncc(S(=O)(=O)N2CCO[C@@H](c3cc(C)nc4ncnn34)C2)s1]

2-Acetylamino-thiazole-5-sulfonyl chloride (CAS: 654072-71-6, 52 mg, 0.22 mmol) was added to a stirred solution of 2-(5-methyl-[1,2,4]triazolo[1,5-a]pyrimidin-7-yl)morpholine (prepared through the procedure described in WO2015/164508; 50 mg, 0.20 mmol, bishydrochloric acid salt) and diisopropylethylamine (0.13 mL, 0.78 mmol) in DCM (0.5 mL) at 0° C. and the mixture was further stirred at 0° C. for 1 h. Then H₂O was added and the organic layer was separated, dried (Na₂SO₄), filtered and the solvents evaporated in vacuo. The resultant solid was triturated with Et₂O and filtered affording product 28 (63 mg, 76% yield).

Product 28 (42.5 mg) was then separated into enantiomers via chiral SFC (Stationary phase: Chiralcel Diacel OJ 20×250 mm, Mobile phase: CO₂, EtOH+0.4 iPrNH₂) yielding product 29 (10 mg) and product 30 (10 mg).

### Preparation of Product 31

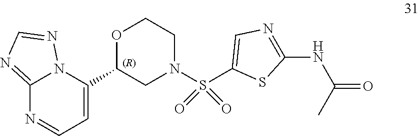

2-Acetylamino-thiazole-5-sulfonyl chloride (CAS: 654072-71-6, 48 mg, 0.20 mmol) was added portion wise to a stirred solution of intermediate 68 (56 mg, 0.20 mmol, bishydrochloric acid salt) and diisopropylethylamine (0.12 mL, 0.70 mmol) in DCM (0.5 mL) at 0° C. and the mixture was further stirred at 0° C. for 1 h. Then H₂O was added and the organic layer was separated, dried (MgSO₄), filtered and the solvents evaporated in vacuo. The product was purified by RP HPLC (Stationary phase: C18 XBridge 30×100 mm 5 μm, Mobile phase: Gradient from 81% 10 mM NH₄CO₃H pH 9 solution in water, 19% CH₃CN to 64% 10 mM NH₄CO₃H pH 9 solution in water, 36% CH₃CN). The desired fractions were collected and concentrated in vacuo affording product 31 as a white solid (23 mg, 28% yield).

### Preparation of Product 32

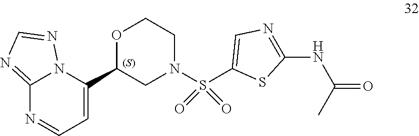

2-Acetylamino-thiazole-5-sulfonyl chloride (CAS: 654072-71-6, 48 mg, 0.20 mmol) was added to a stirred solution of intermediate 69 (55 mg, 0.20 mmol, bishydrochloric acid salt) and diisopropylethylamine (0.12 mL, 0.70 mmol) in DCM (0.5 mL) at 0° C. and the mixture was further stirred at 0° C. for 1 h. Then H₂O was added and organic layer was separated, dried (MgSO₄), filtered and the solvents evaporated in vacuo. The product was triturated with DCM/heptane (1:9) and the solid was purified by flash chromatography (silica, EtOAc in heptane from 0/100 to 80/20). The desired fractions were collected and concentrated in vacuo affording product 32 as a white solid (64 mg, 78% yield).

### Preparation of Product 33

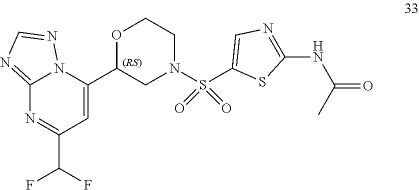 [SMILES: CC(=O)Nc1ncc(S(=O)(=O)N2CCOC(c3cc(C(F)F)nc4ncnn34)C2)s1]

2-Acetylamino-thiazole-5-sulfonyl chloride (CAS: 654072-71-6, 52 mg, 0.22 mmol) was added to a stirred solution of 2-[5-(difluoromethyl)-[1,2,4]triazolo[1,5-a]pyrimidin-7-yl]morpholine (50 mg, 0.20 mmol, hydrochloric acid salt, prepared through a synthetic route analogous to that described for the synthesis of intermediate 20 starting from 4-(tert-butoxycarbonyl)morpholine-2-carboxylic acid [CAS; 189321-66-2] as starting material) and diisopropylethylamine (0.10 mL, 0.59 mmol) in DCM (0.5 mL) at 0° C. and the mixture was further stirred at 0° C. for 1 h. Then H₂O was added and the organic layer was separated, dried (Na₂SO₄), filtered and the solvents evaporated in vacuo. The resultant solid was triturated with Et₂O and filtered affording product 33 (81 mg, 90% yield).

### Preparation of Product 34

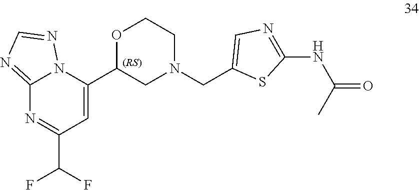 [SMILES: CC(=O)Nc1ncc(CN2CCOC(c3cc(C(F)F)nc4ncnn34)C2)s1]

2-[5-(Difluoromethyl)-[1,2,4]triazolo[1,5-a]pyrimidin-7-yl]morpholine (50 mg, 0.17 mmol, hydrochloric acid salt, prepared through a synthetic route analogous to that described for the synthesis of intermediate 20 starting from 4-(tert-butoxycarbonyl)morpholine-2-carboxylic acid [CAS; 189321-66-2] as starting material) was added to a stirred solution of N-[5-(chloromethyl)thiazol-2-yl]acetamide (prepared through the procedure described in WO2014/159234 A1; 39 mg, 0.17 mmol, hydrochloric acid salt) and triethylamine (0.095 mL, 0.69 mmol) in CH₃CN (0.6 mL). The mixture was stirred at rt for 6 h. Then, additional N-[5-(chloromethyl)thiazol-2-yl]acetamide (11.8 mg, 0.05 mmol, hydrochloric acid salt) was added and the mixture was stirred at 40° C. for 2 h. The mixture was diluted with EtOAc and washed with H₂O. The organic layer was separated, dried (Na₂SO₄), filtered and the solvents evaporated in vacuo. The resultant solid was triturated with MeOH and filtered affording product 34 as a cream solid (37 mg, 53% yield).

### Preparation of Product 35

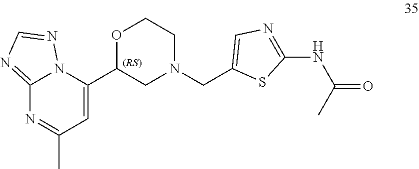 [SMILES: CC(=O)Nc1ncc(CN2CCOC(c3cc(C)nc4ncnn34)C2)s1]

2-(5-Methyl-[1,2,4]triazolo[1,5-a]pyrimidin-7-yl)morpholine (prepared through the procedure described in WO2015/164508; 40 mg, 0.14 mmol, bishydrochloric acid salt) was added to a stirred solution of N-[5-(chloromethyl)thiazol-2-yl]acetamide (prepared through the procedure described in WO2014/159234 A1; 31 mg, 0.14 mmol, hydrochloric acid salt) and triethylamine (0.076 mL, 0.55 mmol) in CH₃CN (0.45 mL). The mixture was stirred at rt for 6 h. The mixture was diluted with EtOAc and washed with H₂O. The organic layer was separated, dried (Na₂SO₄), filtered and the solvents evaporated in vacuo. The resultant solid was triturated with MeOH and filtered affording product 35 as a cream solid (20 mg, 39% yield).

### Preparation of Product 36

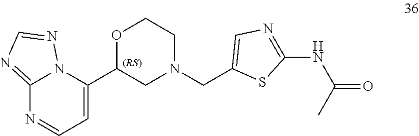 [SMILES: CC(=O)Nc1ncc(CN2CCOC(c3ccnc4ncnn34)C2)s1]

Sodium cyanoborohydride (83 mg, 1.32 mmol) was added to a stirred suspension of intermediate 70 (90 mg, 0.44 mmol, bishydrochloric acid salt), intermediate 38 (97 mg, 0.57 mmol) and triethylamine (0.24 mL, 1.75 mmol) in EtOH (1.5 mL). The mixture was stirred at 80° C. for 16 h. The mixture was quenched with H₂O, concentrated in vacuo and diluted with CH₃CN. The suspension thus obtained was sonicated and filtered affording product 36 (29 mg, 18% yield).

### Preparation of Product 37

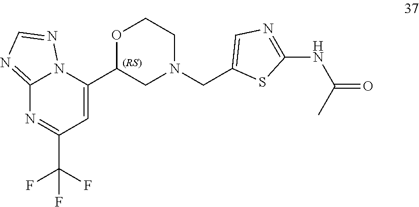 [SMILES: CC(=O)Nc1ncc(CN2CCOC(c3cc(C(F)(F)F)nc4ncnn34)C2)s1]

Triethylamine (0.12 mL, 0.87 mmol) was added to a stirred solution of intermediate 63 (119 mg, 0.43 mmol) and N-[5-(chloromethyl)thiazol-2-yl]acetamide (prepared through the procedure described in WO2014/159234 A1; 83 mg, 0.43 mmol) in CH₃CN (2 mL). The mixture was stirred at rt for 4 h. Then the reaction crude was filtered and the resultant solid was triturated with MeOH and filtered affording product 37 as a white solid (50 mg, 27% yield).

### Preparation of Product 38

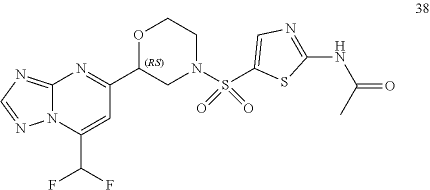 [SMILES: CC(=O)Nc1ncc(S(=O)(=O)N2CCOC(c3cc(C(F)F)n4ncnc4n3)C2)s1]

2-Acetylamino-thiazole-5-sulfonyl chloride (CAS: 654072-71-6, 40 mg, 0.17 mmol) was added to a stirred solution of 2-[7-(difluoromethyl)-[1,2,4]triazolo[1,5-a]pyrimidin-5-yl]morpholine (50 mg, 0.15 mmol, prepared through a synthetic route analogous to that described for the synthesis of intermediate 19 starting from 4-(tert-butoxycarbonyl)morpholine-2-carboxylic acid [CAS; 189321-66-2] as starting material) and diisopropylethylamine (0.079 mL, 0.46 mmol) in DCM (0.4 mL) at 0° C. and the mixture was further stirred at 0° C. for 1 h. Then H₂O was added and the organic layer was separated, dried (Na₂SO₄), filtered and the solvents evaporated in vacuo. The resultant solid was triturated with Et₂O and filtered affording product 38 as a white solid (70 mg, 99% yield).

### Preparation of Product 39

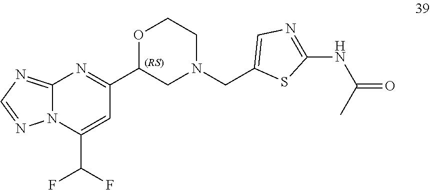 [SMILES: CC(=O)Nc1ncc(CN2CCOC(c3cc(C(F)F)n4ncnc4n3)C2)s1]

2-[7-(Difluoromethyl)-[1,2,4]triazolo[1,5-a]pyrimidin-5-yl]morpholine (50 mg, 0.20 mmol, prepared through a synthetic route analogous to that described for the synthesis of intermediate 19 starting from 4-(tert-butoxycarbonyl)morpholine-2-carboxylic acid [CAS; 189321-66-2] as starting material) was added to a stirred solution of N-[5-(chloromethyl)thiazol-2-yl]acetamide (prepared through the procedure described in WO2014/159234 A1; 37 mg, 0.20 mmol, hydrochloric acid salt) and triethylamine (0.054 mL, 0.39 mmol) in CH₃CN (0.6 mL). The mixture was stirred at rt for 6 h. The mixture was diluted with EtOAc and washed with H₂O. The organic layer was separated, dried (Na₂SO₄), filtered and the solvents evaporated in vacuo. The resultant solid was triturated with MeOH and filtered affording product 39 as a cream solid (50 mg, 62% yield).

### Preparation of Product 40

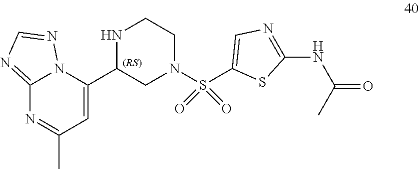 [SMILES: CC(=O)Nc1ncc(S(=O)(=O)N2CCNC(c3cc(C)nc4ncnn34)C2)s1]

2-Acetylamino-thiazole-5-sulfonyl chloride (CAS: 654072-71-6, 40 mg, 0.17 mmol) was added to a stirred solution of 5-methyl-7-piperazin-2-yl-[1,2,4]triazolo[1,5-a]pyrimidine (52 mg, 0.16 mmol, trishydrochloric acid salt, prepared through a synthetic route analogous to that described for the synthesis of intermediate 11 starting from 1,4-bis(tert-butoxycarbonyl)piperazine-2-carboxylic acid [CAS; 173774-48-6] as starting material) and diisopropylethylamine (0.1 mL, 0.58 mmol) in DCM (0.4 mL) at 0° C. and the mixture was further stirred at 0° C. for 1 h. Then H₂O was added and the organic layer was separated, dried (MgSO₄), filtered and the solvents evaporated in vacuo. The product was purified by flash chromatography (silica, EtOAc in heptane from 0/100 to 80/20). The desired fractions were collected and concentrated in vacuo affording product 40 as a yellow solid (27 mg, 40% yield).

### Preparation of Product 41

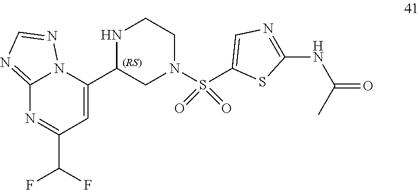 [SMILES: CC(=O)Nc1ncc(S(=O)(=O)N2CCNC(c3cc(C(F)F)nc4ncnn34)C2)s1]

2-Acetylamino-thiazole-5-sulfonyl chloride (CAS: 654072-71-6, 33 mg, 0.14 mmol) was added to a stirred solution of 5-(difluoromethyl)-7-piperazin-2-yl-[1,2,4]triazolo[1,5-a]pyrimidine (50 mg, 0.14 mmol, trishydrochloric acid salt, prepared through a synthetic route analogous to that described for the synthesis of intermediate 20 starting from 1,4-bis(tert-butoxycarbonyl)piperazine-2-carboxylic acid [CAS; 173774-48-6] as starting material) and diisopropylethylamine (0.071 mL, 0.41 mmol) in DCM (0.35 mL) at 0° C. and the mixture was further stirred at 0° C. for 1 h. Then H₂O was added and the organic layer was separated, dried (MgSO₄), filtered and the solvents evaporated in vacuo. The resultant solid was triturated with Et₂O and filtered. The solid was purified by RP HPLC (Stationary phase: C18 XBridge 30×100 mm 5 μm, Mobile phase: Gradient from 81% 10 mM NH₄CO₃H pH 9 solution in water, 19% CH₃CN to 64% 10 mM NH₄CO₃H pH 9 solution in water, 36% CH₃CN). The desired fractions were collected and concentrated in vacuo affording product 41 (35 mg, 55% yield).

### Preparation of Product 42

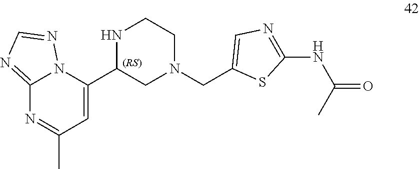 [SMILES: CC(=O)Nc1ncc(CN2CCNC(c3cc(C)nc4ncnn34)C2)s1]

Triethylamine (0.24 mL, 1.74 mmol) was added to a stirred solution of 5-methyl-7-piperazin-2-yl-[1,2,4]triazolo[1,5-a]pyrimidine (143 mg, 0.43 mmol, trishydrochloric acid salt, prepared through a synthetic route analogous to that described for the synthesis of intermediate 11 starting from 1,4-bis(tert-butoxycarbonyl)piperazine-2-carboxylic acid [CAS; 173774-48-6] as starting material), N-[5-(chloromethyl)thiazol-2-yl]acetamide (prepared through the procedure described in WO2014/159234 A1; 83 mg, 0.43 mmol) in CH₃CN (1.5 mL). The mixture was stirred at rt for 16 h. Then the reaction crude was filtered and the resultant solid was purified by flash chromatography (silica, 7M solution of ammonia in MeOH in DCM from 0/100 to 5/95). The desired fractions were collected and concentrated in vacuo. The desired fractions were collected and concentrated in vacuo. The resultant oil was triturated with MeOH and filtered affording product 42 as a white solid (40 mg, 25% yield).

### Preparation of Product 43

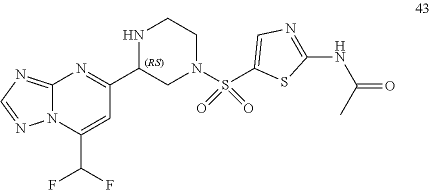 [SMILES: CC(=O)Nc1ncc(S(=O)(=O)N2CCNC(c3cc(C(F)F)n4ncnc4n3)C2)s1]

2-Acetylamino-thiazole-5-sulfonyl chloride (CAS: 654072-71-6, 33 mg, 0.14 mmol) was added to a stirred solution of 7-(difluoromethyl)-5-piperazin-2-yl-[1,2,4]triazolo[1,5-a]pyrimidine (50 mg, 0.14 mmol, trishydrochloric acid salt, prepared through a synthetic route analogous to that described for the synthesis of intermediate 19 starting from 1,4-bis(tert-butoxycarbonyl)piperazine-2-carboxylic acid [CAS; 173774-48-6] as starting material) and diisopropylethylamine (0.095 mL, 0.55 mmol) in DCM (0.35 mL) at 0° C. and the mixture was further stirred at 0° C. for 1 h. Then H₂O was added and the organic layer was separated, dried (Na₂SO₄), filtered and the solvents evaporated in vacuo. The resultant solid was triturated with Et₂O and filtered affording product 43 as a white solid (20 mg, 32% yield).

### Preparation of Product 44

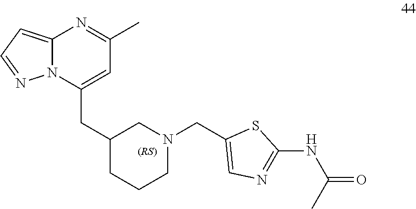 [SMILES: CC(=O)Nc1ncc(CN2CCCC(Cc3cc(C)nc4ccnn34)C2)s1]

Intermediate 38 (37 mg, 0.22 mmol) was added to a solution of intermediate 73 (25 mg, 0.11 mmol) in DCM (0.6 mL) and the mixture was stirred at rt for 1 h. Then sodium triacetoxyborohydride (92 mg, 0.43 mmol) was added and the mixture was stirred at rt overnight. Additional sodium triacetoxyborohydride (46 mg, 0.22 mmol) was added and the reaction was further stirred at rt for 3 h. The reaction was quenched with NH₃/H₂O and extracted with EtOAc. The organic layer was separated, dried (Na₂SO₄), filtered and concentrated in vacuo. The residue was purified by flash chromatography (silica, EtOAc in heptane from 0/100 to 100/0 and then MeOH in EtOAc from 0/100 to 10/90). The desired fractions were collected and concentrated in vacuo and the residue was purified again by RP HPLC (Stationary phase: C18 XBridge 30×100 mm 5 μm, Mobile phase: Gradient from 81% 0.1% NH₄CO₃H/NH₄OH pH 9 solution in water, 19% CH₃CN to 64% 0.1% NH₄CO₃H/NH₄OH pH 9 solution in water, 36% CH₃CN). The desired fractions were collected and concentrated in vacuo affording product 44 as a white sticky solid (5.5 mg, 13% yield).

### Preparation of Product 45

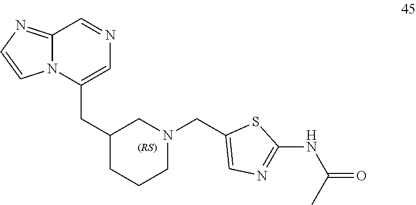 [SMILES: CC(=O)Nc1ncc(CN2CCCC(Cc3cncc4nccn34)C2)s1]

Sodium triacetoxyborohydride (20 mg, 0.095 mmol) was added to a solution of intermediate 75 (20 mg, 0.08 mmol, hydrochloric acid salt), intermediate 38 (16 mg, 0.095 mmol) and triethylamine (0.03 mL, 0.24 mmol) in DCM (0.5 mL) under nitrogen atmosphere. The mixture was stirred at rt for 3 days. Then aqueous NaHCO₃ (aq. sat. soltn.) was added and the organic layer was separated, dried (MgSO₄), filtered and concentrated in vacuo. The crude product was purified by flash chromatography (silica, EtOAc in heptane from 20/80 to 100/0). The desired fractions were collected and concentrated in vacuo. The residue was purified again by RP HPLC (Stationary phase: C18 XBridge 30×100 mm 5 μm, Mobile phase: Gradient from 81% 10 mM NH₄CO₃H pH 9 solution in water, 19% CH₃CN to 64% 10 mM NH₄CO₃H pH 9 solution in water, 36% CH₃CN). The desired fractions were collected and concentrated in vacuo affording product 45 as a yellow oil (2 mg, 6.8% yield).

### Preparation of Product 46

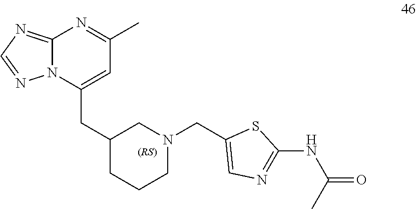 [SMILES: CC(=O)Nc1ncc(CN2CCCC(Cc3cc(C)nc4ncnn34)C2)s1]

Intermediate 38 (131 mg, 0.77 mmol) was added to a solution of intermediate 77 (89 mg, 0.385 mmol) in DCM (2 mL) and the mixture was stirred at rt for 30 minutes. Then sodium triacetoxyborohydride (326 mg, 1.54 mmol) was added and the mixture was stirred at rt for 2 h. Additional sodium triacetoxyborohydride (163 mg, 0.77 mmol) was added and the reaction was further stirred at rt overnight. The reaction was quenched with NH₃/H₂O and extracted with EtOAc. The organic layer was separated, dried (Na₂SO₄), filtered and concentrated in vacuo. The residue was purified by flash chromatography (silica, EtOAc in heptane from 0/100 to 100/0 and then MeOH in EtOAc from 0/100 to 10/90). The desired fractions were collected and concentrated in vacuo and the residue was purified again by flash chromatography (silica, 7M solution of ammonia in MeOH in DCM from 0/100 to 10/90). The desired fractions were collected and concentrated in vacuo affording product 46 as a white solid (10 mg, 6.6% yield).

### Preparation of Product 47

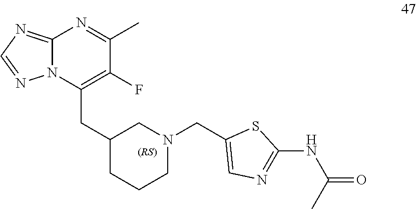 [SMILES: CC(=O)Nc1ncc(CN2CCCC(Cc3c(F)c(C)nc4ncnn34)C2)s1]

Intermediate 38 (48 mg, 0.28 mmol) was added to a solution of intermediate 79 (35 mg, 0.14 mmol) in DCM (0.8 mL) and the mixture was stirred at rt for 1 h. Then sodium triacetoxyborohydride (119 mg, 0.56 mmol) was added and the mixture was stirred at rt overnight. The reaction was quenched with NH₃/H₂O and extracted with EtOAc. The organic layer was separated, dried (Na₂SO₄), filtered and concentrated in vacuo. The residue was purified by flash chromatography (silica, EtOAc in heptane from 0/100 to 100/0 and then MeOH in EtOAc from 0/100 to 10/90). The desired fractions were collected and concentrated in vacuo and the residue was purified again by RP HPLC (Stationary phase: C18 XBridge 30×100 mm 5 μm, Mobile phase: Gradient from 81% 0.1% NH₄CO₃H/NH₄OH pH 9 solution in water, 19% CH₃CN to 64% 0.1% NH₄CO₃H/NH₄OH pH 9 solution in water, 36% CH₃CN). The desired fractions were collected and concentrated in vacuo affording product 47 as a white solid (12.3 mg, 21% yield).

### Preparation of Product 48

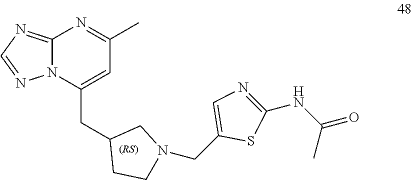 [SMILES: CC(=O)Nc1ncc(CN2CCC(Cc3cc(C)nc4ncnn34)C2)s1]

Intermediate 38 (130.5 mg, 0.77 mmol) was added to a solution of intermediate 82 (127 mg, 0.38 mmol, trifluoroacetate salt) in DCM (2 mL) and the mixture was stirred at rt for 30 minutes. Sodium triacetoxyborohydride (325 mg, 1.53 mmol) was added and the mixture was stirred at rt overnight. Then, AcOH (0.05 mL, 0.09 mmol) and additional sodium triacetoxyborohydride (163 mg, 0.77 mmol) were added and the reaction was further stirred at rt for 3 h. The reaction was quenched with NH₃/H₂O and extracted with EtOAc. The organic layer was separated, dried (Na₂SO₄), filtered and concentrated in vacuo. The residue was purified by flash chromatography (silica, EtOAc in heptane from 0/100 to 100/0 and then MeOH in EtOAc from 0/100 to 10/90). The desired fractions were collected and concentrated in vacuo and the residue was purified again by RP HPLC (Stationary phase: C18 XBridge 30×100 mm 5 μm, Mobile phase: Gradient from 88% 0.1% NH₄CO₃H/NH₄OH pH 9 solution in water, 12% CH₃CN to 72% 0.1% NH₄CO₃H/NH₄OH pH 9 solution in water, 29% CH₃CN). The desired fractions were collected and concentrated in vacuo affording product 48 as a white solid (39 mg, 26% yield).

### Preparation of Product 49

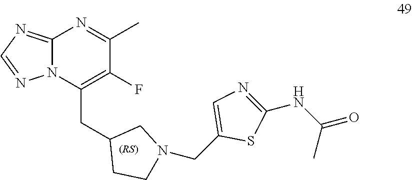 [SMILES: CC(=O)Nc1ncc(CN2CCC(Cc3c(F)c(C)nc4ncnn34)C2)s1]

Intermediate 38 (86 mg, 0.50 mmol) was added to a solution of intermediate 84 (88 mg, 0.25 mmol, trifluoroacetate salt) in DCM (1.3 mL) and the mixture was stirred at rt for 5 minutes. Then sodium triacetoxyborohydride (214 mg, 1.01 mmol) was added and the mixture was stirred at rt overnight. The reaction was quenched with NH₃/H₂O and extracted with EtOAc. The organic layer was separated, dried (Na₂SO₄), filtered and concentrated in vacuo. The residue was purified by flash chromatography (silica, EtOAc in heptane from 0/100 to 100/0 and then MeOH in EtOAc from 0/100 to 10/90). The desired fractions were collected and concentrated in vacuo and the residue was purified again by RP HPLC (Stationary phase: C18 XBridge 30×100 mm 5 μm, Mobile phase: Gradient from 81% 0.1% NH₄CO₃H/NH₄OH pH 9 solution in water, 19% CH₃CN to 64% 0.1% NH₄CO₃H/NH₄OH pH 9 solution in water, 36% CH₃CN). The desired fractions were collected and concentrated in vacuo affording product 49 as a white solid (19 mg, 18% yield).

### Preparation of Product 50

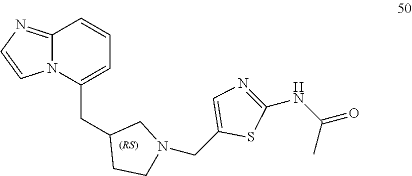 [SMILES: CC(=O)Nc1ncc(CN2CCC(Cc3cccc4nccn34)C2)s1]

Diisopropylethylamine (0.18 mL, 1.05 mmol) was added to a suspension of intermediate 86 (50 mg, 0.21 mmol, hydrochloric acid salt) in DCM (1.1 mL) and the mixture was stirred at rt for 5 minutes. Then intermediate 38 (43 mg, 0.25 mmol) and sodium triacetoxyborohydride (89 mg, 0.42 mmol) were added and the mixture was stirred at rt for 17 h. Then aqueous NaHCO₃ (aq. sat. soltn.) was added and the organic layer was separated, dried (MgSO₄), filtered and concentrated in vacuo. The crude product was purified by RP HPLC (Stationary phase: C18 XBridge 30×100 mm 5 μm, Mobile phase: Gradient from 81% 10 mM NH₄CO₃H pH 9 solution in water, 19% CH₃CN to 64% 10 mM NH₄CO₃H pH 9 solution in water, 36% CH₃CN). The desired fractions were collected and concentrated in vacuo affording product 50 as a yellow oil (20 mg, 27% yield).

### Preparation of Product 51

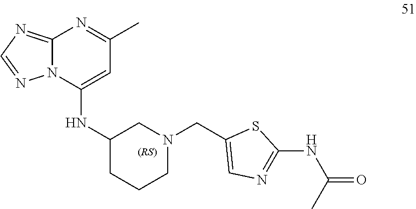 [SMILES: CC(=O)Nc1ncc(CN2CCCC(Nc3cc(C)nc4ncnn34)C2)s1]

Diisopropylethylamine (0.47 mL, 2.75 mmol) was added to a suspension of intermediate 88 (180 mg, 0.55 mmol, bishydrochloric acid salt) in CH₃CN (10 mL) in a sealed tube. The mixture was stirred at rt for 15 minutes. Then 7-chloro-5-methyl-[1,2,4]triazolo[1,5-a]pyrimidine (CAS: 24415-66-5; 102 mg, 0.605 mmol) was added and the resulting solution was stirred at 100° C. for 16 h. The reaction was cooled to rt and the volatiles were evaporated in vacuo. The residue thus obtained was taken up in EtOAc and water was added. The organic layer was separated, dried (Na₂SO₄), filtered and concentrated in vacuo. The residue thus obtained was purified by RP HPLC (Stationary phase: C18 XBridge 50×150 mm 5 μm, Mobile phase: Gradient from 80% 10 mM NH₄CO₃H pH 9 solution in water, 20% CH₃CN to 0% 10 mM NH₄CO₃H pH 9 solution in water, 100% CH₃CN). The desired fractions were collected and concentrated in vacuo affording product 51 as a pale brown solid (35 mg, 16% yield).

### Preparation of Product 52

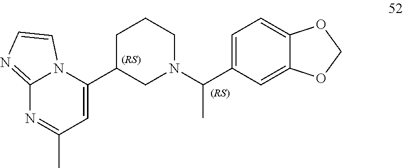 [SMILES: Cc1cc(C2CCCN(C(C)c3ccc4c(c3)OCO4)C2)n2ccnc2n1]

Titanium (IV) isopropoxide (0.22 mL, 0.74 mmol) was added to a stirred solution of intermediate 47 (80 mg, 0.37 mmol) and 3′,4′-(methylenedioxy)acetophenone (CAS: 3162-29-6; 73 mg, 0.44 mmol) in THF (1 mL) and EtOH (0.5 mL) in a sealed tube at rt. The reaction mixture was stirred at 70° C. for 2 h and then sodium cyanoborohydride (70 mg, 1.11 mmol) was added. The reaction mixture was stirred at 70° C. for 16 h. Then the mixture was concentrated in vacuo and the residue purified by flash chromatography (silica, MeOH in DCM from 0/100 to 10/90). The desired fractions were collected and concentrated in vacuo and the residue was purified again by RP HPLC (Stationary phase: C18 XBridge 30×100 mm 5 μm, Mobile phase: Gradient from 74% 0.1% NH₄CO₃H/NH₄OH pH 9 solution in water, 26% CH₃CN to 58% 0.1% NH₄CO₃H/NH₄OH pH 9 solution in water, 42% CH₃CN). The desired fractions were collected and concentrated in vacuo. The residue was purified by flash chromatography (silica, acetone 100%). The desired fractions were collected and concentrated in vacuo. The residue was purified again by ion exchange chromatography (Isolute® SCX2 eluting first with MeOH and then with 7N NH₃ in MeOH). The desired fractions were collected and concentrated in vacuo affording product 52 as a pale yellow solid (21 mg, 27% yield).

### Preparation of Product 53

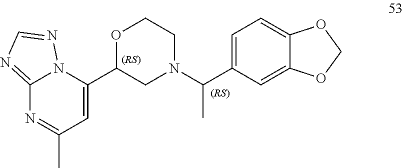 [SMILES: Cc1cc(C2CN(C(C)c3ccc4c(c3)OCO4)CCO2)n2ncnc2n1]

A mixture of 2-(5-methyl-[1,2,4]triazolo[1,5-a]pyrimidin-7-yl)morpholine (prepared through the procedure described in WO2015/164508; 138.5 mg, 0.54 mmol, hydrochloric acid salt), 5-(1-chloroethyl)-1,3-benzodioxole (prepared through the procedure described in PCT Int. App., 2016030443; 100 mg, 0.54 mmol) and triethylamine (0.15 mL, 1.08 mmol) in DCE (3 mL) was stirred at rt for 120 h. Then the mixture was concentrated in vacuo and the residue purified by flash chromatography (silica, 7M solution of ammonia in MeOH in DCM from 0/100 to 3/97). The desired fractions were collected and concentrated in vacuo and the residue was purified again by RP HPLC (Stationary phase: C18 XBridge 30×100 mm 5 μm, Mobile phase: Gradient from 74% 10 mM NH₄CO₃H pH 9 solution in water, 26% CH₃CN to 58% 10 mM NH₄CO₃H pH 9 solution in water, 42% CH₃CN). The desired fractions were collected and concentrated in vacuo affording product 53 as a white solid (31 mg, 16% yield).

### Preparation of Product 54

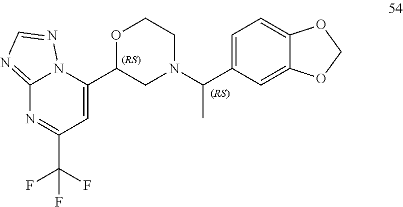 [SMILES: CC(c1ccc2c(c1)OCO2)N1CCOC(c2cc(C(F)(F)F)nc3ncnn23)C1]

Triethylamine (0.06 mL, 0.44 mmol) was added to a stirred solution of intermediate 63 (60 mg, 0.22 mmol) and 5-(1-chloroethyl)-1,3-benzodioxole (prepared through the procedure described in PCT Int. App., 2016030443; 40.5 mg, 0.22 mmol) in CH₃CN (0.5 mL). The mixture was stirred at rt for 16 h. Then additional 5-(1-chloroethyl)-1,3-benzodioxole (40.5 mg, 0.22 mmol) was added and the mixture was further stirred at rt for 72 h. The mixture was concentrated in vacuo and the residue purified by RP HPLC (Stationary phase: C18 XBridge 30×100 mm 5 μm, Mobile phase: Gradient from 81% 10 mM NH₄CO₃H pH 9 solution in water, 19% CH₃CN to 64% 10 mM NH₄CO₃H pH 9 solution in water, 36% CH₃CN). The desired fractions were collected and concentrated in vacuo affording product 54 as a yellow solid (20 mg, 22% yield).

### Preparation of Product 55

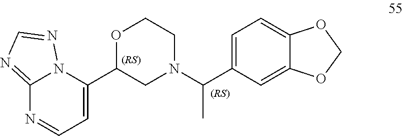 [SMILES: CC(c1ccc2c(c1)OCO2)N1CCOC(c2ccnc3ncnn23)C1]

5-(1-Chloroethyl)-1,3-benzodioxole (prepared through the procedure described in PCT Int. App., 2016030443; 199 mg, 1.08 mmol) was added to a stirred suspension of intermediate 70 (300 mg, 1.08 mmol, bishydrochloric acid salt) and triethylamine (0.75 mL, 5.39 mmol) in CH₃CN (2.3 mL). The mixture was stirred at rt for 16 h. The mixture was concentrated in vacuo and the residue purified by flash chromatography (silica, 7M solution of ammonia in MeOH in DCM from 0/100 to 100/0). The desired fractions were collected and concentrated in vacuo and the residue was purified by flash chromatography (silica, EtOAc in heptane from 0/100 to 100/0). The desired fractions were collected and concentrated in vacuo and the residue was purified by RP HPLC (Stationary phase: C18 XBridge 30×100 mm 5 μm, Mobile phase: Gradient from 74% 0.1% NH₄CO₃H/NH₄OH pH 9 solution in water, 26% CH₃CN to 58% 0.1% NH₄CO₃H/NH₄OH pH 9 solution in water, 42% CH₃CN). The desired fractions were collected and concentrated in vacuo affording product 55 as a white solid (80 mg, 31% yield).

### Preparation of Products 56 and 57

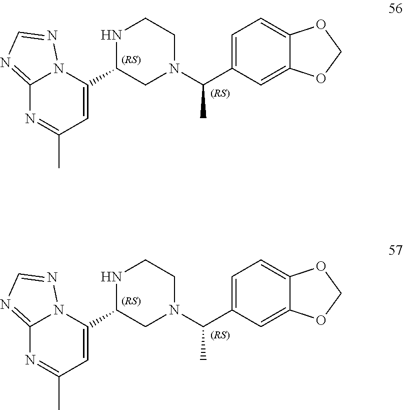 [SMILES: Cc1cc([C@H]2CN([C@H](C)c3ccc4c(c3)OCO4)CCN2)n2ncnc2n1 ; Cc1cc([C@H]2CN([C@@H](C)c3ccc4c(c3)OCO4)CCN2)n2ncnc2n1]

Triethylamine (0.51 mL, 3.66 mmol) was added to a stirred solution of 5-methyl-7-piperazin-2-yl-[1,2,4]triazolo[1,5-a]pyrimidine (300 mg, 0.92 mmol, trishydrochloric acid salt, prepared through a synthetic route analogous to that described for the synthesis of intermediate 11 starting from 1,4-bis(tert-butoxycarbonyl)piperazine-2-carboxylic acid [CAS; 173774-48-6] as starting material) and 5-(1-chloroethyl)-1,3-benzodioxole (prepared through the procedure described in PCT Int. App., 2016030443; 169 mg, 0.92 mmol) in CH₃CN (3 mL). The mixture was stirred at rt for 4 h. Then the reaction crude was filtered. The filtrate was concentrated in vacuo and the residue was purified by RP HPLC (Stationary phase: C18 XBridge 30×100 mm 5 μm, Mobile phase: Gradient from 81% 10 mM NH₄CO₃H pH 9 solution in water, 19% CH₃CN to 64% 10 mM NH₄CO₃H pH 9 solution in water, 36% CH₃CN). The desired fractions were collected and concentrated in vacuo affording product 56 as a white solid (27 mg, 8% yield) and product 57 as a yellow solid (14 mg, 4%).

### Preparation of Product 58

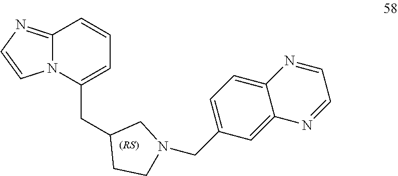 [SMILES: c1cc(CC2CCN(Cc3ccc4nccnc4c3)C2)n2ccnc2c1]

Diisopropylethylamine (0.18 mL, 1.05 mmol) was added to a suspension of intermediate 86 (50 mg, 0.21 mmol, hydrochloric acid salt) in DCM (1.1 mL) and the mixture was stirred at rt for 5 minutes. Then quinoxaline-6-carbaldehyde (CAS: 130345-50-5; 40 mg, 0.25 mmol) and sodium triacetoxyborohydride (89 mg, 0.42 mmol) were added and the mixture was stirred at rt for 17 h. Then aqueous NaHCO₃ (aq. sat. soltn.) was added and the organic layer was separated, dried (MgSO₄), filtered and concentrated in vacuo. The crude product was purified by RP HPLC (Stationary phase: C18 XBridge 30×100 mm 5 μm, Mobile phase: Gradient from 81% 10 mM NH₄CO₃H pH 9 solution in water, 19% CH₃CN to 64% 10 mM NH₄CO₃H pH 9 solution in water, 36% CH₃CN). The desired fractions were collected and concentrated in vacuo affording product 58 as a yellow oil (18 mg, 25% yield).

### Preparation of Product 59

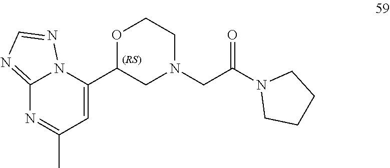 [SMILES: Cc1cc(C2CN(CC(=O)N3CCCC3)CCO2)n2ncnc2n1]

2-Chloro-1-pyrrolidin-1-yl-ethanone (CAS: 20266-00-6; 100 mg, 0.68 mmol) was added to a stirred solution of 2-(5-methyl-[1,2,4]triazolo[1,5-a]pyrimidin-7-yl)morpholine (prepared through the procedure described in WO2015/164508; 124 mg, 0.56 mmol) and triethylamine (0.25 mL, 1.81 mmol) in DCM (3.3 mL). The mixture was stirred at rt for 24 h. The mixture was concentrated in vacuo and the residue purified by flash chromatography (silica, 7M solution of ammonia in MeOH in DCM from 0/100 to 3/97). The desired fractions were collected and concentrated in vacuo and the residue was purified by RP HPLC (Stationary phase: C18 XBridge 30×100 mm 5 μm, Mobile phase: Gradient from 81% 10 mM NH₄CO₃H pH 9 solution in water, 19% CH₃CN to 64% 10 mM NH₄CO₃H pH 9 solution in water, 36% CH₃CN). The desired fractions were collected and concentrated in vacuo affording product 59 (52 mg, 28% yield).

### Preparation of Product 60

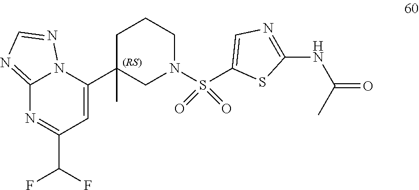 [SMILES: CC(=O)Nc1ncc(S(=O)(=O)N2CCCC(C)(c3cc(C(F)F)nc4ncnn34)C2)s1]

2-Acetylamino-thiazole-5-sulfonyl chloride (CAS: 654072-71-6, 50 mg, 0.21 mmol) was added portion wise to a stirred solution of 5-(difluoromethyl)-7-(3-methyl-3-piperidyl)-[1,2,4]triazolo[1,5-a]pyrimidine (65 mg, 0.21 mmol, hydrochloric acid salt, prepared through a synthetic route analogous to that described for the synthesis of intermediate 20 starting from 1-(1-tert-butoxycarbonyl-3-methyl-piperidine-3-carboxylic acid as starting material) and diisopropylethylamine (0.12 mL, 0.70 mmol) in DCM (0.5 mL) at 0° C. and the mixture was further stirred at 0° C. for 1 h. Then H₂O was added and the organic layer was separated, dried (MgSO₄), filtered and the solvents evaporated in vacuo. The residue was triturated with a mixture of DCM/heptane (1:9). The solid was filtered and purified by flash chromatography (silica, EtOAc in heptane from 0/100 to 80/20). The desired fractions were collected and concentrated in vacuo affording product 60 as a white solid (84 mg, 86% yield). 1-(1-tert-Butoxycarbonyl-3-methyl-piperidine-3-carboxylic acid was prepared through the procedure described in PCT Int. App. 2015083028.

### Preparation of Product 61

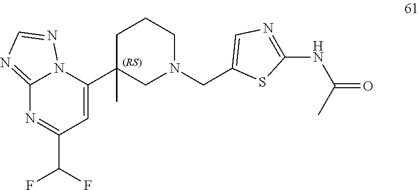 [SMILES: CC(=O)Nc1ncc(CN2CCCC(C)(c3cc(C(F)F)nc4ncnn34)C2)s1]

Sodium triacetoxyborohydride (104 mg, 0.49 mmol) was added to a solution of 5-(difluoromethyl)-7-(3-methyl-3-piperidyl)-[1,2,4]triazolo[1,5-a]pyrimidine (89 mg, 0.33 mmol, prepared through a synthetic route analogous to that described for the synthesis of intermediate 20 starting from 1-(1-tert-butoxycarbonyl-3-methyl-piperidine-3-carboxylic acid as starting material), intermediate 38 (70 mg, 0.41 mmol) and triethylamine (0.14 mL, 1.01 mmol) in DCM (2 mL) under nitrogen atmosphere. The mixture was stirred at rt for 2 days. Then aqueous NaHCO₃ (aq. sat. soltn.) was added and the organic layer was separated, dried (MgSO₄), filtered and concentrated in vacuo. The crude product was purified by RP HPLC (Stationary phase: C18 XBridge 30×150 mm 5 μm, Mobile phase: Gradient from 81% 10 mM NH₄CO₃H pH 9 solution in water, 19% CH₃CN to 64% 10 mM NH₄CO₃H pH 9 solution in water, 36% CH₃CN). The desired fractions were collected and concentrated in vacuo affording product 61 as a colourless film (12 mg, 9% yield). 1-(1-tert-Butoxycarbonyl-3-methyl-piperidine-3-carboxylic acid was prepared through the procedure described in PCT Int. App. 2015083028.

### Preparation of Product 62

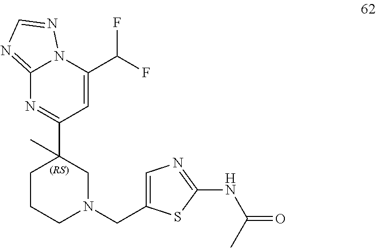 [SMILES: CC(=O)Nc1ncc(CN2CCCC(C)(c3cc(C(F)F)n4ncnc4n3)C2)s1]

Sodium triacetoxyborohydride (48 mg, 0.23 mmol) was added to a solution of 7-(difluoromethyl)-5-(3-methyl-3-piperidyl)-[1,2,4]triazolo[1,5-a]pyrimidine (60 mg, 0.20 mmol, hydrochloric acid salt, prepared through a synthetic route analogous to that described for the synthesis of intermediate 19 starting from 1-(1-tert-butoxycarbonyl-3-methyl-piperidine-3-carboxylic acid as starting material), intermediate 38 (40 mg, 0.235 mmol) and triethylamine (0.08 mL, 0.58 mmol) in DCM (2 mL) under nitrogen atmosphere. The mixture was stirred at rt for 17 h. Then aqueous NaHCO₃ (aq. sat. soltn.) was added and the organic layer was separated, dried (MgSO₄), filtered and concentrated in vacuo. The crude product was purified by RP HPLC (Stationary phase: C18 XBridge 30×100 mm 5 μm, Mobile phase: Gradient from 81% 10 mM NH₄CO₃H pH 9 solution in water, 19% CH₃CN to 64% 10 mM NH₄CO₃H pH 9 solution in water, 36% CH₃CN). The desired fractions were collected and concentrated in vacuo affording product 62 as a white solid (34 mg, 41% yield). 1-(1-tert-Butoxycarbonyl-3-methyl-piperidine-3-carboxylic acid was prepared through the procedure described in PCT Int. App. 2015083028.

### Preparation of Product 63

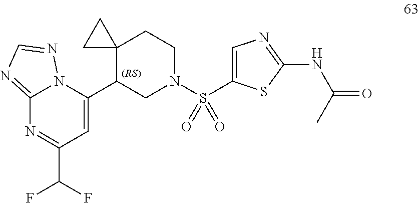 [SMILES: CC(=O)Nc1ncc(S(=O)(=O)N2CCC3(CC3)C(c3cc(C(F)F)nc4ncnn34)C2)s1]

2-Acetylamino-thiazole-5-sulfonyl chloride (CAS: 654072-71-6, 50 mg, 0.21 mmol) was added portion wise to a stirred solution of 7-(6-azaspiro[2.5]octan-8-yl)-5-(difluoromethyl)-[1,2,4]triazolo[1,5-a]pyrimidine (72 mg, 0.23 mmol, hydrochloric acid salt, prepared through a synthetic route analogous to that described for the synthesis of intermediate 20 starting from intermediate 95) and diisopropylethylamine (0.11 mL, 0.64 mmol) in DCM (0.5 mL) at 0° C. and the mixture was further stirred at 0° C. for 1 h. Then H₂O was added and the organic layer was separated, dried (MgSO₄), filtered and the solvents evaporated in vacuo. The residue was purified by flash chromatography (silica, EtOAc in heptane from 0/100 to 80/20). The desired fractions were collected and concentrated in vacuo affording product 63 as a white solid (63 mg, 63% yield).

### Preparation of Product 64

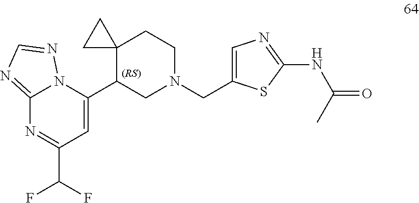 [SMILES: CC(=O)Nc1ncc(CN2CCC3(CC3)C(c3cc(C(F)F)nc4ncnn34)C2)s1]

Sodium triacetoxyborohydride (46 mg, 0.22 mmol) was added to a solution of 7-(6-azaspiro[2.5]octan-8-yl)-5-(difluoromethyl)-[1,2,4]triazolo[1,5-a]pyrimidine (51 mg, 0.16 mmol, hydrochloric acid salt, prepared through a synthetic route analogous to that described for the synthesis of intermediate 20 starting from intermediate 95), intermediate 38 (37 mg, 0.22 mmol) and triethylamine (0.07 mL, 0.50 mmol) in DCM (1 mL) under nitrogen atmosphere. The mixture was stirred at rt for 17 h. Then aqueous NaHCO₃ (aq. sat. soltn.) was added and the organic layer was separated, dried (MgSO₄), filtered and concentrated in vacuo. The residue was purified by flash chromatography (silica, EtOAc in heptane from 0/100 to 80/20). The desired fractions were collected and concentrated in vacuo affording product 64 as a yellow oil (12 mg, 17% yield).

### Preparation of Product 65

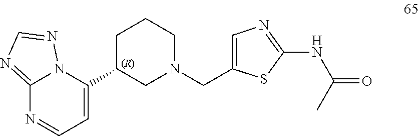

Sodium triacetoxyborohydride (76.74 mg, 0.36 mmol) was added to a solution of intermediate 7 (40 mg, 0.14 mmol, bishydrochloric acid salt), intermediate 38 (37 mg, 0.22 mmol) and triethylamine (0.075 mL, 0.54 mmol) in DCM (1 mL) under nitrogen atmosphere. The mixture was stirred at rt for 3 days. Then the mixture was quenched with water and extracted with EtOAc. The organic layer was washed with brine, separated, dried (MgSO₄), filtered and concentrated in vacuo. The crude product was purified by RP HPLC (Stationary phase: C18 XBridge 30×150 mm 5 μm, Mobile phase: Gradient from 81% 10 mM NH₄CO₃H pH 9 solution in water, 19% CH₃CN to 64% 10 mM NH₄CO₃H pH 9 solution in water, 36% CH₃CN). The desired fractions were collected and concentrated in vacuo affording product 65 as a pale yellow solid (17 mg, 33% yield).

### Preparation of Product 66

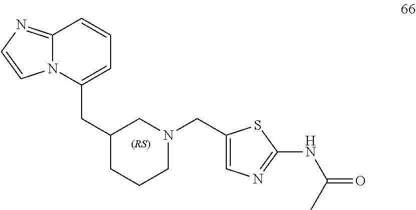 [SMILES: CC(=O)Nc1ncc(CN2CCCC(Cc3cccc4nccn34)C2)s1]

Sodium triacetoxyborohydride (50.51 mg, 0.24 mmol) was added to a solution of intermediate 97 (50 mg, 0.20 mmol, hydrochloric acid salt), intermediate 38 (40.56 mg, 0.24 mmol) and triethylamine (0.08 mL, 0.60 mmol) in DCM (1.2 mL) under nitrogen atmosphere. The mixture was stirred at rt for 3 days. Then NaHCO₃ (aq. sat. soltn.) was added and the mixture was extracted with DCM. The organic layer was washed with brine, separated, dried (MgSO₄), filtered and concentrated in vacuo. The crude product was purified by RP HPLC (Stationary phase: C18 XBridge 30×150 mm 5 μm, Mobile phase: Gradient from 81% 10 mM NH₄CO₃H pH 9 solution in water, 19% CH₃CN to 64% 10 mM NH₄CO₃H pH 9 solution in water, 36% CH₃CN). The desired fractions were collected and concentrated in vacuo affording product 66 as a pale yellow film (5 mg, 7% yield).

### Preparation of Product 67

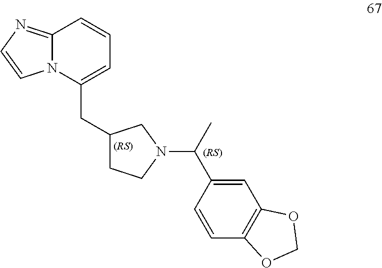 [SMILES: CC(c1ccc2c(c1)OCO2)N1CCC(Cc2cccc3nccn23)C1]

Titanium (IV) isopropoxide (0.27 mL, 0.92 mmol) was added to a stirred solution of intermediate 86 (90 mg, 0.45 mmol), 3′,4′-(methylenedioxy)acetophenone (CAS: 3162-29-6; 83 mg, 0.50 mmol) and DIPEA (0.2 mL, 1.16 mmol) in THF (2.1 mL) in a sealed tube at rt. The reaction mixture was stirred at 85° C. for 5 h and then sodium cyanoborohydride (30 mg, 0.48 mmol) was added. The reaction mixture was stirred at 50° C. for 48 h. Then the mixture was treated with NaHCO₃ (aq. sat. soltn.) and stirred for 1 h. The mixture was extracted with DCM. The organic layer was separated, dried (MgSO₄), filtered and concentrated in vacuo. The residue was purified by RP HPLC (Stationary phase: C18 XBridge 30×100 mm 5 μm, Mobile phase: Gradient from 67% 10 mM NH₄CO₃H pH 9 solution in water, 33% CH₃CN to 50% 10 mM NH₄CO₃H pH 9 solution in water, 50% CH₃CN). The desired fractions were collected and concentrated in vacuo affording product 67 as a pale yellow oil (20 mg, 13% yield).

### Preparation of Product 68

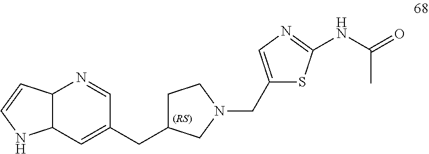 [SMILES: CC(=O)Nc1ncc(CN2CCC(CC3=CC4NC=CC4N=C3)C2)s1]

TFA (0.3 mL) was added to a stirred solution of intermediate 100 (78 mg, 0.16 mmol) in DCM (0.3 mL). The mixture was stirred for 16 h. The volatiles were evaporated in vacuo. Then, TFA (0.3 mL) was added and the mixture was stirred at 50° C. for 16 h. The volatiles were evaporated in vacuo. The residue was purified by ion exchange chromatography (Isolute® SCX2 eluting first with MeOH and then with 7N NH₃ in MeOH). The desired fractions were collected and concentrated in vacuo. The solid thus obtained was purified by flash chromatography (silica, 7M solution of ammonia in MeOH in DCM from 0/100 to 5/95). The desired fractions were collected and concentrated in vacuo. The resultant solid was purified by flash chromatography (silica, 7M solution of ammonia in MeOH in EtOAc from 0/100 to 5/95). The desired fractions were collected and concentrated in vacuo affording product 68 as a white solid (11 mg, 19% yield).

### Preparation of Product 69

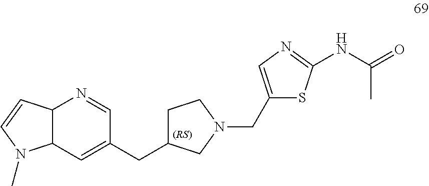 [SMILES: CC(=O)Nc1ncc(CN2CCC(CC3=CC4C(C=CN4C)N=C3)C2)s1]

Sodium triacetoxyborohydride (53.16 mg, 0.25 mmol) was added to a solution of intermediate 102 (36 mg, 0.17 mmol) and intermediate 38 (34.15 mg, 0.20 mmol) in MeOH (0.3 mL) and DCM (0.3 mL). The mixture was stirred at rt for 16 h. Then the mixture was concentrated in vacuo. The residue was purified by flash chromatography (silica, 7M solution of ammonia in MeOH in DCM from 0/100 to 6/94). The desired fractions were collected and concentrated in vacuo affording product 69 as a yellow solid (14 mg, 23% yield).

### Preparation of Products 70, 71 and 72

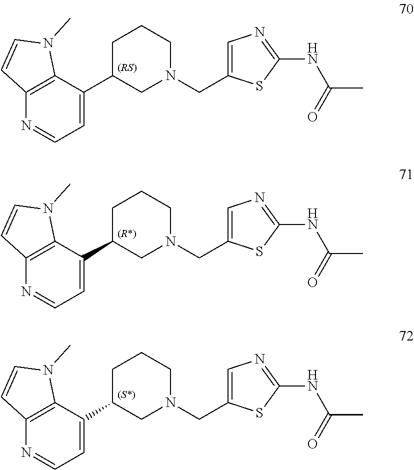

Diisopropylethylamine (0.15 mL, 0.85 mmol) was added to a suspension of intermediate 104 (42.9 mg, 0.17 mmol, hydrochloric acid salt) in DCM (0.9 mL) and the mixture was stirred at rt for 5 minutes. Then intermediate 38 (34.8 mg, 0.20 mmol) and sodium triacetoxyborohydride (54.17 mg, 0.26 mmol) were added and the mixture was stirred at rt for 16 h. Then aqueous NaHCO₃ (aq. sat. soltn.) was added and the organic layer was separated, dried (MgSO₄), filtered and concentrated in vacuo. The residue was purified by flash chromatography (silica, MeOH in DCM from 0/100 to 10/90). The desired fractions were collected and concentrated in vacuo affording product 70 as an orange oil (41.6 mg, 66% yield).

Product 70 was separated into enantiomers by purification by Prep SFC (Stationary phase: Chiralpak AD-H 5 μm 250×30 mm; mobile phase: 60% CO₂, 40% EtOH) affording product 71 (15 mg) and product 72 (17 mg) as yellow solids.

### Preparation of Product 73

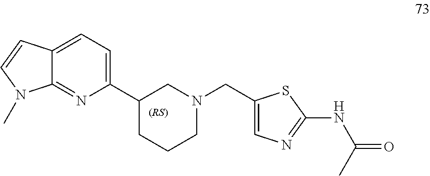 [SMILES: CC(=O)Nc1ncc(CN2CCCC(c3ccc4ccn(C)c4n3)C2)s1]

AcOH (0.02 mL) was added to a stirred mixture of intermediate 107 (38.7 mg, 0.18 mmol) and intermediate 38 (34 mg, 0.20 mmol) in MeOH (1 mL) at rt for 4 h in a sealed tube. Then sodium cyanoborohydride (66.6 mg, 1.06 mmol) was added and the mixture was stirred at rt for 21 h. Additional intermediate 38 (15.1 mg, 0.09 mmol) and sodium cyanoborohydride (46.1 mg, 0.73 mmol) were added and the mixture was stirred at rt for 2 h. Then additional sodium cyanoborohydride (50.2 mg, 0.80 mmol) was added and the mixture was stirred at rt for 15 h. Then the solvents were evaporated in vacuo. The residue was treated with NaHCO₃ (aq. sat. soltn.) and extracted with DCM. The organic layer was separated, dried (MgSO₄), filtered and concentrated in vacuo. The residue was purified by RP HPLC (Stationary phase: C18 XBridge 30×100 mm 5 μm, Mobile phase: Gradient from 75% 10 mM NH₄CO₃H pH 9 solution in water, 25% CH₃CN to 57% 10 mM NH₄CO₃H pH 9 solution in water, 43% CH₃CN). The desired fractions were collected and concentrated in vacuo affording product 73 as a pale yellow oil (11.9 mg, 18% yield).

### Preparation of Product 74, 75 and 76

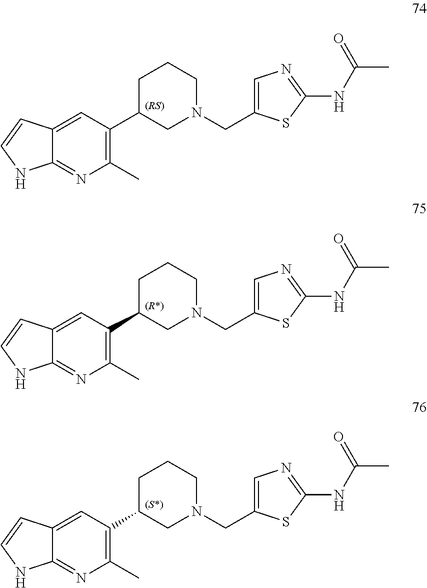 [SMILES: CC(=O)Nc1ncc(CN2CCCC(c3cc4cc[nH]c4nc3C)C2)s1 ; CC(=O)Nc1ncc(CN2CCC[C@H](c3cc4cc[nH]c4nc3C)C2)s1 ; CC(=O)Nc1ncc(CN2CCC[C@@H](c3cc4cc[nH]c4nc3C)C2)s1]

Diisopropylethylamine (0.6 mL, 3.48 mmol) was added to a suspension of intermediate 110 (150 mg, 0.70 mmol) in DCE (10 mL) and the mixture was stirred at rt for 10 minutes. Then intermediate 38 (142.29 mg, 0.84 mmol) was added and the suspension thus obtained was stirred for 2 h. Then sodium triacetoxyborohydride (324.86 mg, 1.53 mmol) was added and the mixture was stirred at rt for 16 h. Then water and DCM were added and the organic layer was separated, dried (Na₂SO₄), filtered and concentrated in vacuo. The residue was purified by RP HPLC (Stationary phase: C18 XBridge 30×100 mm 5 μm, Mobile phase: Gradient from 74% 0.1% NH₄CO₃H/NH₄OH pH 9 solution in water, 26% CH₃CN to 58% 0.1% NH₄CO₃H/NH₄OH pH 9 solution in water, 42% CH₃CN). The desired fractions were collected and concentrated in vacuo affording product 74 as a white solid (57 mg, 22% yield).

Product 74 was separated into enantiomers by purification by Prep SFC (Stationary phase: Chiralcel OJ-H 5 μm 250×20 mm; mobile phase: 80% CO₂, 20% MeOH (0.3% ⁱPrNH₂)) affording product 75 (21 mg) and product 76 (23 mg).

### Preparation of Product 77, 78 and 79

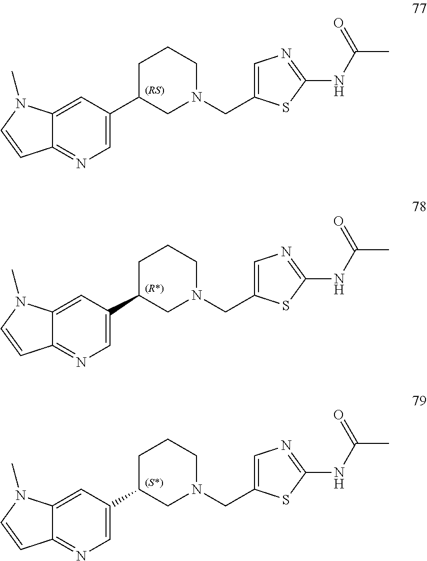 [SMILES: CC(=O)Nc1ncc(CN2CCCC(c3cnc4ccn(C)c4c3)C2)s1 ; CC(=O)Nc1ncc(CN2CCC[C@H](c3cnc4ccn(C)c4c3)C2)s1 ; CC(=O)Nc1ncc(CN2CCC[C@@H](c3cnc4ccn(C)c4c3)C2)s1]

Diisopropylethylamine (0.43 mL, 2.49 mmol) was added to a suspension of intermediate 112 (107.4 mg, 0.50 mmol) in DCM (2.65 mL) and the mixture was stirred at rt for 5 minutes. Then intermediate 38 (101.88 mg, 0.60 mmol) and sodium triacetoxyborohydride (158.59 mg, 0.75 mmol) were added and the mixture was stirred at rt for 16 h. Then aqueous NaHCO₃ (aq. sat. soltn.) was added and the organic layer was separated, dried (MgSO₄), filtered and concentrated in vacuo. The residue was purified by flash chromatography (silica, MeOH in DCM from 0/100 to 10/90). The desired fractions were collected and concentrated in vacuo affording product 77 as a yellow oil (111 mg, 60% yield).

Product 77 was separated into enantiomers by purification by Prep SFC (Stationary phase: Chiralpak AD-H 5 μm 250×30 mm; mobile phase: 55% CO₂, 45% EtOH (0.3% ⁱPrNH₂)) affording product 78 (42 mg) and product 79 (44 mg) as cream solids.

### Preparation of Product 26, 80 and 81

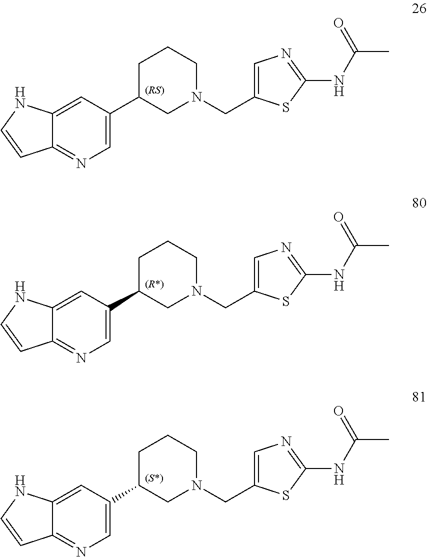 [SMILES: CC(=O)Nc1ncc(CN2CCCC(c3cnc4cc[nH]c4c3)C2)s1 ; CC(=O)Nc1ncc(CN2CCC[C@H](c3cnc4cc[nH]c4c3)C2)s1 ; CC(=O)Nc1ncc(CN2CCC[C@@H](c3cnc4cc[nH]c4c3)C2)s1]

Diisopropylethylamine (0.91 mL, 5.26 mmol) was added to a suspension of intermediate 58 (250 mg, 1.05 mmol, hydrochloric acid salt) in DCM (16 mL) and the mixture was stirred at rt for 5 minutes. Then intermediate 38 (196.87 mg, 1.16 mmol) and sodium triacetoxyborohydride (668.64 mg, 3.15 mmol) were added and the mixture was stirred at rt for 16 h. Then aqueous NaHCO₃ (aq. sat. soltn.) was added and the organic layer was separated, dried (MgSO₄), filtered and concentrated in vacuo. The residue was purified by flash chromatography (silica, MeOH in DCM from 0/100 to 10/90). The desired fractions were collected and concentrated in vacuo affording product 26 as a yellow oil (164.5 mg, 44% yield).

Product 26 was separated into enantiomers by purification by Prep SFC (Stationary phase: Chiralpak AD-H 5 μm 250×30 mm; mobile phase: 55% CO₂, 45% EtOH (0.3% ⁱPrNH₂)) affording product 80 (67 mg) and product 81 (68 mg) as cream solids.

### Preparation of Product 15, 82 and 83

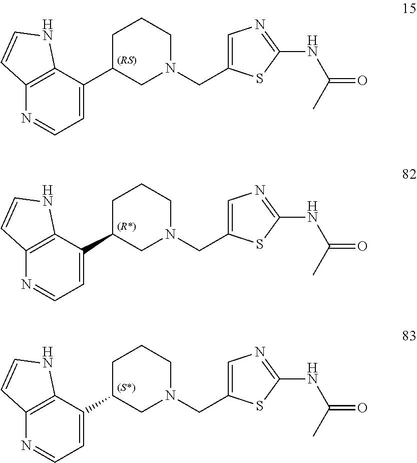 [SMILES: CC(=O)Nc1ncc(CN2CCCC(c3ccnc4cc[nH]c34)C2)s1 ; CC(=O)Nc1ncc(CN2CCC[C@H](c3ccnc4cc[nH]c34)C2)s1 ; CC(=O)Nc1ncc(CN2CCC[C@@H](c3ccnc4cc[nH]c34)C2)s1]

Diisopropylethylamine (0.72 mL, 4.21 mmol) was added to a suspension of intermediate 44 (200 mg, 0.84 mmol, hydrochloric acid salt) in DCM (13 mL) and the mixture was stirred at rt for 5 minutes. Then intermediate 38 (157.5 mg, 0.92 mmol) and sodium triacetoxyborohydride (534.91 mg, 2.52 mmol) were added and the mixture was stirred at rt for 16 h. Then aqueous NaHCO₃ (aq. sat. soltn.) was added and the organic layer was separated, dried (MgSO₄), filtered and concentrated in vacuo. The residue was purified by flash chromatography (silica, MeOH in DCM from 0/100 to 10/90). The desired fractions were collected and concentrated in vacuo affording product 15 as a yellow solid (116.5 mg, 39% yield).

Product 15 was separated into enantiomers by purification by Prep SFC (Stationary phase: Chiralpak AD-H 5 μm 250×30 mm; mobile phase: 60% CO₂, 40% ⁱPrOH (0.3% ⁱPrNH₂)) affording product 82 (44 mg) and product 83 (53 mg) as yellow solids.

### Preparation of Product 84

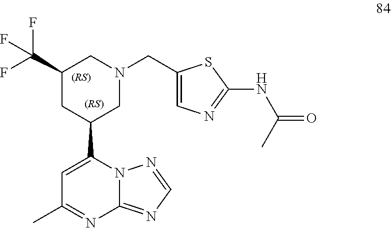 [SMILES: CC(=O)Nc1ncc(CN2C[C@@H](c3cc(C)nc4ncnn34)C[C@@H](C(F)(F)F)C2)s1]

AcOH (0.02 mL) was added to a stirred suspension of intermediate 118 (40 mg, 0.14 mmol) and intermediate 38 (32 mg, 0.19 mmol) in MeOH (1 mL) at rt for 2.5 h in a sealed tube. Then sodium cyanoborohydride (28 mg, 0.44 mmol) was added and the mixture was stirred at rt for 60 h. The residue was treated with NaHCO₃ (aq. sat. soltn.) and extracted with DCM. The organic layer was separated, dried (MgSO₄), filtered and concentrated in vacuo. The residue was purified purified by flash chromatography (silica, 7M solution of ammonia in MeOH in DCM from 0/100 to 5/95). The desired fractions were collected and concentrated in vacuo affording product 84 as a colorless oil (12 mg, 19% yield).

### Preparation of Products 85, 88, 91, 94, 97, 123, 101, 105, 106, 107 and 108

The following compounds were prepared following a reductive amination procedure like the one described for the preparation of product 17 starting from the corresponding amine and intermediate 38 using a reducting agent. Reducting agent, solvent, additives and changes of solvent are mentioned in the Table A below.

TABLE A

| PRODUCT | | INTERMEDIATE AMINE | | COMMENT |
|---|---|---|---|---|
| 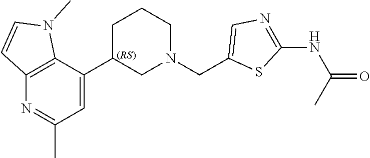 [SMILES: CC(=O)Nc1ncc(CN2CCCC(c3cc(C)nc4ccn(C)c34)C2)s1] | 85 | 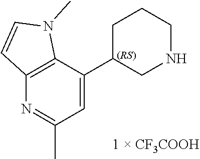 [SMILES: Cc1cc(C2CCCNC2)c2c(ccn2C)n1] | I-119 | Na(CN)BH₃ additives: CH₃CO₂H, CH₃CO₂Na Solvent: MeOH |
| 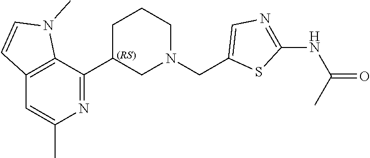 [SMILES: CC(=O)Nc1ncc(CN2CCCC(c3nc(C)cc4ccn(C)c34)C2)s1] | 88 | 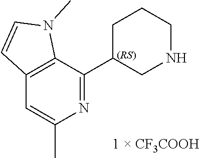 [SMILES: Cc1cc2ccn(C)c2c(C2CCCNC2)n1] | I-120 | Na(CN)BH₃ additives: CH₃CO₂H, CH₃CO₂Na Solvent: MeOH |
| 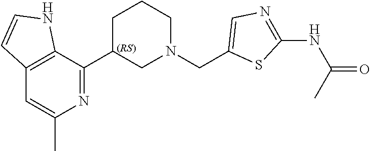 [SMILES: CC(=O)Nc1ncc(CN2CCCC(c3nc(C)cc4cc[nH]c34)C2)s1] | 91 | 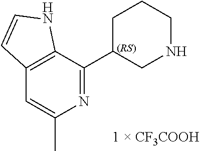 [SMILES: Cc1cc2cc[nH]c2c(C2CCCNC2)n1] | I-121 | Na(OAc)₃BH/ Na(CN)BH₃ additives: CH₃OH Base: DIPEA Solvent: DCM |
| 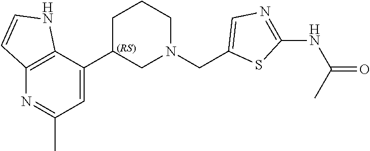 [SMILES: CC(=O)Nc1ncc(CN2CCCC(c3cc(C)nc4cc[nH]c34)C2)s1] | 94 | 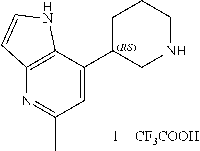 [SMILES: Cc1cc(C2CCCNC2)c2[nH]ccc2n1] | I-122 | Na(OAc)₃BH/ Na(CN)BH₃ CH₃OH Base: DIPEA Solvent: DCM |
| 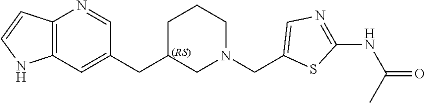 [SMILES: CC(=O)Nc1ncc(CN2CCCC(Cc3cnc4cc[nH]c4c3)C2)s1] | 97 | 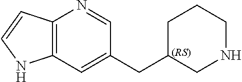 [SMILES: c1cc2ncc(CC3CCCNC3)cc2[nH]1] | I-123 | Na(OAc)₃BH Solvent: DCM |
| 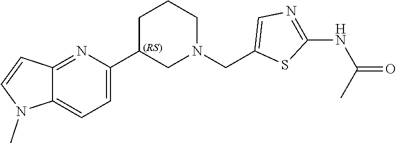 [SMILES: CC(=O)Nc1ncc(CN2CCCC(c3ccc4c(ccn4C)n3)C2)s1] | 100 | 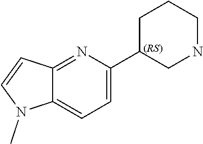 [SMILES: Cn1ccc2nc(C3CCCNC3)ccc21] | I-124 | Na(CN)BH₃ additives: CH₃CO₂H, Solvent: MeOH |
| 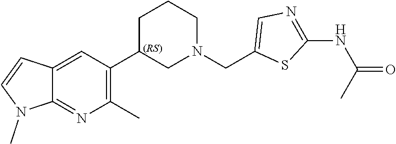 [SMILES: CC(=O)Nc1ncc(CN2CCCC(c3cc4ccn(C)c4nc3C)C2)s1] | 101 | 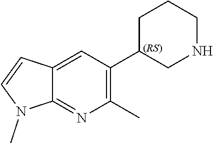 [SMILES: Cc1nc2c(ccn2C)cc1C1CCCNC1] | I-125 | Na(OAc)₃BH base: DIPEA Solvent: 1, 2- dichloroethane |
| 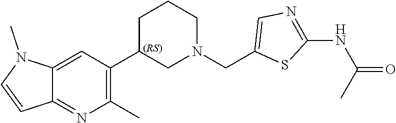 [SMILES: CC(=O)Nc1ncc(CN2CCCC(c3cc4c(ccn4C)nc3C)C2)s1] | 105 | 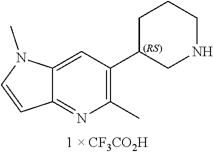 [SMILES: Cc1nc2ccn(C)c2cc1C1CCCNC1] | I-126 | Na(CN)BH₃ additives: CH₃CO₂H, CH₃CO₂Na Base: DIPEA Solvent: MeOH |
| 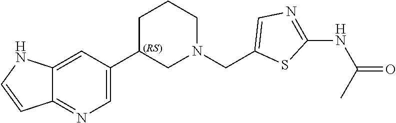 [SMILES: CC(=O)Nc1ncc(CN2CCCC(c3cnc4cc[nH]c4c3)C2)s1] | 106 | 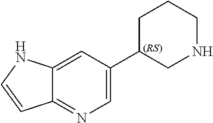 [SMILES: c1cc2ncc(C3CCCNC3)cc2[nH]1] | I-127 | Na(CN)BH₃ additives: CH₃CO₂H Solvent: MeOH |
| 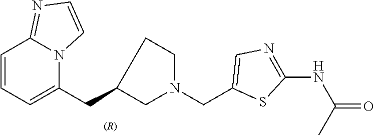 | 107 | 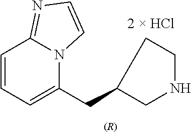 | I-128 | Na(OAc)₃BH Base: NEt₃ Solvent: DCM |
| 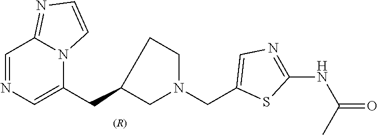 | 108 | 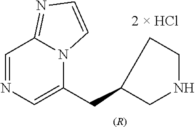 | I-129 | Na(OAc)₃BH Base: NEt₃ Solvent: DCM |

PREPARATION OF PRODUCTS 86 and 87

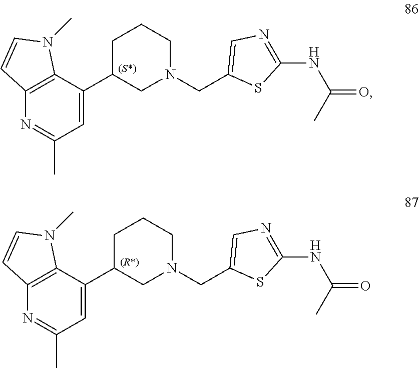

Product 85 (68 mg) was subjected to chiral SFC (stationary phase: CHIRACEL OJ-H 5 μm 250*20 mm, mobile phase: 80% CO₂, 20% EtOH(0.3% iPrNH₂)) to yield product 87 (32 mg) and product 86 (34 mg).

### Preparation of Products 89 and 90

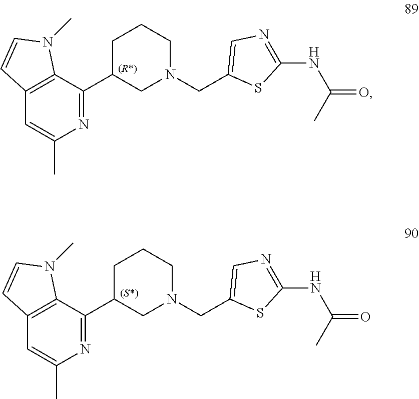 [SMILES: CC(=O)Nc1ncc(CN2CCCC(c3nc(C)cc4ccn(C)c34)C2)s1 ; CC(=O)Nc1ncc(CN2CCCC(c3nc(C)cc4ccn(C)c34)C2)s1]

Product 88 (96 mg) was subjected to chiral SFC (stationary phase: Chiralcel OD-H 5 μm 250×21.2 mm, mobile phase: 60% CO₂, 40% MeOH(0.3% iPrNH₂)) to yield product 89 (26 mg) and product 90 (27 mg).

### Preparation of Products 92 and 93

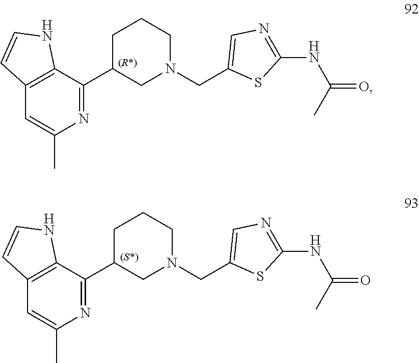 [SMILES: CC(=O)Nc1ncc(CN2CCCC(c3nc(C)cc4cc[nH]c34)C2)s1 ; CC(=O)Nc1ncc(CN2CCCC(c3nc(C)cc4cc[nH]c34)C2)s1]

Product 91 (100 mg) was subjected to chiral SFC (stationary phase: CHIRALPAK AD-H 5 μm 250*30 mm, mobile phase: 75% CO₂, 25% EtOH(0.3% iPrNH₂)) to yield product 92 (35 mg) and product 93 (36 mg).

### Preparation of Products 95 and 96

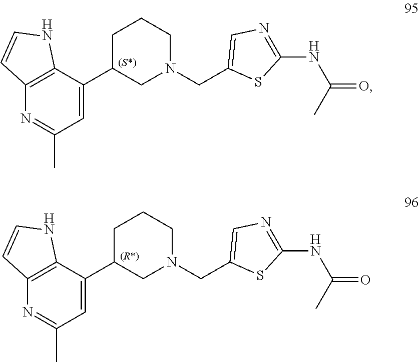 [SMILES: CC(=O)Nc1ncc(CN2CCCC(c3cc(C)nc4cc[nH]c34)C2)s1 ; CC(=O)Nc1ncc(CN2CCCC(c3cc(C)nc4cc[nH]c34)C2)s1]

Product 94 (66 mg) was subjected to chiral SFC (stationary phase: Chiralcel OD-H 5 μm 250×21.2 mm, mobile phase: 70% CO₂, 30% EtOH(0.3% iPrNH₂)) to yield product 95 (27 mg) and product 96 (26 mg).

### Preparation of Products 98 and 99

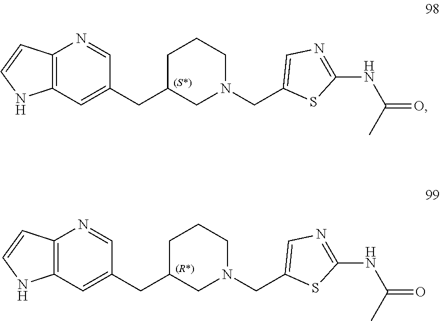 [SMILES: CC(=O)Nc1ncc(CN2CCCC(Cc3cnc4cc[nH]c4c3)C2)s1 ; CC(=O)Nc1ncc(CN2CCCC(Cc3cnc4cc[nH]c4c3)C2)s1]

Product 97 (68 mg) was subjected to chiral SFC (stationary phase: CHIRALPAK AD-H 5 μm 250*30 mm, mobile phase: 50% CO₂, 50% MeOH(0.3% iPrNH₂)) to yield product 98 (26 mg) and product 99 (27 mg).

### Preparation of Products 102 and 103

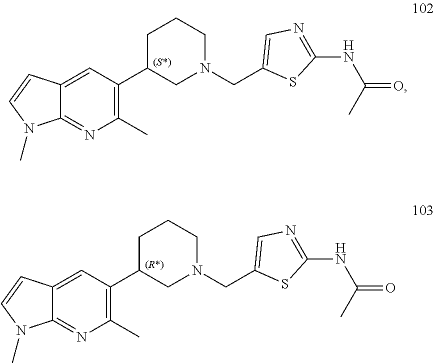 [SMILES: CC(=O)Nc1ncc(CN2CCCC(c3cc4ccn(C)c4nc3C)C2)s1 ; CC(=O)Nc1ncc(CN2CCCC(c3cc4ccn(C)c4nc3C)C2)s1]

Product 101 (80 mg) was subjected to chiral SFC (stationary phase: CHIRALPAK AD-H 5 μm 250*30 mm, mobile phase: 50% CO₂, 50% MeOH(0.3% iPrNH₂)) to yield product 102 (32 mg) and product 103 (35 mg).

### Preparation of Product 104

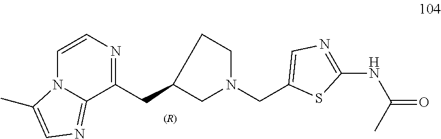

Triethylamine (0.067 mL, 0.49 mmol) was added to a suspension of intermediate 130 (35 mg, 0.121 mmol) in DCM (1 mL) and the mixture was stirred at rt for 2 minutes. Then intermediate 38 (24.7 mg, 0.15 mmol) and sodium triacetoxyborohydride (77 mg, 0.36 mmol) were added and the mixture was stirred at rt for 16 h. Then aqueous NaHCO₃ (aq. sat. soltn.) was added and the organic layer was separated, dried (MgSO₄), filtered and concentrated in vacuo. The residue was purified by RP HPLC (Stationary phase: C18 XBridge 30×100 mm 5 μm), Mobile phase: gradient from 90% NH₄HCO₃ 0.25% solution in water, 10% CH₃CN to 65% NH₄HCO₃ 0.25% solution in water, 35% CH₃CN). The desired fractions were collected and concentrated in vacuo to yield product 104 as a yellow oil (26.7 mg, 60% yield).

### Preparation of Product 109

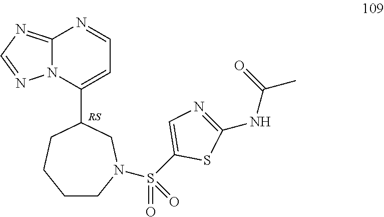 [SMILES: CC(=O)Nc1ncc(S(=O)(=O)N2CCCCC(c3ccnc4ncnn34)C2)s1]

2-Acetylamino-thiazole-5-sulfonyl chloride ([654072-71-6], 41.5 mg, 0.17 mmol) was added to a stirred solution of intermediate 152 (50 mg, 0.17 mmol) and DIPEA (0.095 mL, 0.55 mmol) in DCM (0.482 mL) at 0° C. for 1 h. Then NaHCO₃ (aq. sat. sol.) was added and the organic layer was separated, dried (MgSO₄), filtered and concentrated in vacuo. The crude product was washed with Et₂O and dried in the vacuum oven to yield product 109 (9 mg, 12%) as a pale brown solid.

The following compounds were prepared following the methods exemplified in the Experimental Part. In case no salt form is indicated, the compound was obtained as a free base. ‘Co. No.’ means compound number.

TABLE 1

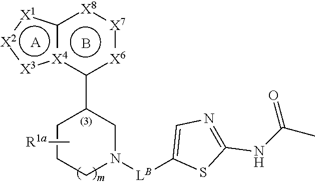

| | | | | | | | | | | | Ste- |
|---|---|---|---|---|---|---|---|---|---|---|---|
| Co. | | | | | | | | | | | reo |
| No. | X¹ | X² | X³ | X⁴ | X⁶ | X⁷ | X⁸ | m | R^{1a} | L^{B} | chem |
| 1 | N | CH | N | N | CH | CH | N | 1 | — | —SO₂— | 3-R |
| 2 | N | CH | CH | N | CH | CH | N | 1 | — | —SO₂— | 3-RS |
| 3 | N | CH | N | N | CH | CCH₃ | N | 1 | — | —SO₂— | 3-RS |
| 4 | N | CH | N | N | CH | CH | N | 1 | — | —SO₂— | 3-S |
| 5 | N | CH | CH | N | CH | CCHF₂ | N | 1 | — | —SO₂— | 3-RS |
| 6 | N | CH | N | N | CH | CCHF₂ | N | 1 | — | —SO₂— | 3-RS |
| 7 | N | CH | N | N | CH | CCHF₂ | N | 1 | 4- | —SO₂— | 3-R, |
| | | | | | | | | | CH₃ | | 4-S |
| 8 | N | CH | N | N | CH | CCH₃ | N | 0 | — | —SO₂— | 3-RS |
| 9 | N | CH | N | N | CH | CH | N | 1 | — | —CH₂— | 3-S |
| 10 | N | CH | CH | N | CH | CH | N | 1 | — | —CH₂— | 3-RS |
| 11 | N | CH | N | N | CH | CCH₃ | N | 1 | — | —CH₂— | 3-RS |
| 12 | N | CH | CH | N | CH | CH | CH | 1 | — | —CH₂— | 3-RS |
| 13 | N | CH | N | N | CH | CCHF₂ | N | 1 | — | —CH₂— | 3-RS |
| 14 | N | CH | CH | N | CH | CCHF₂ | N | 1 | — | —CH₂— | 3-RS |
| 15 | CH | CH | NH | C | CH | CH | N | 1 | — | —CH₂— | 3-RS |
| 16 | N | CH | N | N | CH | CCHF₂ | N | 1 | 4- | —CH₂— | 3-R, |
| | | | | | | | | | CH₃ | | 4-S |
| 17 | N | CH | CH | N | CH | CCH₃ | N | 1 | — | —CH₂— | 3-RS |
| 18 | N | CH | N | N | CH | CCH₃ | N | 0 | — | —CH₂— | 3-RS |
| 19 | N | CH | N | N | CF | CCH₃ | N | 0 | — | —CH₂— | 3-RS |
| 20 | N | CH | N | N | CH | CCF₃ | N | 0 | — | —CH₂— | 3-RS |
| 21 | CH | CH | N | N | CH | CCH₃ | N | 0 | — | —CH₂— | 3-RS |
| 65 | N | CH | N | N | CH | CH | N | 1 | — | —CH₂— | 3-R |
| 70 | CH | CH | NCH₃ | C | CH | CH | N | 1 | — | —CH₂— | 3-RS |
| 71 | CH | CH | NCH₃ | C | CH | CH | N | 1 | — | —CH₂— | 3-R* |
| 72 | CH | CH | NCH₃ | C | CH | CH | N | 1 | — | —CH₂— | 3-S* |
| 82 | CH | CH | NH | C | CH | CH | N | 1 | — | —CH₂— | 3-R* |
| 83 | CH | CH | NH | C | CH | CH | N | 1 | — | —CH₂— | 3-S* |
| 84 | N | CH | N | N | CH | CCH₃ | N | 1 | 5- | —CH₂— | cis |
| | | | | | | | | | CF₃ | | 3-RS, |
| | | | | | | | | | | | 5-RS |
| 85 | CH | CH | NCH₃ | C | CH | CCH₃ | N | 1 | — | —CH₂— | 3-RS |
| 86 | CH | CH | NCH₃ | C | CH | CCH₃ | N | 1 | — | —CH₂— | 3-S* |
| 87 | CH | CH | NCH₃ | C | CH | CCH₃ | N | 1 | — | —CH₂— | 3-R* |
| 88 | CH | CH | NCH₃ | C | N | CCH₃ | CH | 1 | — | —CH₂— | 3-RS |
| 89 | CH | CH | NCH₃ | C | N | CCH₃ | CH | 1 | — | —CH₂— | 3-R* |
| 90 | CH | CH | NCH₃ | C | N | CCH₃ | CH | 1 | — | —CH₂— | 3-S* |
| 91 | CH | CH | NH | C | N | CCH₃ | CH | 1 | — | —CH₂— | 3-RS |
| 92 | CH | CH | NH | C | N | CCH₃ | CH | 1 | — | —CH₂— | 3-R* |
| 93 | CH | CH | NH | C | N | CCH₃ | CH | 1 | — | —CH₂— | 3-S* |
| 94 | CH | CH | NH | C | CH | CCH₃ | N | 1 | — | —CH₂— | 3-RS |
| 95 | CH | CH | NH | C | CH | CCH₃ | N | 1 | — | —CH₂— | 3-S* |
| 96 | CH | CH | NH | C | CH | CCH₃ | N | 1 | — | —CH₂— | 3-R* |
| 109 | N | CH | N | N | CH | CH | N | 2 | — | —SO₂— | 3-RS |

TABLE 2

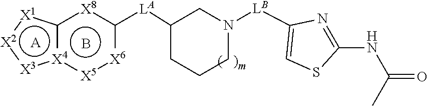

| Co. No. | X¹ | X² | X³ | X⁴ | X⁵ | X⁶ | X⁸ | m | R^{1a} | L^{B} | Stereochem |
|---|---|---|---|---|---|---|---|---|---|---|---|
| 22 | N | CH | N | N | CCHF₂ | CH | N | 1 | bond | —SO₂— | 3-R |
| 23 | N | CH | CH | N | CCHF₂ | CH | N | 1 | bond | —SO₂— | 3-RS |
| 24 | N | CH | CH | N | CCHF₂ | CH | N | 1 | bond | —CH₂— | 3-RS |
| 25 | N | CH | N | N | CCHF₂ | CH | N | 1 | bond | —CH₂— | 3-R |
| 26 | NH | CH | CH | C | N | CH | CH | 1 | bond | —CH₂— | 3-RS |
| 68 | NH | CH | CH | C | N | CH | CH | 0 | —CH₂— | —CH₂— | 3-RS |
| 69 | NCH₃ | CH | CH | C | N | CH | CH | 0 | —CH₂— | —CH₂— | 3-RS |
| 73 | NCH₃ | CH | CH | C | CH | CH | N | 1 | bond | —CH₂— | 3-RS |
| 77 | NCH₃ | CH | CH | C | N | CH | CH | 1 | bond | —CH₂— | 3-RS |
| 78 | NCH₃ | CH | CH | C | N | CH | CH | 1 | bond | —CH₂— | 3-R* |
| 79 | NCH₃ | CH | CH | C | N | CH | CH | 1 | bond | —CH₂— | 3-S* |
| 80 | NH | CH | CH | C | N | CH | CH | 1 | bond | —CH₂— | 3-R* |
| 81 | NH | CH | CH | C | N | CH | CH | 1 | bond | —CH₂— | 3-S* |
| 97 | NH | CH | CH | C | N | CH | CH | 1 | —CH₂— | —CH₂— | 3-RS |
| 98 | NH | CH | CH | C | N | CH | CH | 1 | —CH₂— | —CH₂— | 3-S* |
| 99 | NH | CH | CH | C | N | CH | CH | 1 | —CH₂— | —CH₂— | 3-R* |
| 100 | CH | CH | NC | C | CH | CH | N | 1 | bond | —CH₂— | 3-RS |
| | | | H₃ | | | | | | | | |
| 101 | CH | CH | NC | C | N | CCH₃ | CH | 1 | bond | —CH₂— | 3-RS |
| | | | H₃ | | | | | | | | |
| 102 | CH | CH | NC | C | N | CCH₃ | CH | 1 | bond | —CH₂— | 3-S* |
| | | | H₃ | | | | | | | | |
| 103 | CH | CH | NC | C | N | CCH₃ | CH | 1 | bond | —CH₂— | 3R* |
| | | | H₃ | | | | | | | | |
| 105 | NCH₃ | CH | CH | C | N | CCH₃ | CH | 1 | bond | —CH₂— | 3-RS |
| 106 | NH | CH | CH | C | N | CH | CH | 1 | bond | —CH₂— | 3-RS |

TABLE 3

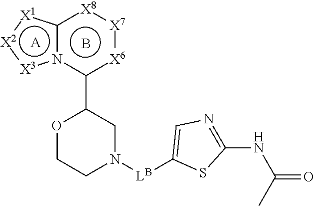

| Co. | | | | | | | | |
|---|---|---|---|---|---|---|---|---|
| No. | X¹ | X² | X³ | X⁶ | X⁷ | X⁸ | L^{B} | Stereochem |
| 27 | N | CH | N | CH | CCF₃ | N | —SO₂— | 2-RS |
| 28 | N | CH | N | CH | CCH₃ | N | —SO₂— | 2-RS |
| 29 | N | CH | N | CH | CCH₃ | N | —SO₂— | 2-S* |
| 30 | N | CH | N | CH | CCH₃ | N | —SO₂— | 2-R* |
| 31 | N | CH | N | CH | CH | N | —SO₂— | 2-R |
| 32 | N | CH | N | CH | CH | N | —SO₂— | 2-S |
| 33 | N | CH | N | CH | CCHF₂ | N | —SO₂— | 2-RS |
| 34 | N | CH | N | CH | CCHF₂ | N | —CH₂— | 2-RS |
| 35 | N | CH | N | CH | CCH₃ | N | —CH₂— | 2-RS |
| 36 | N | CH | N | CH | CH | N | —CH₂— | 2-RS |
| 37 | N | CH | N | CH | CCF₃ | N | —CH₂— | 2-RS |

TABLE 4

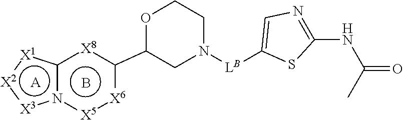

| Co. | | | | | | | | |
|---|---|---|---|---|---|---|---|---|
| No. | X¹ | X² | X³ | X⁵ | X⁶ | X⁸ | L^{B} | Stereochem |
| 38 | N | CH | N | CCHF₂ | CH | N | —SO₂— | 2-RS |
| 39 | N | CH | N | CCHF₂ | CH | N | —CH₂— | 2-RS |

TABLE 5

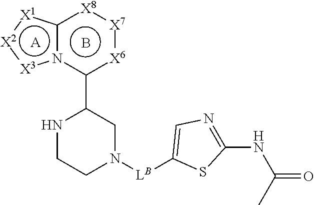

| Co. | | | | | | | | |
|---|---|---|---|---|---|---|---|---|
| No. | X¹ | X² | X³ | X⁶ | X⁷ | X⁸ | L^{B} | Stereochem |
| 40 | N | CH | N | CH | CCH₃ | N | —SO₂— | 2-RS |
| 41 | N | CH | N | CH | CCHF₂ | N | —SO₂— | 2-RS |
| 42 | N | CH | N | CH | CCH₃ | N | —CH₂— | 2-RS |

TABLE 6

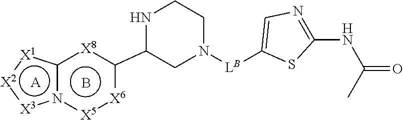

| Co. | | | | | | | | |
|---|---|---|---|---|---|---|---|---|
| No. | X¹ | X² | X³ | X⁵ | X⁶ | X⁸ | L^{B} | Stereochem |
| 43 | N | CH | N | CCHF² | CH | N | —SO₂— | 2-RS |

TABLE 7

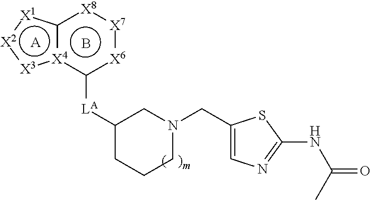

| Co.nr. | X¹ | X² | X³ | X⁴ | X⁶ | X⁷ | X⁸ | m | L^{A} | Stereochem |
|---|---|---|---|---|---|---|---|---|---|---|
| 44 | CH | CH | N | N | CH | CCH₃ | N | 1 | —CH₂— | 3-RS |
| 45 | N | CH | CH | N | CH | N | CH | 1 | —CH₂— | 3-RS |
| 46 | N | CH | N | N | CH | CCH₃ | N | 1 | —CH₂— | 3-RS |
| 47 | N | CH | N | N | CF | CCH₃ | N | 1 | —CH₂— | 3-RS |
| 48 | N | CH | N | N | CH | CCH₃ | N | 0 | —CH₂— | 3-RS |
| 49 | N | CH | N | N | CF | CCH₃ | N | 0 | —CH₂— | 3-RS |
| 50 | N | CH | CH | N | CH | CH | CH | 0 | —CH₂— | 3-RS |
| 51 | N | CH | N | N | CH | CCH₃ | N | 1 | —NH— | 3-RS |
| 66 | N | CH | CH | N | CH | CH | CH | 1 | —CH₂— | 3-RS |
| 107 | N | CH | CH | N | CH | CH | CH | 0 | —CH₂— | 3-R |
| 108 | N | CH | CH | N | CH | N | CH | 0 | —CH₂— | 3-R |

TABLE 8

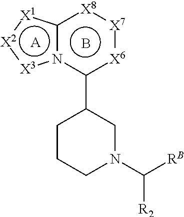

| Co. | | | | | | | | | |
|---|---|---|---|---|---|---|---|---|---|
| No. | X¹ | X² | X³ | X⁶ | X⁷ | X⁸ | R² | R^{B} | Stereochem |
| 52 | N | CH | CH | CH | CCH₃ | N | —CH₃ | b-2 | 1′-RS-3-RS |

TABLE 9

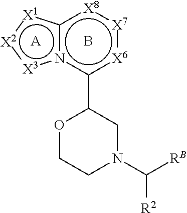

| Co. | | | | | | | | | |
|---|---|---|---|---|---|---|---|---|---|
| No. | X¹ | X² | X³ | X⁶ | X⁷ | X⁸ | R² | R^{B} | Stereochem |
| 53 | N | CH | N | CH | CCH₃ | N | —CH₃ | b-2 | 1′-RS, 2-RS |
| 54 | N | CH | N | CH | CCF₃ | N | —CH₃ | b-2 | 1′-RS, 2-RS |
| 55 | N | CH | N | CH | CH | N | —CH₃ | b-2 | 1′-RS, 2-RS |

TABLE 10

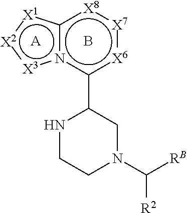

| Co. | | | | | | | | | |
|---|---|---|---|---|---|---|---|---|---|
| No. | X¹ | X² | X³ | X⁶ | X⁷ | X⁸ | R² | R^{B} | Stereochem |
| 56 | N | CH | N | CH | CCH₃ | N | —CH₃ | b-2 | trans, |
| | | | | | | | | | 1′-RS-2-RS |
| 57 | N | CH | N | CH | CCH₃ | N | —CH₃ | b-2 | cis, |
| | | | | | | | | | 1′-RS-2-RS |

TABLE 11

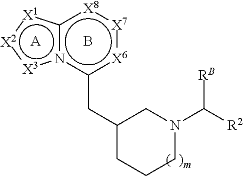

| Co. | | | | | | | | | | |
|---|---|---|---|---|---|---|---|---|---|---|
| No. | X¹ | X² | X³ | X⁶ | X⁷ | X⁸ | m | R² | R^{B} | Stereochem |
| 58 | N | CH | CH | CH | CH | CH | 0 | —H | b-4 | 3-RS |
| 67 | N | CH | CH | CH | CH | CH | 0 | —CH₃ | b-2 | 1′-RS-3-RS |

TABLE 12

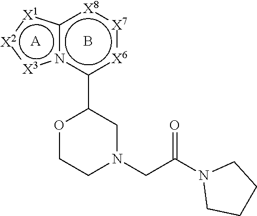

| Co. No. | X¹ | X² | X³ | X⁶ | X⁷ | X⁸ | Stereochem |
|---|---|---|---|---|---|---|---|
| 59 | N | CH | N | CH | CCH₃ | N | 2-RS |

TABLE 13

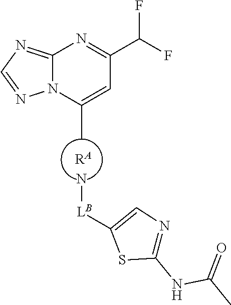

| Co. No. | 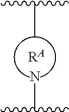 | L^{B} |
|---|---|---|
| 60 | 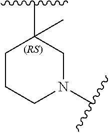 [SMILES: *N1CCCC(*)(C)C1] | —SO₂— |
| 61 | 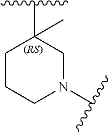 [SMILES: *N1CCCC(*)(C)C1] | —CH₂— |
| 63 | 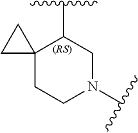 [SMILES: *C1CN(*)CCC12CC2] | —SO₂— |
| 64 | 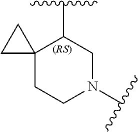 [SMILES: *C1CN(*)CCC12CC2] | —CH₂— |

TABLE 14

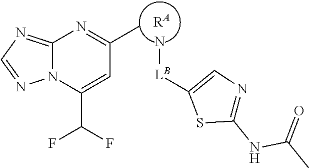

| Co. No. | 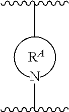 | L^{B} |
|---|---|---|
| 62 | 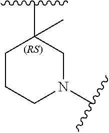 [SMILES: *N1CCCC(*)(C)C1] | —CH₂— |

TABLE 15

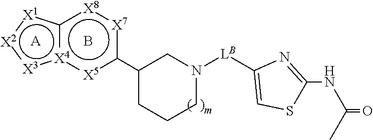

| Co. | | | | | | | | | | Stereo- |
|---|---|---|---|---|---|---|---|---|---|---|
| nr. | X¹ | X² | X³ | X⁴ | X⁵ | X⁷ | X⁸ | m | L^{B} | chem |
| 74 | NH | CH | CH | C | CH | CCH₃ | N | 1 | —CH₂— | 3-RS |
| 75 | NH | CH | CH | C | CH | CCH₃ | N | 1 | —CH₂— | 3-R* |
| 76 | NH | CH | CH | C | CH | CCH₃ | N | 1 | —CH₂— | 3-S* |

TABLE 16

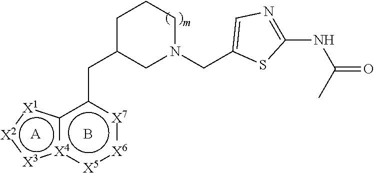

| Co. No. | X¹ | X² | X³ | X⁴ | X⁵ | X⁶ | X⁷ | m | Stereochem |
|---|---|---|---|---|---|---|---|---|---|
| 104 | N | CH | CCH3 | N | CH | CH | N | 0 | 3-R |

### C. Analytical Part

### Melting Points

Values are peak values, and are obtained with experimental uncertainties that are commonly associated with this analytical method.

DSC823e: For a number of compounds, melting points were determined with a DSC823e (Mettler-Toledo) apparatus. Melting points were measured with a temperature gradient of 10° C./minute. Maximum temperature was 300° C. Values are peak values.

### LCMS

### General Procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW) and/or exact mass monoisotopic molecular weight. Data acquisition was performed with appropriate software.

Compounds are described by their experimental retention times (Rₜ) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, [M+CH₃COO]⁻ etc. . . . ). For molecules with multiple isotopic patterns (Br, Cl.), the reported value is the one obtained for the lowest isotope mass. All results were obtained with experimental uncertainties that are commonly associated with the method used.

Hereinafter, “SQD” Single Quadrupole Detector, “MSD” Mass Selective Detector, “QTOF” Quadrupole-Time of Flight, “rt” room temperature, “BEH” bridged ethylsiloxane/silica hybrid, HSS” High Strength Silica, “CSH” charged surface hybrid, “UPLC” Ultra Performance Liquid Chromatography, “DAD” Diode Array Detector.

TABLE 17
LC-MS Methods (Flow expressed in mL/min; column temperature (T) in
° C.; Run time in min).

| Method | Instrument | Column | Mobile Phase | Gradient | $\frac{Flow}{Col T}$ | Run Time |
|---|---|---|---|---|---|---|
| 1 | Waters: Acquity ® UPLC ® - | Waters: BEH C18 (1.7 μm, | A: 95% CH₃COONH₄ 6.5 mM + | From 95% A to 5% A in 4.6 min, held for | $\frac{1}{50}$ | 5 |
| | DAD/SQD | 2.1 × 50 mm) | 5% CH₃CN, | 0.4 min | | |
| | | | B: CH₃CN | | | |
| 2 | Waters: Acquity ® IClass | Waters: BEH C18 (1.7 μm, | A: 95% CH₃COONH₄ 6.5 mM + | From 95% A to 5% A in 4.6 min, held for | $\frac{1}{50}$ | 5 |
| | UPLC ® - | 2.1 × 50 mm) | 5% CH₃CN, | 0.4 min | | |
| | DAD/Xevo | | B: CH₃CN | | | |
| | G2-S QTOF | | | | | |
| 3 | Waters: Acquity ® IClass | Waters: BEH C18 (1.7 μm, | A: 95% CH₃COONH₄ 6.5 mM + | From 95% A to 5% A in 4.6 min, held for | $\frac{1}{50}$ | 5 |
| | UPLC ® - | 2.1 × 50 mm) | 5% CH₃CN, | 0.4 min | | |
| | DAD/SQD | | B: CH₃CN | | | |
| 4 | Waters: Acquity ® UPLC ®- | Waters: HSS T3 column (1.8 | A: 95% CH₃COONH₄ 10 mM + | From 100% A to 95% A in 2.1 min, to 95% | $\frac{0.7}{55}$ | 3.5 |
| | DAD/SQD | μm, 2.1 × | 5% CH₃CN, | A in 0.9 min, | | |
| | | 100 mm) | B: CH₃CN | held for 0.5 min | | |
| 5 | Waters: Acquity UPLC ® | Waters: BEH C18 (1.7 μm, | A: 95% CH₃COONH₄ 7 mM + | From 84.2% A to 10.5% A in 2.2 min, held | $\frac{0.34}{40}$ | 6.1 |
| | H-Class - | 2.1 × 100 mm) | 5% CH₃CN | for 1.9 min, back | | |
| | DAD/SQD2 | | B: CH₃CN | to 84.2% A in | | |
| | | | | 0.7 min, held for | | |
| | | | | 0.7 min. | | |
| 6 | Waters: Acquity UPLC ® - | Waters: BEH C18 (1.7 μm, | A: 95% CH₃COONH₄ 7 mM + | 84.2% A for 0.5 min, to 10.5% A in 2.2 min, | $\frac{0.34}{40}$ | 6.2 |
| | DAD/ | 2.1 × 100 mm) | 5% CH₃CN | held for 1.9 | | |
| | Quattro | | B: CH₃CN | min, back to | | |
| | Micro ™ | | | 84.2% A in 0.7 | | |
| | | | | min, held for | | |
| | | | | 0.7 min. | | |
| 7 | Waters: Acquity ® IClass | Agilent: RRHD (1.8 μm, | A: 95% CH₃COONH₄ 6.5 mM + | From 95% A to 5% A in 4.6 min, held for | $\frac{1}{50}$ | 5 |
| | UPLC ® - | 2.1 × 50 mm) | 5% CH₃CN, | 0.4 min | | |
| | DAD/SQD | | B: CH₃CN | | | |
| 8 | Waters: Acquity ® UPLC ® - | Waters: BEH C18 (1.7 μm, | A: 95% CH₃COONH₄ 6.5 mM + | From 95% A to 40% A in 1.2 min, to 5% | $\frac{1}{50}$ | 2 |
| | DAD/SQD | 2.1 × 50 mm) | 5% CH₃CN, | A in 0.6 min, | | |
| | | | B: CH₃CN | held for 0.2 min | | |

TABLE 18
Analytical data-melting point (M.p.) and LCMS: [M +
H]⁺ means the protonated mass of the free base of the compound,
[M − H]⁻ means the deprotonated mass of the free base of the compound
or the type of adduct specified [M + CH₃COO]⁻).
Rₜ means retention time (in min). For some compounds, exact
mass was determined.

| Co. | | | | | LCMS |
|---|---|---|---|---|---|
| No. | M.p. (° C.) | [M + H]⁺ | [M − H]⁻ | Rₜ | Method |
| 1 | n.d. | — | 406 | 1.07 | 2 |
| 2 | n.m. | 407 | — | 1.05 | 2 |
| 3 | n.m. | 422 | — | 1.16 | 2 |
| 4 | n.d. | — | 406 | 1.06 | 2 |
| 5 | n.d. | 457 | 455 | 1.28 | 1 |
| 6 | 266.14° C. | 458 | — | 1.36 | 3 |
| 7 | 264.57° C. | — | 470 | 1.63 | 2 |
| 8 | n.m. | 408 | — | 0.83 | 1 |
| 9 | 234.60° C.* | 358 | 356 | 0.93 | 2 |
| 10 | n.m. | 357 | — | 1.02 | 2 |
| 11 | 246.95° C. | 372 | — | 1.33 | 2 |
| 12 | n.m. | — | 354 | 1.47 | 2 |
| 13 | 186.01° C. | 408 | | 1.26 | 2 |
| 14 | 226.06° C. | 407 | — | 1.26 | 2 |
| 15 | n.m. | 356 | — | 1.13 | 2 |
| 16 | n.m. | 422 | 420 | 1.49 | 2 |
| 17 | n.d. | 371 | — | 1.23 | 2 |
| 18 | n.m. | 358 | 356 | 0.79 | 1 |
| 19 | n.m. | 376 | — | 0.9 | 1 |
| 20 | 214.29° C. | 412 | 410 | 1.47 | 2 |
| 21 | n.m. | 357 | 355 | 1.31 | 2 |
| 22 | 263.43° C.* | 458 | — | 1.38 | 1 |
| 23 | 280.13° C. | 457 | 455 | 1.38 | 1 |
| 24 | 232.44° C. | 407 | — | 1.12 | 2 |
| 25 | 182.53° C. | 408 | — | 1.16 | 2 |
| 26 | n.m. | 356 | — | 0.94 | 2 |
| 27 | 255.12° C. | — | 476 | 1.65 | 2 |
| 28 | 290.78° C. | 424 | — | 1.1 | 2 |
| 29 | n.m. | 424 | 422 | 1.36 | 4 |
| 30 | n.m. | 424 | 422 | 1.36 | 4 |
| 31 | 263.16° C. | — | 408 | 1 | 2 |
| 32 | 260.51° C. | 410 | — | 1 | 2 |
| 33 | 264.85° C. | 460 | — | 1.39 | 2 |
| 34 | 260.21° C. | 410 | — | 1.21 | 2 |
| 35 | 264.26° C. | 374 | — | 0.93 | 2 |
| 36 | 251.21° C.* | — | 358 | 0.94 | 2 |
| 37 | 242.64° C. | 428 | — | 1.78 | 2 |
| 38 | 229.94° C. | 460 | — | 1.27 | 3 |
| 39 | 209.69° C. | 410 | — | 1.13 | 2 |
| 40 | 226.91° C. | — | 421 | 0.85 | 2 |
| 41 | 227.97° C.* | 459 | — | 1.05 | 2 |
| 42 | 228.95° C. | 373 | — | 0.86 | 2 |
| 43 | 226.22° C. | 459 | — | 0.97 | 1 |
| 44 | n.m. | 385 | — | 1.56 | 2 |
| 45 | n.m. | 371 | — | 0.97 | 2 |
| 46 | n.m. | 386 | — | 0.99 | 1 |
| 47 | n.m. | 404 | 402 | 1.55 | 2 |
| 48 | n.m. | 372 | — | 0.78 | 2 |
| 49 | n.m. | 390 | — | 1.22 | 2 |
| 50 | n.m. | 356 | — | 0.96 | 2 |
| 51 | 249.94° C. | 387 | — | 1.37 | 2 |
| 52 | n.d. | 365 | — | 1.96/1.98^{&} | 2 |
| 53 | n.d. | 368 | — | 2.01/2.06^{&} | 2 |
| 54 | n.d. | 422 | — | 2.66/2.70^{&} | 2 |
| 55 | n.d. | 354 | — | 1.65/1.70^{&} | 2 |
| 56 | 199.25° C. | 367 | — | 1.76 | 2 |
| 57 | 161.31° C.* | 367 | — | 1.76 | 2 |
| 58 | n.m. | 344 | — | 1.23 | 2 |
| 59 | 127.35° C. | 331 | — | 0.89 | 2 |
| 60 | 238.12° C. | 472 | — | 1.55 | 2 |
| 61 | n.m. | 422 | — | 1.97 | 2 |
| 62 | 217.24° C. | — | 420 | 1.48 | 2 |
| 63 | 266.56° C. | 484 | — | 1.59 | 2 |
| 64 | n.m. | 434 | — | 1.76 | 2 |
| 65 | 236.16° C.* | 358 | — | 0.91 | 2 |
| 66 | n.m. | 370 | — | 1.09 | 2 |
| 67 | n.m. | 350 | — | 1.35/1.30^{&} | 2 |
| 68 | 190.79° C. | 356 | — | 0.7 | 2 |
| 69 | n.d. | 370 | — | 1 | 2 |
| 70 | n.m. | 370 | — | 1.25 | 2 |
| 71 | n.m. | 370 | 368 | 1.92 | 5 |
| 72 | n.m. | 370 | 368 | 1.93 | 5 |
| 73 | n.m. | 370 | 368 | 1.75 | 7 |
| 74 | 276.23° C. | 370 | 368 | 0.89 | 8 |
| 75 | 251.29° C. | 370 | 368 | 2.25 | 6 |
| 76 | 250.45° C. | 370 | 368 | 2.24 | 6 |
| 77 | n.m. | 370 | — | 1.26 | 2 |
| 78 | n.d. | 370 | 368 | 2.1 | 6 |
| 79 | n.d. | 370 | 368 | 2.1 | 6 |
| 80 | n.d. | 356 | 354 | 1.7 | 5 |
| 81 | n.d. | 356 | 354 | 1.68 | 5 |
| 82 | n.d. | 356 | 354 | 1.83 | 5 |
| 83 | n.d. | 356 | 354 | 1.83 | 5 |
| 84 | n.m. | 440 | 438 | 1.46 | 2 |
| 86 | n.m. | 384.1 | 382.2 | 2.14 | 6 |
| 87 | n.m. | 384.1 | 382.2 | 2.14 | 6 |
| 88 | n.m. | 384.1 | — | 0.76 | 8 |
| 89 | 208.90° C. | 384.0 | 382.1 | 2.14 | 6 |
| 90 | 199.34° C. | 384.0 | 382.1 | 2.14 | 6 |
| 91 | n.m. | 370.1 | 368.2 | 1.07 | 1 |
| 92 | n.d. | 370.1 | 368.1 | 2.07 | 6 |
| 93 | n.d. | 370.1 | 368.1 | 2.07 | 6 |
| 94 | n.m. | 370.2 | 368.2 | 0.69 | 8 |
| 95 | 242.58° C. | 370.0 | 368.1 | 2.04 | 6 |
| 96 | 239.98° C. | 370.0 | 368.1 | 2.04 | 6 |
| 97 | n.m. | 370.2 | 368.2 | 0.97 | 2 |
| 98 | n.d. | 370.0 | 368.1 | 1.92 | 6 |
| 99 | n.d. | 370.0 | 368.1 | 1.92 | 6 |
| 100 | n.m. | 370.2 | — | 1.19 | 2 |
| 101 | n.m. | 384.2 | 382.1 | 0.95 | 8 |
| 102 | n.m. | 384.1 | 382.1 | 2.54 | 6 |
| 103 | n.m. | 384.1 | 382.1 | 2.54 | 6 |
| 104 | 189.54° C. | 371.2 | — | 0.74 | 2 |
| 105 | n.m. | 384.1 | — | 0.77 | 8 |
| 106 | | 356.2 | — | 0.91 | 2 |
| 107 | n.d. | 356.2 | — | 0.94 | 2 |
| 108 | 202.48° C. | 357.2 | — | 0.68 | 2 |
| 109 | | 422 | — | 1.20 | 2 |

n.d. means not determined. n.m. means not measured.
*Two crystalline forms were detected. The highest value is reported.
^{&}Mixture of diastereomers

### Optical Rotations

Optical rotations were measured on a Perkin-Elmer 341 polarimeter with a sodium lamp and reported as follows: [α]^{o} (λ, c g/100 ml, solvent, T ° C.).

[α]_{λ}^{T}=(100α)/(l×c):

where l is the path length in dm and c is the concentration in g/100 ml for a sample at a temperature T (° C.) and a wavelength λ (in nm). If the wavelength of light used is 589 nm (the sodium D line), then the symbol D might be used instead. The sign of the rotation (+ or −) should always be given. When using this equation the concentration and solvent are always provided in parentheses after the rotation. The rotation is reported using degrees and no units of concentration are given (it is assumed to be g/100 mL).

TABLE 19
Optical Rotation data.

| Co. | | Wavelength | Concentration | | Temp. |
|---|---|---|---|---|---|
| No. | α_{D} (°) | (nm) | w/v % | Solvent | (° C.) |
| 1 | +120.1 | 589 | 0.49 | DMF | 20 |
| 4 | −129.3 | 589 | 0.52 | DMF | 20 |
| 7 | n.m. | — | — | — | — |
| 9 | +24.1 | 589 | 0.50 | DMF | 20 |
| 16 | n.m. | — | — | — | — |
| 22 | −73.8 | 589 | 0.51 | DMF | 20 |
| 25 | +76.0 | 589 | 0.50 | DMF | 20 |
| 29 | n.m. | — | — | — | — |
| 30 | n.m. | — | — | — | — |
| 31 | n.m. | — | — | — | — |
| 32 | −122.4 | 589 | 0.53 | DMF | 20 |
| 65 | −27.0 | 589 | 0.52 | DMF | 20 |
| 71 | +38.2 | 589 | 0.52 | DMF | 20 |
| 72 | −38.4 | 589 | 0.45 | DMF | 20 |
| 75 | +105.4 | 589 | 0.51 | DMF | 20 |
| 76 | −92.2 | 589 | 0.56 | DMF | 20 |
| 78 | −103.1 | 589 | 0.58 | DMF | 20 |
| 79 | +102.4 | 589 | 0.52 | DMF | 20 |
| 80 | +103.6 | 589 | 0.55 | DMF | 20 |
| 81 | −100.0 | 589 | 0.48 | DMF | 20 |
| 82 | +11.6 | 589 | 0.58 | DMF | 20 |
| 83 | −11.8 | 589 | 0.58 | DMF | 20 |
| 86 | +36.1 | 589 | 0.70 | DMF | 20 |
| 87 | −47.6 | 589 | 0.68 | DMF | 20 |
| 89 | +62.8 | 589 | 0.63 | DMF | 20 |
| 90 | −38.6 | 589 | 0.85 | DMF | 20 |
| 92 | −33.4 | 589 | 0.60 | DMF | 20 |
| 93 | +13.8 | 589 | 0.90 | DMF | 20 |
| 95 | +19.7 | 589 | 0.50 | DMF | 20 |
| 96 | −29.8 | 589 | 0.59 | DMF | 20 |
| 98 | +7.6 | 589 | 0.51 | DMF | 20 |
| 99 | −7.0 | 589 | 0.51 | DMF | 20 |
| 102 | −139.7 | 589 | 0.58 | DMF | 20 |
| 103 | +152.0 | 589 | 0.54 | DMF | 20 |
| 104 | +7.7 | 589 | 0.48 | DMF | 20 |
| 107 | −10.7 | 589 | 0.61 | DMF | 20 |
| 108 | −9.2 | 589 | 0.72 | DMF | 20 |

n.m. means not measured.

### SFCMS-Methods

### General Procedure for SFC-MS Methods

The SFC measurement was performed using Analytical Supercritical fluid chromatography (SFC) system composed by a binary pump for delivering carbon dioxide (CO₂) and modifier, an autosampler, a columns oven with switching valve for column heating from room temperature to 80° C., a diode array detector equipped with a high-pressure flow cell standing up to 400 bars. Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

TABLE 20
Analytical SFC-MS Methods (Flow expressed in mL/min; column
temperature (T) in ° C.; Backpressure in bars).

| Method | Column | Mobile Phase | Gradient | $\frac{Flow}{T}$ | $\frac{Run Time}{BPR}$ |
|---|---|---|---|---|---|
| 1 | Daicel Chiralpak ® OJ3 column (3.0 μm, 150 × 4.6 mm) | A: CO₂ B: EtOH (+0.2% iPrNH₂) | 10%-50% B in 6 min, hold 3.5 min | $\frac{2.5}{40}$ | $\frac{9.5}{110}$ |
| 2 | Daicel Chiralpak ® AD-3 column (3 μm, 100 × 4.6 mm) | A: CO₂ B: EtOH (+0.3% iPrNH₂) | 40% B hold 4 min, | $\frac{3.5}{35}$ | $\frac{3.0}{103}$ |
| 3 | Daicel Chiralpak ® AD-3 column (3 μm, 100 × 4.6 mm) | A: CO₂ B: iPrOH (+0.3% iPrNH₂) | 40% B hold 3 min, | $\frac{3.5}{35}$ | $\frac{3.0}{103}$ |
| 4 | Daicel Chiralpak ® AS-3 column (3 μm, 100 × 4.6 mm) | A: CO₂ B: MeOH (+0.3% iPrNH₂) | 20% B hold 3 min, | $\frac{3.5}{35}$ | $\frac{3.0}{103}$ |
| 5 | Daicel Chiralpak ® OJ-3 column (3 μm, 100 × 4.6 mm) | A: CO₂ B: MeOH (+0.3% iPrNH₂) | 20% B hold 3 min, | $\frac{3.5}{35}$ | $\frac{3.0}{103}$ |
| 6 | Daicel Chiralpak ® OJ-3 column (3 μm, 100 × 4.6 mm) | A: CO₂ B: EtOH (+0.3% iPrNH₂) | 20% B hold 3 min, | $\frac{3.5}{35}$ | $\frac{3.0}{103}$ |
| 7 | Daicel Chiralpak ® OD-3 column (3 μm, 100 × 4.6 mm) | A: CO₂ B: MeOH (+0.3% iPrNH₂) | 40% B hold 4 min, | $\frac{3.5}{35}$ | $\frac{4.0}{103}$ |
| 8 | Daicel Chiralpak ® AD-3 column (3 μm, 100 × 4.6 mm) | A: CO₂ B: EtOH (+0.3% iPrNH₂) | 25% B hold 3 min, | $\frac{3.5}{35}$ | $\frac{3.0}{103}$ |
| 9 | Daicel Chiralpak ® OD-3 column (3 μm, 100 × 4.6 mm) | A: CO₂ B: EtOH (+0.3% iPrNH₂) | 30% B hold 3 min, | $\frac{3.5}{35}$ | $\frac{3.0}{103}$ |
| 10 | Daicel Chiralpak ® AD-3 column (3 μm, 100 × 4.6 mm) | A: CO₂ B: MeOH (+0.3% iPrNH₂) | 40% B hold 3 min, | $\frac{3.5}{35}$ | $\frac{3.0}{103}$ |
| 11 | Daicel Chiralpak ® AD-3 column (3 μm, 100 × 4.6 mm) | A: CO₂ B: MeOH (+0.3% iPrNH₂) | 40% B hold 3 min, | $\frac{3.5}{35}$ | $\frac{3}{105}$ |

TABLE 21
Analytical SFC data - Rₜ means retention time (in
minutes), [M + H]⁺ means the protonated
mass of the compound, method refers to the method
used for (SFC)MS analysis of enantiomerically pure compounds.

| Co. | | | | | Isomer Elution |
|---|---|---|---|---|---|
| No. | Rₜ | [M + H]⁺ | UV Area % | Method | Order |
| 29 | 4.12 | 424 | 96.88 | 1 | A |
| 30 | 5.1 | 424 | 99.26 | 1 | B |
| 71 | 1.27 | 370 | 99.47 | 2 | A |
| 72 | 1.62 | 370 | 99.6 | 2 | B |
| 82 | 0.88 | 356 | 100 | 3 | A |
| 83 | 1.26 | 356 | 99.41 | 3 | B |
| 80 | 1.12 | 356 | 100 | 2 | A |
| 81 | 1.94 | 356 | 100 | 2 | B |
| 78 | 1.13 | 370 | 100 | 4 | A |
| 79 | 1.63 | 370 | 100 | 4 | B |
| 75 | 0.91 | 370 | 99.31 | 5 | A |
| 76 | 1.34 | 370 | 99.67 | 5 | B |
| 86 | 1.35 | 384 | 99.12 | 6 | B |
| 87 | 0.94 | 384 | 100 | 6 | A |
| 89 | 1.87 | 384 | 98.86 | 7 | B |
| 90 | 0.81 | 384 | 100 | 7 | A |
| 92 | 2.12 | 370 | 99.16 | 8 | B |
| 93 | 1.67 | 370 | 100 | 8 | A |
| 95 | 1.40 | 370 | 97.29 | 9 | B |
| 96 | 1.04 | 370 | 100 | 9 | A |
| 98 | 2.20 | 370 | 99.68 | 10 | B |
| 99 | 1.36 | 370 | 100 | 10 | A |
| 102 | 2.04 | 384 | 100 | 11 | B |
| 103 | 0.92 | 384 | 100 | 11 | A |

### NMR

For a number of compounds, ¹H NMR spectra were recorded on a Bruker Avance III with a 300 MHz Ultrashield magnet, on a Bruker DPX-400 spectrometer operating at 400 MHz, on a Bruker Avance I operating at 500 MHz, on a Bruker DPX-360 operating at 360 MHz, or on a Bruker Avance 600 spectrometer operating at 600 MHz, using CHLOROFORM-d (deuterated chloroform, CDCl₃) or DMSO-d₆ (deuterated DMSO, dimethyl-d6 sulfoxide) as solvent. Chemical shifts (δ) are reported in parts per million (ppm) relative to tetramethylsilane (TMS), which was used as internal standard.

TABLE 22
¹H NMR results

| Co. | |
|---|---|
| No. | ¹H NMR result |
| 1 | ¹H NMR (400 MHz, DMSO-d₆) δ ppm 1.66-1.94 (m, 3 H), 1.99-2.10 (m, 1 |
| | H), 2.20 (s, 3 H), 2.65-2.74 (m, 1 H), 2.83 (t, J = 10.5 Hz, 1 H), 3.50-3.61 (m, |
| | 1 H), 3.71-3.81 (m, 1 H), 3.93 (br dd, J = 11.3, 3.5 Hz, 1 H), 7.31 (d, J = 4.6 Hz, |
| | 1 H), 8.00 (s, 1 H), 8.72 (s, 1 H), 8.85 (d, J = 4.6 Hz, 1 H), 12.76 (br s, 1 H). |
| 2 | ¹H NMR (400 MHz, DMSO-d₆) δ ppm 1.57-1.69 (m, 1 H), 1.76-1.88 (m, 2 |
| | H), 1.99-2.08 (m, 1 H), 2.21 (s, 3 H), 2.65-2.75 (m, 1 H), 2.87 (dd, J = 11.0, |
| | 10.1 Hz, 1 H), 3.48-3.61 (m, 2 H), 3.76 (br dd, J = 11.1, 3.0 Hz, 1 H), 6.99 (d, |
| | J = 4.4 Hz, 1 H), 7.80 (d, J = 1.6 Hz, 1 H), 8.03 (s, 1 H), 8.10 (d, J = 1.6 Hz, 1 H), |
| | 8.51 (d, J = 4.4 Hz, 1 H), 12.77 (s, 1 H). |
| 3 | ¹H NMR (400 MHz, DMSO-d₆) δ ppm 1.66-1.84 (m, 2 H), 1.85-1.93 (m, 1 |
| | H), 2.00-2.08 (m, 1 H), 2.20 (s, 3 H), 2.57-2.70 (m, 4 H), 2.74 (t, J = 10.6 Hz, |
| | 1 H), 3.57-3.65 (m, 1 H), 3.65-3.74 (m, 1 H), 3.95 (br dd, J = 11.2. 3.6 Hz, 1 |
| | H), 7.20 (s, 1 H), 8.00 (s, 1 H), 8.60 (s, 1 H), 12.75 (s, 1 H). |
| 4 | ¹H NMR (400 MHz, DMSO- d₆) δ ppm 1.65-1.91 (m, 3 H), 1.98-2.09 (m, 1 |
| | H), 2.20 (s, 3 H), 2.65-2.75 (m, 1 H), 2.84 (t, J = 10.5 Hz, 1 H), 3.51-3.60 (m, |
| | 1 H), 3.72-3.82 (m, 1 H), 3.93 (br dd, J = 11.2, 3.4 Hz, 1 H), 7.31 (d, J = 4.6 Hz, |
| | 1 H), 8.00 (s, 1 H), 8.72 (s, 1 H), 8.85 (d, J = 4.6 Hz, 1 H), 12.76 (s, 1 H). |
| 5 | ¹H NMR (400 MHz, DMSO- d₆) δ ppm 1.67-1.86 (m, 3 H), 1.99-2.07 (m, 1 |
| | H), 2.21 (s, 3 H), 2.70-2.79 (m, 1 H), 2.98 (t, J = 10.5 Hz, 1 H), 3.53-3.67 (m, |
| | 2 H), 3.78 (br dd, J = 11.2, 2.9 Hz, 1 H), 6.97 (t, J = 54.6 Hz, 1 H), 7.26 (s, 1 H), |
| | 7.96 (d, J = 1.4 Hz, 1 H), 8.03 (s, 1 H), 8.28 (d, J = 1.4 Hz, 1 H), 12.75 (br s, 1 |
| | H). |
| 6 | ¹H NMR (400 MHz, DMSO- d₆) δ ppm 1.64-1.79 (m, 1 H), 1.82-1.99 (m, 2 |
| | H), 1.99-2.09 (m, 1 H), 2.19 (s, 3 H), 2.68-2.77 (m, 1 H), 2.91 (t, J = 10.4 Hz, |
| | 1 H), 3.52-3.61 (m, 1 H), 3.78-3.88 (m, 1 H), 3.88-3.95 (m, 1 H), 7.09 (t, |
| | J = 54.1 Hz, 1 H), 7.57 (s, 1 H), 7.98 (s, 1 H), 8.86 (s, 1 H), 12.74 (br s, 1 H). |
| 7 | ¹H NMR (400 MHz, DMSO- d₆) δ ppm 0.73 (d, J = 6.5 Hz, 3 H), 1.43-1.58 (m, |
| | 1 H), 1.92-2.00 (m, 1 H), 2.20 (s, 3 H), 2.50 (dt, J = 3.1. 1.8 Hz, 2 H), 2.77 (br |
| | s. 1 H), 3.60-3.69 (m, 1 H), 3.71-3.78 (m, 1 H), 3.83-3.91 (m, 1 H), 7.09 (t, |
| | J = 54.1 Hz, 1 H), 7.65 (s, 1 H), 7.95 (s, 1 H), 8.84 (s, 1 H), 12.76 (br s, 1 H). |
| 8 | ¹H NMR (400 MHz, CDCl₃) δ ppm 2.28 (dq, J = 13.5. 6.8 Hz, 1 H), 2.39 (s, 3 |
| | H), 2.45-2.57 (m, 1 H), 2.69 (s, 3 H), 3.51-3.60 (m, 1 H), 3.61-3.69 (m, 1 |
| | H), 3.73 (dd, J = 10.8, 5.9 Hz, 1 H), 3.90 (dd, J = 10.6, 7.4 Hz, 1 H), 4.06 (quin, |
| | J = 6.7 Hz, 1 H), 6.82 (s, 1 H), 7.88 (s, 1 H), 8.42 (s, 1 H), 11.21 (br s, 1 H). |
| 9 | ¹H NMR (400 MHz, CDCl₃) δ ppm 1.74-1.83 (m, 3 H), 2.08-2.20 (m, 1 H), |
| | 2.33 (s, 3 H), 2.40-2.50 (m, 1 H), 2.61 (br t, J = 9.0 Hz, 1 H), 2.76 (br s, 1 H), |
| | 3.10 (br d, .J = 9.2 Hz, 1 H), 3.72-3.83 (m, 2 H), 3.83-3.92 (m, 1 H), 7.22 (s, 2 |
| | H), 8.49 (s, 1 H), 8.77 (d, J = 4.6 Hz, 1 H), 12.08 (br s, 1 H). |
| 10 | ¹H NMR (400 MHz, CDCl₃) δ ppm 1.54-1.69 (m, 1 H), 1.74-1.95 (m, 2 H), |
| | 2.09-2.18 (m, 1 H), 2.25-2.32 (m, 2 H), 2.33 (s, 3 H), 2.90-3.00 (m, 1 H), |
| | 3.16 (br d, J = 10.9 Hz, 1 H), 3.26 (tt, J = 9.8, 3.5 Hz, 1 H), 3.70-3.83 (m, 2 H), |
| | 6.83 (br d, J = 4.2 Hz, 1 H), 7.20 (s, 1 H), 7.59 (d, J = 1.6 Hz, 1 H), 7.83 (d, J = 1.4 |
| | Hz, 1 H), 8.52 (d, J = 4.4 Hz, 1 H), 12.18 (br s, 1 H). |
| 11 | ¹H NMR (500 MHz, CDCl₃) δ ppm 1.70-1.84 (m, 3 H), 2.03-2.13 (m, 1 H), |
| | 2.30 (s, 3 H), 2.46-2.55 (m, 1 H), 2.60-2.69 (m, 1 H), 2.72 (s, 4 H), 3.00 (br |
| | d, J = 9.8 Hz, 1 H), 3.68-3.76 (m, 1 H), 3.77-3.85 (m, 2 H), 7.16 (br s, 1 H), |
| | 7.22 (s, 1 H), 8.39 (s, 1 H), 11.29 (br s, 1 H). |
| 12 | ¹H NMR(500 MHz, CDCl₃) δ ppm 1.53-1.63 (m, 1 H), 1.76-1.91 (m, 2H), |
| | 2.10-2.27 (m, 3 H), 2.31 (s, 3 H), 2.99 (br d, J = 11.0 Hz, 1 H), 3.17-3.27 (m, |
| | 2 H), 3.70-3.80 (m, 2 H), 6.68 (br d, J = 6.9 Hz, 1 H), 7.16 (dd, J = 9.1, 7.1 Hz, |
| | 1 H), 7.19 (s, 1 H), 7.53 (d, J = 9.0 Hz, 1 H), 7.63 (s, 1 H), 7.65 (d, J = 1.2 Hz, 1 |
| | H), 11.84 (br s, 1 H). |
| 13 | ¹H NMR (500 MHz, DMSO- d₆) δ ppm 1.57-1.74 (m, 2 H), 1.75-1.84 (m, 1 |
| | H), 1.99-2.07 (m, 1 H), 2.12 (s, 3 H), 2.33 (br t, J = 9.0 Hz, 1 H), 2.53-2.63 |
| | (m, 1 H), 2.68-2.80 (m, 1 H), 3.04 (br d, J = 9.5 Hz, 1 H), 3.70-3.82 (m, 3 H), |
| | 7.09 (t, J = 54.3 Hz, 1 H), 7.28 (s, 1 H), 7.66 (s, 1 H), 8.81 (s, 1 H), 11.94 (s, 1 |
| | H). |
| 14 | ¹H NMR (500 MHz, DMSO-d₆) δ ppm 1.53-1.77 (m, 3 H), 1.99-2.07 (m, 1 |
| | H), 2.11 (s, 3 H), 2.30 (br t, J = 9.1 Hz, 1 H), 2.51-2.53 (m, 1 H), 2.71-2.80 |
| | (m, 1 H), 3.01 (br d, J = 9.2 Hz, 1 H), 3.43-3.52 (m, 1 H), 3.70-3.78 (m, 2 H), |
| | 6.97 (t, J = 54.6 Hz, 1 H), 7.28 (s, 1 H), 7.31 (s, 1 H), 7.91 (d, J = 1.4 Hz, 1 H), |
| | 8.22 (d, J = 1.4 Hz, 1 H), 11.92 (br s, 1 H). |
| 15 | ¹H NMR (500 MHz, CDCl₃) δ ppm 1.56-1.72 (m, 2 H), 1.76-1.86 (m, 1 H), |
| | 1.93 (ddt, J = 13.5, 9.1, 4.6, 4.6 Hz, 1 H), 2.32 (s, 3 H), 2.44 (br s, 1 H), 2.73-3.15 |
| | (m, 3H), 3.31 (br t, J = 4.6 Hz, 1 H), 3.73 (d, J = 13.9 Hz, 1 H), 3.83 (d, |
| | J = 13.9 Hz, 1 H), 6.69-6.75 (m, 1 H), 6.86 (d, J = 4.9 Hz, 1 H), 7.25 (s, 1 H), |
| | 7.56 (br s, 1 H), 8.36 (d, J = 4.9 Hz, 1 H), 11.40 (br s, 2 H). |
| 16 | ¹H NMR (400 MHz, CDCl₃) δ ppm 0.87 (br d, J = 6.0 Hz, 3 H), 1.56-1.68 (m, |
| | 4 H), 1.83-1.92 (m, 1 H), 2.29 (s, 3 H), 2.27-2.39 (m, 1 H), 3.02 (br d, |
| | J = 11.1 Hz, 1 H), 3.11 (br d, J = 9.7 Hz, 1 H), 3.76 (s, 2 H), 6.67 (t, J = 54.7 Hz, 1 |
| | H), 7.19 (s, 1 H), 7.30 (s, 1 H), 8.57 (s, 1 H), 11.33 (br s, 1 H). |
| 17 | ¹H NMR (400 MHz, CDCl₃) δ ppm 1.54-1.66 (m, 1 H), 1.71-1.89 (m, 2 H), |
| | 2.05-2.15 (m, 1 H), 2.30 (s, 3 H), 2.29-2.38 (m, 2 H), 2.62 (s, 3 H), 2.86-2.95 |
| | (m, 1 H), 3.06-3.13 (m, 1 H), 3.20 (tt, J = 9.5. 3.6 Hz, 1 H), 3.70-3.80 |
| | (m, 2 H), 6.73 (br s, 1 H), 7.20 (s, 1 H), 7.46 (d, J = 1.6 Hz, 1 H), 7.72 (d, J = 1.4 |
| | Hz, 1 H), 10.80 (br s, 1 H). |
| 18 | ¹H NMR (500 MHz, CDCl₃) δ ppm 1.96-2.05 (m, 1 H), 2.32 (s, 3 H), 2.56 |
| | (ddt, J = 17.4, 9.4, 4.1, 4.1 Hz, 1 H), 2.66 (q, J = 8.3 Hz, 1 H), 2.73 (s, 3 H), 2.92 |
| | (dd, J = 9.5. 7.8 Hz, 1 H), 3.01 (dd, J = 9.5. 4.0 Hz, 1 H), 3.07 (td, J = 8.7, 4.0 Hz, |
| | 1 H), 3.82 (d, J = 13.9 Hz, 1 H), 3.96 (dd, J = 13.9, 0.9 Hz, 1 H), 4.14-4.22 (m, 1 |
| | H), 7.05 (s, 1 H), 7.24 (s, 1 H), 8.40 (s, 1 H), 11.86 (br s, 1 H). |
| 19 | ¹H NMR (400 MHz, CDCl₃) δ ppm 2.30 (s, 3 H), 2.34-2.45 (m, 2 H), 2.69 (d, |
| | J = 3.5 Hz, 3 H), 2.90-3.02 (m, 2 H), 3.03-3.11 (m, 1 H), 3.17 (t, J = 8.4 Hz, 1 |
| | H), 3.92 (s, 2H), 4.34 (quin, J = 8.6 Hz, 1 H), 7.25 (s, 1 H), 8.42 (s, 1 H), 11.31 |
| | (br s, 1 H). |
| 20 | ¹H NMR (500 MHz, CDCl₃) δ ppm 2.00-2.08 (m, 1 H), 2.32 (s, 3 H), 2.58-2.67 |
| | (m, 1 H), 2.68-2.75 (m, 1 H), 3.00-3.12 (m, 3 H), 3.87 (d, J = 13.8 Hz, 1 |
| | H), 3.96 (dd, J = 13.9, 0.9 Hz, 1 H), 4.28-4.36 (m, 1 H), 7.26 (s, 1 H), 7.51 (s, 1 |
| | H), 8.63 (s, 1 H), 11.92 (s, 1 H). |
| 21 | ¹H NMR (400 MHz, CDCl₃) δ ppm 2.00 (br dd, J = 13.2, 6.5 Hz, 1 H), 2.30 (s, 3 |
| | H), 2.48-2.60 (m, 1 H), 2.62 (s, 3 H), 2.62-2.72 (m, 1 H), 2.90-3.10 (m, 3 |
| | H), 3.83 (d, J = 13.9 Hz, 1 H), 3.94 (d, J = 13.9 Hz, 1 H), 4.20-4.31 (m, 1 H), |
| | 6.55 (d, J = 2.3 Hz, 1 H), 6.78 (s, 1 H), 7.25 (s, 1 H), 8.04 (d, J = 2.3 Hz, 1 H), |
| | 11.23 (br s, 1 H). |
| 22 | ¹H NMR (500 MHz, DMSO-d₆) δ ppm 1.56-1.77 (m, 2 H), 1.85-1.94 (m, 1 |
| | H), 2.00-2.09 (m, 1 H), 2.20 (s, 3 H), 2.51-2.56 (m, 1 H), 2.78 (t, J = 11.1 Hz, |
| | 1 H), 3.35 (tt, J = 11.0, 3.8 Hz, 1 H), 3.65 (br d, J = 11.6 Hz, 1 H), 3.83-3.90 (m, |
| | 1 H), 7.59 (t, J = 52.3 Hz, 1 H), 7.77 (s, 1 H), 8.00 (s, 1 H), 8.75 (s, 1 H), 12.76 |
| | (s, 1 H). |
| 23 | ¹H NMR (400 MHz, DMSO-d₆) δ ppm 1.50-1.78 (m, 2 H), 1.90 (br d, J = 12.9 |
| | Hz, 1 H), 2.02 (br d, J = 12.3 Hz, 1 H), 2.20 (s, 3 H), 2.43-2.51 (m, 1 H), 2.72 |
| | (br t, J = 11.1 Hz, 1 H), 3.13-3.27 (m, 1 H), 3.65 (br d, J = 11.3 Hz, 1 H), 3.86 |
| | (br d, J = 8.8 Hz, 1 H), 7.49 (t, J = 52.4 Hz, 1 H), 7.47 (s, 1 H), 7.84 (s, 1 H), 7.98 |
| | (s, 1 H), 8.00 (s, 1 H), 12.75 (br s, 1 H). |
| 24 | ¹H NMR (500 MHz, DMSO-d₆) δ ppm 1.48-1.68 (m, 2 H), 1.71-1.81 (m, 1 |
| | H), 1.98 (br dd, J = 8.5, 2.7 Hz, 1 H), 2.02-2.08 (m, 1 H), 2.11 (s, 3 H), 2.18-2.33 |
| | (m, 1 H), 2.86 (br d, J = 11.3 Hz, 1 H), 3.02-3.12 (m, 2 H), 3.66-3.76 (m, |
| | 2 H), 7.26 (s, 1 H), 7.45 (t, J = 52.5 Hz, 1 H), 7.40 (s, 1 H), 7.81 (d, J = 1.4 Hz, 1 |
| | H), 7.94 (d, J = 1.2 Hz, 1 H), 11.93 (s, 1 H). |
| 25 | ¹H NMR (500 MHz, DMSO-d₆) δ ppm 1.51-1.68 (m, 2 H), 1.71-1.80 (m, 1 |
| | H), 1.96-2.03 (m, 1 H), 2.04-2.15 (m, 4 H), 2.33 (br t, J = 10.8 Hz, 1 H), 2.86 |
| | (br d, J = 11.3 Hz, 1 H), 3.04-3.11 (m, 1 H), 3.15-3.25 (m, 1 H), 3.68-3.76 |
| | (m, 2 H), 7.26 (s, 1 H), 7.55 (t, J = 52.3 Hz, 1 H), 7.69 (s, 1 H), 8.72 (s, 1 H), |
| | 11.93 (s, 1 H). |
| 26 | ¹H NMR (500 MHz, CDCl₃) δ ppm 1.48-1.61 (m, 1 H), 1.69-1.86 (m, 2 H), |
| | 1.90-2.01 (m, 1 H), 2.12-2.26 (m, 2 H), 2.29 (s, 3 H), 2.84-3.04 (m, 3 H), |
| | 3.66-3.72 (m, 1 H), 3.72-3.78 (m, 1 H), 6.69 (ddd, J = 3.2, 2.0, 0.9 Hz, 1 H), |
| | 7.16 (s, 1 H), 7.38-7.41 (m, 1 H), 7.66 (br s, 1 H), 8.35 (d, J = 1.7 Hz, 1 H), |
| | 8.75 (br s, 1 H), 11.80 (br s, 1 H). |
| 27 | ¹H NMR (500 MHz, DMSO-d₆) δ ppm 2.17 (s, 3 H), 2.46-2.50 (m, 1 H), 2.69 |
| | (td, J = 11.7, 3.5 Hz, 1 H), 3.63 (br d, J = 12.1 Hz, 1 H), 3.97 (td, J = 11.6, 2.6 Hz, |
| | 1 H), 4.21-4.27 (m, 2 H), 5.46 (dd, J = 10.1, 2.0 Hz, 1 H), 7.59 (s, 1 H), 7.95 (s, |
| | 1 H), 9.03 (s, 1 H), 12.76 (br s, 1 H). |
| 28 | ¹H NMR (400 MHz, DMSO-d₆) δ ppm 2.18 (s, 3 H), 2.38-2.46 (m, 1 H), 2.61 |
| | (s, 3H), 2.63-2.70 (m, 1 H), 3.62 (br d, J = 11.6 Hz, 1 H), 3.93 (td, J = 11.6, 2.4 |
| | Hz, 1 H), 4.19 (dd, J = 11.9, 2.2 Hz, 1 H), 4.24 (br d, J = 11.3 Hz, 1 H), 5.33 (dd, |
| | J = 10.1. 2.2 Hz, 1 H), 7.20 (s, 1 H), 7.98 (s, 1 H), 8.65 (s, 1 H), 12.76 (s, 1 H). |
| 29 | ¹H NMR (360 MHz, DMSO-d₆) δ ppm 2.18 (s, 3 H), 2.35-2.45 (m, 1 H), 2.57-2.68 |
| | (m, 4H), 3.62 (br d, J = 11.3 Hz, 1 H), 3.93 (td, J = 11.6, 2.4 Hz, 1 H), |
| | 4.14-4.29 (m, 2 H), 5.33 (dd, J = 10.1, 2.0 Hz, 1 H), 7.20 (s, 1 H), 7.98 (s, 1 H), |
| | 8.66 (s, 1 H), 12.79 (br s, 1 H). |
| 30 | ¹H NMR (360 MHz, DMSO-d₆) δ ppm 2.18 (s, 3 H), 2.40 (br t, J = 11.0 Hz, 1 |
| | H), 2.61 (s, 4H), 3.58-3.67 (m, 1 H), 3.87-4.02 (m, 1 H), 4.13-4.31 (m, 2 |
| | H), 5.34 (dd, J = 9.9. 1.8 Hz, 1 H), 7.20 (s, 1 H), 7.98 (s, 1 H), 8.67 (s, 1 H), |
| | 12.80 (br s, 1 H). |
| 31 | ¹H NMR (500 MHz, DMSO-d₆) δ ppm 2.18 (s, 3 H), 2.44 (br dd, J = 11.6, 10.1 |
| | Hz, 1 H), 2.63-2.70 (m, 1 H), 3.62 (br d, J = 11.8 Hz, 1 H), 3.93 (td, J = 11.7, |
| | 2.6 Hz, 1 H), 4.20 (br dd, J = 11.8, 2.0 Hz, 1 H), 4.25-4.30 (m, 1 H), 5.37 (dd, |
| | J = 10.0, 2.2 Hz, 1 H), 7.30 (dd, J = 4.5, 0.7 Hz, 1 H), 7.97 (s, 1 H), 8.77 (s, 1 H), |
| | 8.89 (d, J = 4.6 Hz, 1 H), 12.75 (s, 1 H). |
| 32 | ¹H NMR (500 MHz, DMSO-d₆) δ ppm 2.17 (s, 3 H), 2.44 (dd, J = 11.7, 10.1 |
| | Hz, 1H), 2.66 (td, J = 11.8, 3.6 Hz, 1 H), 3.61 (br d, J = 11.8 Hz, 1 H), 3.94 (td, |
| | J = 11.7, 2.6 Hz, 1 H), 4.18-4.23 (m, 1 H), 4.27 (dt, J = 11.7. 2.0,Hz, 1 H), 5.38 |
| | (dd, J = 10.1, 2.0 Hz, 1 H), 7.28-7.32 (m, 1 H), 7.97 (s, 1 H), 8.77 (s, 1 H), 8.89 |
| | (d, J = 4.6 Hz, 1 H), 12.76 (br s, 1 H). |
| 33 | ¹H NMR (400 MHz, DMSO-d₆) δ ppm 2.17 (s, 3 H), 2.43-2.50 (m, 1 H), 2.69 |
| | (td, J = 11.6, 3.4 Hz, 1 H), 3.63 (br d, J = 11.8 Hz, 1 H), 3.96 (td, J = 11.6, 2.5 Hz, |
| | 1 H), 4.19-4.31 (m, 2 H), 5.43 (dd, J = 9.9, 2.3 Hz, 1 H), 7.13 (t, J = 54.1 Hz, 1 |
| | H), 7.47 (s, 1 H), 7.95 (s, 1 H), 8.92 (s, 1 H), 12.74 (br s, 1 H). |
| 34 | ¹H NMR (500 MHz, DMSO-d₆) δ ppm 2.09-2.15 (m, 4 H), 2.31 (td, J = 11.5, |
| | 3.3 Hz, 1 H), 2.82 (br d, J = 11.6 Hz, 1 H), 3.47 (br d, J = 11.0 Hz, 1 H), 3.71-3.79 |
| | (m, 2 H), 3.82 (td, J = 11.4, 2.3 Hz, 1 H), 4.06-4.12 (m, 1 H), 5.31 (dd, |
| | J = 9.7, 2.2 Hz, 1 H), 7.14 (t, J = 54.0 Hz, 1 H), 7.26 (s, 1 H), 7.51 (s, 1 H), 8.84 |
| | (s, 1 H), 11.97 (s, 1 H). |
| 35 | ¹H NMR (500 MHz, DMS0-d₆) δ ppm 2.05-2.11 (m, 1 H), 2.12 (s, 3 H), 2.28 |
| | (td, J = 11.5, 3.3 Hz, 1 H), 2.64 (s, 3 H), 2.80 (br d, J = 11.6 Hz, 1 H), 3.42 (br d, |
| | J = 10.7 Hz, 1 H), 3.69-3.74 (m, 1 H), 3.75-3.79 (m, 1 H), 3.80 (br td, J = 11.3, |
| | 2.3 Hz, 1 H), 4.02-4.07 (m, 1 H), 5.22 (dd, J = 9.7, 1.9 Hz, 1 H), 7.25 (s, 1 H), |
| | 7.26 (s, 1 H), 8.58 (s, 1 H), 11.97 (s, 1 H). |
| 36 | ¹H NMR (500 MHz, DMSO-d₆) δ ppm 2.04-2.10 (m, 1 H), 2.12 (s, 3 H), 2.24-2.33 |
| | (m, 1 H), 2.81 (br d, J = 11.3 Hz, 1 H), 3.46 (br d, J = 11.0 Hz, 1 H), 3.67-3.86 |
| | (m, 3 H), 4.06 (br d, J = 11.3 Hz, 1 H), 5.26 (br d, J = 8.7 Hz, 1 H), 7.26 (s, |
| | 1 H), 7.34 (br d, J = 4.0 Hz, 1 H), 8.70 (s, 1 H), 8.89 (d, J = 4.3 Hz, 1 H), 11.97 |
| | (br s, 1 H). |
| 37 | ¹H NMR (400 MHz, DMSO-d₆) δ ppm 2.12 (s, 3 H), 2.13-2.19 (m, 1 H), 2.28-2.39 |
| | (m, 1 H), 2.81 (br d, J = 11.1 Hz, 1 H), 3.45 (br d, J = 11.3 Hz, 1 H), 3.70-3.87 |
| | (m, 3 H), 4.05-4.13 (m, 1 H), 5.33 (dd, J = 9.6, 2.0 Hz, 1 H), 7.26 (s, 1 H), |
| | 7.62 (s, 1 H), 8.94 (s, 1 H), 11.98 (s, 1 H). |
| 38 | ¹H NMR (400 MHz, DMSO-d₆) δ ppm 2.19 (s, 3 H), 2.58-2.72 (m, 2 H), 3.58 |
| | (br d, J = 11.6 Hz, 1 H), 3.85-3.98 (m, 2 H), 4.19 (dd, J = 11.6, 1.6 Hz, 1 H), |
| | 4.95 (dd, J = 10.2, 2.8 Hz, 1 H), 7.62 (t, J = 52.0 Hz, 1 H), 7.65 (s, 1 H), 8.04 (s, |
| | 1 H), 8.84 (s, 1 H), 12.69 (br s, 1 H). |
| 39 | ¹H NMR (500 MHz, DMSO-d₆) δ ppm 2.06-2.17 (m, 4 H), 2.26 (td, J = 11.5, |
| | 3.3 Hz, 1 H), 2.81 (br d, J = 11.6 Hz, 1 H), 3.20-3.26 (m, 1 H), 3.70-3.76 (m, |
| | 1 H), 3.76-3.83 (m, 2 H), 4.04-4.09 (m, 1 H), 4.75 (dd, J = 10.1, 2.6 Hz, 1 H), |
| | 7.29 (s, 1 H), 7.61 (t, J = 52.0 Hz, 1 H), 7.62 (s, 1 H), 8.80 (s, 1 H), 11.98 (br s, |
| | 1H). |
| 40 | ¹H NMR (500 MHz, DMSO-d₆) δ ppm 2.19 (s, 3 H), 2.33-2.40 (m, 1 H), 2.53-2.57 |
| | (m, 1 H), 2.60 (s, 3 H), 2.92 (br t, J = 11.1 Hz, 1 H), 3.13 (br d, J = 12.4 |
| | Hz, 1 H), 3.23 (br s, 1 H), 3.51-3.58 (m, 1 H), 3.99-4.06 (m, 1 H), 4.58 (br d, |
| | J = 9.0 Hz, 1 H), 7.24 (s, 1 H), 7.96 (s, 1 H), 8.64 (s, 1 H), 12.75 (br s, 1 H). |
| 41 | ¹H NMR (500 MHz, DMSO-d₆) δ ppm 2.18 (s, 3 H), 2.41 (dd, J = 11.1. 9.7 Hz, |
| | 1 H), 2.54-2.60 (m, 1 H), 2.89-2.98 (m, 1 H), 3.12-3.18 (m, 1 H), 3.32- |
| | 3.37 (m, 1 H), 3.52-3.58 (m, 1 H), 4.04-4.09 (m, 1 H), 4.65-4.72 (m, 1 H), |
| | 7.13 (t, J = 54.0 Hz, 1 H), 7.57 (s, 1 H), 7.94 (s, 1 H), 8.91 (s, 1 H), 12.74 (br s, |
| | 1H). |
| 42 | ¹H NMR (400 MHz, CDCl₃) δ ppm 2.30 (s, 3 H), 2.44-2.64 (m, 3 H), 2.73 (s, |
| | 3 H), 2.74-2.81 (m, 1 H), 2.93-3.07 (m, 2 H), 3.13-3.20 (m, 1 H), 3.68-3.74 |
| | (m, 1 H), 3.78-3.85 (m, 1 H), 4.71 (dd, J = 7.4. 3.0 Hz, 1 H), 7.23 (s, 1 H), |
| | 7.24-7.26 (m, 1 H), 8.42 (s, 1 H), 10.71 (br s, 1 H). |
| 43 | ¹H NMR (500 MHz, DMSO-d₆) δ ppm 2.19 (s, 3 H), 2.49-2.54 (m, 1 H), 2.55-2.61 |
| | (m, 1 H), 2.90 (br t, J = 10.3 Hz, 1 H), 3.03 (br s, 1 H), 3.09-3.15 (m, 1 |
| | H), 3.46 (br d, J = 11.3 Hz, 1 H), 3.80 (dd, J = 11.1, 2.2 Hz, 1 H), 4.15-4.22 (m, |
| | 1 H), 7.62 (t, J = 52.0 Hz, 1 H), 7.78 (s, 1 H), 7.99 (s, 1 H), 8.80 (s, 1 H), 12.75 |
| | (br s, 1 H). |
| 44 | ¹H NMR (500 MHz, CDCl₃) δ ppm 1.04-1.16 (m, 1 H), 1.46-1.56 (m, 1 H), |
| | 1.62-1.69 (m, 2H), 1.94-1.99 (m, 1 H), 2.05-2.14 (m, 1 H), 2.23 (s, 3 H), |
| | 2.25-2.34 (m, 1 H), 2.48 (s, 3 H), 2.61-2.72 (m, 2 H), 3.00 (qd, J = 14.4, 7.2 |
| | Hz, 2 H), 3.52-3.62 (m, 2 H), 6.45 (s, 1 H), 6.47 (d, J = 2.3 Hz, 1 H), 7.09 (s, 1 |
| | H), 7.98 (d, J = 2.3 Hz, 1 H), 11.62 (br s, 1 H). |
| 45 | ¹H NMR (500 MHz, CDCl₃) δ ppm 1.13-1.23 (m, 1 H), 1.28-1.37 (m, 1 H), |
| | 1.68-1.77 (m, 2 H), 2.02-2.10 (m, 1 H), 2.16-2.25 (m, 2 H), 2.30 (s, 3 H), |
| | 2.68-2.74 (m, 1 H), 2.77 (br d, J = 9.5 Hz, 1 H), 2.83 (dd, J = 14.9, 7.4 Hz, 1 H), |
| | 2.95 (dd, J = 15.0, 6.9 Hz, 1 H), 3.60-3.71 (m, 2 H), 7.19 (s, 1 H), 7.67-7.68 |
| | (m, 1 H), 7.69 (s, 1 H), 7.86 (d, J = 1.2 Hz, 1 H), 9.02 (s, 1 H), 10.53 (br s, 1 H). |
| 46 | ¹H NMR (500 MHz, CDCl₃) δ ppm 1.14-1.25 (m, 1 H), 1.54-1.64 (m, 1 H), |
| | 1.66-1.78 (m, 2H), 2.01-2.12 (m, 1 H), 2.24 (br s, 1 H), 2.32 (s, 3 H), 2.35-2.42 |
| | (m, 1 H), 2.65 (s, 3 H), 2.68-2.77 (m, 2 H), 3.04-3.19 (m, 2 H), 3.60-3.72 |
| | (m, 2 H), 6.79 (s, 1 H), 7.18 (s, 1 H), 8.42 (s, 1 H), 11.93 (br s, 1 H). |
| 47 | ¹H NMR (500 MHz, CDCl₃) δ ppm 1.03-1.15 (m, 1 H), 1.46 (dtt, J = 13.6, |
| | 10.2, 10.2, 3.7, 3.7 Hz, 1 H), 1.54-1.61 (m, 1 H), 1.65 (dt, J = 13.0, 3.8 Hz, 1 |
| | H), 1.99-2.13 (m, 2 H), 2.25 (s, 3 H), 2.25-2.32 (m, 1 H), 2.63 (d, J = 3.2 Hz, |
| | 3 H), 2.60-2.66 (m, 1 H), 2.69 (br d, J = 9.5 Hz, 1 H), 3.08-3.20 (m, 2 H), |
| | 3.53-3.64 (m, 2 H), 7.10 (s, 1 H), 8.33-8.38 (m, 1 H), 12.14 (br s, 1 H). |
| 48 | ¹H NMR (500 MHz, CDCl₃) δ ppm 1.55-1.65 (m, 1 H), 2.05-2.14 (m, 1 H), |
| | 2.32 (s, 3 H), 2.44 (dd, J = 9.2, 5.2 Hz, 1 H), 2.53-2.63 (m, 1 H), 2.64 (dd, |
| | J = 9.2, 7.5 Hz, 1 H), 2.67 (s, 3 H), 2.80 (td, J = 8.7, 4.9 Hz, 1 H), 2.85-2.95 (m, |
| | 1 H), 3.16-3.27 (m, 2 H), 3.72-3.78 (m, 1 H), 3.80-3.85 (m, 1 H), 6.81 (s, 1 |
| | H), 7.20 (s, 1 H), 8.41 (s, 1 H), 11.97 (br s, 1 H). |
| 49 | ¹H NMR (400 MHz, CDCl₃) δ ppm 1.56-1.71 (m, 1 H), 1.95-2.10 (m, 1 H), |
| | 2.32 (s, 3 H), 2.44 (dd, J = 9.2, 5.8 Hz, 1 H), 2.58-2.66 (m, 1 H), 2.66-2.80 |
| | (m, 2 H), 2.70 (d, J = 3.2 Hz, 3 H), 2.82-2.99 (m, 1 H), 3.24-3.43 (m, 2 H), |
| | 3.70-3.88 (m, 2 H), 7.20 (s, 1 H), 8.42 (s, 1 H), 12.34 (s, 1 H). |
| 50 | ¹H NMR (500 MHz, CDCl₃) δ ppm 1.56-1.66 (m, 1 H), 2.06-2.17 (m, 1 H), |
| | 2.31 (s, 3 H), 2.45 (dd, J = 9.1, 5.1 Hz, 1 H), 2.56-2.65 (m, 1 H), 2.68-2.74 |
| | (m, 1 H), 2.74-2.82 (m, 2 H), 2.90-3.02 (m, 2 H), 3.74-3.84 (m, 2 H), 6.62 |
| | (d, J = 6.4 Hz, 1 H), 7.15 (dd, J = 9.0, 6.9 Hz, 1 H), 7.21 (s, 1 H), 7.53-7.57 (m, |
| | 2 H), 7.68 (d, J = 1.4 Hz, 1 H), 12.37 (br s, 1 H). |
| 51 | ¹H NMR (500 MHz, CDCl₃) δ ppm 1.61-1.77 (m, 2 H), 1.79-1.91 (m, 2 H), |
| | 2.31 (s, 3 H), 2.47-2.70 (m, 3 H), 2.54 (s, 3 H), 2.73-2.82 (m, 1 H), 3.80 (br |
| | s, 2 H), 3.81 (br s, 1 H), 6.00 (s, 1 H), 6.56 (br s, 1 H), 7.22 (s, 1 H), 8.30 (s, 1 |
| | H), 11.87 (br s, 1 H). |
| 52^{&} | ¹H NMR (400 MHz, CDCl₃) δ ppm 1.36 (d, J = 6.2 Hz, 1.5 H), 1.38 (d, J = 6.2 |
| | Hz, 1.5 H), 1.47-1.59 (m, 1 H), 1.64-1.92 (m, 2 H), 2.00-2.25 (m, 3 H), |
| | 2.58 (s, 1.5 H), 2.61 (s, 1.5 H), 2.80-2.89 (m, 0.5 H), 3.01-3.21 (m, 2.5 H), |
| | 3.44 (q, J = 6.7 Hz, 0.5 H), 3.50 (q, J = 6.7 Hz, 0.5 H), 5.94 (s, 1 H), 5.96 (s, 1 |
| | H), 6.59 (s, 0.5 H), 6.67 (s, 0.5 H), 6.71-6.80 (m, 2 H), 6.85 (s, 0.5 H), 6.87 |
| | (d, J = 1.2 Hz, 0.5 H), 7.35 (d, J = 1.4 Hz, 0.5 H), 7.43 (d, J = 1.6 Hz, 0.5 H), 7.69 |
| | (d, J = 1.6 Hz, 0.5 H), 7.72 (d, J = 1.4 Hz, 0.5 H). |
| 53^{&} | ¹H NMR (400 MHz, DMSO-d₆) δ ppm 1.27 (d, J = 6.7 Hz, 1.35 H), 1.30 (d, |
| | J = 6.7 Hz, 1.65 H), 1.92 (dd, J = 11.3, 9.9 Hz, 0.55 H), 1.95-2.02 (m, 0.45 H), |
| | 2.09 (td, J = 11.6, 3.2 Hz, 0.45 H), 2.24 (td, J = 11.6, 3.2 Hz, 0.55 H), 2.61 (s, |
| | 1.65 H), 2.63 (s, 1.35 H), 2.64-2.70 (m, 0.45 H), 2.86 (br d, J = 10.6 Hz, 0.55 |
| | H), 3.29-3.36 (m, 0.55 H), 3.41 (q, J = 6.7 Hz, 0.45 H), 3.48-3.58 (m, 1 H), |
| | 3.73 (td, J = 11.3, 2.3 Hz, 0.45 H), 3.81 (td, J = 11.3, 2.5 Hz, 0.55 H), 3.95-4.01 |
| | (m, 0.45 H), 4.01-4.07 (m, 0.55 H), 5.16 (dd, J = 9.7, 1.6 Hz, 0.55 H), 5.22 (dd, |
| | J = 9.8, 2.0 Hz, 0.45 H), 5.95-6.00 (m, 2 H), 6.73 (dd, J = 7.9, 1.4 Hz, 0.55 H), |
| | 6.74 (dd, J = 7.9, 1.4 Hz, 0.45 H), 6.81 (d, J = 7.9 Hz, 0.55 H), 6.83 (d, J = 7.9 Hz, |
| | 0.45 H), 6.87 (d, J = 1.4 Hz, 0.55 H), 6.89 (d, J = 1.4 Hz, 0.45 H), 7.19 (br.s, 0.55 |
| | H), 7.22 (br.s, 0.45 H), 8.55 (s, 0.55 H), 8.62 (s, 0.45 H). |
| 54^{&} | ¹H NMR (500 MHz, CDCl₃) δ ppm 1.36 (d, J = 6.6 Hz, 1.35 H), 1.38 (d, J = 6.6 |
| | Hz, 1.65 H), 1.97 (dd, J = 11.0, 9.8 Hz, 0.55 H), 2.02 (dd, J = 10.7, 9.8 Hz, 0.45 |
| | H), 2.22 (td, J = 11.6, 3.3 Hz, 0.45 H), 2.41 (td, J = 11.5, 3.3 Hz, 0.55 H), 2.70-2.76 |
| | (m, 0.45 H), 2.89-2.95 (m, 0.55 H), 3.36 (q, J = 6.6 Hz, 0.45 H), 3.45-3.52 |
| | (m, 1.1 H), 3.77 (dt, J = 10.9, 2.1 Hz, 0.45 H), 3.86 (td, J = 11.3, 2.6 Hz, |
| | 0.45 H), 3.95 (td, J = 11.4, 2.6 Hz, 0.55 H), 4.01-4.07 (m, 0.45 H), 4.10-4.16 |
| | (m, 0.55 H), 5.36 (dd, J = 9.7, 1.9 Hz, 0.55 H), 5.43 (dd, J = 9.8, 2.0 Hz, 0.45 H), |
| | 5.91-5.98 (m, 2 H), 6.70-6.77 (m, 2 H), 6.84 (d, J = 1.2 Hz, 0.55 H), 6.86 (d, |
| | J = 1.4 Hz, 0.45 H), 7.59 (d, J = 0.6 Hz, 0.55 H), 7.64 (d, J = 0.9 Hz, 0.45 H), 8.59 |
| | (s, 0.55 H), 8.68 (s, 0.45 H). |
| 55^{&} | ¹H NMR (500 MHz, CDCl₃) δ ppm 1.35 (d, J = 6.9 Hz, 1.5 H), 1.38 (d, J = 6.6 |
| | Hz, 1.5 H), 1.98 (q, J = 10.4 Hz, 1 H), 2.18 (td, J = 11.6, 3.3 Hz, 0.5 H), 2.38 (td, |
| | J = 11.5, 3.3 Hz, 0.5 H), 2.68-2.75 (m, 0.5 H), 2.85-2.92 (m, 0.5 H), 3.33 (q, |
| | J = 6.6 Hz, 0.5 H), 3.42-3.51 (m, 1 H), 3.76 (dt, J = 10.7, 2.0 Hz, 0.5 H), 3.85 |
| | (td, J = 11.3, 2.5 Hz, 0.5 H), 3.94 (td, J = 11.3, 2.5 Hz, 0.5 H), 3.99-4.05 (m, 0.5 |
| | H), 4.05-4.16 (m, 0.5 H), 5.32 (dd, J = 9.7, 2.2 Hz, 0.5 H), 5.38 (dd, J = 9.8, 2.3 |
| | Hz, 0.5 H), 5.91-5.98 (m, 2 H), 6.69-6.78 (m, 2 H), 6.82-6.89 (m, 1 H), |
| | 7.23 (d, J = 4.6 Hz, 0.5 H), 7.28 (d, J = 4.9 Hz, 0.5 H), 8.45 (s, 0.5 H), 8.53 (s, 0.5 |
| | H), 8.79 (d, J = 4.6 Hz, 0.5 H), 8.83 (d, J = 4.6 Hz, 0.5 H). |
| 56 | ¹H NMR (400 MHz, CDCl₃) δ ppm 1.38 (d, J = 6.7 Hz, 3 H), 2.30-2.42 (m, 2 |
| | H), 2.68 (s, 3 H), 2.82-2.91 (m, 1 H), 2.98-3.09 (m, 2 H), 3.11-3.19(m, 1 |
| | H), 3.50 (q, J = 6.7 Hz, 1 H), 4.64 (dd, J = 8.1, 2.8 Hz, 1 H), 5.94 (s, 2 H), 6.75 |
| | (s, 2 H), 6.86 (s, 1 H), 7.10 (s, 1 H), 8.39 (s, 1 H); 1 H exchanged. |
| 57 | ¹H NMR (400 MHz, CDCl₃) δ ppm 1.36 (d, J = 6.7 Hz, 3 H), 2.30-2.41 (m, 2 |
| | H), 2.65-2.72 (m, 1 H), 2.70 (s, 3 H), 2.92-3.04 (m, 2 H), 3.28 (br d, J = 10.2 |
| | Hz, 1 H), 3.37 (q, J = 6.7 Hz, 1 H), 4.68 (dd, J = 7.7, 2.9 Hz, 1 H), 5.93-5.98 (m, |
| | 2 H), 6.75 (s, 2 H), 6.88 (s, 1 H), 7.17 (s, 1 H), 8.43 (s, 1 H); 1 H exchanged. |
| 58 | ¹H NMR (500 MHz, CDCl₃) δ ppm 1.59-1.69 (m, 1 H), 2.09-2.20 (m, 1 H), |
| | 2.46 (dd, J = 8.7, 4.9 Hz, 1 H), 2.65 (td, J = 8.5 6.6 Hz, 1 H), 2.73-2.86 (m, 3 |
| | H), 2.92-3.06 (m, 2 H), 3.82-3.91 (m, 2 H), 6.61 (d, J = 6.6 Hz, 1 H), 7.13 |
| | (dd, J = 9.0, 6.9 Hz, 1 H), 7.53 (d, J = 9.0 Hz, 1 H), 7.55 (s, 1 H), 7.66 (d, J = 1.2 |
| | Hz, 1 H), 7.83 (dd, J = 8.5, 1.9 Hz, 1 H), 8.03 (d, J = 1.2 Hz, 1 H), 8.08 (d, J = 8.7 |
| | Hz, 1 H), 8.80-8.86 (m, 2 H). |
| 59 | ¹H NMR (400 MHz, DMSO-d₆) δ ppm 1.70-1.80 (m, 2 H), 1.82-1.92 (m, 2 |
| | H), 2.32 (dd, J = 11.0, 9.8 Hz, 1 H), 2.59-2.70 (m, 3 H), 2.84 (br d, J = 11.6 Hz, |
| | 1 H), 3.16-3.25 (m, 2 H), 3.27 (t, J = 6.9 Hz, 2 H), 3.34-3.50 (m, 4 H), 3.81 |
| | (td, J = 11.2, 2.5 Hz, 1 H), 3.98-4.07 (m, 1 H), 5.21 (dd, J = 9.6, 2.0 Hz, 1 H), |
| | 7.29 (s, 1 H), 8.61 (s, 1 H). |
| 60 | ¹H NMR (500 MHz, DMSO-d₆) δ ppm 1.12 (br s, 1 H), 1.37-1.47 (m, 1 H), |
| | 1.53 (s, 3 H), 1.64-1.76 (m, 2 H), 2.21 (s, 3 H), 2.68-2.80 (m, 1 H), 2.89- |
| | 3.07 (m, 2 H), 4.20 (br d, J = 11.8 Hz, 1 H), 7.13 (t, J = 54.3 Hz, 1 H), 7.67 (s, 1 |
| | H), 8.01 (s, 1 H), 8.86 (s, 1 H), 12.77 (br s, 1 H). |
| 61 | ¹H NMR (400 MHz, CDCl₃) δ ppm 1.34-1.49 (m, 1 H), 1.57 (s, 3 H), 1.65 (br |
| | s. 2 H), 2.23-2.35 (m, 1 H), 2.33 (s, 3 H), 2.38-2.52 (m, 1 H), 2.73-2.85 (m, |
| | 1 H), 2.91 (br s, 1 H), 3.60-3.76 (m, 3 H), 6.73 (t, J = 54.7 Hz, 1 H), 7.24 (s, 1 |
| | H), 7.51 (s, 1 H), 8.47 (s, 1 H), 11.74 (br s, 1 H). |
| 62 | ¹H NMR (500 MHz, DMSO-d₆) δ ppm 1.27 (s, 3 H), 1.41-1.64 (m, 3 H), 2.11 |
| | (s, 3 H), 2.21-2.45 (m, 3 H), 2.52 (br s, 1 H), 3.14 (br s, 1 H), 3.64-3.72 (m, |
| | 2 H), 7.27 (s, 1 H), 7.52 (t, J = 52.3 Hz, 1 H), 7.66 (s, 1 H), 8.74 (s, 1 H), 11.93 |
| | (s, 1 H). |
| 63 | ¹H NMR (400 MHz, DMSO-d₆) δ ppm 0.45-0.55 (m, 3 H), 0.69-0.79 (m, 1 |
| | H), 1.17-1.27 (m, 1 H), 2.15-2.24 (m, 1 H), 2.22 (s, 3 H), 2.81 (td, J = 11.1, |
| | 2.5 Hz, 1 H), 3.16 (dd, J = 12.1, 3.8 Hz, 1 H), 3.40-3.45 (m, 1 H), 3.61-3.70 |
| | (m, 1 H), 4.34 (br s, 1 H), 7.17 (t, J = 54.1 Hz, 1 H), 7.75 (s, 1 H), 7.93 (s, 1 H), |
| | 8.85 (s, 1 H), 12.78 (br s, 1 H). |
| 64 | ¹H NMR (400 MHz, CDCl₃) δ ppm 0.33 (dt, J = 9.3, 5.3 Hz, 1 H), 0.47-0.57 |
| | (m, 1 H), 0.56-0.65 (m, 1 H), 0.85-0.89 (m, 1 H), 0.89-0.94 (m, 1 H), 2.23-2.34 |
| | (m, 1 H), 2.30 (s, 3 H), 2.44 (td, J = 11.2, 2.5 Hz, 1 H), 2.72 (dd, J = 11.8, |
| | 3.7 Hz, 1 H), 3.08 (br d, J = 10.6 Hz, 1 H), 3.32 (br d, J = 12.0 Hz, 1 H), 3.37 (br |
| | s, 1 H), 3.67 (d, J = 14.1 Hz, 1 H), 3.79 (d, J = 14.3 Hz, 1 H), 6.80 (t, J = 54.8 Hz, |
| | 1 H), 7.19 (s, 1 H), 8.37 (s, 1 H), 8.45 (s, 1 H), 12.10 (br s, 1 H). |
| 65 | ¹H NMR (400 MHz, CDCl₃) δ ppm 1.69-1.85 (m, 3 H), 2.07-2.20 (m, 1 H), |
| | 2.33 (s, 3 H), 2.39-2.52 (m, 1 H), 2.54-2.68 (m, 1 H), 2.78 (br d, J = 8.8 Hz, 1 |
| | H), 3.11 (br d, J = 9.7 Hz, 1 H), 3.72-3.83 (m, 2 H), 3.83-3.92 (m, 1 H), 7.23 |
| | (s, 2 H), 8.49 (s, 1 H), 8.77 (d, J = 4.6 Hz, 1 H), 12.31 (br s, 1 H). |
| 66 | ¹H NMR (500 MHz, CDCl₃) δ ppm 1.44 (qd, J = 12.4, 3.8 Hz, 2 H) 1.67 (br d, |
| | J = 12.7 Hz, 2 H) 1.83 (ttt. J = 11.2, 11.2, 7.4, 7.4, 3.7, 3.7 Hz, 1 H) 1.98 (td, |
| | J = 11.6, 2.0 Hz, 2 H) 2.29 (s, 3 H) 2.84 (d, J = 6.9 Hz, 2 H) 2.92 (br d, J = 11.6 |
| | Hz, 2 H) 3.66 (s, 2 H) 6.58 (d, J = 6.8 Hz, 1 H) 7.14 (dd, J = 9.1, 6.8 Hz, 1 H) |
| | 7.17 (s, 1 H) 7.51 (s, 1 H) 7.54 (d, J = 9.1 Hz, 1 H) 7.68 (d, J = 1.4 Hz, 1 H) |
| | 11.53 (br s, 1 H). |
| 67^{&} | ¹H NMR (500 MHz, CDCl₃) δ ppm 1.30-1.36 (m, 3 H), 1.49-1.62 (m, 1 H), |
| | 1.99-2.12 (m, 1 H), 2.20-2.28 (m, 0.6 H), 2.32 (dd, J = 9.2, 5.8 Hz, 0.4 H), |
| | 2.40-2.47 (m, 0.6 H), 2.50 (td, J = 8.8, 5.8 Hz, 0.4 H), 2.61-2.77 (m, 3 H), |
| | 2.86-3.01 (m, 2H), 3.14 (qd, J = 6.5, 2.5 Hz, 1 H), 5.91-5.97 (m, 2 H), 6.58 |
| | (d, J = 6.6 Hz, 0.6 H), 6.60 (d, J = 6.9 Hz, 0.4 H), 6.70-6.77 (m, 2 H), 6.88 (d, |
| | J = 1.2 Hz, 1 H), 7.10-7.17 (m, 1 H), 7.51-7.56 (m, 2 H), 7.67 (d, J = 1.2 Hz, |
| | 0.6 H), 7.67 (d, J = 1.4 Hz, 0.4 H). |
| 68 | ¹H NMR (500 MHz, DMSO-d₆) δ ppm 1.46 (ddt, J = 12.5, 8.1, 6.3, 6.3 Hz, 1 |
| | H), 1.80-1.90 (m, 1 H), 2.10 (s, 3 H), 2.20 (dd, J = 9.0, 6.6 Hz, 1 H), 2.40-2.46 |
| | (m, 1 H), 2.52-2.62 (m, 3 H), 2.72 (d, J = 7.8 Hz, 2 H), 3.62-3.75 (m, 2 |
| | H), 6.45-6.48 (m, 1 H), 7.22 (s, 1 H), 7.52 (t, J = 3.0 Hz, 1 H), 7.54-7.56 (m, 1 |
| | H), 8.15 (d, J = 1.7 Hz, 1 H), 11.12 (br s, 1 H), 11.91 (br s, 1 H). |
| 69 | ¹H NMR (500 MHz, CDCl₃) δ ppm 1.53-1.62 (m, 1 H), 1.95-2.07 (m, 1 H), |
| | 2.29 (s, 3 H), 2.30-2.39 (m, 1 H), 2.50-2.62 (m, 1 H), 2.62-2.75 (m, 3 H), |
| | 2.76-2.89 (m, 2 H), 3.71-3.83 (m, 5 H), 6.64 (d, J = 2.9 Hz, 1 H), 7.19 (s, 1 |
| | H), 7.22 (d, J = 3.2 Hz, 1 H), 7.41 (s, 1 H), 8.31 (d, J = 1.7 Hz, 1 H), 11.47 (br s, |
| | 1 H). |
| 70 | ¹H NMR (500 MHz, CDCl₃) δ ppm 1.64 (qd, J = 12.3, 4.3 Hz, 1 H), 1.75-1.91 |
| | (m, 2 H), 2.02 (br d, J = 12.7 Hz, 1 H), 2.10-2.20 (m, 2 H), 2.28 (s, 3 H), 3.05 |
| | (br d, J = 11.0 Hz, 1 H), 3.09 (br dt. J = 11.2, 1.3 Hz, 1 H), 3.63 (tt, J = 11.3, 3.2 |
| | Hz, 1 H), 3.66 (d, J = 14.4 Hz, 1 H), 3.78 (dd, J = 13.9, 0.9 Hz, 1 H), 3.99 (s, 3 |
| | H), 6.63 (d, J = 3.2 Hz, 1 H), 6.94 (d, J = 4.9 Hz, 1 H), 7.14 (d, J = 3.2 Hz, 1 H), |
| | 7.16 (s, 1 H), 8.32 (d, J = 4.9 Hz, 1 H), 11.34 (br s, 1 H). |
| 71 | ¹H NMR (500 MHz, CDCl₃) δ ppm 1.59-1.69 (m, 1 H) 1.76-1.92 (m, 2 H) |
| | 2.02 (br d, J = 13.0 Hz, 1 H) 2.09-2.20 (m, 2 H) 2.28 (s, 3 H) 3.05 (br d, J = 11.0 |
| | Hz, 1 H) 3.09 (br d, J = 11.3 Hz, 1 H) 3.57-3.66 (m, 1 H) 3.66 (d, J = 14.2 Hz, 1 |
| | H) 3.78 (d, J = 14.2 Hz, 1 H) 3.99 (s, 3 H) 6.63 (d, J = 3.5 Hz, 1 H) 6.95 (d, J = 4.9 |
| | Hz, 1 H) 7.14 (d, J = 3.2 Hz, 1 H) 7.16 (s, 1 H) 8.33 (d, J = 5.2 Hz, 1 H) 11.22 (br |
| | s, 1 H). |
| 72 | ¹H NMR (500 MHz, CDCl₃) δ ppm 1.59-1.69 (m, 1 H) 1.75-1.91 (m, 2 H) |
| | 2.02 (br d, J = 12.4 Hz, 1 H) 2.09-2.21 (m, 2 H) 2.28 (s, 3 H) 3.05 (br d, J = 11.3 |
| | Hz, 1 H) 3.09 (br d, J = 11.0 Hz, 1 H) 3.59-3.66 (m, 1 H) 3.66 (d, J = 14.2 Hz, 1 |
| | H) 3.78 (d, J = 14.4 Hz, 1 H) 3.99 (s, 3 H) 6.63 (d, J = 3.5 Hz, 1 H) 6.95 (d, J = 4.9 |
| | Hz, 1 H) 7.14 (d, J = 3.2 Hz, 1 H) 7.17 (s, 1 H) 8.33 (d, J = 4.9 Hz, 1 H) 11.22 (br |
| | s, 1 H) |
| 73 | ¹H NMR (400 MHz, CDCl₃) δ ppm 1.60-1.84 (m, 3 H) 2.01 (br d, J = 12.3 Hz, |
| | 1 H) 2.10 (td, J = 10.8, 3.7 Hz, 1 H) 2.26-2.36 (m, 4 H) 2.98 (br d, J = 10.9 Hz, |
| | 1 H) 3.03-3.11 (m, 1 H) 3.11-3.20 (m, 1 H) 3.74 (s, 2 H) 3.85 (s, 3 H) 6.37 |
| | (d, J = 3.5 Hz, 1 H) 6.92 (d, J = 8.1 Hz, 1 H) 7.10 (d, J = 3.5 Hz, 1 H) 7.19 (s, 1 H) |
| | 7.78 (d, J = 8.1 Hz, 1 H) 12.28 (br s, 1 H). |
| 74 | ¹H NMR (500 MHz, DMSO-d₆) δ ppm 1.49 (qd, J = 12.1, 3.8 Hz, 1 H), 1.65 (qt, |
| | J = 12.2, 3.6 Hz, 1 H), 1.71-1.77 (m, 1 H), 1.80 (br d, J = 12.1 Hz, 1 H), 2.00 (t, |
| | J = 10.8 Hz, 1 H), 2.05 (td, J = 11.3, 2.3 Hz, 1 H), 2.11 (s, 3 H), 2.50 (s, 3 H), |
| | 2.84-2.92 (m, 2 H), 2.96 (tt, J = 11.1, 3.2 Hz, 1 H), 3.62-3.74 (m, 2 H), 6.30 |
| | (dd, J = 3.5, 1.7 Hz, 1 H), 7.23 (s, 1 H), 7.28 (dd, J = 3.2, 2.6 Hz, 1 H), 7.75 (s, 1 |
| | H), 11.29 (br s, 1 H), 11.92 (br s, 1 H). |
| 75 | ¹H NMR (500 MHz, CDCl₃) δ ppm 1.46-1.57 (m, 1 H), 1.84 (br s, 2 H), 1.93 |
| | (br d, J = 12.1 Hz, 1 H), 2.07 (br t, J = 10.8 Hz, 1 H), 2.14-2.24 (m, 1 H), 2.29 |
| | (s, 3 H), 2.65 (s, 3 H), 2.99 (br d, J = 10.4 Hz, 1 H), 3.05 (br d, J = 10.4 Hz, 1 H), |
| | 3.15 (br t, J = 10.8 Hz, 1 H), 3.72 (d, J = 14.2 Hz, 1 H), 3.81 (d, J = 13.9 Hz, 1 H), |
| | 6.40 (d, J = 3.5 Hz, 1 H), 7.17-7.24 (m, 2 H), 7.77 (s, 1 H), 10.02 (br s, 1 H), |
| | 11.66 (br s, 1 H). |
| 76 | ¹H NMR (500 MHz, CDCl₃) δ ppm 1.51 (qd, J = 11.9, 5.3 Hz, 1 H), 1.75-1.88 |
| | (m, 2 H), 1.93 (br d, J = 11.8 Hz, 1 H), 2.05 (br t, J = 11.0 Hz, 1 H), 2.16 (td, |
| | J = 10.8, 4.0 Hz, 1 H), 2.29 (s, 3 H), 2.65 (s, 3 H), 2.97 (br d, J = 10.7 Hz, 1 H), |
| | 3.03 (br d, J = 10.7 Hz, 1 H), 3.11 (br t, J = 11.1 Hz, 1 H), 3.69 (d, J = 14.2 Hz, 1 |
| | H), 3.79 (d, J = 14.2 Hz, 1 H), 6.39 (d, J = 3.5 Hz, 1 H), 7.19 (s, 1 H), 7.22 (d, |
| | J = 3.5 Hz, 1 H), 7.78 (s, 1 H), 10.36 (br s, 1 H), 11.94 (br s, 1 H). |
| 77 | ¹H NMR (500 MHz, CDCl₃) δ ppm 1.56 (ddd, J = 24.1, 11.6, 4.2 Hz, 1 H) 1.67-1.85 |
| | (m, 2 H) 1.93-2.01 (m, 1 H) 2.13-2.25 (m, 2 H) 2.28 (s, 3 H) 2.94 (br d, |
| | J = 11.0 Hz, 1 H) 2.97-3.06 (m, 2 H) 3.68-3.76 (m, 2 H) 3.80 (s, 3 H) 6.63 |
| | (dd, J = 3.3, 0.7 Hz, 1 H) 7.17 (s, 1 H) 7.22 (d, J = 3.5 Hz, 1 H) 7.51 (br s, 1 H) |
| | 8.34 (d, J = 2.0 Hz, 1 H) 11.19 (br s, 1 H). |
| 78 | ¹H NMR (500 MHz, CDCl₃) δ ppm 1.51-1.61 (m, 1 H) 1.69-1.85 (m, 2 H) |
| | 1.93-2.03 (m, 1 H) 2.13-2.25 (m, 2 H) 2.28 (s, 3 H) 2.94 (br d, J = 11.0 Hz, 1 |
| | H) 2.97-3.05 (m, 2 H) 3.68-3.77 (m, 2 H) 3.80 (s, 3 H) 6.63 (dd, J = 3.2, 0.6 |
| | Hz, 1 H) 7.18 (s, 1 H) 7.22 (d, J = 3.2 Hz, 1 H) 7.51 (br s, 1 H) 8.34 (d, J = 1.7 |
| | Hz, 1 H) 11.44 (br s, 1 H). |
| 79 | ¹H NMR (500 MHz, CDCl₃) δ ppm 1.50-1.61 (m, 1 H), 1.69-1.85 (m, 2 H), |
| | 1.94-2.01 (m, 1 H), 2.14-2.25 (m, 2 H), 2.28 (s, 3 H), 2.94 (br d, J = 11.0 Hz, |
| | 1 H), 2.97-3.06 (m, 2 H), 3.68-3.76 (m, 2 H), 3.80 (s, 3 H), 6.63 (dd, J = 3.2, |
| | 0.6 Hz, 1 H), 7.17 (s, 1 H), 7.22 (d, J = 3.2 Hz, 1 H), 7.51 (br. s, 1 H), 8.34 (d, |
| | J = 1.7 Hz, 1 H), 11.42 (br s, 1 H). |
| 80 | ¹H NMR (500 MHz, CDCl₃) δ ppm 1.50-1.61 (m, 1 H), 1.65-1.85 (m, 2 H), |
| | 1.91-2.00 (m, 1 H), 2.14-2.26 (m, 2 H), 2.29 (s, 3 H), 2.85-3.02 (m, 3 H), |
| | 3.65-3.77 (m, 2 H), 6.67-6.72 (m, 1 H), 7.16 (s, 1 H), 7.40 (t, J = 3.0 Hz, 1 H), |
| | 7.66 (br s, 1 H), 8.35 (d, J = 1.7 Hz, 1 H), 8.70 (br s, 1 H), 11.68 (br s, 1 H). |
| 81 | ¹H NMR (500 MHz, CDCl₃) δ ppm 1.48-1.61 (m, 1 H), 1.66-1.85 (m, 2 H), |
| | 1.92-2.00 (m, 1 H), 2.13-2.26 (m, 2 H), 2.29 (s, 3 H), 2.86-3.02 (m, 3 H), |
| | 3.65-3.78 (m, 2H), 6.69 (br s, 1 H), 7.16 (s, 1 H), 7.40 (t, J = 2.6 Hz, 1 H), |
| | 7.66 (br s, 1 H), 8.35 (d, J = 1.7 Hz, 1 H), 8.72 (br s, 1 H), 11.74 (br s, 1 H). |
| 82 | ¹H NMR (500 MHz, CDCl₃) δ ppm 1.55-1.73 (m, 2 H) 1.73-1.87 (m, 1 H) |
| | 1.88-1.98 (m, 1 H) 2.32 (s, 3 H) 2.45 (br s, 1 H) 2.66-3.16 (m, 3 H) 3.27- |
| | 3.35 (m, 1 H) 3.73 (d, J = 13.9 Hz, 1 H) 3.83 (d, J = 13.9 Hz, 1 H) 6.72 (dd, |
| | J = 3.2. 2.0 Hz, 1 H) 6.86 (d, J = 4.9 Hz, 1 H) 7.25 (s, 1 H) 7.54-7.59 (m, 1 H) |
| | 8.36 (d, J = 4.9 Hz, 1 H) 11.57 (br s, 2 H). |
| 83 | ¹H NMR (500 MHz, CDCl₃) δ ppm 1.53-1.68 (m, 2 H) 1.77-1.86 (m, 1 H) |
| | 1.87-1.98 (m, 1 H) 2.32 (s, 3 H) 2.44 (br s, 1 H) 2.64-3.12 (m, 3 H) 3.28-3.34 |
| | (m, 1 H) 3.73 (d, J = 14.2 Hz, 1 H) 3.83 (d, J = 14.2 Hz, 1 H) 6.72 (dd, |
| | J = 2.9, 2.0 Hz, 1 H) 6.86 (d, J = 4.6 Hz, 1 H) 7.25 (s, 1 H) 7.56 (br s, 1 H) 8.36 |
| | (d, J = 4.6 Hz, 1 H) 11.28 (br s, 2 H). |
| 84 | ¹H NMR (400 MHz, CDCl₃) δ ppm 1.69 (q, J = 12.6 Hz, 1 H), 2.19 (t, J = 11.2 |
| | Hz, 1 H), 2.31 (t, J = 10.9 Hz, 1 H), 2.33 (s, 3 H), 2.46 (br d, J = 12.5 Hz, 1 H), |
| | 2.61-2.77 (m, 4 H), 3.24 (br dd, J = 11.0, 3.4 Hz, 1 H), 3.34-3.44 (m, 1 H), |
| | 3.74-3.92 (m, 3 H), 6.76 (s, 1 H), 7.24 (s, 1 H), 8.43 (s, 1 H), 12.03 (br s, 1 |
| | H). |

^{&}Mixture of diastereomers

### D. Pharmacological Examples

### 1) OGA—Biochemical Assay

The assay is based on the inhibition of the hydrolysis of fluorescein mono-ß-D-N-Acetyl-Glucosamine (FM-GlcNAc) (Mariappa et al. 2015, Biochem J 470:255) by the recombinant human Meningioma Expressed Antigen 5 (MGEA5), also referred to as O-GlcNAcase (OGA). The hydrolysis FM-GlcNAc (Marker Gene technologies, cat # M1485) results in the formation of ß-D-N-glucosamineacetate and fluorescein. The fluorescence of the latter can be measured at excitation wavelength 485 nm and emission wavelength 538 nm. An increase in enzyme activity results in an increase in fluorescence signal. Full length OGA enzyme was purchased at OriGene (cat # TP322411). The enzyme was stored in 25 mM Tris.HCl, pH 7.3, 100 mM glycine, 10% glycerol at −20° C. Thiamet G and GlcNAcStatin were tested as reference compounds (Yuzwa et al. 2008 Nature Chemical Biology 4:483; Yuzwa et al. 2012 Nature Chemical Biology 8:393). The assay was performed in 200 mM Citrate/phosphate buffer supplemented with 0.005% Tween-20. 35.6 g Na₂HPO₄ 2 H₂O (Sigma, # C0759) were dissolved in 1 L water to obtain a 200 mM solution. 19.2 g citric acid (Merck, #1.06580) was dissolved in 1 L water to obtain a 100 mM solution. pH of the sodiumphosphate solution was adjusted with the citric acid solution to 7.2. The buffer to stop the reaction consists of a 500 mM Carbonate buffer, pH 11.0. 734 mg FM-GlcNAc were dissolved in 5.48 mL DMSO to obtain a 250 mM solution and was stored at −20° C. OGA was used at a 10 nM (protocol A) or 2 nM (protocol B) concentration and FM-GlcNAc at a 100 uM final concentration. Dilutions were prepared in assay buffer.

50 nl of a compound dissolved in DMSO was dispensed on Black Proxiplate™ 384 Plus Assay plates (Perkin Elmer, #6008269) and 3 μl fl-OGA enzyme mix added subsequently. Plates were pre-incubated for 60 min at room temperature and then 2 μl FM-GlcNAc substrate mix added. Final DMSO concentrations did not exceed 1%. Plates were briefly centrifuged for 1 min at 1000 rpm and incubate at room temperature for 1 h (10 nM OGA, protocol A) or 6 h (2 nM OGA, protocol B). To stop the reaction 5 μl STOP buffer were added and plates centrifuge again 1 min at 1000 rpm. Fluorescence was quantified in the Thermo Scientific Fluoroskan Ascent or the PerkinElmer EnVision with excitation wavelength 485 nm and emission wavelength 538 nm.

For analysis a best-fit curve is fitted by a minimum sum of squares method. From this an IC₅₀ value and Hill coefficient was obtained. High control (no inhibitor) and low control (saturating concentrations of standard inhibitor) were used to define the minimum and maximum values.

### 2) OGA—Cellular Assay

HEK293 cells inducible for P301L mutant human Tau (isoform 2N4R) were established at Janssen. Thiamet-G was used for both plate validation (high control) and as reference compound (reference EC₅₀ assay validation). OGA inhibition is evaluated through the immunocytochemical (ICC) detection of O-GlcNAcylated proteins by the use of a monoclonal antibody (CTD110.6; Cell Signaling, #9875) detecting O-GlcNAcylated residues as previoulsy described (Dorfmueller et al. 2010 Chemistry & biology, 17:1250). Inhibition of OGA will result in an increase of O-GlcNAcylated protein levels resulting in an increased signal in the experiment. Cell nuclei are stained with Hoechst to give a cell culture quality control and a rough estimate of immediate compounds toxicity, if any. ICC pictures are imaged with a Perkin Elmer Opera Phenix plate microscope and quantified with the provided software Perkin Elmer Harmony 4.1.

Cells were propagated in DMEM high Glucose (Sigma, # D5796) following standard procedures. 2 days before the cell assay cells are split, counted and seeded in Poly-D-Lysine (PDL) coated 96-wells (Greiner, #655946) plate at a cell density of 12,000 cells per cm² (4,000 cells per well) in 100 μl of Assay Medium (Low Glucose medium is used to reduce basal levels of GlcNAcylation) (Park et al. 2014 The Journal of biological chemistry 289:13519). At the day of compound test medium from assay plates was removed and replenished with 90 μl of fresh Assay Medium. 10 μl of compounds at a 10 fold final concentration were added to the wells. Plates were centrifuged shortly before incubation in the cell incubator for 6 hours. DMSO concentration was set to 0.2%. Medium is discarded by applying vacuum. For staining of cells medium was removed and cells washed once with 100 μl D-PBS (Sigma, # D8537). From next step onwards unless other stated assay volume was always 50 μl and incubation was performed without agitation and at room temperature. Cells were fixed in 50 μl of a 4% paraformaldehyde (PFA, Alpha aesar, #043368) PBS solution for 15 minutes at room temperature. The PFA PBS solution was then discarded and cells washed once in 10 mM Tris Buffer (LifeTechnologies, #15567-027), 150 mM NaCl (LifeTechnologies, #24740-0110, 0.1% Triton X (Alpha aesar, # A16046), pH 7.5 (ICC buffer) before being permeabilized in same buffer for 10 minutes. Samples are subsequently blocked in ICC containing 5% goat serum (Sigma, # G9023) for 45-60 minutes at room temperature. Samples were then incubated with primary antibody (1/1000 from commercial provider, see above) at 4° C. overnight and subsequently washed 3 times for 5 minutes in ICC buffer. Samples were incubated with secondary fluorescent antibody (1/500 dilution, Lifetechnologies, # A-21042) and nuclei stained with Hoechst 33342 at a final concentration of 1 μg/ml in ICC (Lifetechnologies, # H3570) for 1 hour. Before analysis samples were washed 2 times manually for 5 minutes in ICC base buffer.

Imaging is performed using Perkin Elmer Phenix Opera using a water 20× objective and recording 9 fields per well. Intensity readout at 488 nm is used as a measure of O-GlcNAcylation level of total proteins in wells. To assess potential toxicity of compounds nuclei were counted using the Hoechst staining. IC₅₀-values are calculated using parametric non-linear regression model fitting. As a maximum inhibition Thiamet G at a 200 uM concentration is present on each plate. In addition, a concentration response of Thiamet G is calculated on each plate.

TABLE 23
Results in the biochemical and cellular assays.

| | | Enzymatic | | Cellular | |
|---|---|---|---|---|---|
| Co. | Enzymatic | hOGA; | Enzymatic | hOGA; | Cellular |
| No. | protocol | pIC₅₀ | Eₘₐₓ (%) | pEC₅₀ | Eₘₐₓ (%) |
| 1 | A | 6.06 | 94.20 | | |
| 2 | A | 6.73 | 98.36 | | |
| 3 | A | 7.49 | 95.98 | | |
| 4 | A | 7.51 | 106.82 | | |
| 5 | A | 7.17 | 99.41 | | |
| 6 | A | 8.03 | 101.27 | <5 | 39.37 |
| 7 | A | 5.56 | 82.22 | | |
| 8 | A | 5.81 | 89.04 | | |
| 9 | A | 7.93 | 100.73 | 7.41 | 108.71 |
| 10 | B | 7.45 | 100.01 | 6.68 | 98.99 |
| 11 | B | 8.21 | 100.41 | 7.56 | 112.67 |
| 12 | B | 7.97 | 104.74 | 7.68 | 103.05 |
| 13 | A | 8.07 | 101.31 | 7.45 | 129.67 |
| 14 | A | 8.06 | 102.07 | 7.15 | 107.83 |
| 15 | B | 7.43 | 97.77 | 7.91 | 95.17 |
| 16 | A | 5.74 | 92.55 | | |
| | B | 6.02 | 94.53 | | |
| 17 | B | 8.07 | 101.02 | 7.26 | 106.27 |
| 18 | B | 6.53 | 97.33 | | |
| 19 | B | 6.69 | 97.25 | | |
| 20 | B | 6.22 | 93.66 | | |
| 21 | B | 6.39 | 98.31 | | |
| 22 | A | 6.21 | 99.07 | | |
| 23 | A | 6.53 | 101.78 | | |
| 24 | A | 7.50 | 102.06 | 6.32 | 108.83 |
| 25 | A | 7.87 | 102.90 | 6.34 | 112.94 |
| 26 | B | 7.39 | 123.00 | 7.62 | 99.02 |
| 27 | A | 7.77 | 100.44 | <5 | 19.50 |
| 28 | A | 8.04 | 103.17 | <5 | 21.98 |
| | B | 8.07 | 101.01 | | |
| 29 | A | 8.20 | 106.42 | <5 | 23.67 |
| 30 | A | 6.78 | 99.24 | <5 | 8.12 |
| 31 | A | 7.80 | 102.71 | <5 | 11.08 |
| 32 | A | 7.84 | 101.80 | <5 | 21.77 |
| 33 | A | 8.19 | 101.43 | <5 | 15.69 |
| 34 | A | 5.80 | 97.41 | | |
| 35 | A | 5.80 | 98.35 | | |
| | B | 5.83 | 86.67 | | |
| 36 | B | 6.13 | 93.57 | | |
| 37 | B | 5.57 | 79.62 | | |
| 38 | A | 6.33 | 100.96 | | |
| 39 | A | 5.46 | 88.42 | | |
| 40 | A | 7.78 | 99.98 | | |
| 41 | A | 8.21 | 102.35 | <5 | 10.50 |
| 42 | B | 6.36 | 93.64 | | |
| 43 | A | 5.73 | 93.08 | | |
| 44 | B | 8.42 | 101.01 | 7.45 | 129.49 |
| 45 | B | 8.33 | 100.39 | 7.87 | 96.86 |
| 46 | B | 8.63 | 99.85 | 7.83 | 114.40 |
| 47 | B | 8.90 | 102.13 | 7.81 | 108.52 |
| 48 | B | 8.76 | 103.32 | 7.77 | 104.21 |
| 49 | B | 8.76 | 100.76 | 7.59 | 105.38 |
| 50 | B | 8.03 | 99.56 | 7.47 | 103.00 |
| 51 | B | 6.94 | 98.46 | | |
| 52 | B | 7.73 | 101.15 | 5.58 | 130.74 |
| 53 | B | 5.19 | 64.63 | | |
| 54 | B | <5 | 25.13 | | |
| 55 | B | 5.02 | 51.79 | <5 | 1.01 |
| 56 | B | <5 | 25.81 | | |
| 57 | B | 6.21 | 92.95 | | |
| 58 | B | 6.61 | 97.32 | <5 | 4.10 |
| 59 | B | <5 | −1.34 | | |
| 60 | A | 5.73 | 98.67 | | |
| 61 | B | <5 | 27.39 | | |
| 62 | B | <5 | 16.19 | | |
| 63 | A | 6.16 | 93.75 | | |
| 64 | A | 7.16 | 100.74 | 5.32 | 84.48 |
| 65 | B | 6.91 | 99.20 | | |
| 66 | B | 7.53 | 100.83 | 7.74 | 97.30 |
| 67 | B | 6.99 | 95.67 | | |
| 68 | B | 8.27 | 100.88 | 7.38 | 91.19 |
| 69 | B | 8.52 | 99.60 | 7.63 | 108.21 |
| 71 | B | 7.90 | 100.16 | 7.06 | 79.25 |
| 72 | B | 6.09 | 93.19 | | |
| 73 | B | 7.09 | 100.65 | 6.71 | 87.16 |
| 74 | A | 8.2 | 100.66 | 7.87 | 102.19 |
| 75 | B | 8.57 | 100.81 | 7.90 | 119.60 |
| 76 | B | 6.06 | 96.53 | | |
| 78 | B | 5.93 | 90.73 | | |
| 79 | B | 8.14 | 102.15 | 7.87 | 101.35 |
| 80 | B | 7.69 | 99.58 | 7.72 | 109.88 |
| 81 | B | 6.06 | 97.25 | | |
| 82 | B | 7.66 | 101.11 | | |
| 83 | B | 5.43 | 76.38 | | |
| 84 | B | 5.83 | 88.48 | | |
| 86 | B | 8.38 | 102.93 | 7.18 | 100.91 |
| 87 | B | 5.87 | 94.20 | | |
| 88 | B | 7.08 | 101.41 | 6.39 | 57.43 |
| 89 | B | 7.37 | 102.02 | | |
| 90 | B | 5.42 | 74.60 | | |
| 91 | B | 7.02 | 102.80 | 7.35 | 84.31 |
| 92 | B | 5.63 | 88.90 | | |
| 93 | B | 7.35 | 101.08 | 7.61 | 87.83 |
| 95 | B | 8.19 | 101.27 | 8.59 | 99.79 |
| 96 | B | 6.15 | 99.16 | 6.40 | 70.38 |
| 97 | B | 8.76 | 101.95 | 8.11 | 108.93 |
| 98 | B | 7.42 | 101.69 | 6.12 | 49.85 |
| 99 | B | 8.96 | 101.31 | 8.17 | 107.53 |
| 100 | B | 6.51 | 98.45 | 6.71 | 85.74 |
| 101 | B | 8.18 | 102.01 | 8.03 | 114.06 |
| 102 | B | 5.76 | 88.86 | | |
| 103 | B | 8.53 | 100.60 | 8.24 | 102.71 |
| 104 | B | 8.83 | 100.03 | 7.21 | 91.48 |
| 105 | B | 8.13 | 101.55 | | |
| 106 | B | 6.56 | 101.32 | 6.55 | 71.02 |
| 107 | B | 8.09 | 100.93 | 7.26 | 81.95 |
| 108 | B | 8.31 | 101.19 | 7.01 | 92.67 |
| 109 | A | 6.36 | 95.41 | | |

n.t. means not tested.

1. A compound of Formula (I) 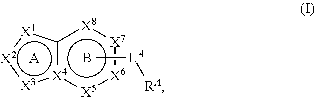
or a tautomer or a stereoisomeric form thereof, wherein
A-B represent a 9-membered bicyclic heteroaryl system having from 1 to 4 nitrogen atoms, wherein
X¹ and X³ are each independently selected from the group consisting of CR^{XA}, N, and NR^{YA};
X² is CH;
X⁴ is C or N; and
X⁵, X⁶, X⁷, and X⁸ are each independently selected from the group consisting of C, CR^{XB} and N;
with the proviso that at least one of X¹ and X³ is N or NR^{YA};
wherein each R^{XA}, and R^{XB}, when present, is independently selected from the group consisting of hydrogen; halo; —CN; C₁₋₄alkyl optionally substituted with 1, 2 or 3 independently selected halo substituents; and C₁₋₄alkyloxy optionally substituted with 1, 2 or 3 independently selected halo substituents;
each R^{YA}, when present, is independently selected from the group consisting of hydrogen and C₁₋₄alkyl optionally substituted with 1, 2 or 3 independently selected halo substituents;
L^{A} is bound to any available carbon atom at the 6-membered B ring of the A-B bicycle, and is selected from the group consisting of a bond, CHR¹, O, and NR¹; wherein
R¹ is selected from the group consisting of hydrogen and C₁₋₄alkyl optionally substituted with 1, 2 or 3 independently selected halo substituents;
R^{A} is a radical (a-1) when L^{A} is a bond, CHR¹, O, or NR¹; or is a radical selected from the group consisting of (a-2) and (a-3) when L^{A} is a bond or CHR¹ 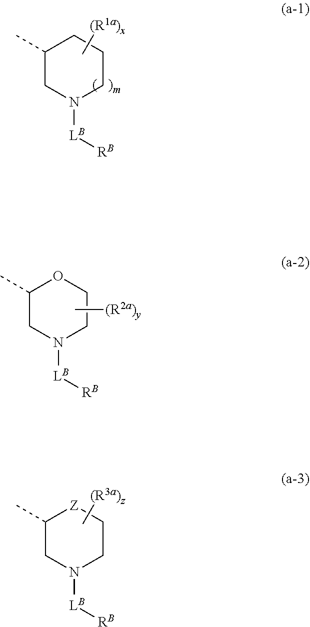
wherein
m represents 0, 1 or 2;
x, y and z, each independently represent 0, 1 or 2;
each R^{1a} and R^{2a} when present, is bound to any available carbon atom and is independently selected from the group consisting of halo and C₁₋₄alkyl optionally substituted with 1, 2, or 3 independently selected halo substituents; or two R^{1a} or two R^{2a} substituents are bound to the same carbon atom and form together a cyclopropylidene radical;
Z is N when substituted with R^{3a}, or NH;
each R^{3a} is bound to any available carbon atom or nitrogen atom when present, and is independently selected from C₁₋₃alkyl optionally substituted with 1, 2 or 3 independently selected halo substituents; or two R^{3a} substituents are bound to the same carbon atom and form together a cyclopropylidene radical;
L^{B} is selected from the group consisting of >CHR² and >SO₂;
wherein R² is selected from the group consisting of hydrogen, and C₁₋₄alkyl optionally substituted with 1, 2 or 3 independently selected halo substituents; and
R^{B} represents a heterocyclic ring or ring system selected from the group consisting of (b-1), (b-2), (b-3), (b-4), (b-5), (b-6), (b-7), (b-8), (b-9), (b-10), (b-11) and (b-12) 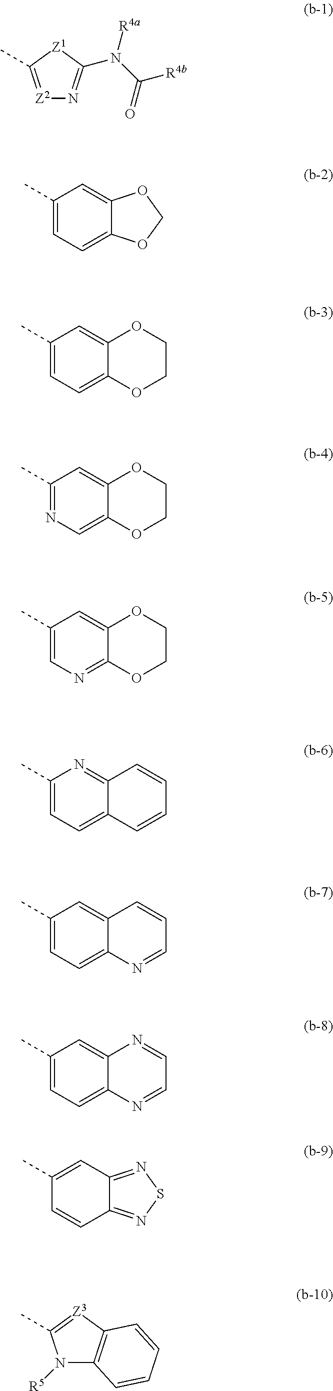 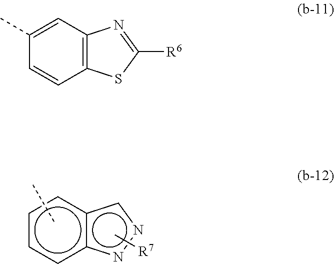
wherein
Z¹ is O, NR^{1z} or S; wherein R^{1z} is hydrogen or C₁₋₄alkyl;
Z² and Z³ each independently represent CH or N;
R^{4b} is C₁₋₄alkyl;
R^{4a}, R⁵, R⁶ and R⁷ each independently represent hydrogen or C₁₋₄alkyl; or
L^{B}-R^{B} is a radical of formula (b-13) 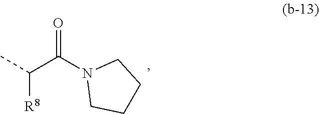
wherein R⁸ is hydrogen or C₁₋₄alkyl;
or a pharmaceutically acceptable salt or a solvate thereof.

2. The compound according to claim 1, wherein R^{B} is a radical selected from the group consisting of (b-1), (b-2), (b-3) and (b-8); or -L^{B}-R^{B} is a radical of formula (b-13).

3. The compound according to claim 1, wherein
X¹ is selected from the group consisting of CH, N, and NR^{YA}; wherein R^{YA}, when present, is hydrogen or C₁₋₄alkyl;
X² is CH;
X³ is CH or N;
X⁴ is C or N;
X⁵ is C, CR^{XB} or N; wherein R^{XB}, when present, is hydrogen or C₁₋₄alkyl optionally substituted with 1, 2 or 3 independently selected halo substituents;
X⁶ is C, CH, or C(halo);
X⁷ is C, CR^{XB} or N; wherein R^{XB}, when present, is hydrogen or C₁₋₄alkyl optionally substituted with 1, 2 or 3 independently selected halo substituents; and
X⁸ is C, CH or N;
with the proviso that at least one of X¹ and X³ is N or NR^{YA};
L^{A} is bound to any available carbon atom at the 6-membered B ring of the A-B bicycle, and is selected from the group consisting of a bond, CHR¹, and NR¹; wherein
R¹ is hydrogen or C₁₋₄alkyl;
R^{A} is a radical (a-1) when L^{A} is a bond, CHR¹, NR¹; or is a radical selected from the group consisting of (a-2) and (a-3) when L^{A} is a bond or CHR¹ 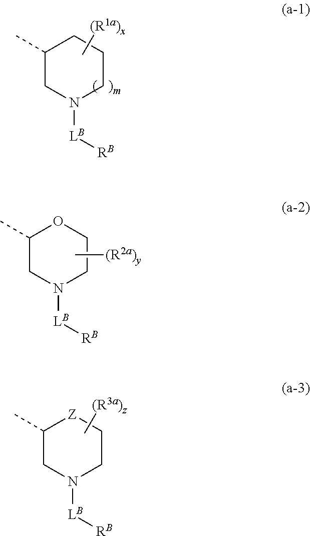
wherein
m represents 0 or 1;
x is 0, 1 or 2;
y and z, each independently represent 0;
each R^{1a} when present, is bound to any available carbon atom and is C₁₋₄alkyl optionally substituted with 1, 2, or 3 independently selected halo substituents; or two R^{1a} substituents are bound to the same carbon atom and form together a cyclopropylidene radical;
Z is NH;
L^{B} is selected from the group consisting of >CHR² and >SO₂; wherein R² is hydrogen or C₁₋₄alkyl; and
R^{B} is a radical selected from the group consisting of (b-1), (b-2), (b-3) and (b-8) 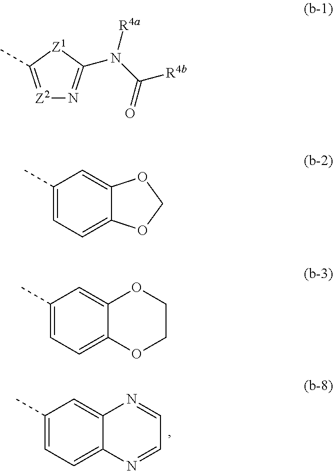
wherein
Z¹ is S;
Z² is CH;
R^{4a} is H or CH₃;
R^{4b} is C₁₋₄alkyl; or
L^{B}-R^{B} is a radical of formula (b-13), wherein R⁸ is hydrogen or C₁₋₄alkyl.

4. The compound according to claim 1, wherein L^{B} is CH₂.

5. The compound according to claim 1, wherein L^{A} is a bond, CH₂ or NH; and R^{A} is (a-1).

6. The compound according to claim 1, wherein R^{A} is (a-1); m is 0 or 1; x is 0, 1 or 2; and R^{1a} is methyl or two R^{1a} substituents are bound to the same carbon atom and form together a cyclopropylidene radical.

7. The compound according to claim 1, wherein L^{A} is a bond or CH₂.

8. The compound according to of claim 1 having the Formula (I′)

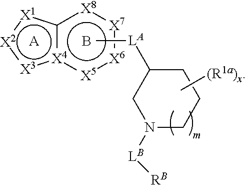

9. A compound according to claim 1, wherein the compound is 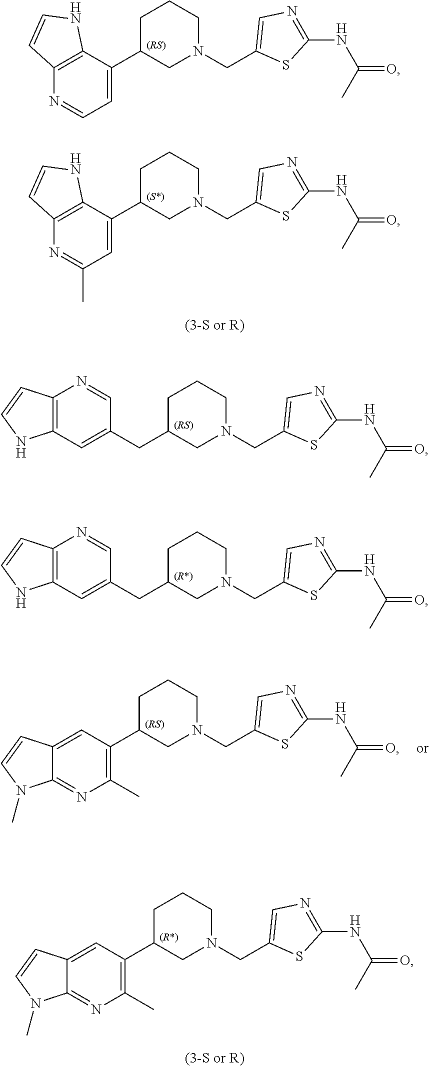 [SMILES: CC(=O)Nc1ncc(CN2CCCC(c3ccnc4cc[nH]c34)C2)s1 ; CC(=O)Nc1ncc(CN2CCCC(c3cc(C)nc4cc[nH]c34)C2)s1 ; CC(=O)Nc1ncc(CN2CCCC(Cc3cnc4cc[nH]c4c3)C2)s1 ; CC(=O)Nc1ncc(CN2CCCC(Cc3cnc4cc[nH]c4c3)C2)s1 ; CC(=O)Nc1ncc(CN2CCCC(c3cc4ccn(C)c4nc3C)C2)s1 ; CC(=O)Nc1ncc(CN2CCCC(c3cc4ccn(C)c4nc3C)C2)s1]
or a pharmaceutically acceptable salt or a solvate thereof.

10. A pharmaceutical composition comprising a prophylactically or a therapeutically effective amount of a compound according to claim 1 and a pharmaceutically acceptable carrier.

11. A process for preparing a pharmaceutical composition comprising mixing a pharmaceutically acceptable carrier with a prophylactically or a therapeutically effective amount of a compound according to claim 1.

12. (canceled)

13. (canceled)

14. A method of preventing or treating a disorder selected from the group consisting of tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, comprising administering to a subject in need thereof, a prophylactically or a therapeutically effective amount of a compound according to claim 1.

15. A method for inhibiting O-GlcNAc hydrolase, comprising administering to a subject in need thereof, a prophylactically or a therapeutically effective amount of a compound according to claim 1.
